(19) United States
(12) Patent Application Publication
(10) Pub. No.: US 2025/0064844 A1
(43) Pub. Date: Feb. 27, 2025

(54) N4-HYDROXYCYTIDINE AND DERIVATIVES AND ANTI-VIRAL USES RELATED THERETO

(71) Applicant: Emory University, Atlanta, GA (US)

(72) Inventors: George R. PAINTER, Atlanta, GA (US); Gregory R. BLUEMLING, Decatur, GA (US); Michael G. NATCHUS, Alpharetta, GA (US); Shuli MAO, Marietta, GA (US); Jose MARENGO, Atlanta, GA (US); Michael W. HAGER, Kennesaw, GA (US); David PERRYMAN, Decatur, GA (US)

(21) Appl. No.: 18/940,253

(22) Filed: Nov. 7, 2024

Related U.S. Application Data
(63) Continuation of application No. 17/170,172, filed on Feb. 8, 2021.
(60) Provisional application No. 63/006,625, filed on Apr. 7, 2020.
(60) Provisional application No. 62/994,604, filed on Mar. 25, 2020.
(60) Provisional application No. 62/988,133, filed on Mar. 11, 2020.
(60) Provisional application No. 62/971,559, filed on Feb. 7, 2020.

(51) Int. Cl. A61K 31/7068; A61K 9/00; A61K 31/34; A61K 31/341; A61K 31/403; A61K 31/404; A61K 31/4178; A61K 31/4406; A61K 31/496; A61K 31/4965; A61K 31/513; A61K 31/52; A61K 31/522; A61K 31/536; A61K 31/665; A61K 31/675; A61K 31/7076; A61K 45/06; A61M 11/00; A61P 31/14; C07H 19/06; C07H 19/10

(52) U.S. Cl. CPC A61K 31/7068; A61K 9/0075; A61K 9/0078; A61K 9/008; A61K 31/34; A61K 31/341; A61K 31/403; A61K 31/404; A61K 31/4178; A61K 31/4406; A61K 31/496; A61K 31/4965; A61K 31/513; A61K 31/52; A61K 31/522; A61K 31/536; A61K 31/665; A61K 31/675; A61K 31/7076; A61K 45/06; A61M 11/005; A61M 11/007; A61P 31/14; C07H 19/06; C07H 19/10

(57) ABSTRACT

This disclosure relates to certain N4-hydroxycytidine derivatives, pharmaceutical compositions, and methods related thereto. In certain embodiments, the disclosure relates to the treatment or prophylaxis of human coronavirus 2019-nCoV.

### CROSS REFERENCE TO RELATED APPLICATIONS

This application claims the benefit of priority to U.S. Provisional Application Nos. 62/971,559, filed Feb. 7, 2020, 62/988,133, filed Mar. 11, 2020, 62/994,604, filed Mar. 25, 2020, and 63/006,625, filed Apr. 7, 2020, each of which are incorporated by reference herein in their entireties.

### ACKNOWLEDGEMENT OF GOVERNMENT SUPPORT

This invention was made with government support under grant nos. HDTRA1-13-C-0072 and HDTRA1-15-C-0075 awarded by the Department of Defense and grant nos. HHSN272201500008C and 75N93019C00058 awarded by National Institutes of Health. The government has certain rights in the invention.

### FIELD

This disclosure relates to N4-hydroxycytidine nucleoside and derivatives, as well as compositions and methods related thereto. In certain embodiments, the disclosure relates to the treatment or prophylaxis of viral infections, in particular, 2019-nCoV.

### BACKGROUND

Coronaviruses are enveloped positive-sense RNA viruses that cause a large percentage of respiratory illness in humans. The two previous coronaviruses to emerge and cause human illness were SARS and MERS. There were more than 8,000 human cases of SARS with 774 deaths. Since 2012, there have been more than 2,500 cases of MERS with 919 deaths. In 2019, a new coronavirus, 2019-nCoV and now known as SARS-CoV-2, was discovered in humans in Wuhan, China. Reports from early February 2020 indicate more than 28,000 people have been infected with the novel coronavirus, with more than 560 deaths documented. In addition, human-to-human transmission of 2019-nCov has been documented. Analysis of a single completed full-genome sequence revealed 2019-nCov belongs to betacoronavirus but is divergent from SARS and MERS. The 2019-nCoV is a highly pathogenic human pathogen that relatively little is known about. SARS-CoV-2/2019-nCoV, causes disease referred to as COVID-19. COVID-19 can include severe respiratory disease in humans and appears to also cause neurological disease that includes dizziness, impaired consciousness, acute cerebrovascular disease, epilepsy, hyposmia, hypopsia, and neuralgia (medRxiv, 2020, 1-26). SARS-CoV-2 entry into the CNS may be promoted through viral interaction with ACE2 receptors after dissemination of the virus in the systemic circulation or across the cribriform plate. Additional studies are needed to further characterize the virus and to identify ways to prevent and treat disease.

Stuyver et al. report β-D-N(4)-hydroxycytidine (NHC) was found to have antipestivirus and antihepacivirus activities. Antimicrob Agents Chemother, 2003, 47(1):244-54. Constantini et al. report evaluations on the efficacy of 2′-C-MeC, 2′-F-2′-C-MeC, and NHC on Norwalk virus. See also Purohit et al., J Med Chem, 2012, 55(22):9988-9997; Ivanov et al., Collection of Czechoslovak Chem Commun, 2006, 71(7):1099-1106; and Fox et al., JACS, 1959, 81:178-87.

What are needed are new compounds and treatments for viral infections. The compounds and methods disclosed herein addressed these needs.

### SUMMARY

This disclosure relates to certain N4-hydroxycytidine and derivatives, combinations, pharmaceutical compositions, and methods related thereto. In certain embodiments, the disclosure relates to a compound having Formula I,

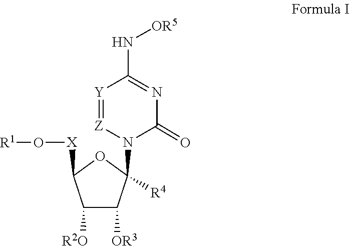

or a pharmaceutically acceptable salt, derivative, or prodrug thereof, as defined herein.

In certain embodiments, the disclosure contemplates derivatives of compounds disclosed herein, such as those containing one or more, the same or different, substituents.

In certain embodiments, the disclosure contemplates pharmaceutical compositions comprising a pharmaceutically acceptable excipient and a compound disclosed herein. In certain embodiments, the pharmaceutical composition is in the form of a tablet, capsule, pill, or aqueous buffer, such as a saline or phosphate buffer.

In certain embodiments, the disclosed pharmaceutical compositions can comprise a compound disclosed herein and a propellant. In certain embodiments, the propellant is an aerosolizing propellant such as compressed air, ethanol, nitrogen, carbon dioxide, nitrous oxide, hydrofluoroalkanes (HFAs), 1,1,1,2,-tetrafluoroethane, 1,1,1,2,3,3,3-heptafluoropropane, or combinations thereof.

In certain embodiments, the disclosure contemplates a pressurized or unpressurized container comprising a compound or pharmaceutical composition as described herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, the disclosure relates to methods of increasing bioavailability for treating or preventing a viral infection comprising administering an effective amount of a compound or pharmaceutical composition disclosed herein to a subject in need thereof.

In certain embodiments, the viral infection is a human coronavirus, SARS, MERS, or 2019-nCoV infection.

In certain embodiments, the compound or pharmaceutical composition is administered orally, intravenously, or through the lungs, i.e., pulmonary administration.

In certain embodiments, the disclosure relates to the use of a compound as described herein in the production of a medicament for the treatment or prevention of a viral infection, such as 2019-nCoV virus infection.

In certain embodiments, the disclosure relates to method of making compounds disclosed herein by mixing starting materials and reagents disclosed herein under conditions such that the compounds are formed. The compounds made by the methods disclosed can be used to treat or prevent COVID-19 caused by 2019-nCoV/SARS-CoV-2 as disclosed herein.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 is a scheme illustrating the preparation of β-D-N-hydroxycytidine. The steps of the synthesis are a.) tert-butyldimethylsilyl chloride, 4-dimethylaminopyridine, diisopropylethylamine, dichloromethane; b.) (2,4,6-iPr)PhSO₂Cl, diisopropylethylamine, 4-dimethylaminopyridine, dichloromethane; c.) NH₂OH—HCl, diisopropylethylamine, dichloromethane; d.) F— source; and e.) aq NH₂OH, AcOH, 50° C.

FIG. 2 illustrates certain exemplary compounds.

FIG. 3 illustrates certain exemplary compounds.

FIG. 4 shows mean plasma concentrations and pharmacokinetic parameters from mice treated with an exemplary compound.

FIG. 5 shows nucleoside accumulation in mouse organs in mice treated with an exemplary compound.

FIG. 6 shows triphosphate accumulation in mouse organs in mice treated with an exemplary compound.

FIG. 7 shows the N4-hydroxycytidine nucleoside tissue concentrations from a cynomolgus macaque orally administered EIDD-1931 (100 mg/kg).

FIG. 8 shows the N4-hydroxycytidine nucleoside tissue concentrations from a cynomolgus macaque intravenously administered EIDD-1931 (10 mg/kg).

FIG. 9 shows the structure of compounds orally administered to cynomolgus macaques.

FIG. 10 shows the mean N4-hydroxycytidine nucleoside plasma concentrations from cynomolgus macaques orally administered with an ester derivative.

FIG. 11 shows the mean maximum concentration of N4-hydroxycytidine nucleoside in plasma from cynomolgus macaques orally administered with an ester derivative.

FIG. 12 shows the effect of EIDD-2801 prophylactic treatment on lung viral titers of SARS infected mice.

FIG. 13 shows the effect of EIDD-2801 time of treatment on lung hemorrhage scores of SARS infected mice.

FIG. 14 shows the effect of EIDD-2801 time of treatment on lung viral titers of SARS infected mice.

FIG. 15 shows the effect of EIDD-2801 treatment on lung hemorrhage scores of MERS infected mice.

FIG. 16 shows the arithmetic mean plasma concentrations of EIDD-1931 (50-1600 mg EIDD-2801 single ascending doses).

FIG. 17 shows the arithmetic mean plasma concentrations of EIDD-1931 (50-800 mg EIDD-2801 twice-daily multiple ascending doses) on Day 1 (top) and Day 6 (bottom).

FIG. 18 shows the arithmetic mean plasma concentration of EIDD-1931 (food effect).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features, which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

In certain embodiments, a pharmaceutical agent, which may be in the form of a salt or prodrug, is administered in methods disclosed herein that is specified by a weight. This refers to the weight of the recited compound. If in the form of a salt or prodrug, then the weight is the molar equivalent of the corresponding salt or prodrug.

It must be noted that, as used in the specification and the appended claims, the singular forms “a,” “an,” and “the” include plural referents unless the context clearly dictates otherwise.

“Subject” refers any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent can be administered prior to, together with, or after the additional treatment, or a combination thereof.

As used herein, “alkyl” means a straight or branched chain saturated hydrocarbon moieties such as those containing from 1 to 10 carbon atoms. A “higher alkyl” refers to saturated hydrocarbon having 11 or more carbon atoms. A “C₆-C₁₆” refers to an alkyl containing 6 to 16 carbon atoms. Likewise, a “C₆-C₂₂” refers to an alkyl containing 6 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like.

As used herein, the term “alkenyl” refers to unsaturated, straight or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C.

As used herein, the term “alkynyl” represents straight or branched hydrocarbon moieties containing a triple bond. Unless otherwise specified, C₂-C₂₄ (e.g., C₂-C₂₄, C₂-C₂₀, C₂-C₁₈, C₂-C₁6, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur, which can be saturated or unsaturated (but not aromatic), monocyclic or polycyclic, and wherein the nitrogen and sulfur heteroatoms can be optionally oxidized, and the nitrogen heteroatom can be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl, and biphenyl. Phenyl is a preferred aryl group. The term “substituted aryl” refers to aryl groups substituted with one or more groups, preferably selected from alkyl, substituted alkyl, alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, lactam, urea, urethane, sulfonyl, and the like, where optionally one or more pair of substituents together with the atoms to which they are bonded form a 3 to 7 member ring.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems can, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems can be aromatic, non-aromatic or mixtures of aromatic and non-aromatic rings. Heterocycle includes heterocarbocycles, heteroaryls, and the like.

“Alkylthio” refers to an alkyl group as defined above with the indicated number of carbon atoms attached through a sulfur bridge. An example of an alkylthio is methylthio, (i.e., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH₃).

“Alkanoyl” refers to an alkyl as defined above with the indicated number of carbon atoms attached through a carbonyl bride (i.e., —(C═O)alkyl).

“Alkylsulfonyl” refers to an alkyl as defined above with the indicated number of carbon atoms attached through a sulfonyl bridge (i.e., —S(═O)₂alkyl) such as mesyl and the like, and “arylsulfonyl” refers to an aryl attached through a sulfonyl bridge (i.e., —S(═O)₂aryl).

“Alkylsulfamoyl” refers to an alkyl as defined above with the indicated number of carbon atoms attached through a sulfamoyl bridge (i.e., —NHS(═O)₂alkyl), and an “arylsulfamoyl” refers to an alkyl attached through a sulfamoyl bridge (i.e., —NHS(═O)₂aryl).

“Alkylsulfinyl” refers to an alkyl as defined above with the indicated number of carbon atoms attached through a sulfinyl bridge (i.e. —S(═O)alkyl).

The terms “cycloalkyl” and “cycloalkenyl” refer to mono-, bi-, or tri homocyclic ring groups of 3 to 15 carbon atoms which are, respectively, fully saturated and partially unsaturated. The term “cycloalkenyl” includes bi- and tricyclic ring systems that are not aromatic as a whole but contain aromatic portions (e.g., fluorene, tetrahydronapthalene, dihydroindene, and the like). The rings of multi-ring cycloalkyl groups can be either fused, bridged, and/or joined through one or more spiro unions. The terms “substituted cycloalkyl” and “substituted cycloalkenyl” refer, respectively, to cycloalkyl and cycloalkenyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), cyano, nitro, amino, substituted amino, amido, lactam, urea, urethane, sulfonyl, and the like.

The terms “halogen” and “halo” refer to fluorine, chlorine, bromine, and iodine.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NR^{a}R^{b}, —NR^{a}C(═O)R^{b}, —NR^{a}C(═O)NR^{a}NR^{b}, —NR^{a}C(═O)OR^{b}, —NR^{a}SO₂R^{b}, —C(═O)R^{a}, —C(═O)OR^{a}, —C(═O)NR^{a}R^{b}, —OC(═O)NR^{a}R^{b}, —OR^{a}, —SR^{a}, —SOR^{a}, —S(═O)₂R^{a}, —OS(═O)₂R^{a}, and —S(═O)₂OR^{a}. R^{a} and R^{b} in this context can be the same or different and independently can be hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

The term “optionally substituted,” as used herein, means that substitution with an additional group is optional, and therefore it is possible for the designated atom to be unsubstituted. Thus, by use of the term “optionally substituted,” the disclosure includes examples where the group is substituted and examples where it is not.

Examples of prodrugs that can be used to improve bioavailability include esters, optionally substituted esters, branched esters, optionally substituted branched esters, carbonates, optionally substituted carbonates, carbamates, optionally substituted carbamates, thioesters, optionally substituted thioesters, branched thioesters, optionally substituted branched thioesters, thiocarbonates, optionally substituted thiocarbonates, sulfenyl thiocarbonates, optionally substituted sulfenyl thiocarbonates, 2-hydroxypropanoate ester, optionally substitute 2-hydroxypropanoate ester, S-thiocarbonate, optionally substituted S-thiocarbonate, dithiocarbonates, optionally substituted dithiocarbonates, thiocarbamates, optionally substituted thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, sulfenyl, optionally substituted sulfenyl, sulfinyl, sulfonyl, sulfite, sulfate, sulfonamide, imidate, optionally substituted imidate, hydrazonate, optionally substituted hydrazonate, oximyl, optionally substituted oximyl, imidinyl, optionally substituted imidinyl, imidyl, optionally substituted imidyl, aminal, optionally substituted aminal, hemiaminal, optionally substituted hemiaminal, acetal, optionally substituted acetal, hemiacetal, optionally substituted hemiacetal, carbonimidate, optionally substituted carbonimidate, thiocarbonimidate, optionally substituted thiocarbonimidate, carbonimidyl, optionally substituted carbonimidyl, carbamimidate, optionally substituted carbamimidate, carbamimidyl, optionally substituted carbamimidyl, thioacetal, optionally substituted thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, optionally substituted bis-(acyloxybenzyl)esters, (acyloxybenzyl)esters, optionally substituted (acyloxybenzyl)esters, or BAB-esters.

The term “subject” (alternatively “patient” or “participant”, as in a clinical trial participant) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), Lagomorpha (rabbits), rodents (e.g., rats or mice), Procyon lotor (e.g., raccoons). In particular embodiments, the subject is human.

The term “subject in need thereof” (alternatively “patient in need thereof”) as used herein refers to a subject diagnosed with, or suspected of having, a viral infection, such as infection by SARS-CoV-2 (either symptomatic or asymptomatic); a subject at risk of being exposed to a viral infection, such as at risk of being exposed to a viral infection, such as infection by SARS-CoV-2 (such as, for example, health care workers who may be at risk of exposure to SARS-CoV-2); a subject exposed to a viral infection, such as infection by SARS-CoV-2 (such as household contacts of COVID-19 patients or asymptomatic patients infected with SARS-CoV-2), as defined herein.

For clarity, the terms “2019-nCoV,” “SARS-CoV-2”, “SARS-CoV-2/2019-nCoV” and “2019-nCoV/SARS-CoV-2” are interchangeable and, individually and collectively, refer to the novel coronavirus discovered in Wuhan, China in 2019, and varients thereof, including, but not limited to the more virulent strains that recently appeared in Brasil (known as P.1), the United Kingdom (known as 20I/501Y.V1, VOC 202012/01, or B.1.1.7) and in South Africa (known as 20H/501Y.V2 or B.1.351) as well as further varients and lineages that derive therefrom. Certain varients are more particularly described in the following references, each of which is incorporated by reference herein in its entirety for its teaching of viral variants: Horby P, Huntley C, Davies N, et al. NERVTAG note on B.1.1.7 severity. SAGE meeting report. Jan. 21, 2021; Wu K, Werne A P, Moliva J I, et al. mRNA-1273 vaccine induces neutralizing antibodies against spike mutants from global SARS-CoV-2 variants. bioRxiv. Posted Jan. 25, 2021; Xie X, Zou J, Fontes-Garfias C R, et al. Neutralization of N501Y mutant SARS-CoV-2 by BNT162b2 vaccine-elicited sera. bioRxiv. Posted Jan. 7, 2021. Greaney A J, Loes A N, Crawford K H D, et al. Comprehensive mapping of mutations to the SARS-CoV-2 receptor-binding domain that affect recognition by polyclonal human serum antibodies. bioRxiv. Jan. 4, 2021; Weisblum Y, Schmidt F, Zhang F, et al. Escape from neutralizing antibodies by SARS-CoV-2 spike protein variants. eLife 2020; 9:e61312; Resende P C, Bezerra J F, de Vasconcelos R H T, at al. Spike E484K mutation in the first SARS-CoV-2 reinfection case confirmed in Brazil, 2020. Jan. 10, 2021.

The term “COVID-19” refers to the disease caused by viral infection by SARS-CoV-2/2019-nCoV.

Those skilled in the art will recognize that certain compounds, and in particular compounds containing certain heteroatoms and double or triple bonds, can be tautomers, structural isomers that readily interconvert. Thus, tautomeric compounds can be drawn in a number of different ways that are equivalent. Non-limiting examples of such tautomers include those exemplified below.

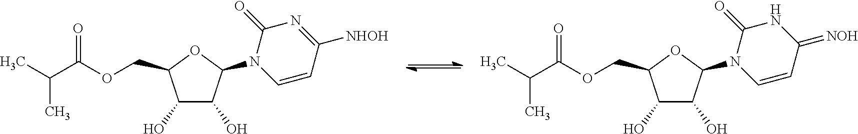

### Compounds

In certain embodiments, the disclosure relates to a compound of Formula I,

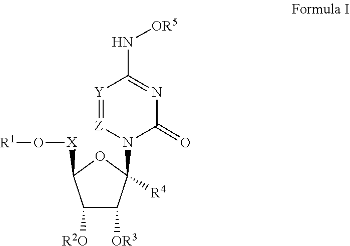

or a tautomer thereof, or pharmaceutical salt or physiological salt thereof, wherein
X is CH₂, CHCH₃, C(CH₃)₂, CHF, CF₂, or CD₂;
Y is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or carbonyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, hydroxyl, thiol, or carbonyl, wherein R″ is optionally substituted with one or more, the same or different, R¹⁰;
R¹, R², R³, and R⁵ are each independently selected from H,

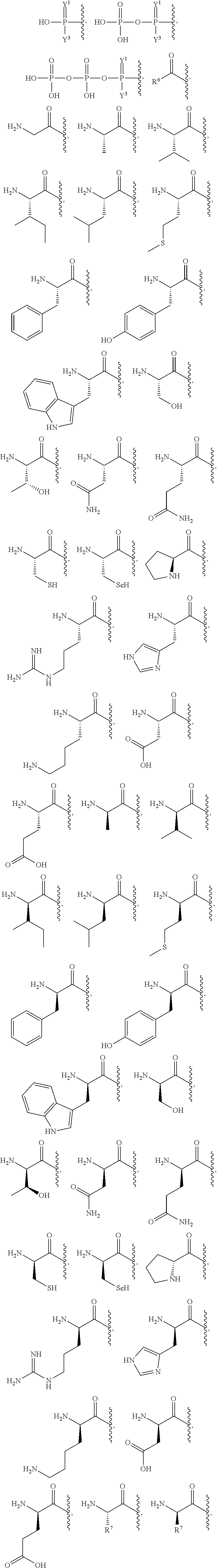

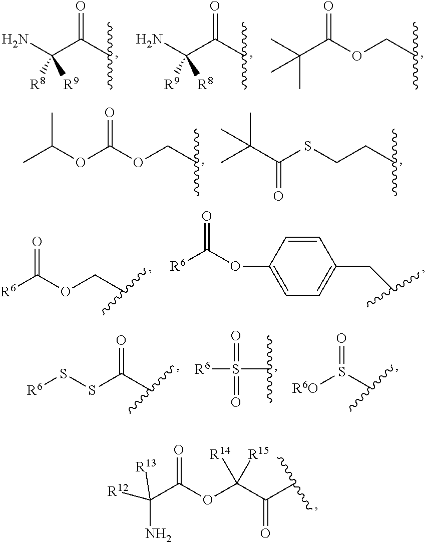

together with the oxygen to which each is bound, R¹, R², R³, and R⁵ form optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substituted 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, N-substituted L-amino acid esters, N,N-disubstituted L-amino acid esters, N-substituted D-amino acid esters, N,N-disubstituted D-amino acid esters, oxymethoxy amino ester, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R², R³, and R⁵ are optionally substituted with one or more, the same or different, R¹⁰;
Y is O or S;
Y³ is OH or BH₃⁻M⁺, where M is Li, Na, K, NH₄, (CH₃CH₂)₃NH, (CH₃CH₂CH₂CH₂)₄N;
R⁶ is hydrogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, cyano, or lipid, wherein R⁶ is optionally substituted with one or more, the same or different, R¹⁰;
R⁷ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R⁸, and R⁹ can form a ring with the α-carbon they are attached to and the amino group attached to the α-carbon;
R⁸ and R⁹ can form a ring with the α-carbon to which they are attached;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl;
R¹² is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹² is optionally substituted with one or more, the same or different, R¹⁶;
R¹³ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹³ is optionally substituted with one or more, the same or different, R¹⁶;
R¹⁴ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁴ is optionally substituted with one or more, the same or different, R¹⁶;
R¹⁵ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁵ is optionally substituted with one or more, the same or different, R¹⁶;
R¹⁶ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁶ is optionally substituted with one or more, the same or different, R¹⁷;
R¹⁷ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and Lipid is a C₁₁-C₂₂ higher alkyl, C₁₁-C₂₂ higher alkoxy, polyethylene glycol, or aryl substituted with an alkyl group, or a lipid as described herein.

In certain embodiments, the lipid is a fatty alcohol, fatty amine, or fatty thiol derived from essential and/or non-essential fatty acids.

In certain embodiments, the lipid is an unsaturated, polyunsaturated, omega unsaturated, or omega polyunsaturated fatty alcohol, fatty amine, or fatty thiol derived from essential and/or non-essential fatty acids.

In certain embodiments, the lipid is a fatty alcohol, fatty amine, or fatty thiol derived from essential and non-essential fatty acids that have one or more of its carbon units substituted with an oxygen, nitrogen, or sulfur.

In certain embodiments, the lipid is an unsaturated, polyunsaturated, omega unsaturated, or omega polyunsaturated fatty alcohol, fatty amine, or fatty thiol derived from essential and/or non-essential fatty acids that have one or more of its carbon units substituted with an oxygen, nitrogen, or sulfur.

In certain embodiments, the lipid is a fatty alcohol, fatty amine, or fatty thiol derived from essential and/or non-essential fatty acids that is optionally substituted.

In certain embodiments, the lipid is an unsaturated, polyunsaturated, omega unsaturated, or omega polyunsaturated fatty alcohol, fatty amine, or fatty thiol derived from essential and/or non-essential fatty acids that is optionally substituted.

In certain embodiments, the lipid is a fatty alcohol, fatty amine, or fatty thiol derived from essential and/or non-essential fatty acids that have one or more of its carbon units substituted with an oxygen, nitrogen, or sulfur that is optionally substituted.

In certain embodiments, the lipid is an unsaturated, polyunsaturated, omega unsaturated, or omega polyunsaturated fatty alcohol, fatty amine, or fatty thiol derived from essential and/or non-essential fatty acids that have one or more of its carbon units substituted with an oxygen, nitrogen, or sulfur that is also optionally substituted.

In certain embodiments, the lipid is hexadecyloxypropyl.

In certain embodiments, the lipid is 2-aminohexadecyloxypropyl.

In certain embodiments, the lipid is 2-aminoarachidyl.

In certain embodiments, the lipid is 2-benzyloxyhexadecyloxypropyl.

In certain embodiments, the lipid is lauryl, myristyl, palmityl, stearyl, arachidyl, behenyl, or lignoceryl.

In certain embodiments, the lipid is a sphingolipid of the formula:

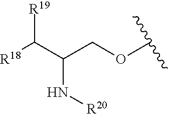

wherein,
R²⁰ of the sphingolipid is hydrogen, alkyl, C(═O)R²¹, C(═O)OR²¹, or C(═O)NHR²¹;
R¹⁹ of the sphingolipid is hydrogen, fluoro, OR²¹, OC(═O)R²¹, OC(═O)OR²¹, or OC(═O)NHR²¹;
R¹⁸ of the sphingolipid is a saturated or unsaturated alkyl chain of greater than 6 and less than 22 carbons optionally substituted with one or more halogen or hydroxy or a structure of the following formula:

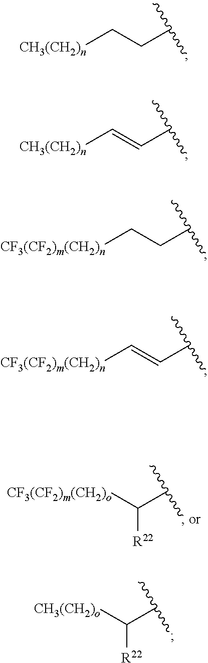

wherein n is 8 to 14 or less than or equal to 8 to less than or equal to 14, o is 9 to 15 or less than or equal to 9 to less than or equal to 15, the total of m and n is 8 to 14 or less than or equal to 8 to less than or equal to 14, the total of m and o is 9 to 15 or less than or equal to 9 to less than or equal to 15; or

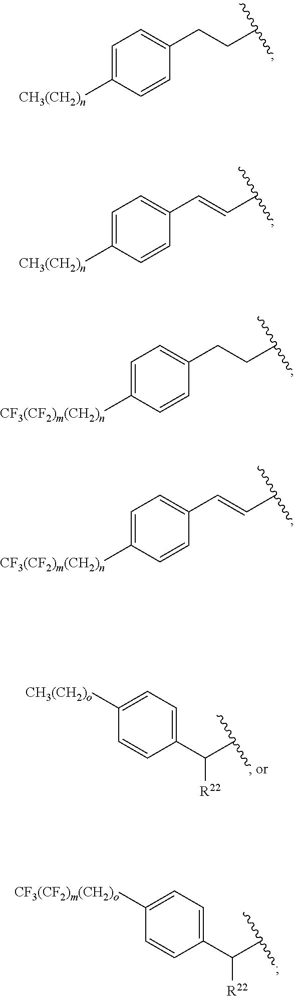

wherein n is 4 to 10 or less than or equal to 4 to less than or equal to 10, o is 5 to 11 or less than or equal to 5 to less than or equal to 11, the total of m and n is 4 to 10 or less than or equal to 4 to less than or equal to 10, and the total of m and o is 5 to 11 or less than or equal to 5 to less than or equal to 11; or

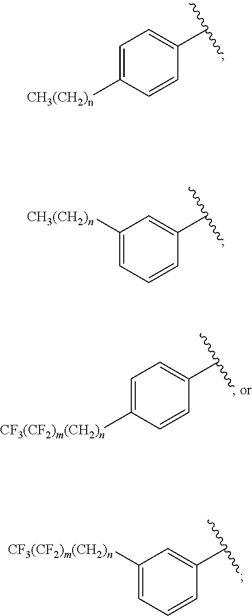

wherein n is 6 to 12 or n is less than or equal to 6 to less than or equal to 12, the total of m and n is 6 to 12 or n is less than or equal to 6 to less than or equal to 12;
R²² of the sphingolipid is OR²¹, OC(═O)R²¹, OC(═O)OR²¹, or OC(═O)NHR²¹;
R²¹ of the sphingolipid is hydrogen, cyano, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, or lipid; wherein R²¹ is optionally substituted with one or more, the same or different R²³; and
R²³ of the sphingolipid is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl.

In certain embodiments, R²⁰ of the sphingolipid is H, methyl, ethyl, propyl, n-butyl, isopropyl, 2-butyl, 1-ethylpropyl,1-propylbutyl, cyclopropyl, cyclobutyl, cyclopentyl, cyclohexyl, benzyl, or phenyl.

In certain embodiments, the sphingolipid is a sphingolipid of the formula:

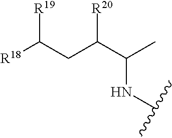

wherein,
R²⁰ of the sphingolipid is hydrogen, hydroxy, fluoro, OR²¹, OC(═O)R²¹, OC(═O)OR²¹, or OC(═O)NHR²¹;
R¹⁹ of the sphingolipid is hydrogen, hydroxy, fluoro, OR²¹, OC(═O)R²¹, OC(═O)OR²¹, or OC(═O)NHR²¹;
R¹⁸ of the sphingolipid is a saturated or unsaturated alkyl chain of greater than 6 and less than 22 carbons optionally substituted with one or more halogens or a structure of the following formula:

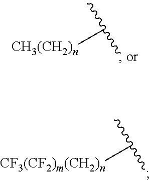

wherein n is 8 to 14 or less than or equal to 8 to less than or equal to 14, the total of m and n is 8 to 14 or less than or equal to 8 to less than or equal to 14;
R²¹ of the sphingolipid is hydrogen, cyano, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, or lipid; wherein R²¹ is optionally substituted with one or more, the same or different R²³; and
R²³ of the sphingolipid is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, esteryl, formyl, carboxy, carbamoyl, amido, or acyl.

In certain embodiments, R²¹ of the sphingolipid is H, methyl, ethyl, propyl, n-butyl, isopropyl, 2-butyl, 1-ethylpropyl,1-propylbutyl, cyclopropyl, cyclobutyl, cyclopentyl, cyclohexyl, or benzyl.

Suitable sphingolipids include, but are not limited to, sphingosine, ceramide, or sphingomyelin, and 2-aminoalkyl optionally substituted with one or more substituents.

Other suitable sphingolipids include, but are not limited to, 2-aminooctadecane-3,5-diol; (2S,3S,5S)-2-aminooctadecane-3,5-diol; (2S,3R,5S)-2-aminooctadecane-3,5-diol; 2-(methylamino)octadecane-3,5-diol; (2S,3R,5S)-2-(methylamino)octadecane-3,5-diol; 2-(dimethylamino)octadecane-3,5-diol; (2R,3S,5S)-2-(dimethylamino)octadecane-3,5-diol; 1-(pyrrolidin-2-yl)hexadecane-1,3-diol; (1S,3S)-1-((S)-pyrrolidin-2-yl)hexadecane-1,3-diol; 2-amino-11,11-difluorooctadecane-3,5-diol; (2S,3S,5S)-2-amino-11,11-difluorooctadecane-3,5-diol; 11,11-difluoro-2-(methylamino)octadecane-3,5-diol; (2S,3S,5S)-11,11-difluoro-2-(methylamino)octadecane-3,5-diol; N-((2S,3S,5S)-3,5-dihydroxyoctadecan-2-yl)acetamide; N-((2S,3S,5S)-3,5-dihydroxyoctadecan-2-yl)palmitamide;1-(1-aminocyclopropyl)hexadecane-1,3-diol; (1S,3R)-1-(1-aminocyclopropyl)hexadecane-1,3-diol; (1S,3S)-1-(1-aminocyclopropyl)hexadecane-1,3-diol; 2-amino-2-methyloctadecane-3,5-diol; (3S,5S)-2-amino-2-methyloctadecane-3,5-diol; (3S,5R)-2-amino-2-methyloctadecane-3,5-diol; (3S,5S)-2-methyl-2-(methylamino)octadecane-3,5-diol; 2-amino-5-hydroxy-2-methyloctadecan-3-one; (Z)-2-amino-5-hydroxy-2-methyloctadecan-3-one oxime; (2S,3R,5R)-2-amino-6,6-difluorooctadecane-3,5-diol; (2S,3S,5R)-2-amino-6,6-difluorooctadecane-3,5-diol; (2S,3S,5S)-2-amino-6,6-difluorooctadecane-3,5-diol; (2S,3R,5S)-2-amino-6,6-difluorooctadecane-3,5-diol; and (2S,3S,5S)-2-amino-18,18,18-trifluorooctadecane-3,5-diol, which can be optionally substituted with one or more substituents.

In exemplified embodiments of Formula I, R¹ is hydrogen,

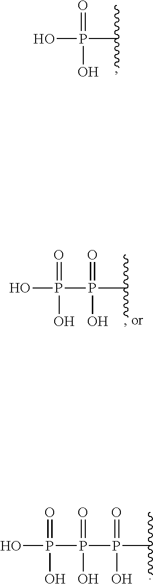

In exemplified embodiments of Formula I, R′ is methyl, fluoro, hydroxymethyl, fluoromethyl, difluoromethyl, trifluoromethyl, trideuteromethyl, thiomethyl, carboxylic acid, formyl, vinyl, or ethynyl.

In exemplified embodiments of Formula I, R″ is methyl, fluoro, hydroxymethyl, fluoromethyl, difluoromethyl, trifluoromethyl, trideuteromethyl, thiomethyl, carboxylic acid, formyl, vinyl, or ethynyl.

In exemplified embodiments of Formula I, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula I, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula I, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula I, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula II,

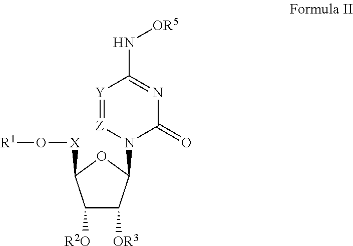

or a tautomer thereof, or a pharmaceutical salt or physiological salt thereof, wherein
X is CH₂, CHCH₃, C(CH₃)₂, CHF, CF₂, or CD₂;
Y is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or carbonyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, hydroxyl, thiol, or carbonyl, wherein R″ is optionally substituted with one or more, the same or different, R¹⁰;
R¹, R², R³ and R⁵ are each independently selected from H,

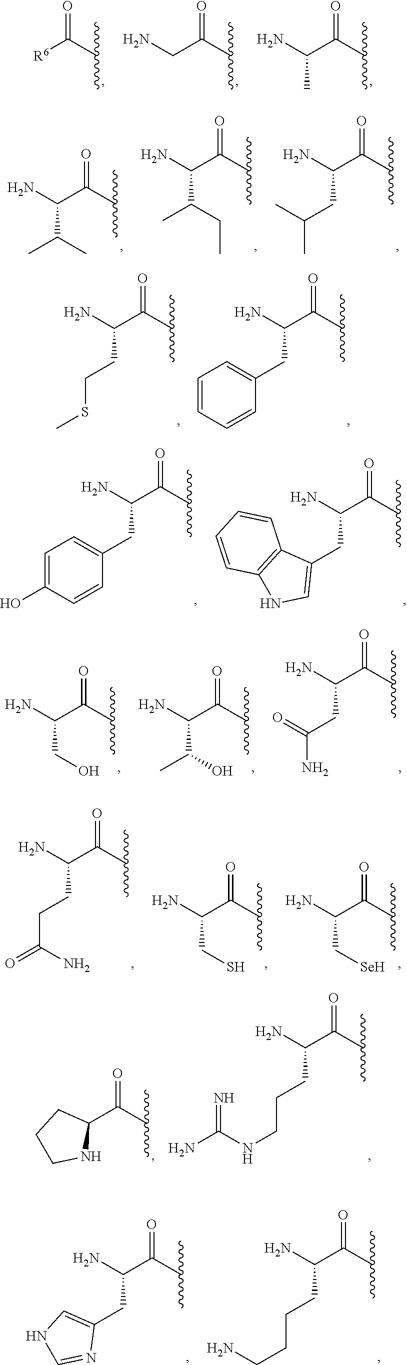

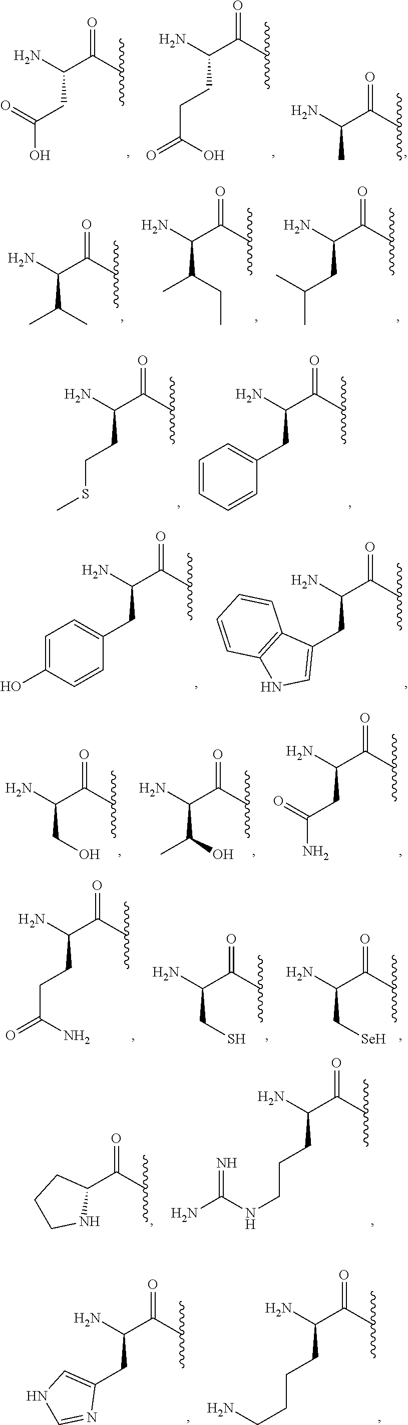

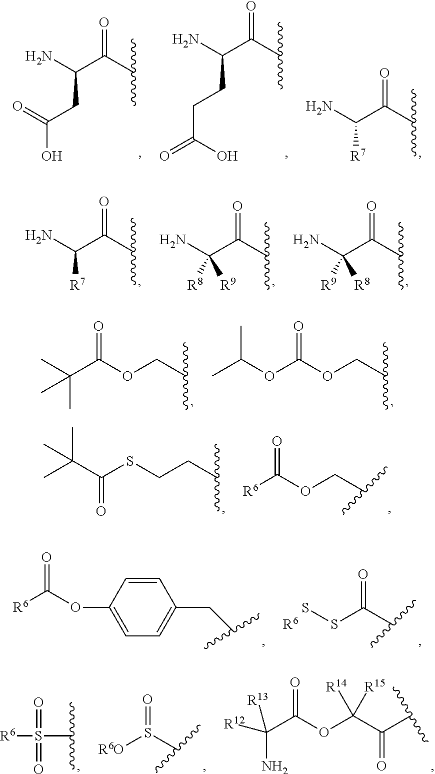

or together with the oxygen to which each is bound, R¹, R², R³, and R⁵ form optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R², R³, and R⁵ are optionally substituted with one or more, the same or different, R¹⁰;
with the proviso that R¹, R², R³, and R⁵ are not all H; and
R⁶-R¹⁷ and lipid are as defined in Formula I.

In exemplified embodiments of Formula II, R′ is methyl, fluoro, hydroxymethyl, fluoromethyl, difluoromethyl, trifluoromethyl, trideuteromethyl, thiomethyl, carboxylic acid, formyl, vinyl, or ethynyl.

In exemplified embodiments of Formula II, R″ is methyl, fluoro, hydroxymethyl, fluoromethyl, difluoromethyl, trifluoromethyl, trideuteromethyl, thiomethyl, carboxylic acid, formyl, vinyl, or ethynyl.

In exemplified embodiments of Formula II, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula II, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula II, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula II, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula III,

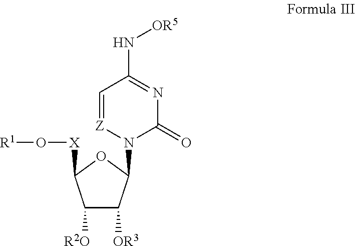

or a tautomer thereof, or a pharmaceutical salt or physiological salt thereof, wherein
X is CH₂, CHCH₃, C(CH₃)₂, CHF, CF₂, or CD₂;
Z is N or CR″;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, hydroxyl, thiol, or carbonyl, wherein R″ is optionally substituted with one or more, the same or different, R¹⁰;
R¹-R³ and R⁵ are as defined in Formula II, with the proviso that R¹, R², R³, and R⁵ are not all H; and
R⁶-R¹⁷ and lipid are as defined in Formula I and II.

In exemplified embodiments of Formula III, R″ is methyl, fluoro, hydroxymethyl, fluoromethyl, difluoromethyl, trifluoromethyl, trideuteromethyl, thiomethyl, carboxylic acid, formyl, vinyl, or ethynyl.

In exemplified embodiments of Formula III, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula III, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula III, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula III, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula IV,

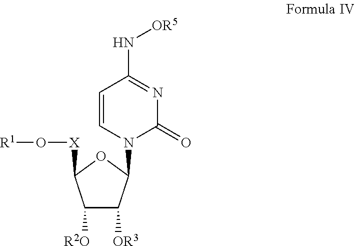

or a pharmaceutical salt or physiological salt thereof, wherein
X is CHCH₃, C(CH₃)₂, CHF, CF₂, or CD₂;
R¹-R³ and R⁵ are as defined in Formula II and III, with the proviso that R¹, R², R³, and R⁵ are not all H; and
R⁶-R¹⁷ and lipid are as defined in Formulas I-III.

In exemplified embodiments of Formula IV, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula IV, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula IV, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula IV, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula V,

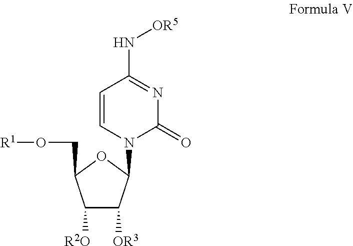

or a tautomer thereof, or a pharmaceutical salt or physiological salt thereof,
R¹-R³ and R⁵ are as defined in Formulas II-IV; and
R⁶-R¹⁷ and lipid are as defined in Formulas I-IV.

In exemplified embodiments of Formula V, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula V, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula V, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula V, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula VI,

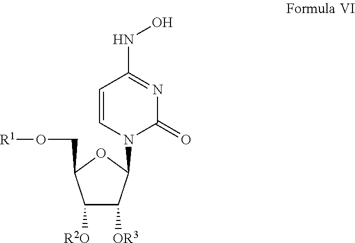

or a tautomer thereof, or a pharmaceutical salt or physiological salt thereof, wherein R¹-R³ are as defined in Formulas II-V; and
R⁶-R¹⁷ and lipid are as defined in Formulas I-V.

In exemplified embodiments of Formula VI, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VI, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VI, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VI, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula VIa-f,

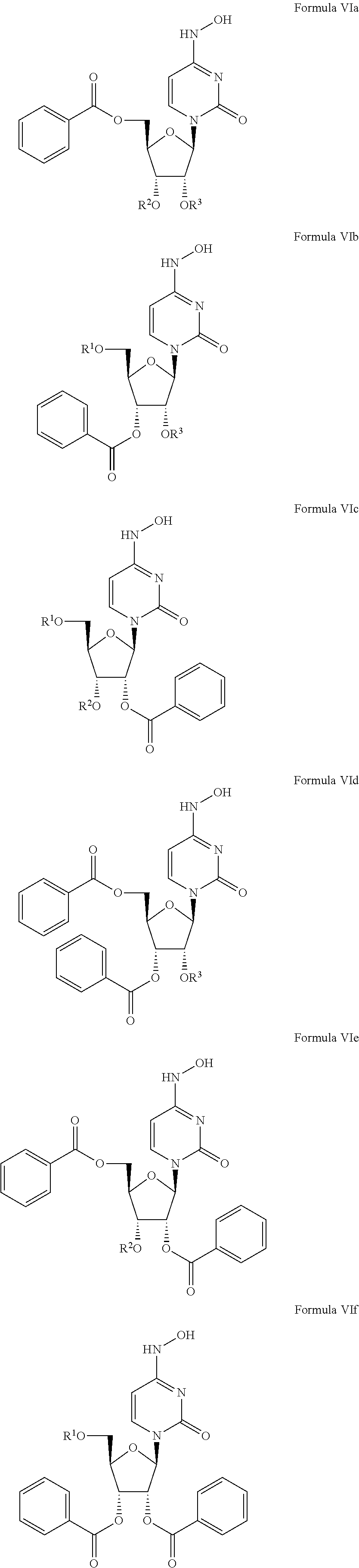

or a tautomer thereof, or a pharmaceutical salt or physiological salt thereof, wherein R¹-R³ are as defined in Formulas II-VI and R⁶-R¹⁷ and lipid are as defined in Formulas I-VI.

In exemplified embodiments of Formula VIa-f, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VIa-f, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VIa-f, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VIa-f, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula VII,

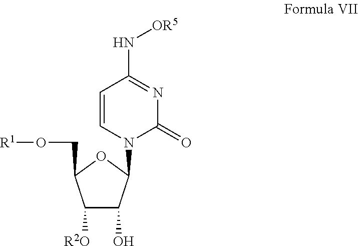

or a tautomer thereof, or a pharmaceutical salt or physiological salt thereof, wherein R¹, R², and R⁵ are as defined in Formulas II-VI and R⁶-R¹⁷ and lipid are as defined in Formulas I-VI.

In exemplified embodiments of Formula VII, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula VIII,

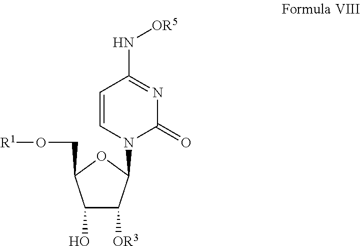

or a tautomer thereof, or a pharmaceutical salt or physiological salt thereof, wherein R¹, R³, and R⁵ are as defined in Formulas II-VII and R⁶-R¹⁷ and lipid are as defined in Formulas I-VII.

In exemplified embodiments of Formula VIII, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VIII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VIII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula VIII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula IX,

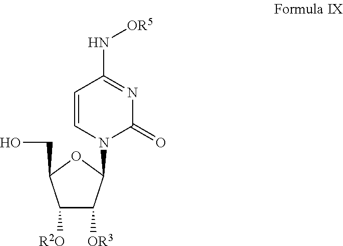

or a tautomer thereof, or a pharmaceutical salt or physiological salt thereof, wherein
R², R³, and R⁵ are as defined in Formulas II-VIII and R⁶-R¹⁷ and lipid are as defined in Formulas I-VIII.

In exemplified embodiments of Formula IX, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula IX, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula IX, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula IX, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula X,

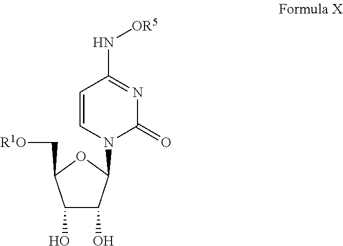

or a tautomer thereof, or a pharmaceutical salt or physiological salt thereof, wherein
R¹ and R⁵ are as defined in Formulas II-IX and R⁶-R¹⁷ and lipid are as defined in Formulas I-IX.

In exemplified embodiments of Formula X, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula X, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula X, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula X, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XI,

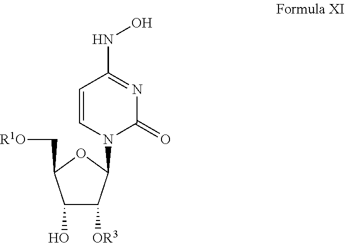

or a tautomer thereof, or a pharmaceutical salt or physiological salt thereof, wherein
R¹ and R³ are as defined in Formulas II-X and R⁶-R¹⁷ and lipid are as defined in Formulas I-X.

In exemplified embodiments of Formula XI, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XI, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XI, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XI, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XII,

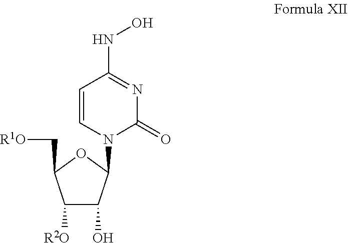

or a tautomer thereof, or a pharmaceutical salt or physiological salt thereof, wherein
R¹ and R² are as defined in Formulas II-XII and R⁶-R¹⁷ and lipid are as defined in Formulas I-XI.

In exemplified embodiments of Formula XII, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XIII,

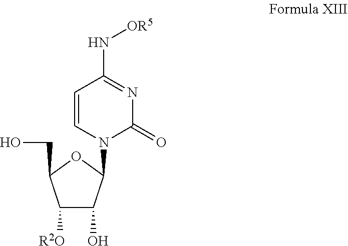

or a tautomer thereof, or a pharmaceutical salt or physiological salt thereof, wherein
R² and R⁵ are as defined in Formulas II-XII and R⁶-R¹⁷ and lipid are as defined in Formulas I-XII.

In exemplified embodiments of Formula XIII, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XIII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XIII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XIII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XIV,

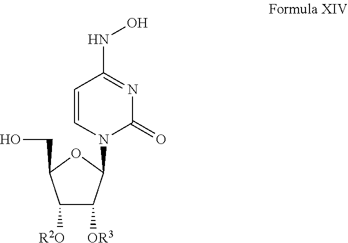

or a tautomer thereof, or a pharmaceutical salt or physiological salt thereof, wherein
R² and R³ are as defined in Formulas II-XIII and R⁶-R¹⁷ and lipid are as defined in Formulas I-XIII.

In exemplified embodiments of Formula XIV, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XIV, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XIV, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XIV, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XV,

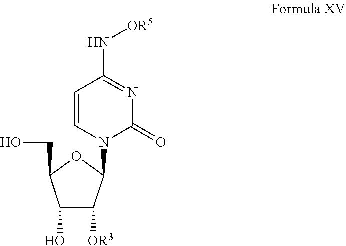

or a tautomer thereof, or a pharmaceutical salt or physiological salt thereof, wherein
R³ and R⁵ are as defined in Formulas II-XIV and R⁶-R¹⁷ and lipid are as defined in Formulas I-XIV.

In exemplified embodiments of Formula XV, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XV, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XV, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XV, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XVI,

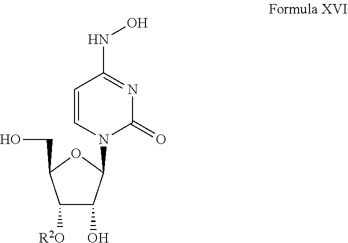

or a tautomer thereof, or a pharmaceutical salt or physiological salt thereof, wherein
R² is as defined in Formulas II-XV and R⁶-R¹⁷ and lipid are as defined in Formulas I-XV. In exemplified embodiments of Formula XVI, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XVI, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XVI, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XVI, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XVII,

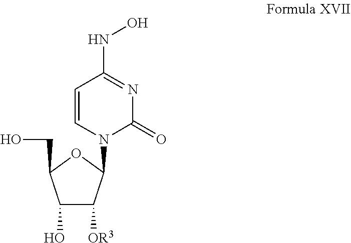

or a tautomer thereof, or a pharmaceutical salt or physiological salt thereof, wherein
R³ is as defined in Formulas II-XVI and R⁶-R¹⁷ and lipid are as defined in Formulas I-XVI.

In exemplified embodiments of Formula XVII, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XVII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XVII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XVII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XVIII,

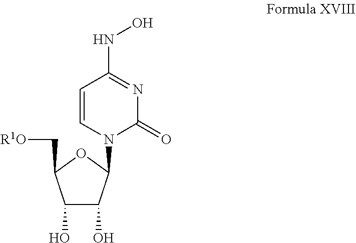

or a tautomer thereof, or a pharmaceutical salt or physiological salt thereof, wherein
R¹ is as defined in Formulas II-XVII and R⁶-R¹⁷ and lipid are as defined in Formulas I-XVII.

In exemplified embodiments of Formula XVIII, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XVIII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XVIII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XVIII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XIX,

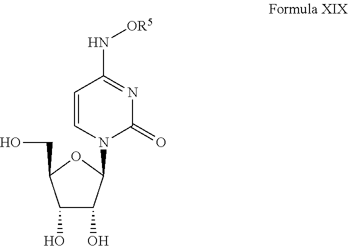

or a tautomer thereof, or a pharmaceutical salt or physiological salt thereof, wherein
R⁵ is as defined in Formulas II-XVIII and R⁶-R¹⁷ and lipid are as defined in Formulas I-XVIII.

In exemplified embodiments of Formula XIX, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XIX, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XIX, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XIX, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, the disclosure relates to a compound of Formula XX,

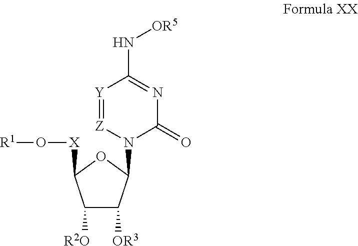

or a tautomer thereof, or a pharmaceutical salt or physiological salt thereof, wherein
X is CH₂, CHCH₃, C(CH₃)₂, CHF, CF₂, or CD₂;
Y is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or carbonyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, hydroxyl, thiol, or carbonyl, wherein R″ is optionally substituted with one or more, the same or different, R¹⁰; wherein R¹-R³ and R⁵ are as defined in Formulas II-XIX with the proviso that R¹, R², R³ and R⁵ are not all H;
and R⁶-R¹⁷ and Lipid are as defined in Formulas I-XIX In exemplified embodiments of Formula XX, R⁶ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XX, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XX, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In exemplified embodiments of Formula XX, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, or N,N-dipropylamino.

In certain embodiments, a compound of Formula XX is not one of the following structures:

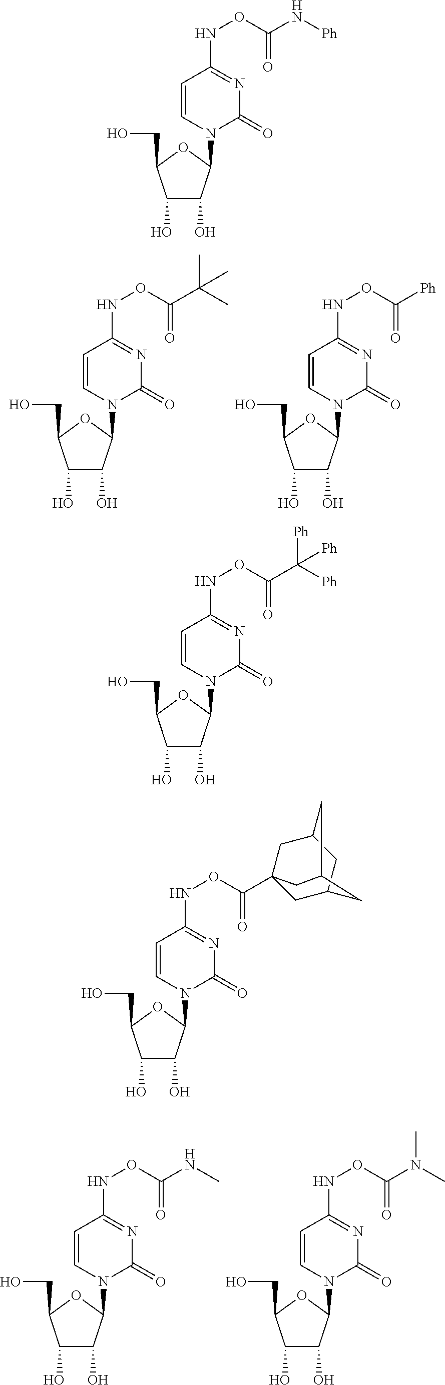

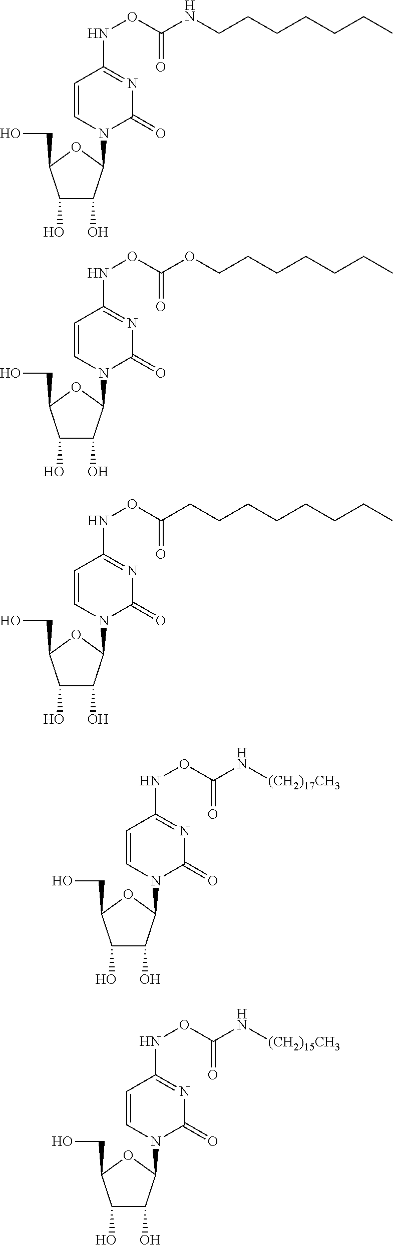

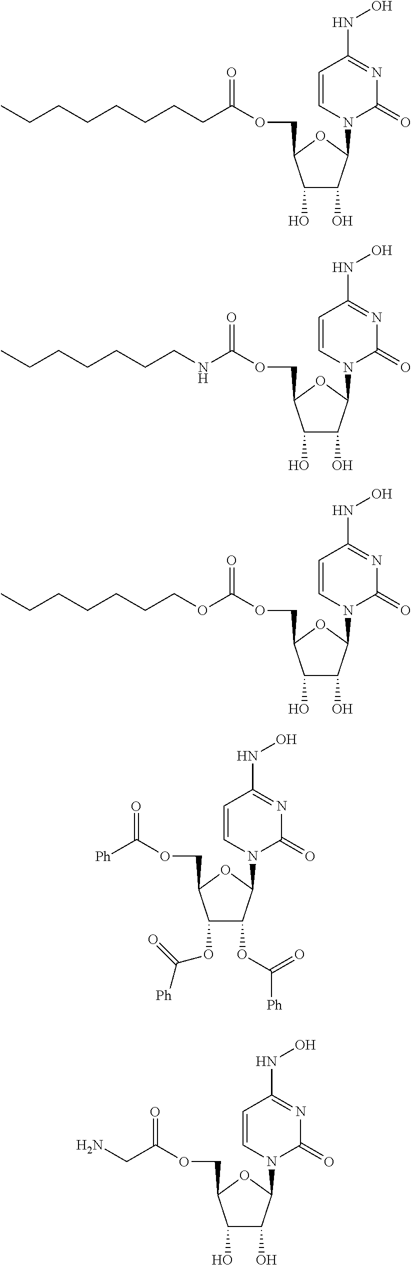

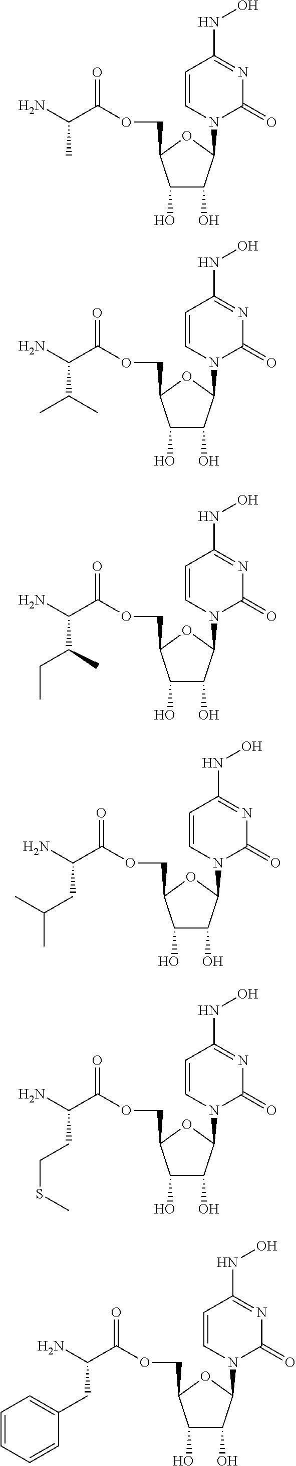

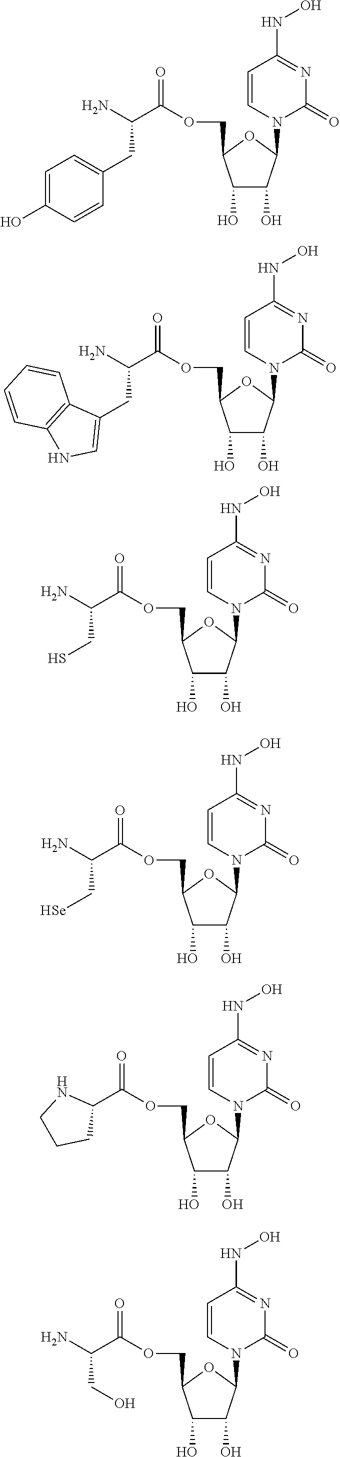

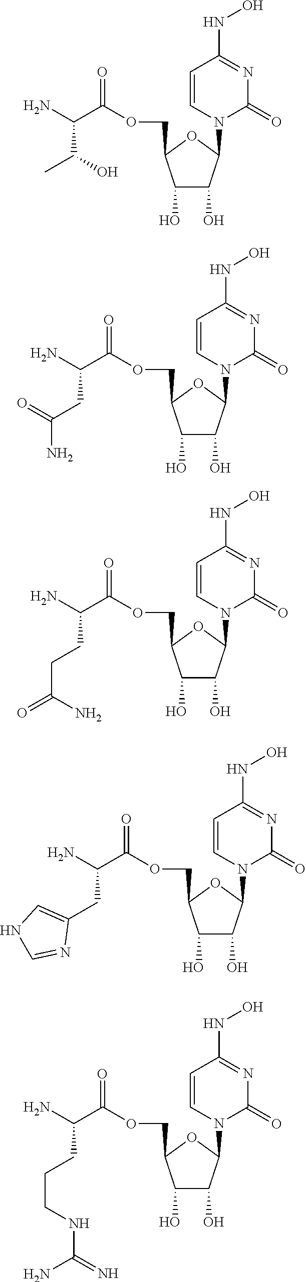

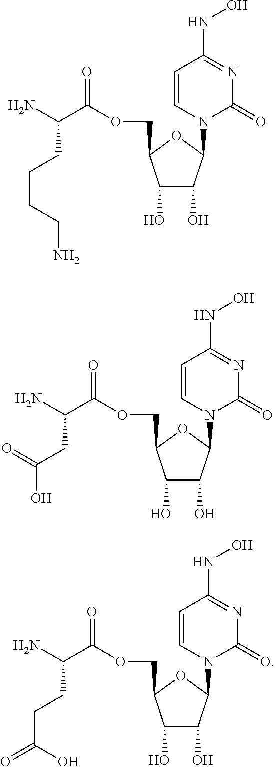 [SMILES: NCCCC[C@H](N)C(=O)OC[C@H]1O[C@@H](n2ccc(NO)nc2=O)[C@H](O)[C@@H]1O ; N[C@@H](CC(=O)O)C(=O)OC[C@H]1O[C@@H](n2ccc(NO)nc2=O)[C@H](O)[C@@H]1O ; N[C@@H](CCC(=O)O)C(=O)OC[C@H]1O[C@@H](n2ccc(NO)nc2=O)[C@H](O)[C@@H]1O]

In exemplary embodiments, the compound is selected from:

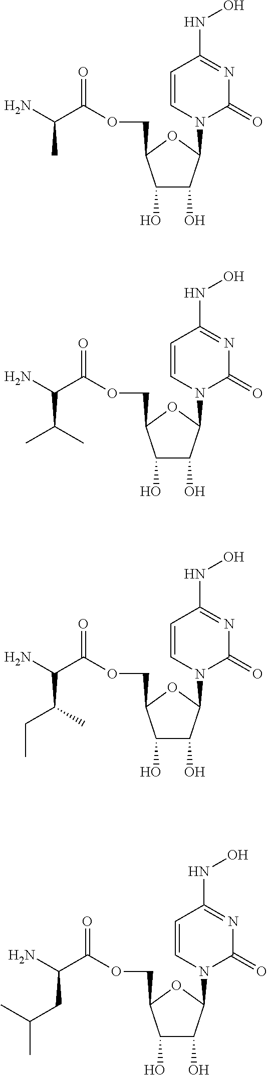

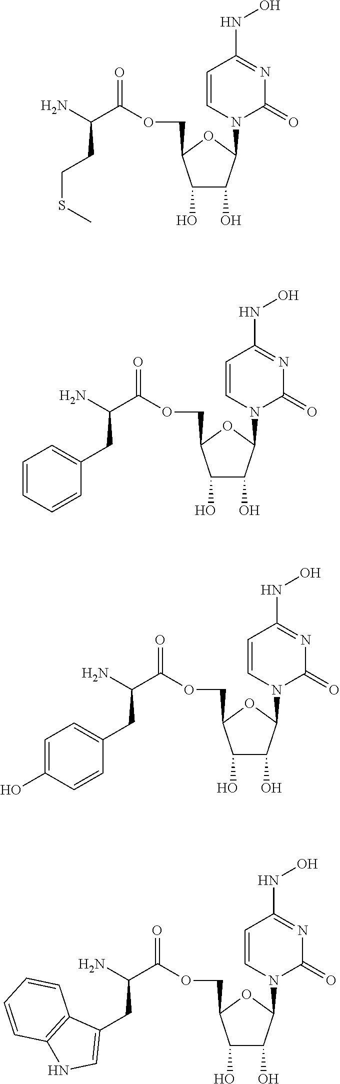 [SMILES: CSCC[C@@H](N)C(=O)OC[C@H]1O[C@@H](n2ccc(NO)nc2=O)[C@H](O)[C@@H]1O ; N[C@H](Cc1ccccc1)C(=O)OC[C@H]1O[C@@H](n2ccc(NO)nc2=O)[C@H](O)[C@@H]1O ; N[C@H](Cc1ccc(O)cc1)C(=O)OC[C@H]1O[C@@H](n2ccc(NO)nc2=O)[C@H](O)[C@@H]1O ; N[C@H](Cc1c[nH]c2ccccc12)C(=O)OC[C@H]1O[C@@H](n2ccc(NO)nc2=O)[C@H](O)[C@@H]1O]

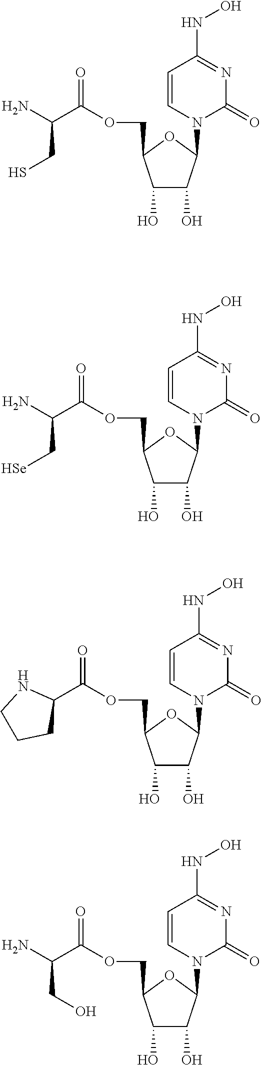

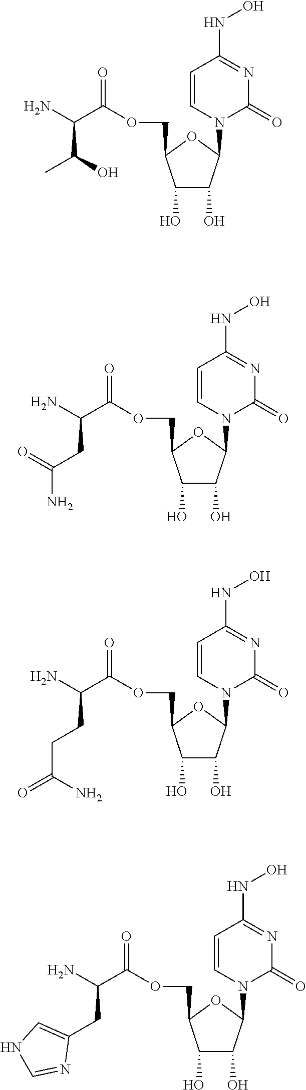 [SMILES: C[C@H](O)[C@@H](N)C(=O)OC[C@H]1O[C@@H](n2ccc(NO)nc2=O)[C@H](O)[C@@H]1O ; NC(=O)C[C@@H](N)C(=O)OC[C@H]1O[C@@H](n2ccc(NO)nc2=O)[C@H](O)[C@@H]1O ; NC(=O)CC[C@@H](N)C(=O)OC[C@H]1O[C@@H](n2ccc(NO)nc2=O)[C@H](O)[C@@H]1O ; N[C@H](Cc1c[nH]cn1)C(=O)OC[C@H]1O[C@@H](n2ccc(NO)nc2=O)[C@H](O)[C@@H]1O]

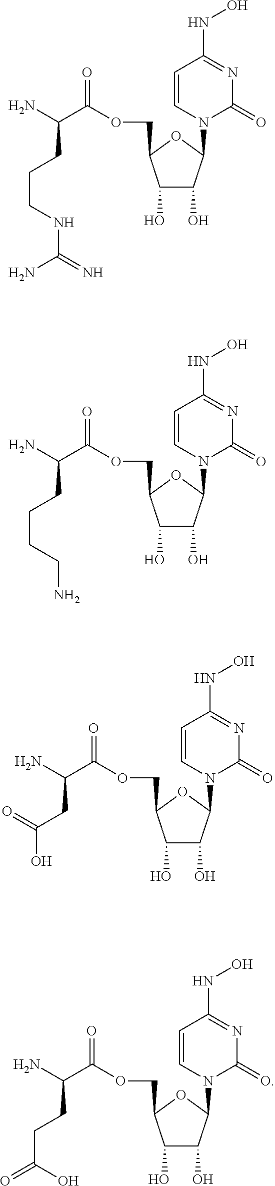 [SMILES: N=C(N)NCCC[C@@H](N)C(=O)OC[C@H]1O[C@@H](n2ccc(NO)nc2=O)[C@H](O)[C@@H]1O ; NCCCC[C@@H](N)C(=O)OC[C@H]1O[C@@H](n2ccc(NO)nc2=O)[C@H](O)[C@@H]1O ; N[C@H](CC(=O)O)C(=O)OC[C@H]1O[C@@H](n2ccc(NO)nc2=O)[C@H](O)[C@@H]1O ; N[C@H](CCC(=O)O)C(=O)OC[C@H]1O[C@@H](n2ccc(NO)nc2=O)[C@H](O)[C@@H]1O]

In exemplary embodiments, the compound is selected from:

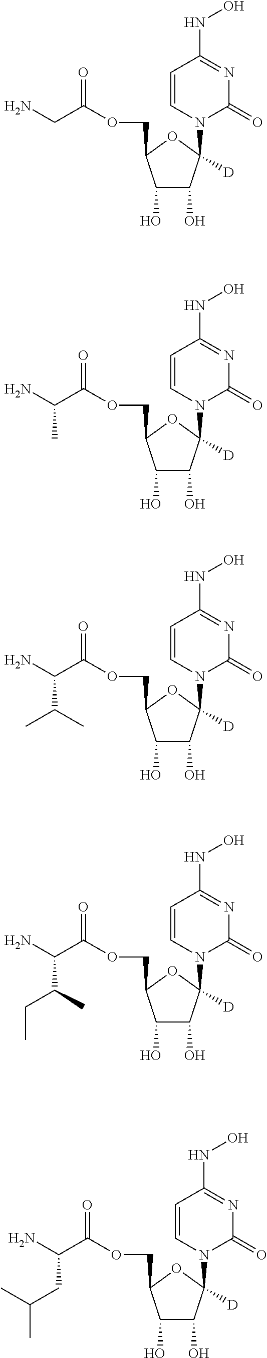 [SMILES: [2H][C@@]1(n2ccc(NO)nc2=O)O[C@H](COC(=O)CN)[C@@H](O)[C@H]1O ; [2H][C@@]1(n2ccc(NO)nc2=O)O[C@H](COC(=O)[C@H](C)N)[C@@H](O)[C@H]1O ; [2H][C@@]1(n2ccc(NO)nc2=O)O[C@H](COC(=O)[C@@H](N)C(C)C)[C@@H](O)[C@H]1O ; [2H][C@@]1(n2ccc(NO)nc2=O)O[C@H](COC(=O)[C@@H](N)[C@@H](C)CC)[C@@H](O)[C@H]1O ; [2H][C@@]1(n2ccc(NO)nc2=O)O[C@H](COC(=O)[C@@H](N)CC(C)C)[C@@H](O)[C@H]1O]

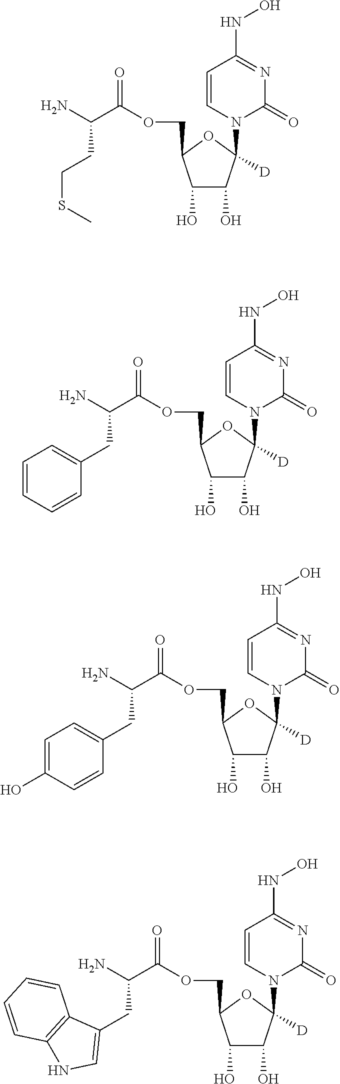 [SMILES: [2H][C@@]1(n2ccc(NO)nc2=O)O[C@H](COC(=O)[C@@H](N)CCSC)[C@@H](O)[C@H]1O ; [2H][C@@]1(n2ccc(NO)nc2=O)O[C@H](COC(=O)[C@@H](N)Cc2ccccc2)[C@@H](O)[C@H]1O ; [2H][C@@]1(n2ccc(NO)nc2=O)O[C@H](COC(=O)[C@@H](N)Cc2ccc(O)cc2)[C@@H](O)[C@H]1O ; [2H][C@@]1(n2ccc(NO)nc2=O)O[C@H](COC(=O)[C@@H](N)Cc2c[nH]c3ccccc23)[C@@H](O)[C@H]1O]

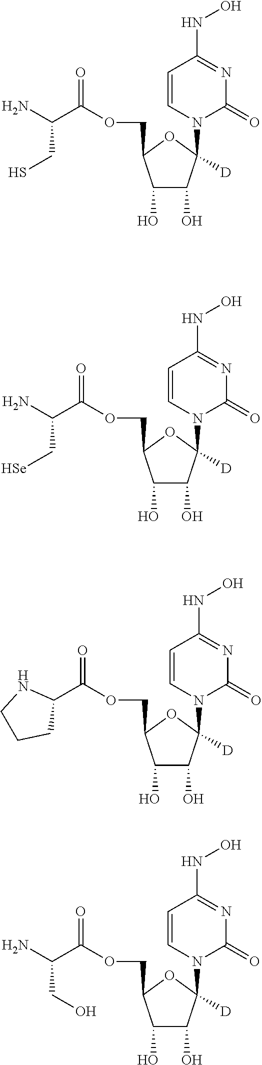

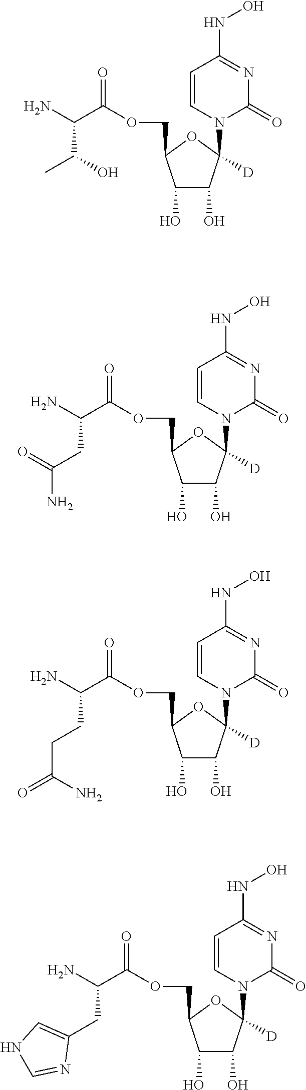 [SMILES: [2H][C@@]1(n2ccc(NO)nc2=O)O[C@H](COC(=O)[C@@H](N)[C@@H](C)O)[C@@H](O)[C@H]1O ; [2H][C@@]1(n2ccc(NO)nc2=O)O[C@H](COC(=O)[C@@H](N)CC(N)=O)[C@@H](O)[C@H]1O ; [2H][C@@]1(n2ccc(NO)nc2=O)O[C@H](COC(=O)[C@@H](N)CCC(N)=O)[C@@H](O)[C@H]1O ; [2H][C@@]1(n2ccc(NO)nc2=O)O[C@H](COC(=O)[C@@H](N)Cc2c[nH]cn2)[C@@H](O)[C@H]1O]

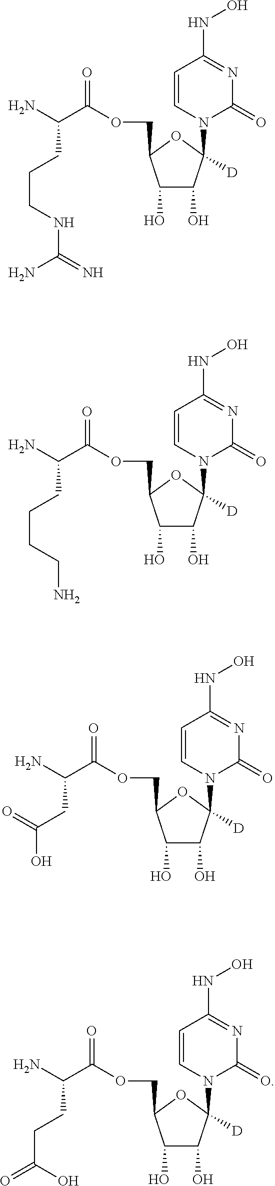 [SMILES: [2H][C@@]1(n2ccc(NO)nc2=O)O[C@H](COC(=O)[C@@H](N)CCCNC(=N)N)[C@@H](O)[C@H]1O ; [2H][C@@]1(n2ccc(NO)nc2=O)O[C@H](COC(=O)[C@@H](N)CCCCN)[C@@H](O)[C@H]1O ; [2H][C@@]1(n2ccc(NO)nc2=O)O[C@H](COC(=O)[C@@H](N)CC(=O)O)[C@@H](O)[C@H]1O ; [2H][C@@]1(n2ccc(NO)nc2=O)O[C@H](COC(=O)[C@@H](N)CCC(=O)O)[C@@H](O)[C@H]1O]

In exemplary embodiments, the compound is selected from:

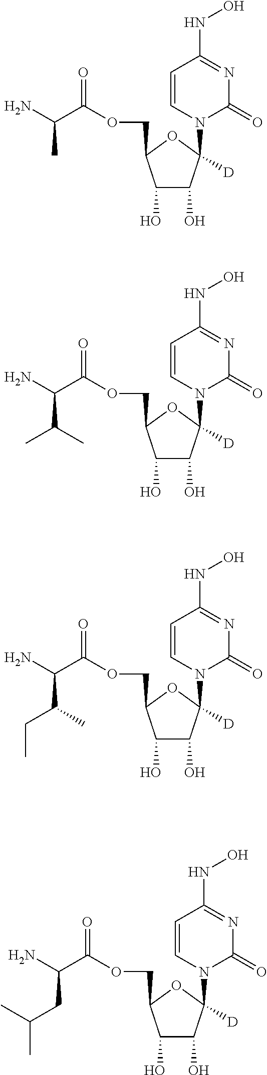 [SMILES: [2H][C@@]1(n2ccc(NO)nc2=O)O[C@H](COC(=O)[C@@H](C)N)[C@@H](O)[C@H]1O ; [2H][C@@]1(n2ccc(NO)nc2=O)O[C@H](COC(=O)[C@H](N)C(C)C)[C@@H](O)[C@H]1O ; [2H][C@@]1(n2ccc(NO)nc2=O)O[C@H](COC(=O)[C@H](N)[C@H](C)CC)[C@@H](O)[C@H]1O ; [2H][C@@]1(n2ccc(NO)nc2=O)O[C@H](COC(=O)[C@H](N)CC(C)C)[C@@H](O)[C@H]1O]

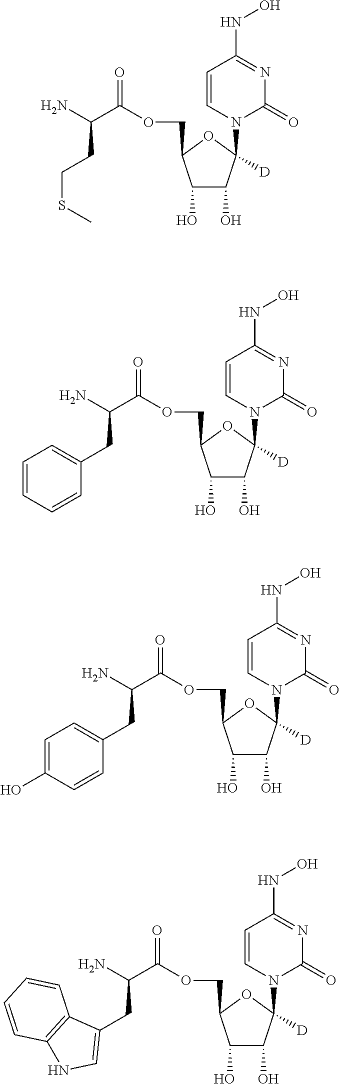 [SMILES: [2H][C@@]1(n2ccc(NO)nc2=O)O[C@H](COC(=O)[C@H](N)CCSC)[C@@H](O)[C@H]1O ; [2H][C@@]1(n2ccc(NO)nc2=O)O[C@H](COC(=O)[C@H](N)Cc2ccccc2)[C@@H](O)[C@H]1O ; [2H][C@@]1(n2ccc(NO)nc2=O)O[C@H](COC(=O)[C@H](N)Cc2ccc(O)cc2)[C@@H](O)[C@H]1O ; [2H][C@@]1(n2ccc(NO)nc2=O)O[C@H](COC(=O)[C@H](N)Cc2c[nH]c3ccccc23)[C@@H](O)[C@H]1O]

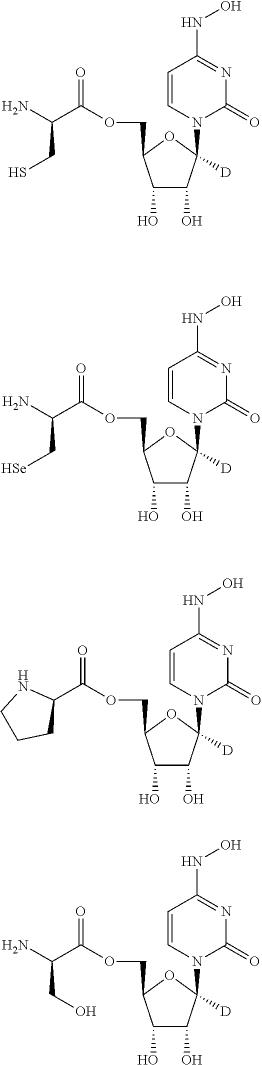

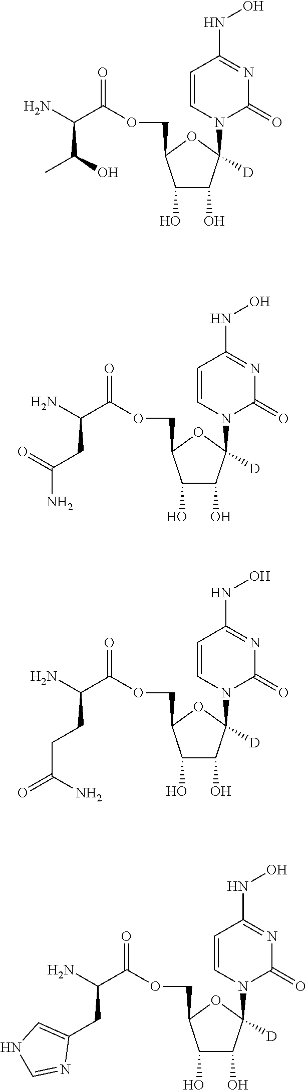 [SMILES: [2H][C@@]1(n2ccc(NO)nc2=O)O[C@H](COC(=O)[C@H](N)[C@H](C)O)[C@@H](O)[C@H]1O ; [2H][C@@]1(n2ccc(NO)nc2=O)O[C@H](COC(=O)[C@H](N)CC(N)=O)[C@@H](O)[C@H]1O ; [2H][C@@]1(n2ccc(NO)nc2=O)O[C@H](COC(=O)[C@H](N)CCC(N)=O)[C@@H](O)[C@H]1O ; [2H][C@@]1(n2ccc(NO)nc2=O)O[C@H](COC(=O)[C@H](N)Cc2c[nH]cn2)[C@@H](O)[C@H]1O]

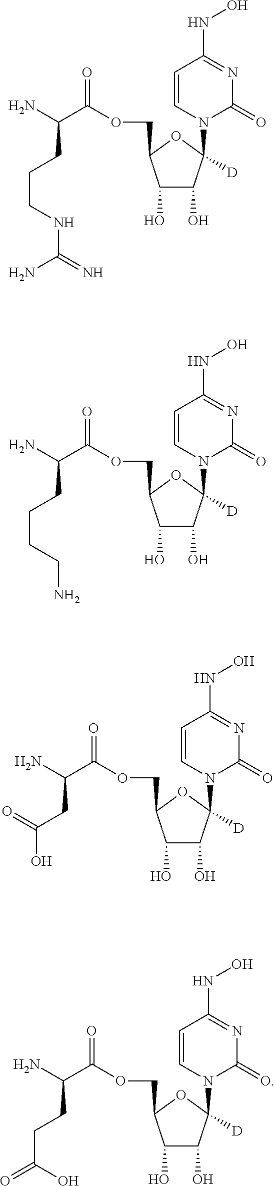 [SMILES: [2H][C@@]1(n2ccc(NO)nc2=O)O[C@H](COC(=O)[C@H](N)CCCNC(=N)N)[C@@H](O)[C@H]1O ; [2H][C@@]1(n2ccc(NO)nc2=O)O[C@H](COC(=O)[C@H](N)CCCCN)[C@@H](O)[C@H]1O ; [2H][C@@]1(n2ccc(NO)nc2=O)O[C@H](COC(=O)[C@H](N)CC(=O)O)[C@@H](O)[C@H]1O ; [2H][C@@]1(n2ccc(NO)nc2=O)O[C@H](COC(=O)[C@H](N)CCC(=O)O)[C@@H](O)[C@H]1O]

In exemplary embodiments, the compound is selected from:

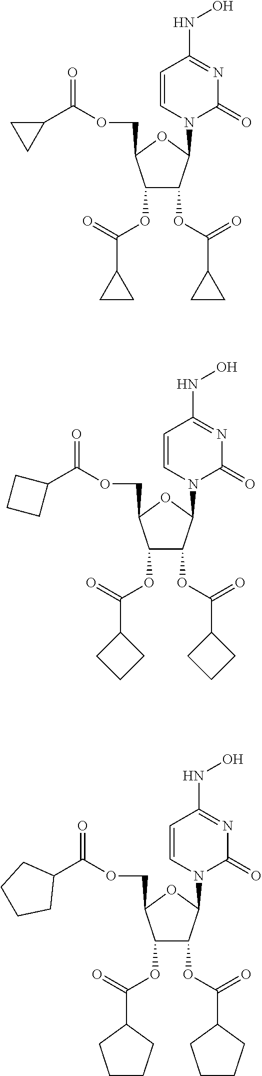 [SMILES: O=C(OC[C@H]1O[C@@H](n2ccc(NO)nc2=O)[C@H](OC(=O)C2CC2)[C@@H]1OC(=O)C1CC1)C1CC1 ; O=C(OC[C@H]1O[C@@H](n2ccc(NO)nc2=O)[C@H](OC(=O)C2CCC2)[C@@H]1OC(=O)C1CCC1)C1CCC1 ; O=C(OC[C@H]1O[C@@H](n2ccc(NO)nc2=O)[C@H](OC(=O)C2CCCC2)[C@@H]1OC(=O)C1CCCC1)C1CCCC1]

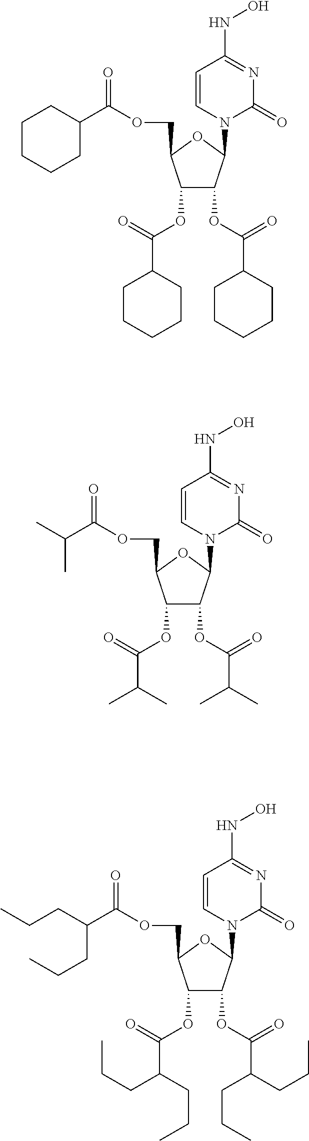 [SMILES: O=C(OC[C@H]1O[C@@H](n2ccc(NO)nc2=O)[C@H](OC(=O)C2CCCCC2)[C@@H]1OC(=O)C1CCCCC1)C1CCCCC1 ; CC(C)C(=O)OC[C@H]1O[C@@H](n2ccc(NO)nc2=O)[C@H](OC(=O)C(C)C)[C@@H]1OC(=O)C(C)C ; CCCC(CCC)C(=O)OC[C@H]1O[C@@H](n2ccc(NO)nc2=O)[C@H](OC(=O)C(CCC)CCC)[C@@H]1OC(=O)C(CCC)CCC]

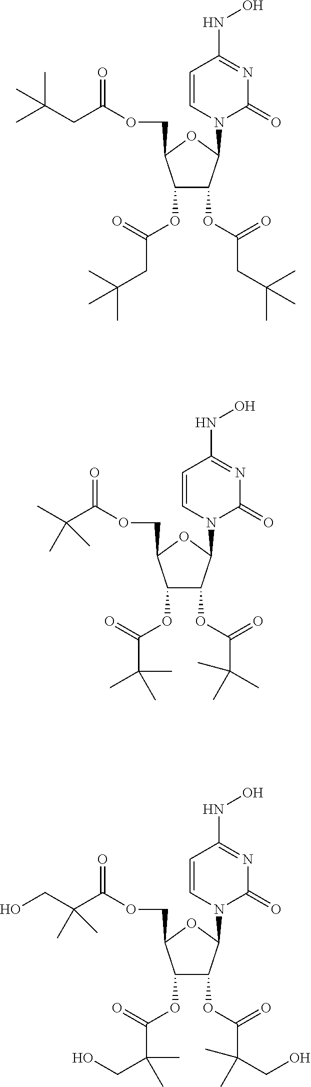

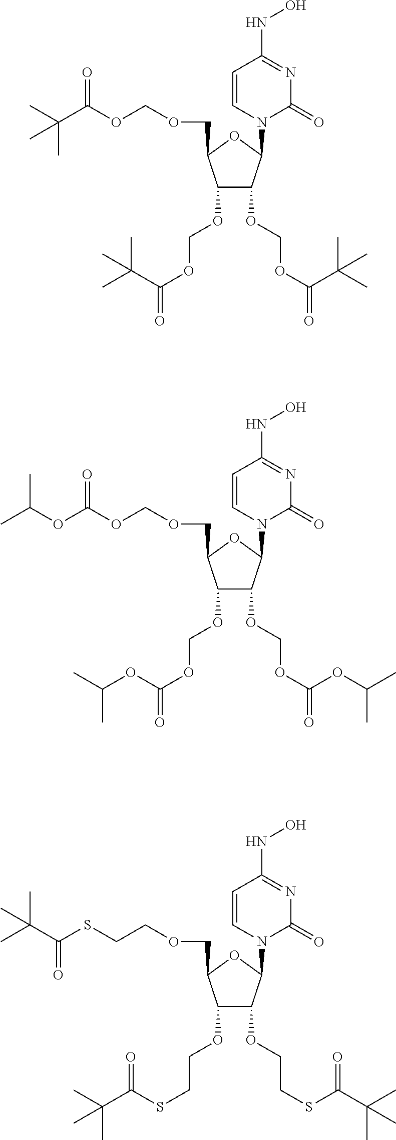

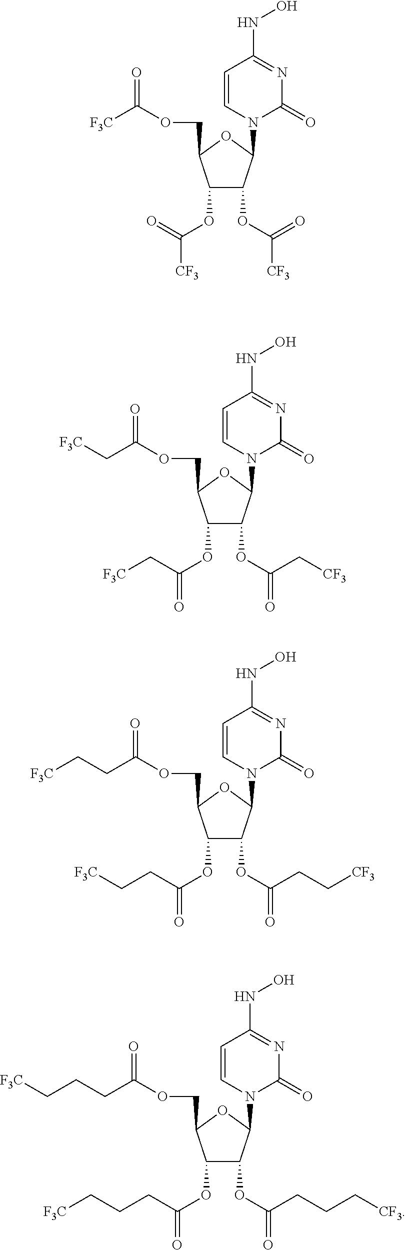 [SMILES: O=C(OC[C@H]1O[C@@H](n2ccc(NO)nc2=O)[C@H](OC(=O)C(F)(F)F)[C@@H]1OC(=O)C(F)(F)F)C(F)(F)F ; O=C(CC(F)(F)F)OC[C@H]1O[C@@H](n2ccc(NO)nc2=O)[C@H](OC(=O)CC(F)(F)F)[C@@H]1OC(=O)CC(F)(F)F ; O=C(CCC(F)(F)F)OC[C@H]1O[C@@H](n2ccc(NO)nc2=O)[C@H](OC(=O)CCC(F)(F)F)[C@@H]1OC(=O)CCC(F)(F)F ; O=C(CCCC(F)(F)F)OC[C@H]1O[C@@H](n2ccc(NO)nc2=O)[C@H](OC(=O)CCCC(F)(F)F)[C@@H]1OC(=O)CCCC(F)(F)F]

In exemplary embodiments, the compound is selected from:

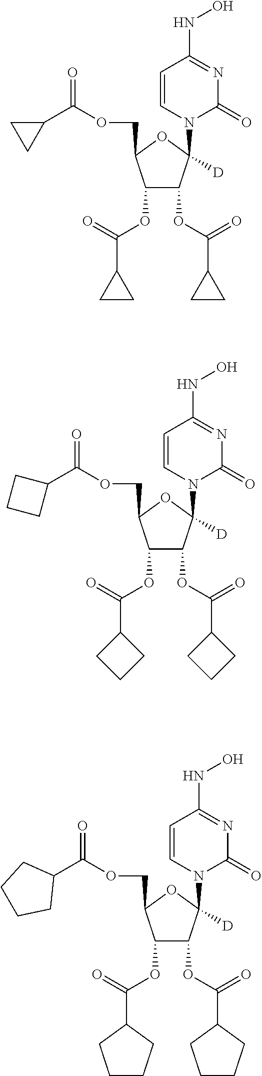 [SMILES: [2H][C@@]1(n2ccc(NO)nc2=O)O[C@H](COC(=O)C2CC2)[C@@H](OC(=O)C2CC2)[C@H]1OC(=O)C1CC1 ; [2H][C@@]1(n2ccc(NO)nc2=O)O[C@H](COC(=O)C2CCC2)[C@@H](OC(=O)C2CCC2)[C@H]1OC(=O)C1CCC1 ; [2H][C@@]1(n2ccc(NO)nc2=O)O[C@H](COC(=O)C2CCCC2)[C@@H](OC(=O)C2CCCC2)[C@H]1OC(=O)C1CCCC1]

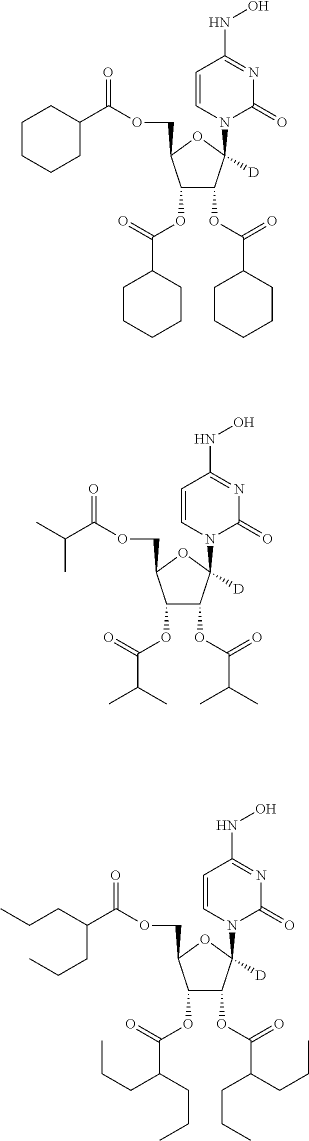 [SMILES: [2H][C@@]1(n2ccc(NO)nc2=O)O[C@H](COC(=O)C2CCCCC2)[C@@H](OC(=O)C2CCCCC2)[C@H]1OC(=O)C1CCCCC1 ; [2H][C@@]1(n2ccc(NO)nc2=O)O[C@H](COC(=O)C(C)C)[C@@H](OC(=O)C(C)C)[C@H]1OC(=O)C(C)C ; [2H][C@@]1(n2ccc(NO)nc2=O)O[C@H](COC(=O)C(CCC)CCC)[C@@H](OC(=O)C(CCC)CCC)[C@H]1OC(=O)C(CCC)CCC]

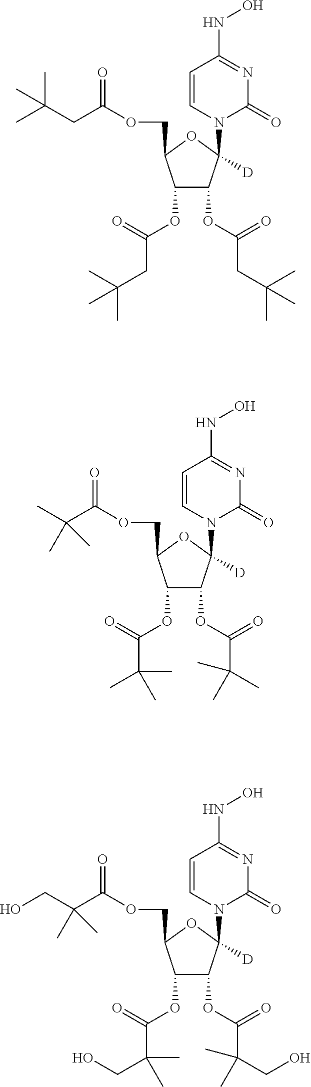

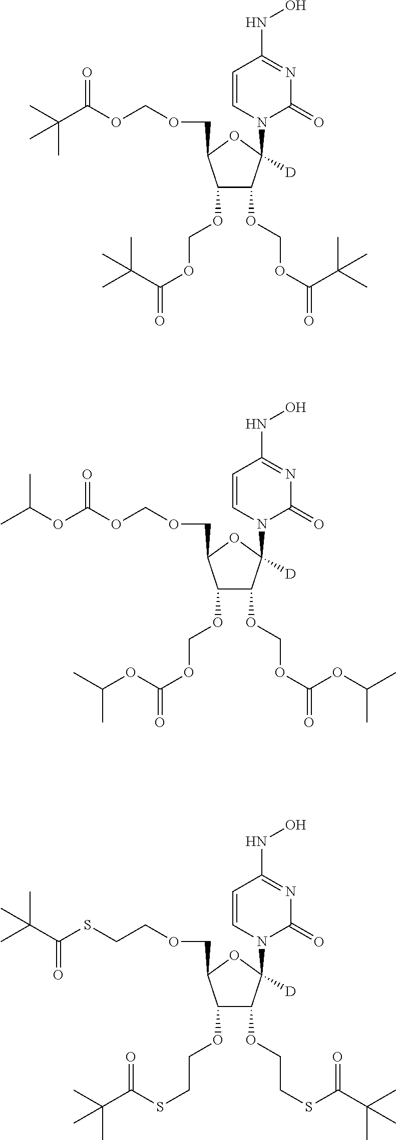

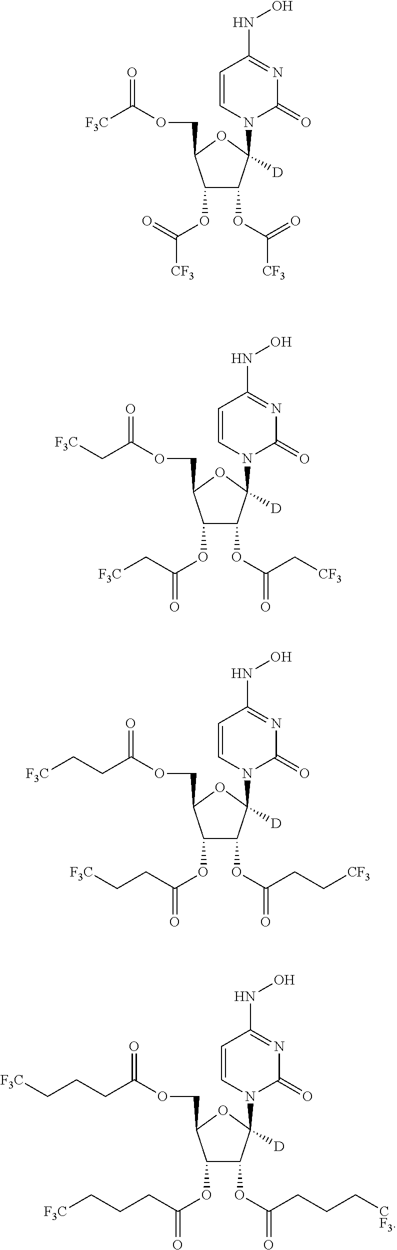 [SMILES: [2H][C@@]1(n2ccc(NO)nc2=O)O[C@H](COC(=O)C(F)(F)F)[C@@H](OC(=O)C(F)(F)F)[C@H]1OC(=O)C(F)(F)F ; [2H][C@@]1(n2ccc(NO)nc2=O)O[C@H](COC(=O)CC(F)(F)F)[C@@H](OC(=O)CC(F)(F)F)[C@H]1OC(=O)CC(F)(F)F ; [2H][C@@]1(n2ccc(NO)nc2=O)O[C@H](COC(=O)CCC(F)(F)F)[C@@H](OC(=O)CCC(F)(F)F)[C@H]1OC(=O)CCC(F)(F)F ; [2H][C@@]1(n2ccc(NO)nc2=O)O[C@H](COC(=O)CCCC(F)(F)F)[C@@H](OC(=O)CCCC(F)(F)F)[C@H]1OC(=O)CCCC(F)(F)F]

In exemplary embodiments, the compound is selected from:

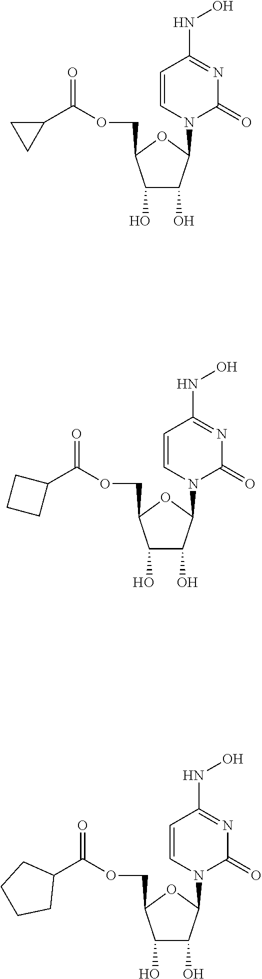 [SMILES: O=C(OC[C@H]1O[C@@H](n2ccc(NO)nc2=O)[C@H](O)[C@@H]1O)C1CC1 ; O=C(OC[C@H]1O[C@@H](n2ccc(NO)nc2=O)[C@H](O)[C@@H]1O)C1CCC1 ; O=C(OC[C@H]1O[C@@H](n2ccc(NO)nc2=O)[C@H](O)[C@@H]1O)C1CCCC1]

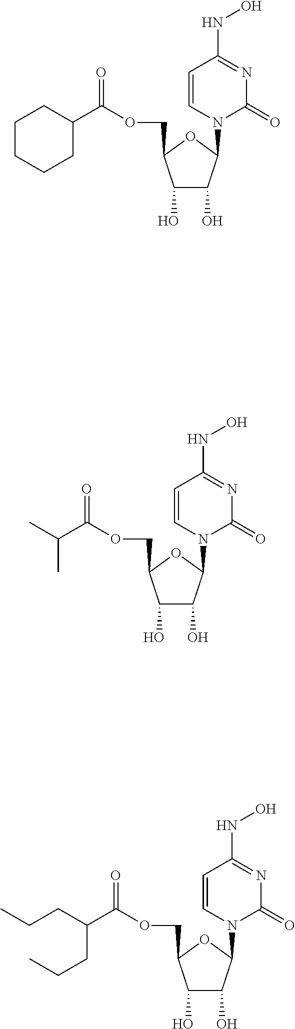 [SMILES: O=C(OC[C@H]1O[C@@H](n2ccc(NO)nc2=O)[C@H](O)[C@@H]1O)C1CCCCC1 ; CC(C)C(=O)OC[C@H]1O[C@@H](n2ccc(NO)nc2=O)[C@H](O)[C@@H]1O ; CCCC(CCC)C(=O)OC[C@H]1O[C@@H](n2ccc(NO)nc2=O)[C@H](O)[C@@H]1O]

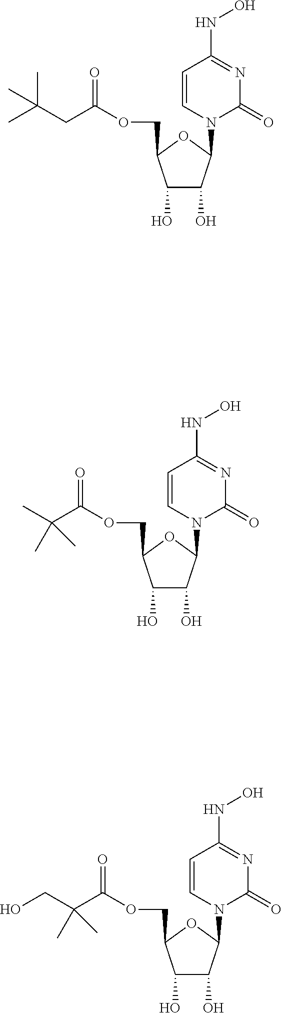 [SMILES: CC(C)(C)CC(=O)OC[C@H]1O[C@@H](n2ccc(NO)nc2=O)[C@H](O)[C@@H]1O ; CC(C)(C)C(=O)OC[C@H]1O[C@@H](n2ccc(NO)nc2=O)[C@H](O)[C@@H]1O ; CC(C)(CO)C(=O)OC[C@H]1O[C@@H](n2ccc(NO)nc2=O)[C@H](O)[C@@H]1O]

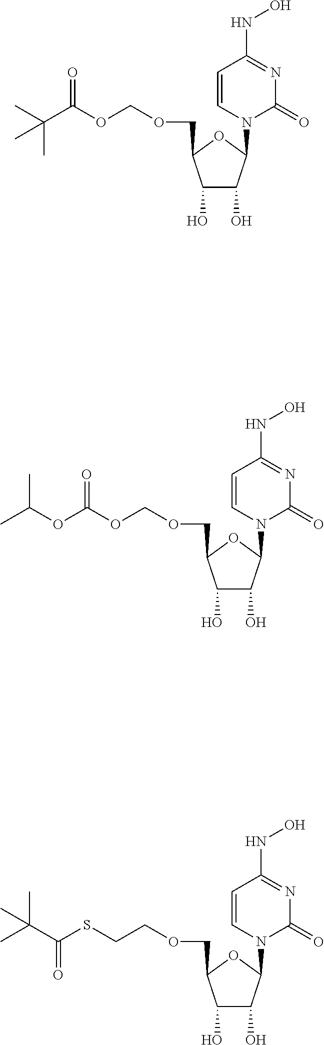 [SMILES: CC(C)(C)C(=O)OCOC[C@H]1O[C@@H](n2ccc(NO)nc2=O)[C@H](O)[C@@H]1O ; CC(C)OC(=O)OCOC[C@H]1O[C@@H](n2ccc(NO)nc2=O)[C@H](O)[C@@H]1O ; CC(C)(C)C(=O)SCCOC[C@H]1O[C@@H](n2ccc(NO)nc2=O)[C@H](O)[C@@H]1O]

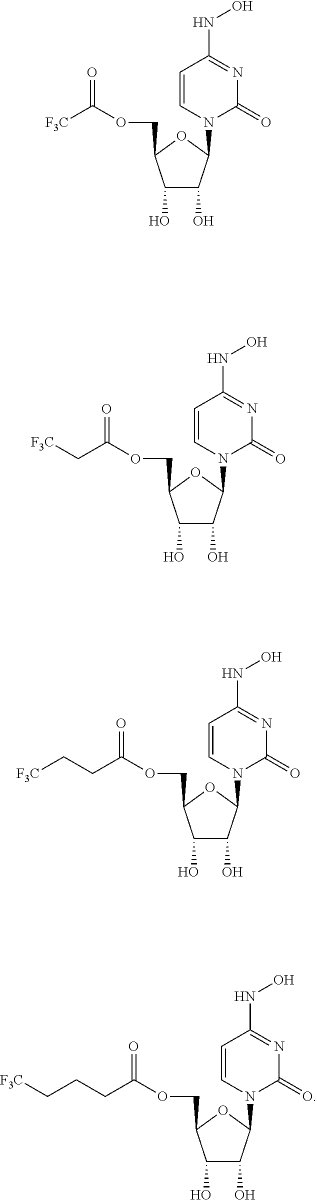 [SMILES: O=C(OC[C@H]1O[C@@H](n2ccc(NO)nc2=O)[C@H](O)[C@@H]1O)C(F)(F)F ; O=C(CC(F)(F)F)OC[C@H]1O[C@@H](n2ccc(NO)nc2=O)[C@H](O)[C@@H]1O ; O=C(CCC(F)(F)F)OC[C@H]1O[C@@H](n2ccc(NO)nc2=O)[C@H](O)[C@@H]1O ; O=C(CCCC(F)(F)F)OC[C@H]1O[C@@H](n2ccc(NO)nc2=O)[C@H](O)[C@@H]1O]

In exemplary embodiments, the compound is selected from:

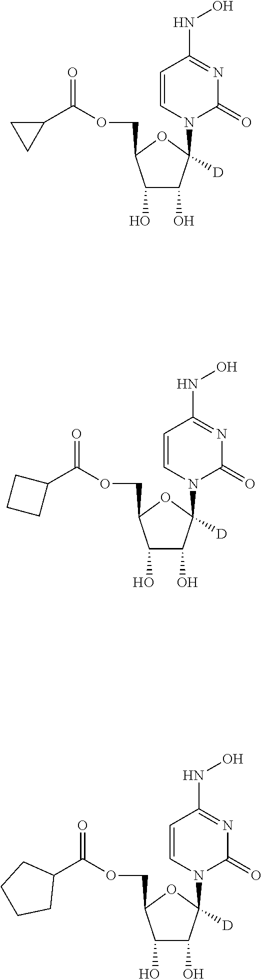 [SMILES: [2H][C@@]1(n2ccc(NO)nc2=O)O[C@H](COC(=O)C2CC2)[C@@H](O)[C@H]1O ; [2H][C@@]1(n2ccc(NO)nc2=O)O[C@H](COC(=O)C2CCC2)[C@@H](O)[C@H]1O ; [2H][C@@]1(n2ccc(NO)nc2=O)O[C@H](COC(=O)C2CCCC2)[C@@H](O)[C@H]1O]

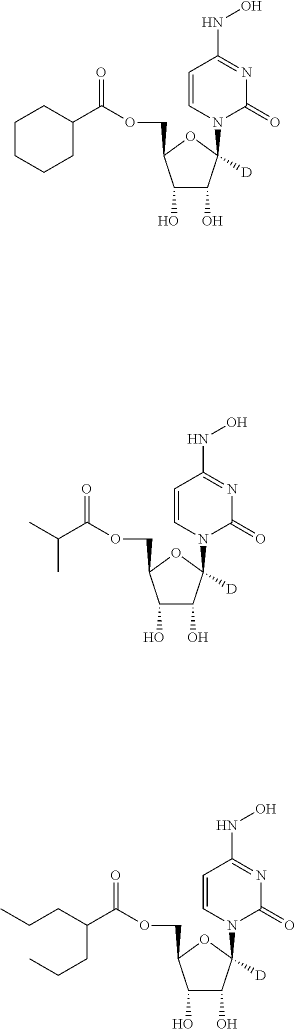 [SMILES: [2H][C@@]1(n2ccc(NO)nc2=O)O[C@H](COC(=O)C2CCCCC2)[C@@H](O)[C@H]1O ; [2H][C@@]1(n2ccc(NO)nc2=O)O[C@H](COC(=O)C(C)C)[C@@H](O)[C@H]1O ; [2H][C@@]1(n2ccc(NO)nc2=O)O[C@H](COC(=O)C(CCC)CCC)[C@@H](O)[C@H]1O]

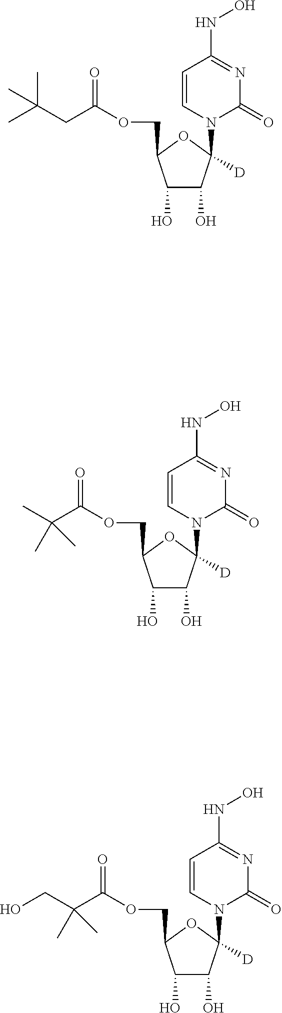 [SMILES: [2H][C@@]1(n2ccc(NO)nc2=O)O[C@H](COC(=O)CC(C)(C)C)[C@@H](O)[C@H]1O ; [2H][C@@]1(n2ccc(NO)nc2=O)O[C@H](COC(=O)C(C)(C)C)[C@@H](O)[C@H]1O ; [2H][C@@]1(n2ccc(NO)nc2=O)O[C@H](COC(=O)C(C)(C)CO)[C@@H](O)[C@H]1O]

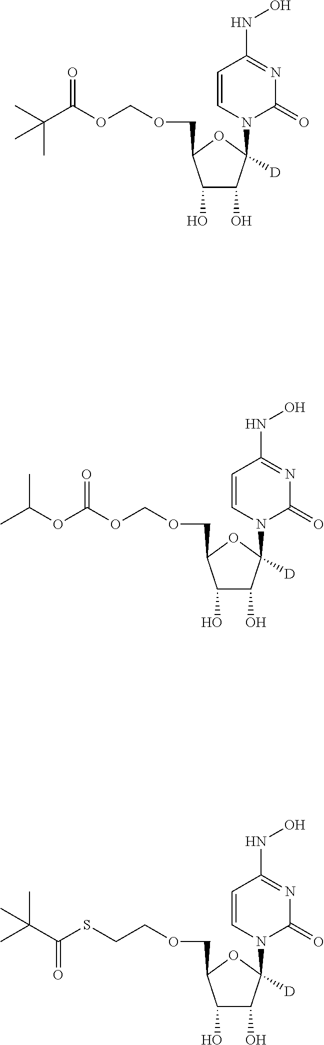 [SMILES: [2H][C@@]1(n2ccc(NO)nc2=O)O[C@H](COCOC(=O)C(C)(C)C)[C@@H](O)[C@H]1O ; [2H][C@@]1(n2ccc(NO)nc2=O)O[C@H](COCOC(=O)OC(C)C)[C@@H](O)[C@H]1O ; [2H][C@@]1(n2ccc(NO)nc2=O)O[C@H](COCCSC(=O)C(C)(C)C)[C@@H](O)[C@H]1O]

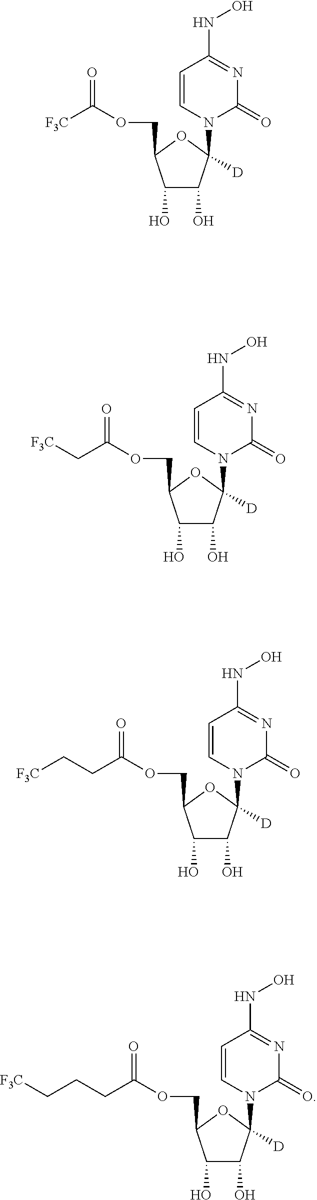 [SMILES: [2H][C@@]1(n2ccc(NO)nc2=O)O[C@H](COC(=O)C(F)(F)F)[C@@H](O)[C@H]1O ; [2H][C@@]1(n2ccc(NO)nc2=O)O[C@H](COC(=O)CC(F)(F)F)[C@@H](O)[C@H]1O ; [2H][C@@]1(n2ccc(NO)nc2=O)O[C@H](COC(=O)CCC(F)(F)F)[C@@H](O)[C@H]1O ; [2H][C@@]1(n2ccc(NO)nc2=O)O[C@H](COC(=O)CCCC(F)(F)F)[C@@H](O)[C@H]1O]

In certain examples, the disclosure relates to compounds of Formula XXI,

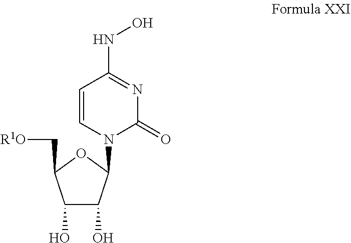

or a tautomer thereof, or a pharmaceutical salt or physiological salt thereof, wherein R¹ is H,

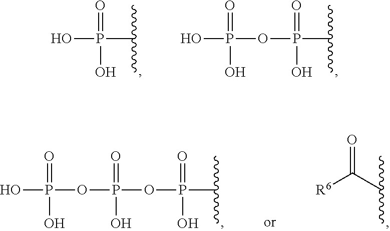

and R⁶ is alkyl or carbocyclyl. In particular instances, R⁶ is selected from C₃-C₆ cycloalkyl, C₃-C₇ n-alkyl, and C₃-C₇ branched alkyl. In more particular instances, R⁶ is isopropyl.

### Methods of Use

In certain embodiments, the disclosure relates to methods of treating or preventing a viral infection, comprising administering an effective amount of a compound or pharmaceutical composition disclosed herein to a subject in need thereof.

In certain embodiments, the disclosure relates to methods of treating or preventing a viral infection in the central nervous system (CNS), comprising administering an effective amount of a compound or pharmaceutical composition disclosed herein to a subject in need thereof.

In certain embodiments, the disclosure relates to methods of treating or preventing a coronavirus infection in the CNS, comprising administering an effective amount of a compound or pharmaceutical composition disclosed herein to a subject in need thereof.

In certain embodiments, the disclosure relates to methods of treating or preventing a SARS-CoV-2 infection in the CNS, comprising administering an effective amount of a compound or pharmaceutical composition disclosed herein to a subject in need thereof.

In certain embodiments, the disclosure relates to methods of treating or preventing a viral infection in the CNS, comprising administering an effective amount of EIDD-1931 to a subject in need thereof.

In certain embodiments, the disclosure relates to methods of treating or preventing a coronavirus infection in the CNS, comprising administering an effective amount of EIDD-1931 to a subject in need thereof.

In certain embodiments, the disclosure relates to methods of treating or preventing a SARS-CoV-2 infection in the CNS, comprising administering an effective amount of EIDD-1931 to a subject in need thereof.

In certain embodiments, the disclosure relates to methods of treating or preventing a viral infection in the CNS comprising administering an effective amount of EIDD-2801 to a subject in need thereof.

In certain embodiments, the disclosure relates to methods of treating or preventing a coronavirus infection in the CNS comprising administering an effective amount of EIDD-2801 to a subject in need thereof.

In certain embodiments, the disclosure relates to methods of treating or preventing a SARS-CoV-2 infection in the CNS comprising administering an effective amount of EIDD-2801 to a subject in need thereof.

In certain embodiments, the disclosure relates to methods of treating or preventing a viral infection in the CNS, comprising administering an effective amount of EIDD-2801 to a subject in need thereof.

In certain embodiments, the disclosure relates to methods of treating or preventing a coronavirus infection in the CNS, comprising administering an effective amount of EIDD-2801 to a subject in need thereof.

In certain embodiments, the disclosure relates to methods of treating or preventing a SARS-CoV-2 infection in the CNS, comprising administering an effective amount of EIDD-2801 to a subject in need thereof.

In certain embodiments, the disclosure relates to methods of treating or preventing a viral infection in the CNS, comprising administering an effective amount of EIDD-2898 to a subject in need thereof.

In certain embodiments, the disclosure relates to methods of treating or preventing a coronavirus infection in the CNS, comprising administering an effective amount of EIDD-2898 to a subject in need thereof.

In certain embodiments, the disclosure relates to methods of treating or preventing a SARS-CoV-2 infection in the CNS, comprising administering an effective amount of EIDD-2898 to a subject in need thereof.

In certain embodiments, the disclosure relates to methods of treating or preventing neurological signs of disease caused by a viral infection in the CNS, comprising administering an effective amount of a compound or pharmaceutical composition disclosed herein to a subject in need thereof.

In certain embodiments, the disclosure relates to methods of treating or preventing neurological signs of disease caused by a coronavirus infection in the CNS, comprising administering an effective amount of a compound or pharmaceutical composition disclosed herein to a subject in need thereof.

In certain embodiments, the disclosure relates to methods of treating or preventing neurological signs of disease caused by a SARS-CoV-2 infection in the CNS, comprising administering an effective amount of a compound or pharmaceutical composition disclosed herein to a subject in need thereof.

In certain embodiments, the disclosure relates to methods of treating or preventing neurological signs of disease caused by a viral infection in the CNS, comprising administering an effective amount of EIDD-1931 to a subject in need thereof.

In certain embodiments, the disclosure relates to methods of treating or preventing neurological signs of disease caused by a coronavirus infection in the CNS, comprising administering an effective amount of EIDD-1931 to a subject in need thereof.

In certain embodiments, the disclosure relates to methods of treating or preventing neurological signs of disease caused by a SARS-CoV-2 infection in the CNS, comprising administering an effective amount of EIDD-1931 to a subject in need thereof.

In certain embodiments, the disclosure relates to methods of treating or preventing neurological signs of disease caused by a viral infection in the CNS, comprising administering an effective amount of EIDD-2801 to a subject in need thereof.

In certain embodiments, the disclosure relates to methods of treating or preventing neurological signs of disease caused by a coronavirus infection in the CNS, comprising administering an effective amount of EIDD-2801 to a subject in need thereof.

In certain embodiments, the disclosure relates to methods of treating or preventing neurological signs of disease caused by a SARS-CoV-2 infection in the CNS, comprising administering an effective amount of EIDD-2801 to a subject in need thereof.

In certain embodiments, the disclosure relates to methods of treating or preventing neurological signs of disease caused by a viral infection in the CNS, comprising administering an effective amount of EIDD-2801 to a subject in need thereof.

In certain embodiments, the disclosure relates to methods of treating or preventing neurological signs of disease caused by a coronavirus infection in the CNS, comprising administering an effective amount of EIDD-2801 to a subject in need thereof.

In certain embodiments, the disclosure relates to methods of treating or preventing neurological signs of disease caused by a SARS-CoV-2 infection in the CNS, comprising administering an effective amount of EIDD-2801 to a subject in need thereof.

In certain embodiments, the disclosure relates to methods of treating or preventing neurological signs of disease caused by a viral infection in the CNS, comprising administering an effective amount of EIDD-2898 to a subject in need thereof.

In certain embodiments, the disclosure relates to methods of treating or preventing neurological signs of disease caused by a coronavirus infection in the CNS, comprising administering an effective amount of EIDD-2898 to a subject in need thereof.

In certain embodiments, the disclosure relates to methods of treating or preventing neurological signs of disease caused by a SARS-CoV-2 infection in the CNS, comprising administering an effective amount of EIDD-2898 to a subject in need thereof.

In certain embodiments, the compound is administered by inhalation through the lungs.

In some embodiments, the subject is at risk of, exhibiting symptoms of, or diagnosed with infection by human coronavirus, SARS coronavirus, MERS coronavirus, or 2019-nCoV.

In certain embodiments, the subject is diagnosed with gastroenteritis, acute respiratory disease, severe acute respiratory syndrome, post-viral fatigue syndrome, viral hemorrhagic fevers, acquired immunodeficiency syndrome, or hepatitis.

### Formulations

In exemplary embodiments, a pharmaceutical composition comprises a pharmaceutically acceptable excipient, such as a pharmaceutically acceptable carrier, and an exemplary compound described herein.

In certain exemplary embodiments, the pharmaceutical composition comprises, or is in the form of, a pharmaceutically acceptable salt, as generally described below. Some, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the exemplary compounds contain an acidic group as well as a basic group, the compounds can form internal salts, which can also be used in the compositions and methods described herein. When an exemplary compound contains a hydrogen-donating heteroatom (e.g., NH), salts are contemplated to cover isomers formed by transfer of said hydrogen atom to a basic group or atom within the molecule.

Pharmaceutically acceptable salts of the exemplary compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases can also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

Physiologically acceptable salts of the exemplary compounds are those that are formed internally in a subject administered compound for the treatment or prevention of disease. Suitable salts include those of lithium, sodium, potassium, magnesium, calcium, manganese, bile salts.

The exemplary compounds can be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a subject, cleaves to form a free hydroxyl group. Examples of prodrugs include, but are not limited to, esters, optionally substituted esters, branched esters, optionally substituted branched esters, carbonates, optionally substituted carbonates, carbamates, optionally substituted carbamates, thioesters, optionally substituted thioesters, branched thioesters, optionally substituted branched thioesters, thiocarbonates, optionally substituted thiocarbonates, sulfenyl thiocarbonates, optionally substituted sulfenyl thiocarbonates, 2-hydroxypropanoate ester, optionally substitute 2-hydroxypropanoate ester, S-thiocarbonate, optionally substituted S-thiocarbonate, dithiocarbonates, optionally substituted dithiocarbonates, thiocarbamates, optionally substituted thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, sulfenyl, optionally substituted sulfenyl, sulfinyl, sulfonyl, sulfite, sulfate, sulfonamide, imidate, optionally substituted imidate, hydrazonate, optionally substituted hydrazonate, oximyl, optionally substituted oximyl, imidinyl, optionally substituted imidinyl, imidyl, optionally substituted imidyl, aminal, optionally substituted aminal, hemiaminal, optionally substituted hemiaminal, acetal, optionally substituted acetal, hemiacetal, optionally substituted hemiacetal, carbonimidate, optionally substituted carbonimidate, thiocarbonimidate, optionally substituted thiocarbonimidate, carbonimidyl, optionally substituted carbonimidyl, carbamimidate, optionally substituted carbamimidate, carbamimidyl, optionally substituted carbamimidyl, thioacetal, optionally substituted thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, optionally substituted bis-(acyloxybenzyl)esters, (acyloxybenzyl)esters, optionally substituted (acyloxybenzyl)esters, or BAB-esters, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

In exemplary embodiments, the pharmaceutical composition comprises an effective amount of an exemplary compound and a pharmaceutically acceptable carrier. Generally, for pharmaceutical use, the compounds can be formulated as a pharmaceutical preparation comprising at least one compound and at least one pharmaceutically acceptable carrier, diluent or excipient and/or adjuvant, and optionally one or more further pharmaceutically active compounds. The preparations can be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is again made to U.S. Pat. Nos. 6,372,778, 6,369,086, 6,369,087 and 6,372,733 and the further references mentioned above, as well as to the standard handbooks, such as the latest edition of Remington's Pharmaceutical Sciences. The disclosed pharmaceutical compositions can be in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain from 1 and 1000 mg, and usually from 5 and 500 mg, of the at least one compound of the disclosure, e.g., about 10, 25, 50, 100, 200, 300, 400, 800 mg per unit dosage.

The compounds can be administered by a variety of routes including the oral, ocular, rectal, transdermal, subcutaneous, intravenous, intramuscular or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be from 0.01 to 1000 mg per kilogram body weight of the patient per day, more often from 0.1 and 500 mg, such as from 1 and 250 mg, for example about 5, 10, 20, 50, 100, 150, 200 or 250 mg, per kilogram body weight of the patient per day, which can be administered as a single daily dose, divided over one or more daily doses. The amount(s) to be administered, the route of administration and the further treatment regimen can be determined by the treating clinician, depending on factors such as the age, gender and general condition of the patient and the nature and severity of the disease/symptoms to be treated. Reference is again made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

Depending upon the manner of introduction, the compounds described herein can be formulated in a variety of ways. Formulations containing one or more compounds can be prepared in various pharmaceutical forms, such as granules, tablets, capsules, suppositories, powders, controlled release formulations, suspensions, emulsions, creams, gels, ointments, salves, lotions, or aerosols and the like. In certain embodiments, the formulations are employed in solid dosage forms suitable for simple, and preferably oral, administration of precise dosages. Solid dosage forms for oral administration include, but are not limited to, tablets, soft or hard gelatin or non-gelatin capsules, and caplets. However, liquid dosage forms, such as solutions, syrups, suspension, shakes, etc. can also be utilized. In another embodiment, the formulation is administered topically. Suitable topical formulations include, but are not limited to, lotions, ointments, creams, and gels. In one embodiment, the topical formulation is a gel. In another embodiment, the formulation is administered intranasally.

Formulations containing one or more of the compounds described herein can be prepared using a pharmaceutically acceptable carrier composed of materials that are considered safe and effective and can be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition, which can include plasticizers, pigments, colorants, stabilizing agents, and glidants. Delayed release, extended release, and/or pulsatile release dosage formulations can be prepared as described in standard references such as “Pharmaceutical dosage form tablets”, eds. Liberman et al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, methacrylic resins that are commercially available under the trade name EUDRAGIT™ (Roth Pharma, Westerstadt, Germany), zein, shellac, and polysaccharides.

Additionally, the coating material can contain conventional carriers such as plasticizers, pigments, colorants, glidants, stabilization agents, pore formers, and surfactants.

Optional pharmaceutically acceptable excipients present in the drug-containing tablets, beads, granules, or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose, and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid, and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums, and cross linked polymers, such as cross-linked PVP (Polyplasdone XL from GAF Chemical Corp).

Stabilizers are used to inhibit or retard drug decomposition reactions which include, by way of example, oxidative reactions.

Surfactants can be anionic, cationic, amphoteric, or nonionic surface active agents. Suitable anionic surfactants include, but are not limited to, those containing carboxylate, sulfonate, and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER™ 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles can also contain minor amount of nontoxic auxiliary substances such as wetting or emulsifying agents, dyes, pH buffering agents, or preservatives.

The concentration of the exemplary compound to pharmaceutically acceptable carrier, excipient and/or other substances can vary from about 0.5 to about 100 wt. % (weight percent). For oral use, the pharmaceutical composition can generally contain from about 5 to about 100% by weight of the active material. For other uses, the pharmaceutical composition can generally have from about 0.5 to about 50 wt. % of the active material.

The compositions described herein can be formulated for modified or controlled release. Examples of controlled release dosage forms include extended release dosage forms, delayed release dosage forms, pulsatile release dosage forms, and combinations thereof.

The extended release formulations are generally prepared as diffusion or osmotic systems, for example, as described in “Remington—The science and practice of pharmacy” (20th ed., Lippincott Williams & Wilkins, Baltimore, MD, 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and CARBOPOL™ 934 polyethylene oxides and mixtures thereof. Fatty compounds include, but are not limited to, various waxes, such as carnauba wax and glyceryl tristearate, and wax-type substances including hydrogenated castor oil, hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the trade name EUDRAGIT™. In further preferred embodiments, the acrylic polymer comprises a mixture of two acrylic resin lacquers commercially available from Rohm Pharma under the trade names EUDRAGIT™ RL30D and EUDRAGIT™ RS30D, respectively. EUDRAGIT™ RL30D and EUDRAGIT™ RS30D are copolymers of acrylic and methacrylic esters with a low content of quaternary ammonium groups, the molar ratio of ammonium groups to the remaining neutral (meth)acrylic esters being 1:20 in EUDRAGIT™ RL30D and 1:40 in EUDRAGIT™ RS30D. The mean molecular weight is about 150,000. EUDRAGIT™ S-100 and EUDRAGIT™ L-100 are also preferred. The code designations RL (high permeability) and RS (low permeability) refer to the permeability properties of these agents. EUDRAGIT™ RL/RS mixtures are insoluble in water and in digestive fluids. However, multiparticulate systems formed to include the same are swellable and permeable in aqueous solutions and digestive fluids.

The polymers described above such as EUDRAGIT™ RL/RS can be mixed together in any desired ratio in order to ultimately obtain a sustained-release formulation having a desirable dissolution profile. Desirable sustained-release multiparticulate systems can be obtained, for instance, from 100% EUDRAGIT™ RL, 50% EUDRAGIT™ RL and 50% EUDRAGIT™ RS, and 10% EUDRAGIT™ RL and 90% EUDRAGIT™ RS. One skilled in the art will recognize that other acrylic polymers can also be used, such as, for example, EUDRAGIT™ L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules, etc. An immediate release portion can be added to the extended release system by means of either applying an immediate release layer on top of the extended release core using a coating or compression process or in a multiple unit system, such as a capsule containing extended and immediate release beads.

Extended release tablets containing hydrophilic polymers are prepared by techniques commonly known in the art such as direct compression, wet granulation, or dry granulationprocesses. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours, and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium stearate, calcium stearate, stearic acid, and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed release formulations are created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition can be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and can be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the trade name EUDRAGIT™ (Rohm Pharma; Westerstadt, Germany), including EUDRAGIT™ L30D-55 and L100-55 (soluble at pH 5.5 and above), EUDRAGIT™ L-100 (soluble at pH 6.0 and above), EUDRAGIT™ S (soluble at pH 7.0 and above, as a result of a higher degree of esterification), and EUDRAGIT™ NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein; and shellac. Combinations of different coating materials can also be used. Multi-layer coatings using different polymers can also be applied.

The preferred coating weights for particular coating materials can be readily determined by those skilled in the art by evaluating individual release profiles for tablets, beads and granules prepared with different quantities of various coating materials. It is the combination of materials, method, and form of application that produce the desired release characteristics, which one can determine only from the clinical studies.

The coating composition can include conventional additives, such as plasticizers, pigments, colorants, stabilizing agents, glidants, etc. A plasticizer is normally present to reduce the fragility of the coating and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides. A stabilizing agent is preferably used to stabilize particles in the dispersion. Typical stabilizing agents are nonionic emulsifiers such as sorbitan esters, polysorbates, and polyvinylpyrrolidone. Glidants are recommended to reduce sticking effects during film formation and drying and will generally represent approximately 25 wt. % to 100 wt. % of the polymer weight in the coating solution. One effective glidant is talc. Other glidants such as magnesium stearate and glycerol monostearates can also be used. Pigments such as titanium dioxide can also be used. Small quantities of an anti-foaming agent, such as a silicone (e.g., simethicone), can also be added to the coating composition.

The formulation can provide pulsatile delivery of the one or more compounds. By “pulsatile” is meant that a plurality of drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses can be designed. The duration of the nearly drug release-free time interval will vary depending upon the dosage form design, e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In one embodiment, the pulsatile release profile is achieved with dosage forms that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the active agent in a controlled release polymer matrix. Each dosage unit can comprise a compressed or molded tablet, wherein each tablet within the capsule provides a different drug release profile. For dosage forms mimicking a twice a day dosing profile, a first tablet releases drug substantially immediately following ingestion of the dosage form, while a second tablet releases drug approximately 3 hours to less than 14 hours following ingestion of the dosage form. For dosage forms mimicking a three times daily dosing profile, a first tablet releases drug substantially immediately following ingestion of the dosage form, a second tablet releases drug approximately 3 hours to less than 10 hours following ingestion of the dosage form, and the third tablet releases drug at least 5 hours to approximately 18 hours following ingestion of the dosage form. It is possible that the dosage form includes more than three tablets. While the dosage form will not generally include more than a third tablet, dosage forms housing more than three tablets can be utilized.

Alternatively, each dosage unit in the capsule can comprise a plurality of drug-containing beads, granules or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that can or can not include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles can be formulated to provide immediate release, beads and granules are generally employed to provide delayed release.

In one embodiment, the compound is formulated for topical administration. Suitable topical dosage forms include lotions, creams, ointments, and gels. A “gel” is a semisolid system containing a dispersion of the active agent, i.e., compound, in a liquid vehicle that is rendered semisolid by the action of a thickening agent or polymeric material dissolved or suspended in the liquid vehicle. The liquid can include a lipophilic component, an aqueous component or both. Some emulsions can be gels or otherwise include a gel component. Some gels, however, are not emulsions because they do not contain a homogenized blend of immiscible components. Methods for preparing lotions, creams, ointments, and gels are well known in the art.

### Combination Therapies

The compound described herein can be administered adjunctively with other active compounds. These compounds include but are not limited to analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics, anti-narcoleptics, and antiviral agents. In a particular embodiment, the antiviral agent is a non-CNS targeting antiviral compound. “Adjunctive administration”, as used herein, means the compound can be administered in the same dosage form or in separate dosage forms with one or more other active agents. The additional active agent(s) can be formulated for immediate release, controlled release, or combinations thereof.

Specific examples of compounds that can be adjunctively administered with the compounds include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carfilzomib, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, Polygonum cuspidatum, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone, and isomers, salts, and combinations thereof.

In certain embodiments, the exemplary compounds and pharmaceutical compositions can be administered in combination with another antiviral agent(s) such as abacavir, acyclovir, acyclovir, adefovir, amantadine, amprenavir, ampligen, arbidol, AT-527, atazanavir, atripla, balapiravir, BCX4430/Galidesivir, boceprevir, cidofovir, combivir, daclatasvir, darunavir, dasabuvir, delavirdine, didanosine, ocosanol, edoxudine, efavirenz, emtricitabine, enfuvirtide, entecavir, famciclovir, favipiravir, fomivirsen, fosamprenavir, foscarnet, fosfonet, ganciclovir, GS-5734/Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, or zidovudine, and combinations thereof.

In certain embodiments, the exemplary compounds and pharmaceutical compositions can be administered in combination with another agent(s) such as chloroquine, chloroquine phosphate, hydroxychloroquine, hydroxychloroquine sulfate, Ampligen, APNO1, Ganovo, IFX-1, BXT-25, CYNK-001, Tocilizumab, Leronlimab, Ii-key, COVID-19 S-Trimer, Camrelizumab, thymosin, Brilacidin, INO-4800, Prezcobix, cobicistat, mRNA-1273, Arbidol, umifenovir, REGN3048, REGN3051, TNX-1800, fingolimod, methylprednisolone, nitazoxanide, benzopurpin B, C-467929, C-473872, NSC-306711, N-65828, C-21, CGP-42112A, L-163491, xanthoangelol, or bevacizumab, and combinations thereof.

In certain embodiments, the exemplary compounds and pharmaceutical compositions disclosed herein can be administered in combination with any of the compounds disclosed in: WO2003090690A2, WO2003090690A3, WO2003090691A2, WO2003090691A3, WO2004005286A2, WO2004005286A3, WO2004006843A2, WO2004006843A3, WO2004031224A2, WO2004031224A3, WO2004035576A2, WO2004035576A3, WO2004035577A2, WO2004035577A3, WO2004050613A2, WO2004050613A3, WO2004064845A1, WO2004064846A1, WO2004096286A2, WO2004096286A3, WO2004096287A2, WO2004096287A3, WO2004096818A2, WO2004096818A3, WO2004100960A2, WO2005002626A2, WO2005002626A3, WO2005012324A2, WO2005012324A3, WO2005028478A1, WO2005039552A2, WO2005039552A3, WO2005042772A1, WO2005042773A1, WO2005047898A2, WO2005047898A3, WO2005063744A2, WO2005063744A3, WO2005063751A1, WO2005064008A1, WO2005064008A9, WO2005066189A1, WO2005070901A2, WO2005070901A3, WO2005072748A1, WO2005117904A2, WO2005117904A3, WO2006015261A2, WO2006015261A3, WO2006017044A2, WO2006017044A3, WO2006020276A2, WO2006020276A3, WO2006033703A1, WO2006047661A2, WO2006047661A3, WO2006069193A2, WO2006069193A3, WO2006091905A1, WO2006110157A2, WO2006110157A3, WO2006110157A9, WO2006125048A2, WO2006125048A3, WO2007009109A2, WO2007009109A3, WO2007011658A1, WO2007014174A2, WO2007014174A3, WO2007014352A2, WO2007014352A3, WO2007079260A1, WO2007079260A9, WO2007126812A2, WO2007126812A3, WO2008003149A2, WO2008003149A3, WO2008005519A2, WO2008005519A3, WO2008005542A2, WO2008005542A3, WO2008005555A1, WO2008009076A2, WO2008009076A3, WO2008009077A2, WO2008009077A3, WO2008009078A2, WO2008009078A3, WO2008009079A2, WO2008009079A3, WO2008010921A2, WO2008010921A3, WO2008011116A2, WO2008011116A3, WO2008011117A2, WO2008011117A3, WO2008013834A1, WO2008016522A2, WO2008016522A3, WO2008077649A1, WO2008077650A1, WO2008077651A1, WO2008100447A2, WO2008100447A3, WO2008103949A1, WO2008133669A2, WO2008133669A3, WO2009005674A2, WO2009005674A3, WO2009005676A2, WO2009005676A3, WO2009005677A2, WO2009005677A3, WO2009005687A1, WO2009005690A2, WO2009005690A3, WO2009005693A1, WO2009006199A1, WO2009006203A1, WO2009009001A1, WO2009009001A9, WO2009088719A1, WO2009105513A2, WO2009105513A3, WO2009132123A1, WO2009132135A1, WO2010002998A1, WO2010005986A1, WO2010011959A1, WO2010075127A1, WO2010077613A1, WO2010080389A1, WO2010093608A1, WO2010132601A1, WO2010151472A1, WO2010151487A1, WO2010151488A1, WO2011005842A1, WO2011011303A1, WO2011031669A1, WO2011031965A1, WO2011035231A1, WO2011049825A1, WO2011079016A1, WO2011088303A1, WO2011088345A1, WO2011106445A1, WO2011139820A1, WO2011143105A1, WO2011143106A1, WO2011146817A1, WO2011150288A1, WO2011156416A1, WO2011156610A2, WO2011156610A3, WO2011156757A1, WO2011163518A1, WO2012003497A1, WO2012003498A1, WO2012012465A1, WO2012012776A1, WO2012037038A1, WO2012039787A1, WO2012039791A1, WO2012068234A2, WO2012068234A3, WO2012068535A1, WO2012078915A1, WO2012087596A1, WO2012088153A1, WO2012088156A1, WO2012088178A1, WO2012138669A1, WO2012138670A1, WO2012142523A2, WO2012142523A3, WO2012145728A1, WO2012151165A1, WO2013006721A1, WO2013006722A1, WO2013006738A1, WO2013010112A1, WO2013025788A1, WO2013040492A2, WO2013040492A3, WO2013066748A1, WO2013075029A1, WO2013082003A1, WO2013090840A1, WO2013090929A1, WO2013096512A1, WO2013096681A1, WO2013103724A1, WO2013103738A1, WO2013106732A1, WO2013115916A1, WO2013116720A1, WO2013116730A1, WO2013138236A1, WO2013158776A1, WO2013159064A1, WO2013173488A1, WO2013173492A1, WO2013185090A1, WO2013185093A1, WO2013185103A1, WO2014008285A1, WO2014028343A1, WO2014055618A1, WO2014070939A1, WO2014074620A1, WO2014100323A1, WO2014100500A1, WO2014110296A1, WO2014110297A1, WO2014110298A1, WO2014134566A2, WO2014134566A3, WO2014145095A1, WO2015023893A1, WO2015069939A1, WO2015084741A2, WO2015084741A3, WO2015099989A1, WO2015100144A1, WO2015108780A1, WO2015120057A1, WO2015130964A1, WO2015130966A1, WO2015179448A1, WO2015191526A2, WO2015191526A3, WO2015191726A1, WO2015191743A1, WO2015191745A1, WO2015191752A1, WO2015191754A2, WO2015191754A3, WO2015196137A1, WO2016007765A1, WO2016018697A1, WO2016028866A1, WO2016033243A1, WO2016033243A9, WO2016036759A1, WO2016096116A1, WO2016096116A1, WO2016105532A1, WO2016105534A1, WO2016105564A1, WO2016106237A1, WO2016141092A1, WO2016161382A1, WO2016168349A1, WO2016186967A1, WO2016205141A1, WO2017004012A1, WO2017004244A1, WO2017035230A1, WO2017048727A1, WO2017049060A1, WO2017059120A1, WO2017059224A2, WO2017059224A3, WO2017083304A1, WO2017106346A2, WO2017106346A3, WO2017106556A1, WO2017184668A1, WO2017184670A2, WO2017184670A3, WO2017205078A1, WO2017205115A1, WO2017223268A1, WO9015065A1, WO9209705A1, WO9307157A1, WO9310820A1, WO9403467A2, WO9403467A3, WO9424144A2, WO9424144A3, WO9507919A1, WO9507920A1, WO9626933A1, WO9817647A1, WO2009114512, WO2014028756, JP5971830, US20160122374, US20170071964, WO2007075145, WO2005021518, WO2007120160, WO2009119167, WO2013049382, WO2018042343, WO2007067515, EP2112164, WO2009128 WO2018115527 963, WO2009128963, WO2008035894, WO2008060331, WO2007044695, CN1911963, CN1903878, WO2006095180, WO2006086561, CN1664100, CN1660912, WO2006051091, WO2006051091, CN1673231, US20060240551, WO2005054469, WO2005060520, US20050106563, US20050069869, WO2005012360, CN1566155, WO2005007671, WO2005058815, WO2017095875, WO200505824, WO2011072487, WO2016180335, WO2004096852, WO2005097165, US20090053173, CN101942026, CN101173275, CN1648249, US20050004063, JP2007043942, WO2005023083, US20060039926, WO2005081716, WO2015081155, WO2010063685, US20070003577, US20060002947, WO2015042373, WO2017070626, WO2018048937A1, WO2019200005A1, WO2020117966A1, or WO2019173310, each of which is incorporated by reference herein for their teachings of exemplary compounds and pharmaceutical compositions that can be administered in combination with any of the compounds disclosed herein.

EIDD-1931 and prodrugs thereof, e.g., EIDD-2801, can be administered in combination with, or formulated with, another antiviral agent(s) such as:
Nucleoside reverse transcriptase inhibitors (NRTIs)
Non-nucleoside reverse transcriptase inhibitors (NNRTIs)
Protease inhibitors (PIs)
Integrase inhibitors (INSTIs)
Fusion inhibitors (FIs)
Chemokine receptor antagonists
Entry inhibitors.

Specific examples of agents include abacavir, acyclovir, acyclovir, adefovir, amantadine, amprenavir, ampligen, arbidol, AT-527, atazanavir, atripla, balapiravir, BCX4430/Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, GS-5734/remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, CD24Fc, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, zidovudine, or chloroquine, chloroquine phosphate, hydroxychloroquine, hydroxychloroquine sulfate, Ampligen, APNO1, Ganovo, IFX-1, BXT-25, CYNK-001, Tocilizumab, Leronlimab, li-key, COVID-19 S-Trimer, Camrelizumab, thymosin, Brilacidin, INO-4800, Prezcobix, cobicistat, mRNA-1273, Arbidol, umifenovir, REGN3048, REGN3051, TNX-1800, fingolimod, methylprednisolone, nitazoxanide, benzopurpin B, C-467929, C-473872, NSC-306711, N-65828, C-21, CGP-42112A, L-163491, xanthoangelol, bevacizumab, polyclonal antibodies derived from patients and monoclonal antibodies (including those antibodies from patients of COVID-19 or monoclonal or polyclonal antibodies that bind SARS-CoV-2), and combinations thereof. In addition, the compounds of this invention can be combined with compounds that are favorable to preventing lung damage associated with COVID-19, including for example anti-IL-6 and TNF inhibitors, specifically including, for example, tocilizumab (Actemra), siltuximab (Sylvant), Tocilizumab, Sarilumab, olokizumab (CDP6038), elsilimomab, BMS-945429 (ALD518), sirukumab (CNTO 136), levilimab (BCD-089), and CPSI-2364 and ALX-0061, ARGX-109, FE301, FM10, infliximab (Remicade), adalimumab (Humira), certolizumab pegol (Cimzia), and golimumab (Simponi), etanercept (Enbrel), CD24Fc, Thalidomide (Immunoprin) and its derivatives lenalidomide (Revlimid) and pomalidomide (Pomalyst, Imnovid), xanthine derivatives (e.g., pentoxifylline) and bupropion and 5-HT, agonist hallucinogens including (R)-DOI, TCB-2, LSD, and LA-SS-Az.

In embodiments, the exemplary compounds and pharmaceutical compositions can be administered in combination with

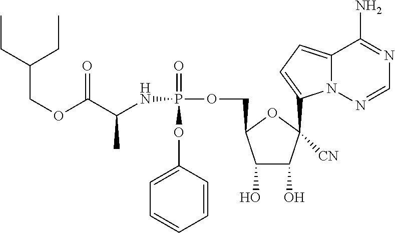 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

and derivatives and prodrugs thereof.

In embodiments,

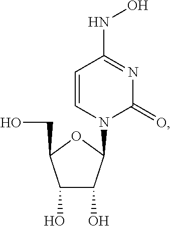 [SMILES: O=c1nc(NO)ccn1[C@@H]1O[C@H](CO)[C@@H](O)[C@H]1O]

and derivatives and prodrugs thereof, can be administered in combination with

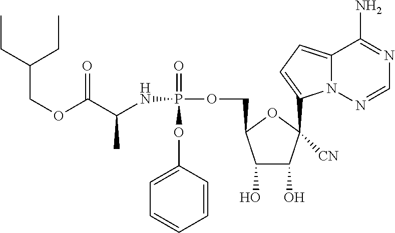 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

and derivatives and prodrugs thereof.

In embodiments,

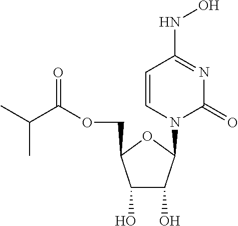 [SMILES: CC(C)C(=O)OC[C@H]1O[C@@H](n2ccc(NO)nc2=O)[C@H](O)[C@@H]1O]

can be administered in combination with

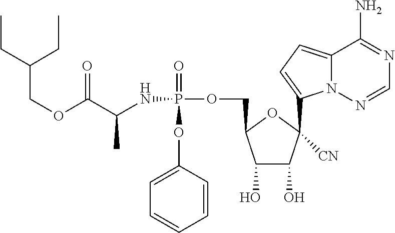 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

and derivatives and prodrugs thereof.

In embodiments,

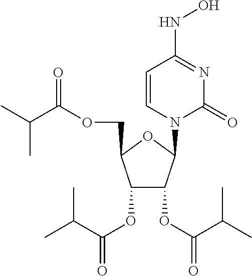 [SMILES: CC(C)C(=O)OC[C@H]1O[C@@H](n2ccc(NO)nc2=O)[C@H](OC(=O)C(C)C)[C@@H]1OC(=O)C(C)C]

can be administered in combination with

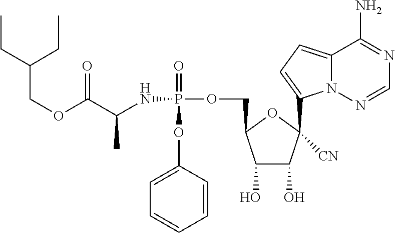 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

and derivatives and prodrugs thereof.

In embodiments,

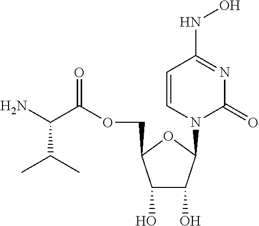 [SMILES: CC(C)[C@H](N)C(=O)OC[C@H]1O[C@@H](n2ccc(NO)nc2=O)[C@H](O)[C@@H]1O]

can be administered in combination with

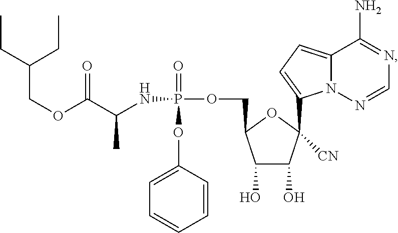 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

and derivatives and prodrugs thereof.

### Specific Embodiments

In specific embodiments, disclosed herein is a pharmaceutical composition for the treatment of 2019nCoV/SARS-CoV-2 infection, comprising a pharmaceutically acceptable excipient and a compound of Formula XXI,

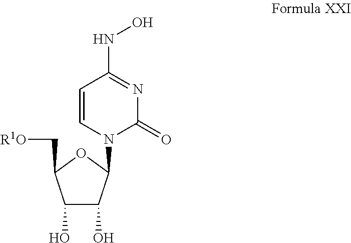

or a tautomer thereof, or a pharmaceutical salt or physiological salt thereof, wherein

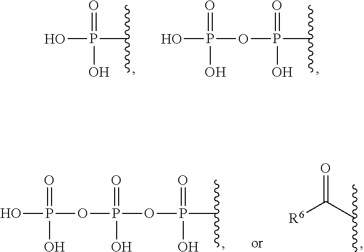

where R⁶ is alkyl or carbocyclyl. In some examples, the compound can have the following structure,

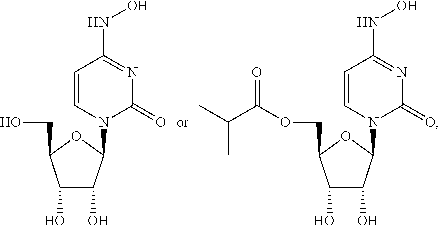

or a tautomer thereof, or pharmaceutical salt or physiological salt thereof.

In another aspect, disclosed is a pharmaceutical composition for the treatment of 2019nCoV/SARS-CoV-2 infection, comprising a pharmaceutically acceptable excipient and a compound of Formula I,

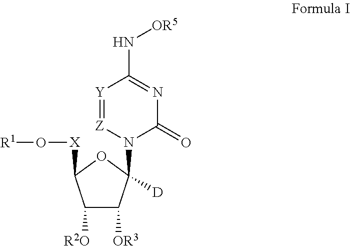

or a tautomer thereof, or a pharmaceutical salt or physiological salt thereof, wherein
X is CH₂, CHCH₃, C(CH₃)₂, CHF, CF₂, or CD₂;
Y is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or carbonyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, hydroxyl, thiol, or carbonyl, wherein R″ is optionally substituted with one or more, the same or different, R¹⁰;
R¹, R², R³, and R⁵ are each independently selected from H, or together with the oxygen to which they are attached form optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R², R³, and R⁵ are optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R″ ^{D};
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In some examples, R¹, R², R³, and R⁵ can be each independently selected from H,

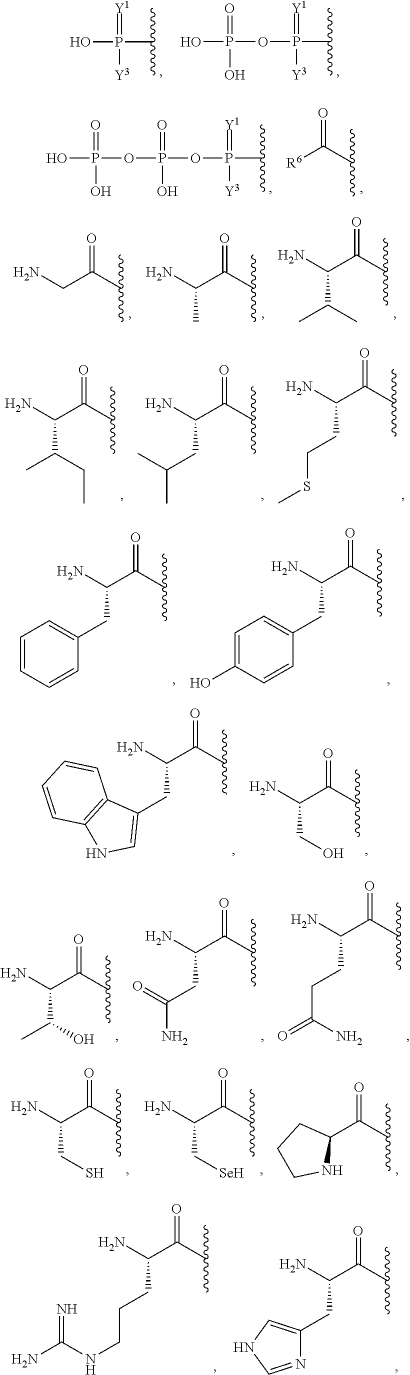

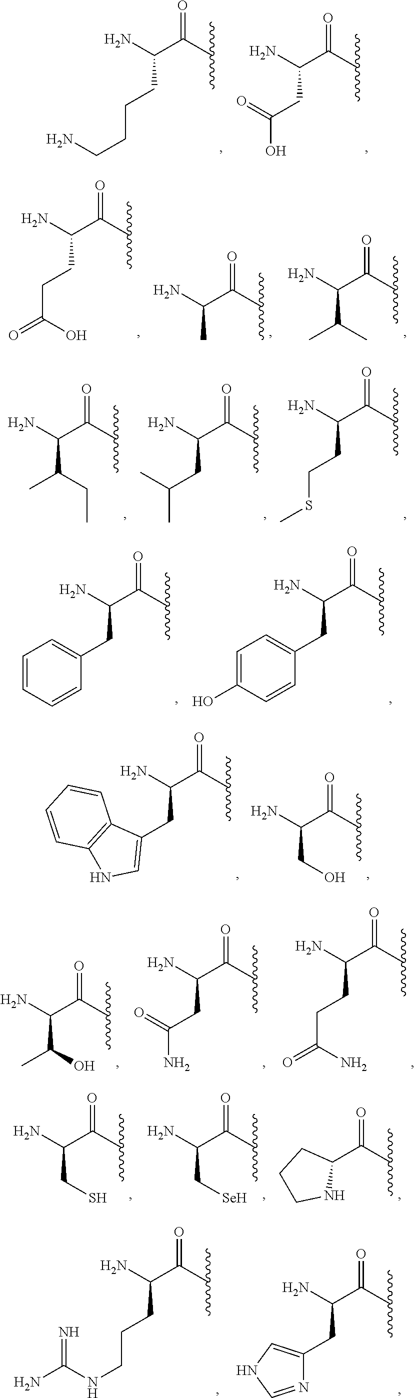

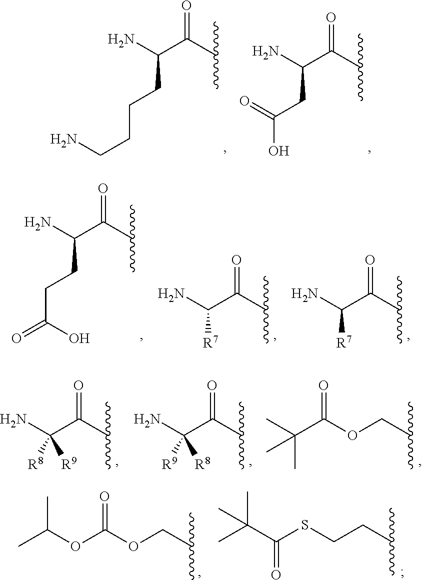

Y is O or S;
Y³ is OH or BH₃⁻M⁺, where M is Li, Na, K, NH₄, (CH₃CH₂)₃NH, (CH₃CH₂CH₂CH₂)₄N;
R⁶ is hydrogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, cyano, or lipid, wherein R⁶ is optionally substituted with one or more, the same or different, R¹⁰;
R⁷ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R⁸, and R⁹ can form a ring with the α-carbon they are attached to and the amino group attached to the α-carbon; and
R⁸ and R⁹ can form a ring with the α-carbon to which they are attached.

In other examples, R¹ is hydrogen,

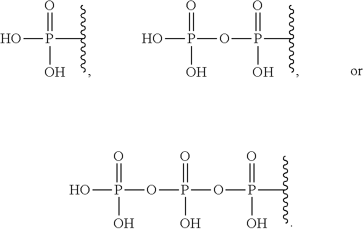 [SMILES: *P(=O)(O)O ; *P(=O)(O)OP(=O)(O)O ; *P(=O)(O)OP(=O)(O)OP(=O)(O)O]

In some examples, R′ is methyl, fluoro, hydroxymethyl, fluoromethyl, difluoromethyl, trifluoromethyl, trideuteromethyl, thiomethyl, carboxylic acid, formyl, vinyl, or ethynyl. In some examples, R″ is methyl, fluoro, hydroxymethyl, fluoromethyl, difluoromethyl, trifluoromethyl, trideuteromethyl, thiomethyl, carboxylic acid, formyl, vinyl, or ethynyl. In some examples, the compound is selected from the following:

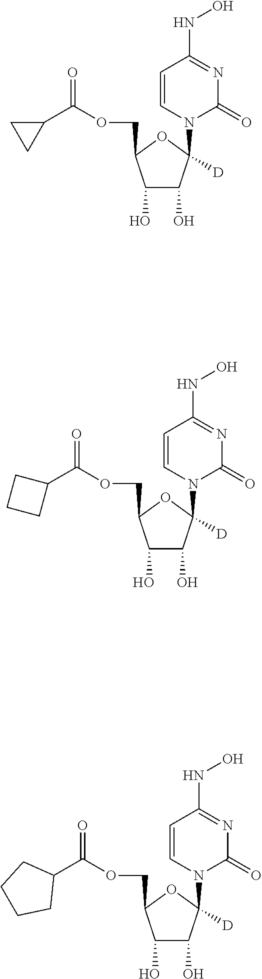 [SMILES: [2H][C@@]1(n2ccc(NO)nc2=O)O[C@H](COC(=O)C2CC2)[C@@H](O)[C@H]1O ; [2H][C@@]1(n2ccc(NO)nc2=O)O[C@H](COC(=O)C2CCC2)[C@@H](O)[C@H]1O ; [2H][C@@]1(n2ccc(NO)nc2=O)O[C@H](COC(=O)C2CCCC2)[C@@H](O)[C@H]1O]

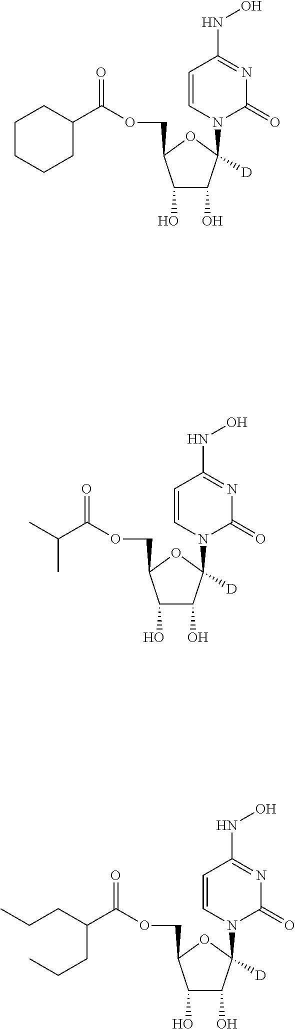 [SMILES: [2H][C@@]1(n2ccc(NO)nc2=O)O[C@H](COC(=O)C2CCCCC2)[C@@H](O)[C@H]1O ; [2H][C@@]1(n2ccc(NO)nc2=O)O[C@H](COC(=O)C(C)C)[C@@H](O)[C@H]1O ; [2H][C@@]1(n2ccc(NO)nc2=O)O[C@H](COC(=O)C(CCC)CCC)[C@@H](O)[C@H]1O]

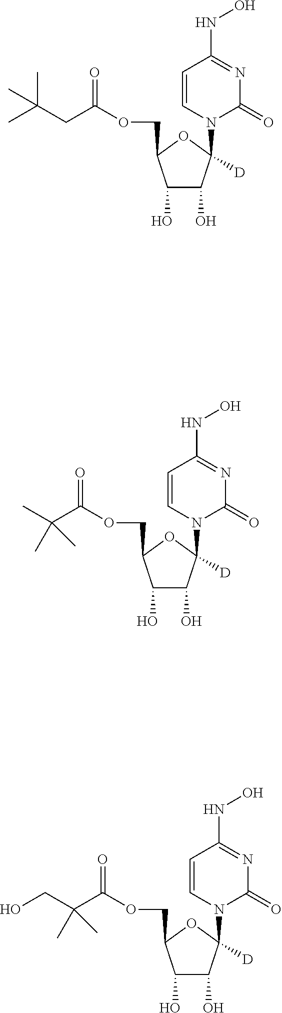 [SMILES: [2H][C@@]1(n2ccc(NO)nc2=O)O[C@H](COC(=O)CC(C)(C)C)[C@@H](O)[C@H]1O ; [2H][C@@]1(n2ccc(NO)nc2=O)O[C@H](COC(=O)C(C)(C)C)[C@@H](O)[C@H]1O ; [2H][C@@]1(n2ccc(NO)nc2=O)O[C@H](COC(=O)C(C)(C)CO)[C@@H](O)[C@H]1O]

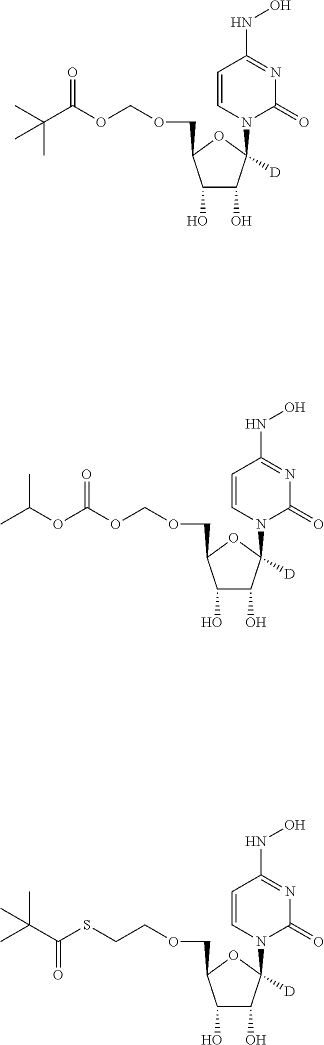 [SMILES: [2H][C@@]1(n2ccc(NO)nc2=O)O[C@H](COCOC(=O)C(C)(C)C)[C@@H](O)[C@H]1O ; [2H][C@@]1(n2ccc(NO)nc2=O)O[C@H](COCOC(=O)OC(C)C)[C@@H](O)[C@H]1O ; [2H][C@@]1(n2ccc(NO)nc2=O)O[C@H](COCCSC(=O)C(C)(C)C)[C@@H](O)[C@H]1O]

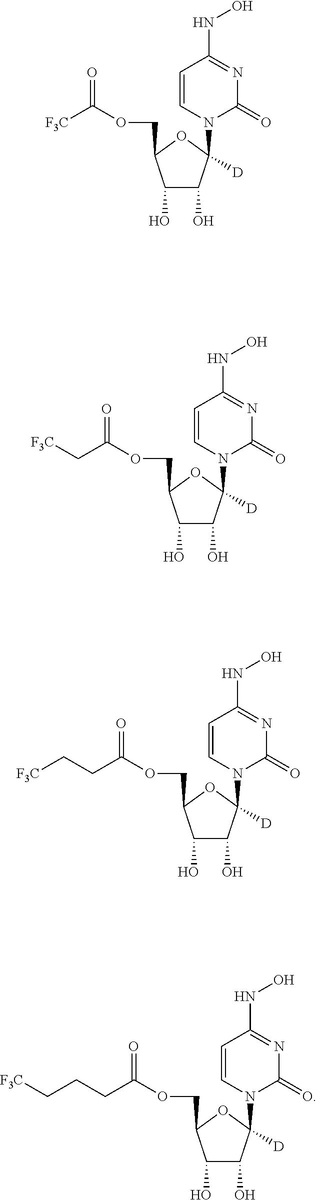 [SMILES: [2H][C@@]1(n2ccc(NO)nc2=O)O[C@H](COC(=O)C(F)(F)F)[C@@H](O)[C@H]1O ; [2H][C@@]1(n2ccc(NO)nc2=O)O[C@H](COC(=O)CC(F)(F)F)[C@@H](O)[C@H]1O ; [2H][C@@]1(n2ccc(NO)nc2=O)O[C@H](COC(=O)CCC(F)(F)F)[C@@H](O)[C@H]1O ; [2H][C@@]1(n2ccc(NO)nc2=O)O[C@H](COC(=O)CCCC(F)(F)F)[C@@H](O)[C@H]1O]

In some examples, the compound is selected from the following:

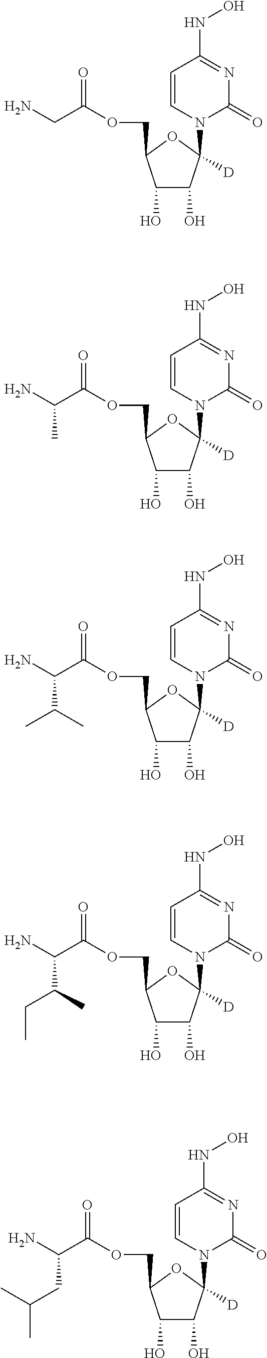 [SMILES: [2H][C@@]1(n2ccc(NO)nc2=O)O[C@H](COC(=O)CN)[C@@H](O)[C@H]1O ; [2H][C@@]1(n2ccc(NO)nc2=O)O[C@H](COC(=O)[C@H](C)N)[C@@H](O)[C@H]1O ; [2H][C@@]1(n2ccc(NO)nc2=O)O[C@H](COC(=O)[C@@H](N)C(C)C)[C@@H](O)[C@H]1O ; [2H][C@@]1(n2ccc(NO)nc2=O)O[C@H](COC(=O)[C@@H](N)[C@@H](C)CC)[C@@H](O)[C@H]1O ; [2H][C@@]1(n2ccc(NO)nc2=O)O[C@H](COC(=O)[C@@H](N)CC(C)C)[C@@H](O)[C@H]1O]

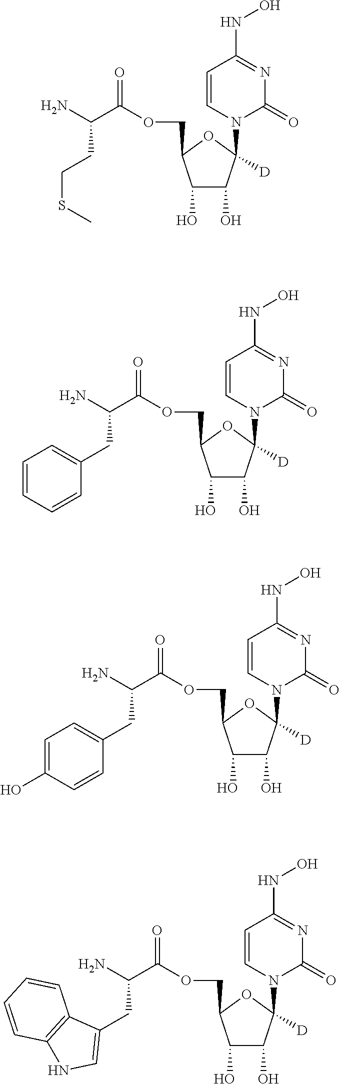 [SMILES: [2H][C@@]1(n2ccc(NO)nc2=O)O[C@H](COC(=O)[C@@H](N)CCSC)[C@@H](O)[C@H]1O ; [2H][C@@]1(n2ccc(NO)nc2=O)O[C@H](COC(=O)[C@@H](N)Cc2ccccc2)[C@@H](O)[C@H]1O ; [2H][C@@]1(n2ccc(NO)nc2=O)O[C@H](COC(=O)[C@@H](N)Cc2ccc(O)cc2)[C@@H](O)[C@H]1O ; [2H][C@@]1(n2ccc(NO)nc2=O)O[C@H](COC(=O)[C@@H](N)Cc2c[nH]c3ccccc23)[C@@H](O)[C@H]1O]

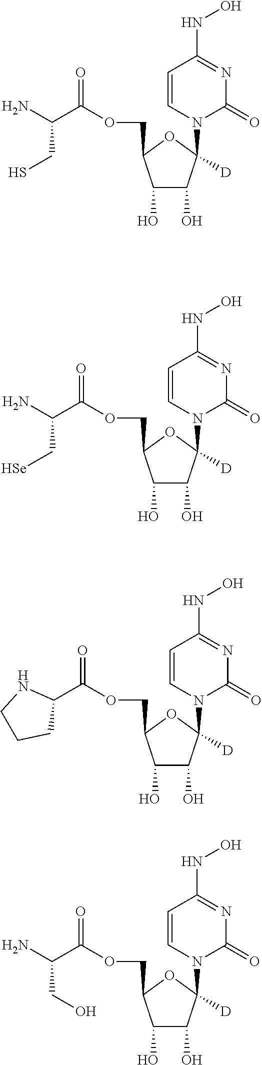

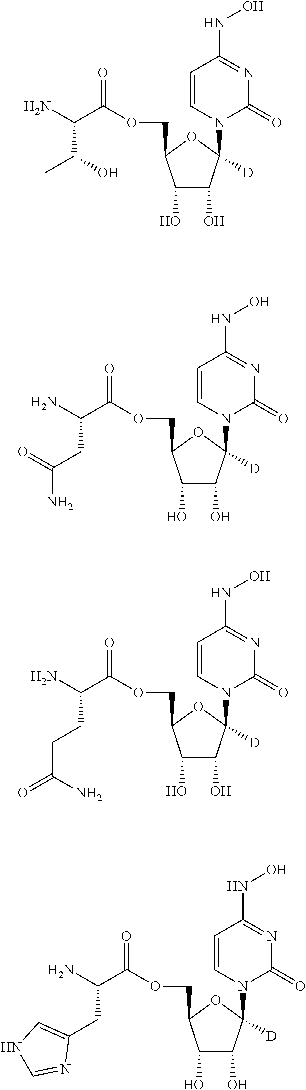 [SMILES: [2H][C@@]1(n2ccc(NO)nc2=O)O[C@H](COC(=O)[C@@H](N)[C@@H](C)O)[C@@H](O)[C@H]1O ; [2H][C@@]1(n2ccc(NO)nc2=O)O[C@H](COC(=O)[C@@H](N)CC(N)=O)[C@@H](O)[C@H]1O ; [2H][C@@]1(n2ccc(NO)nc2=O)O[C@H](COC(=O)[C@@H](N)CCC(N)=O)[C@@H](O)[C@H]1O ; [2H][C@@]1(n2ccc(NO)nc2=O)O[C@H](COC(=O)[C@@H](N)Cc2c[nH]cn2)[C@@H](O)[C@H]1O]

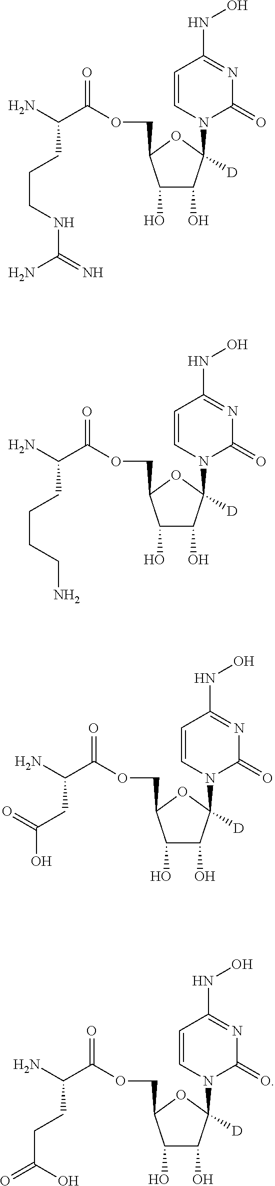 [SMILES: [2H][C@@]1(n2ccc(NO)nc2=O)O[C@H](COC(=O)[C@@H](N)CCCNC(=N)N)[C@@H](O)[C@H]1O ; [2H][C@@]1(n2ccc(NO)nc2=O)O[C@H](COC(=O)[C@@H](N)CCCCN)[C@@H](O)[C@H]1O ; [2H][C@@]1(n2ccc(NO)nc2=O)O[C@H](COC(=O)[C@@H](N)CC(=O)O)[C@@H](O)[C@H]1O ; [2H][C@@]1(n2ccc(NO)nc2=O)O[C@H](COC(=O)[C@@H](N)CCC(=O)O)[C@@H](O)[C@H]1O]

In some examples, the compound is selected from the following:

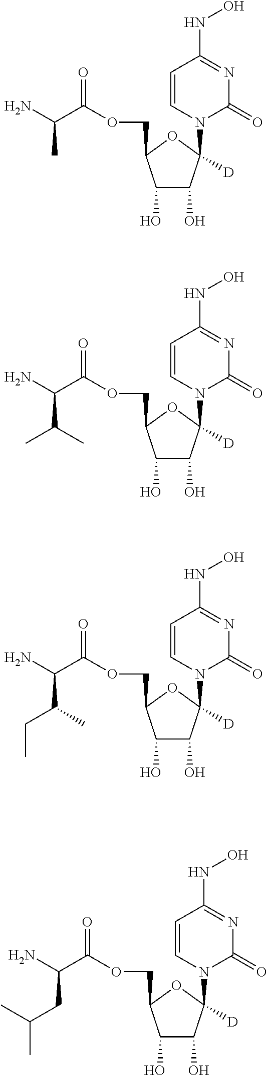 [SMILES: [2H][C@@]1(n2ccc(NO)nc2=O)O[C@H](COC(=O)[C@@H](C)N)[C@@H](O)[C@H]1O ; [2H][C@@]1(n2ccc(NO)nc2=O)O[C@H](COC(=O)[C@H](N)C(C)C)[C@@H](O)[C@H]1O ; [2H][C@@]1(n2ccc(NO)nc2=O)O[C@H](COC(=O)[C@H](N)[C@H](C)CC)[C@@H](O)[C@H]1O ; [2H][C@@]1(n2ccc(NO)nc2=O)O[C@H](COC(=O)[C@H](N)CC(C)C)[C@@H](O)[C@H]1O]

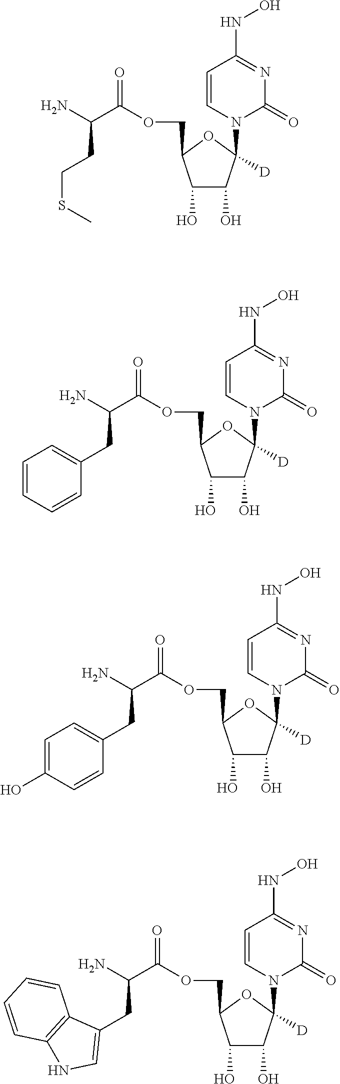 [SMILES: [2H][C@@]1(n2ccc(NO)nc2=O)O[C@H](COC(=O)[C@H](N)CCSC)[C@@H](O)[C@H]1O ; [2H][C@@]1(n2ccc(NO)nc2=O)O[C@H](COC(=O)[C@H](N)Cc2ccccc2)[C@@H](O)[C@H]1O ; [2H][C@@]1(n2ccc(NO)nc2=O)O[C@H](COC(=O)[C@H](N)Cc2ccc(O)cc2)[C@@H](O)[C@H]1O ; [2H][C@@]1(n2ccc(NO)nc2=O)O[C@H](COC(=O)[C@H](N)Cc2c[nH]c3ccccc23)[C@@H](O)[C@H]1O]

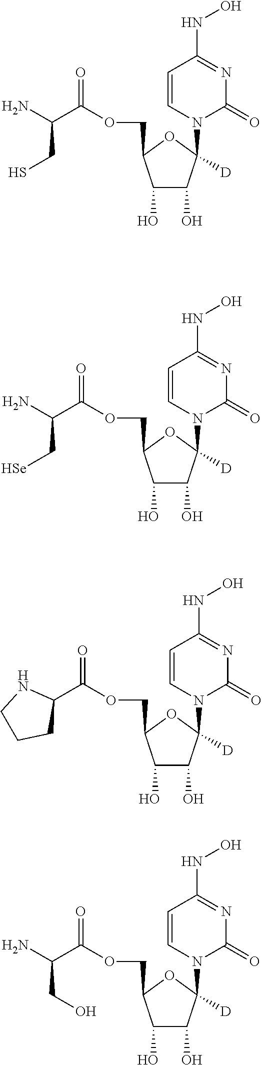

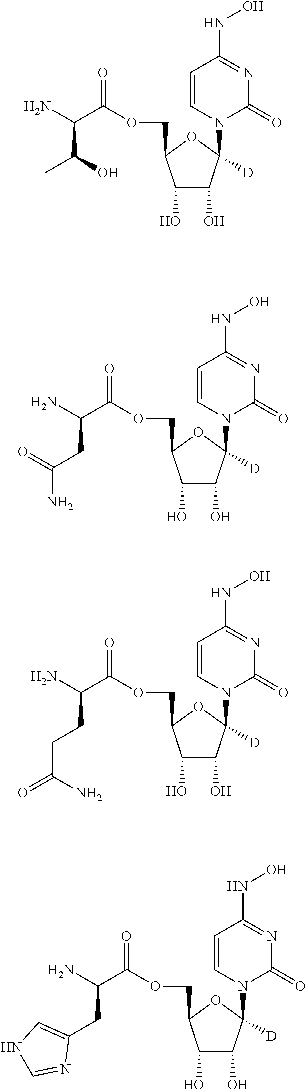 [SMILES: [2H][C@@]1(n2ccc(NO)nc2=O)O[C@H](COC(=O)[C@H](N)[C@H](C)O)[C@@H](O)[C@H]1O ; [2H][C@@]1(n2ccc(NO)nc2=O)O[C@H](COC(=O)[C@H](N)CC(N)=O)[C@@H](O)[C@H]1O ; [2H][C@@]1(n2ccc(NO)nc2=O)O[C@H](COC(=O)[C@H](N)CCC(N)=O)[C@@H](O)[C@H]1O ; [2H][C@@]1(n2ccc(NO)nc2=O)O[C@H](COC(=O)[C@H](N)Cc2c[nH]cn2)[C@@H](O)[C@H]1O]

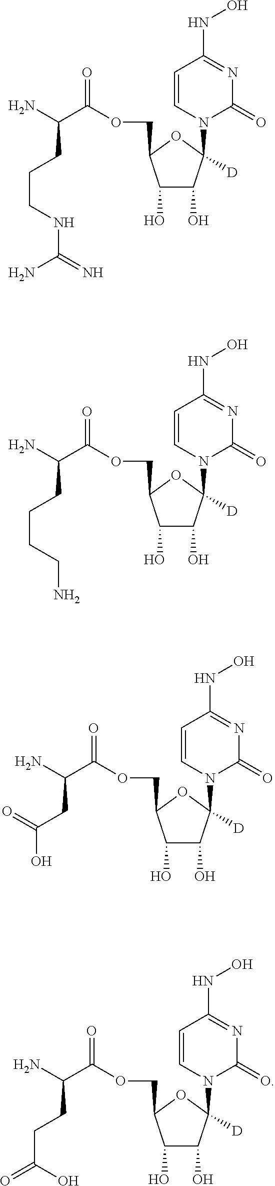 [SMILES: [2H][C@@]1(n2ccc(NO)nc2=O)O[C@H](COC(=O)[C@H](N)CCCNC(=N)N)[C@@H](O)[C@H]1O ; [2H][C@@]1(n2ccc(NO)nc2=O)O[C@H](COC(=O)[C@H](N)CCCCN)[C@@H](O)[C@H]1O ; [2H][C@@]1(n2ccc(NO)nc2=O)O[C@H](COC(=O)[C@H](N)CC(=O)O)[C@@H](O)[C@H]1O ; [2H][C@@]1(n2ccc(NO)nc2=O)O[C@H](COC(=O)[C@H](N)CCC(=O)O)[C@@H](O)[C@H]1O]

In some examples, the compound is selected from the following:

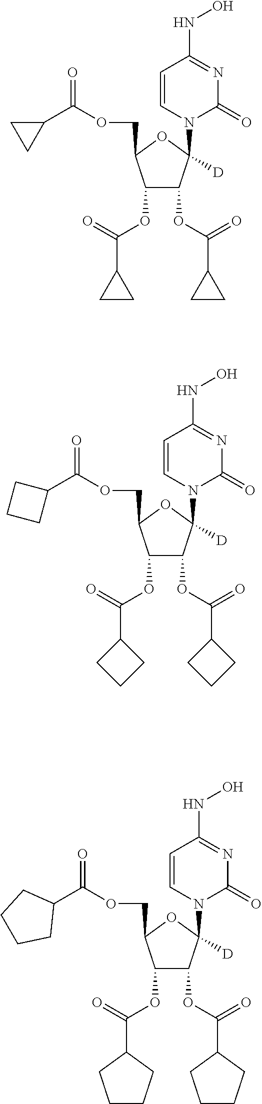 [SMILES: [2H][C@@]1(n2ccc(NO)nc2=O)O[C@H](COC(=O)C2CC2)[C@@H](OC(=O)C2CC2)[C@H]1OC(=O)C1CC1 ; [2H][C@@]1(n2ccc(NO)nc2=O)O[C@H](COC(=O)C2CCC2)[C@@H](OC(=O)C2CCC2)[C@H]1OC(=O)C1CCC1 ; [2H][C@@]1(n2ccc(NO)nc2=O)O[C@H](COC(=O)C2CCCC2)[C@@H](OC(=O)C2CCCC2)[C@H]1OC(=O)C1CCCC1]

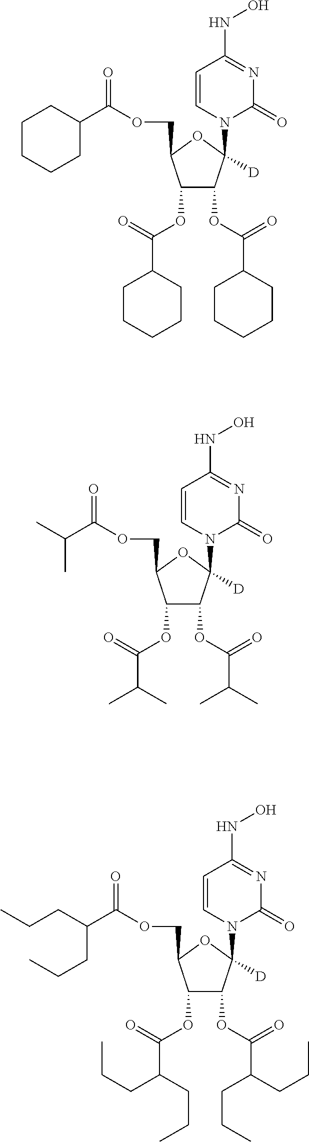 [SMILES: [2H][C@@]1(n2ccc(NO)nc2=O)O[C@H](COC(=O)C2CCCCC2)[C@@H](OC(=O)C2CCCCC2)[C@H]1OC(=O)C1CCCCC1 ; [2H][C@@]1(n2ccc(NO)nc2=O)O[C@H](COC(=O)C(C)C)[C@@H](OC(=O)C(C)C)[C@H]1OC(=O)C(C)C ; [2H][C@@]1(n2ccc(NO)nc2=O)O[C@H](COC(=O)C(CCC)CCC)[C@@H](OC(=O)C(CCC)CCC)[C@H]1OC(=O)C(CCC)CCC]

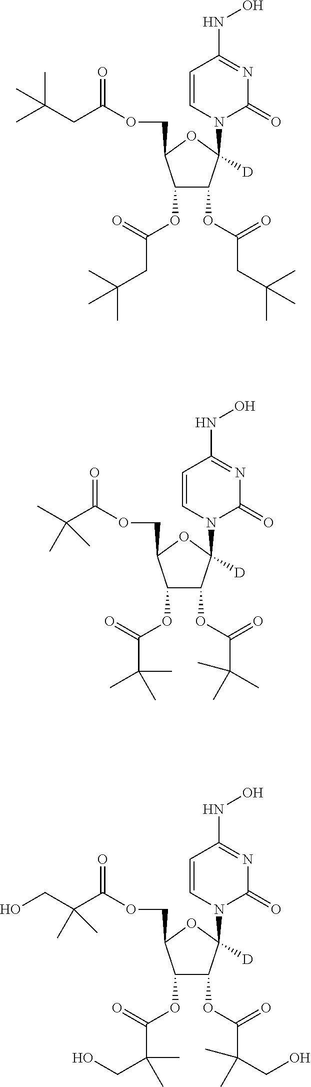

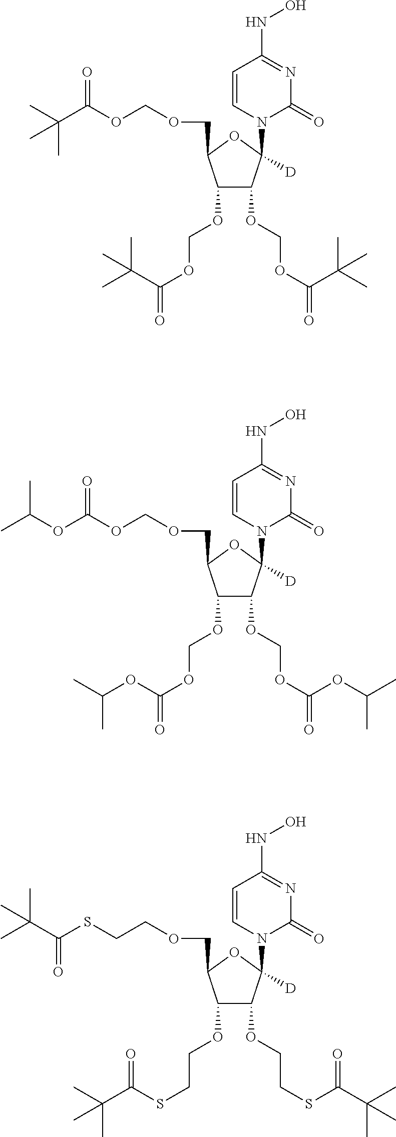

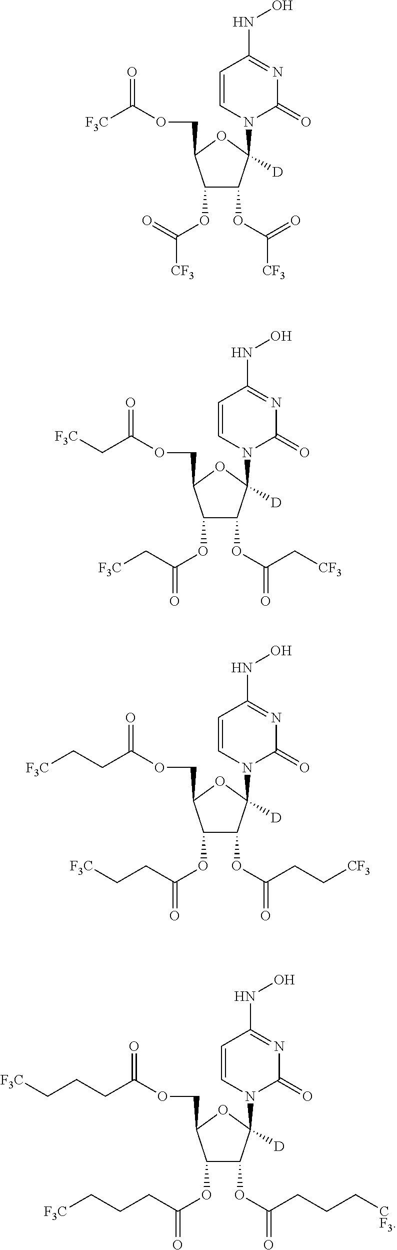 [SMILES: [2H][C@@]1(n2ccc(NO)nc2=O)O[C@H](COC(=O)C(F)(F)F)[C@@H](OC(=O)C(F)(F)F)[C@H]1OC(=O)C(F)(F)F ; [2H][C@@]1(n2ccc(NO)nc2=O)O[C@H](COC(=O)CC(F)(F)F)[C@@H](OC(=O)CC(F)(F)F)[C@H]1OC(=O)CC(F)(F)F ; [2H][C@@]1(n2ccc(NO)nc2=O)O[C@H](COC(=O)CCC(F)(F)F)[C@@H](OC(=O)CCC(F)(F)F)[C@H]1OC(=O)CCC(F)(F)F ; [2H][C@@]1(n2ccc(NO)nc2=O)O[C@H](COC(=O)CCCC(F)(F)F)[C@@H](OC(=O)CCCC(F)(F)F)[C@H]1OC(=O)CCCC(F)(F)F]

Also disclosed herein is a pharmaceutical composition for the treatment of 2019nCoV/SARS-CoV-2 infection, wherein the compound is a compound of Formula II,

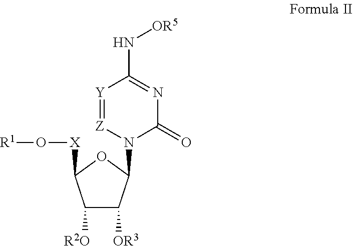

or a tautomer thereof, or a pharmaceutical salt or physiological salt thereof, wherein
X is CH₂, CHCH₃, C(CH₃)₂, CHF, CF₂, or CD₂;
Y is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or carbonyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, hydroxyl, thiol, or carbonyl, wherein R″ is optionally substituted with one or more, the same or different, R¹⁰;
R¹, R², R³, and R⁵ are each independently selected from H, or together with the oxygen to which they are attached form optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R², R³, and R⁵ are optionally substituted with one or more, the same or different, R¹⁰;
with the proviso that R¹, R², R³, and R⁵ are not all H;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In some examples, R′ is methyl, fluoro, hydroxymethyl, fluoromethyl, difluoromethyl, trifluoromethyl, trideuteromethyl, thiomethyl, carboxylic acid, formyl, vinyl, or ethynyl. In other examples, R″ is methyl, fluoro, hydroxymethyl, fluoromethyl, difluoromethyl, trifluoromethyl, trideuteromethyl, thiomethyl, carboxylic acid, formyl, vinyl, or ethynyl.

Also disclosed herein is a pharmaceutical composition for the treatment of 2019nCoV/SARS-CoV-2 infection, comprising a pharmaceutically acceptable excipient and a compound of Formula III,

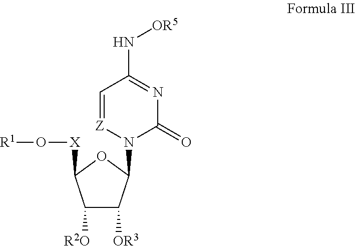

or a tautomer thereof, or a pharmaceutical salt or physiological salt thereof, wherein
X is CH₂, CHCH₃, C(CH₃)₂, CHF, CF₂, or CD₂;
Z is N or CR″;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, hydroxyl, thiol, or carbonyl, wherein R″ is optionally substituted with one or more, the same or different, R¹⁰;
R¹, R², R³, and R⁵ are each independently selected from H, or together with the oxygen to which they are attached form optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R², R³, and R⁵ are optionally substituted with one or more, the same or different, R¹⁰;
with the proviso that R¹, R², R³, and R⁵ are not all H;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein. In some examples, R″ is methyl, fluoro, hydroxymethyl, fluoromethyl, difluoromethyl, trifluoromethyl, trideuteromethyl, thiomethyl, carboxylic acid, formyl, vinyl, or ethynyl.

Also disclosed herein is a pharmaceutical composition for the treatment of 2019nCoV/SARS-CoV-2 infection, comprising a pharmaceutically acceptable excipient and a compound of Formula IV,

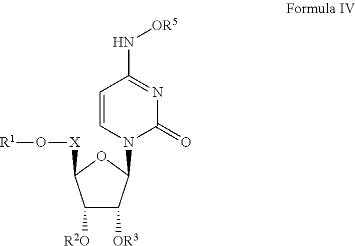

or a tautomer thereof, or a pharmaceutical salt or physiological salt thereof, wherein
X is CHCH₃, C(CH₃)₂, CHF, CF₂, or CD₂;
R¹, R², R³, and R⁵ are each independently selected from H, or together with the oxygen to which they are attached form optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R², R³, and R⁵ are optionally substituted with one or more, the same or different, R¹⁰;
with the proviso that R¹, R², R³, and R⁵ are not all H;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula V,

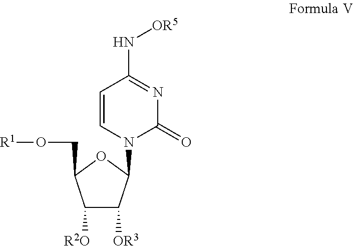

or a tautomer thereof, or a pharmaceutical salt or physiological salt thereof, wherein
R¹, R², R³, and R⁵ together with the oxygen to which they are attached form are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R², R³, and R⁵ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula VI,

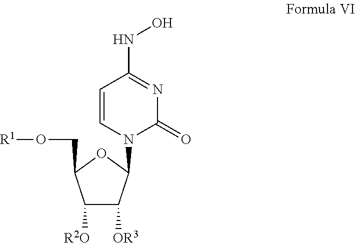

or a tautomer thereof, or a pharmaceutical salt or physiological salt thereof, wherein
R¹, R², and R³, together with the oxygen to which they are attached, are each independently selected from the following: optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, N-substituted L-amino acid esters, N,N-disubstituted L-amino acid esters, N-substituted D-amino acid esters, N,N-disubstituted D-amino acid esters, oxymethoxy amino ester, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R², and R³ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein. In some examples, the compound has Formula VIa-f,

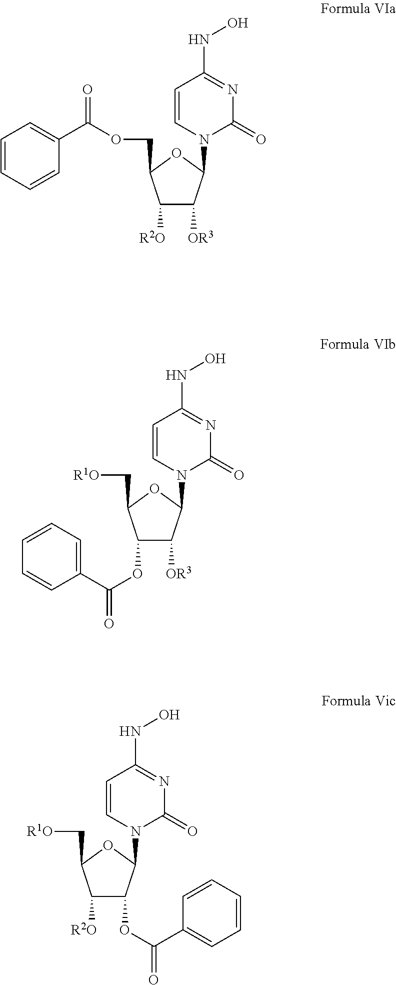

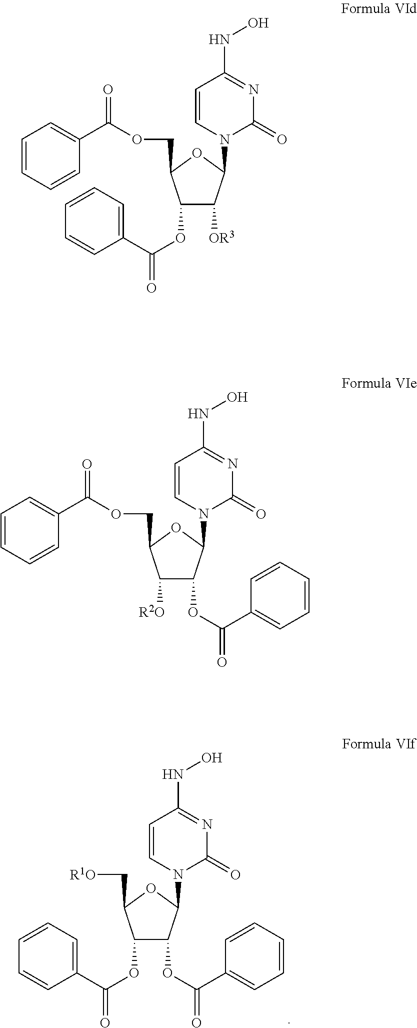

In other examples, the compound is selected from the following:

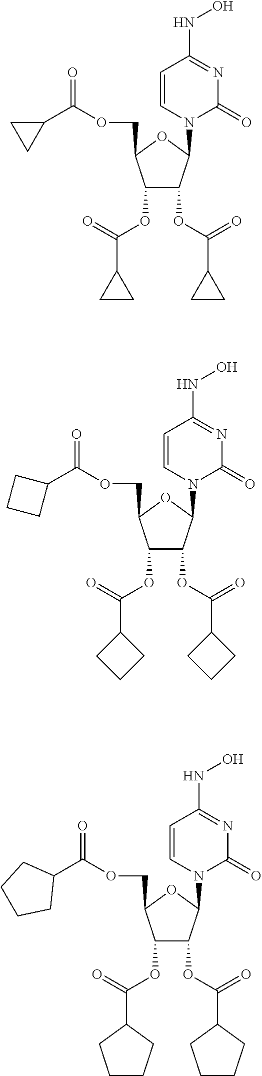 [SMILES: O=C(OC[C@H]1O[C@@H](n2ccc(NO)nc2=O)[C@H](OC(=O)C2CC2)[C@@H]1OC(=O)C1CC1)C1CC1 ; O=C(OC[C@H]1O[C@@H](n2ccc(NO)nc2=O)[C@H](OC(=O)C2CCC2)[C@@H]1OC(=O)C1CCC1)C1CCC1 ; O=C(OC[C@H]1O[C@@H](n2ccc(NO)nc2=O)[C@H](OC(=O)C2CCCC2)[C@@H]1OC(=O)C1CCCC1)C1CCCC1]

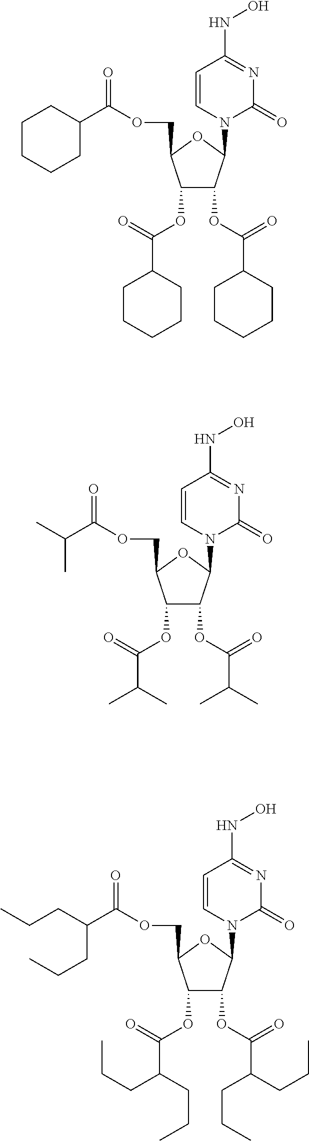 [SMILES: O=C(OC[C@H]1O[C@@H](n2ccc(NO)nc2=O)[C@H](OC(=O)C2CCCCC2)[C@@H]1OC(=O)C1CCCCC1)C1CCCCC1 ; CC(C)C(=O)OC[C@H]1O[C@@H](n2ccc(NO)nc2=O)[C@H](OC(=O)C(C)C)[C@@H]1OC(=O)C(C)C ; CCCC(CCC)C(=O)OC[C@H]1O[C@@H](n2ccc(NO)nc2=O)[C@H](OC(=O)C(CCC)CCC)[C@@H]1OC(=O)C(CCC)CCC]

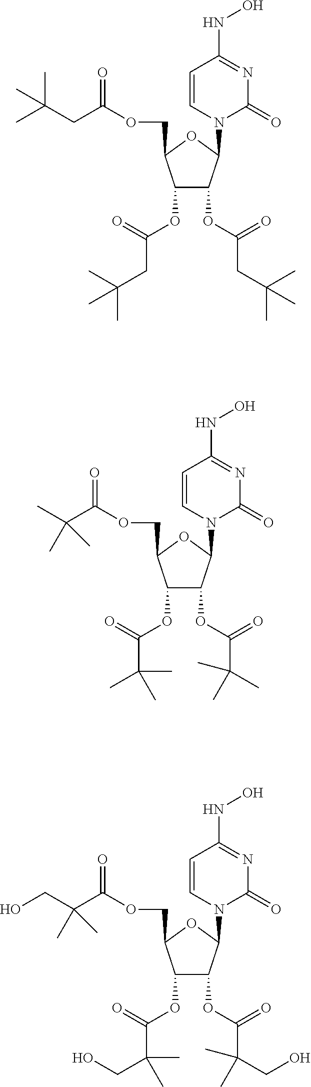

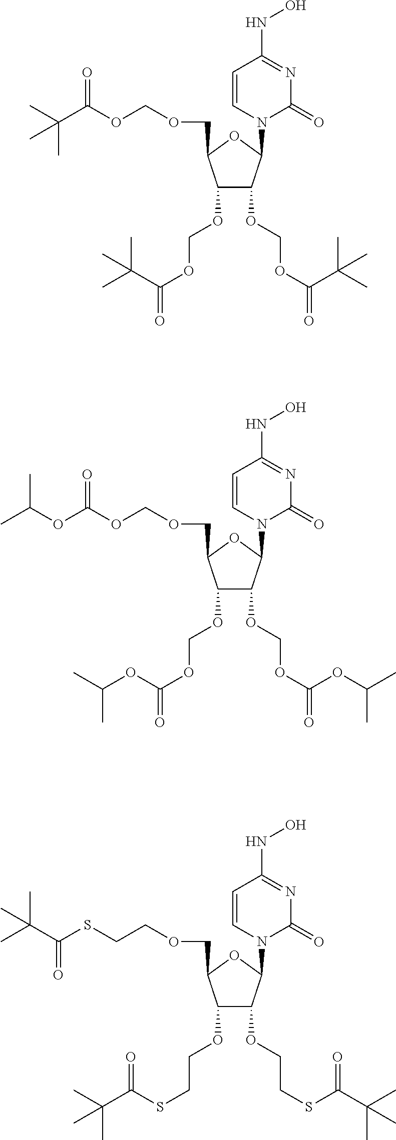

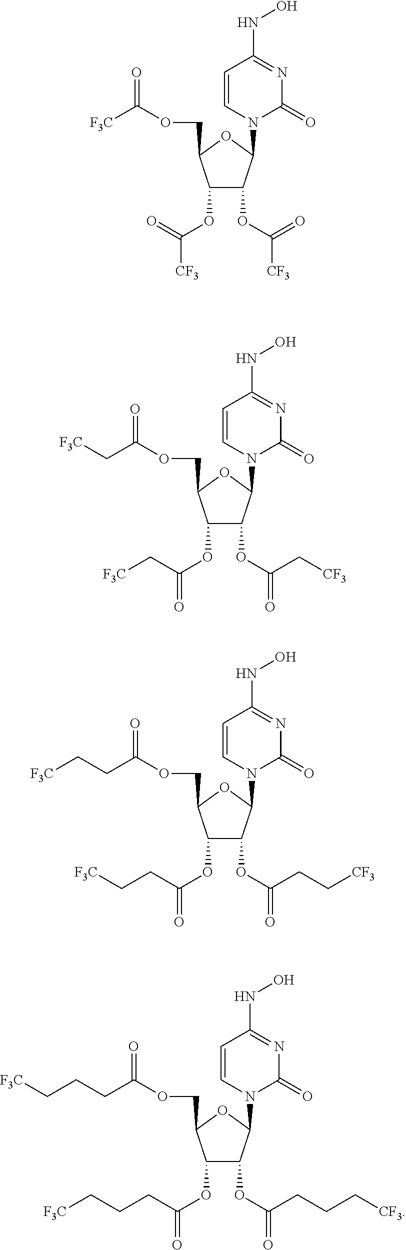 [SMILES: O=C(OC[C@H]1O[C@@H](n2ccc(NO)nc2=O)[C@H](OC(=O)C(F)(F)F)[C@@H]1OC(=O)C(F)(F)F)C(F)(F)F ; O=C(CC(F)(F)F)OC[C@H]1O[C@@H](n2ccc(NO)nc2=O)[C@H](OC(=O)CC(F)(F)F)[C@@H]1OC(=O)CC(F)(F)F ; O=C(CCC(F)(F)F)OC[C@H]1O[C@@H](n2ccc(NO)nc2=O)[C@H](OC(=O)CCC(F)(F)F)[C@@H]1OC(=O)CCC(F)(F)F ; O=C(CCCC(F)(F)F)OC[C@H]1O[C@@H](n2ccc(NO)nc2=O)[C@H](OC(=O)CCCC(F)(F)F)[C@@H]1OC(=O)CCCC(F)(F)F]

Also disclosed herein is a pharmaceutical composition for the treatment of 2019nCoV/SARS-CoV-2 infection, comprising a pharmaceutically acceptable excipient and a compound of Formula VII,

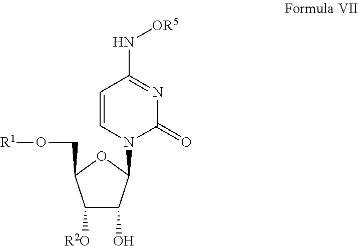

or a tautomer thereof, or a pharmaceutical salt or physiological salt thereof, wherein
R¹, R², and R⁵, together with the oxygen to which they are attached, are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R², and R⁵ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula VIII,

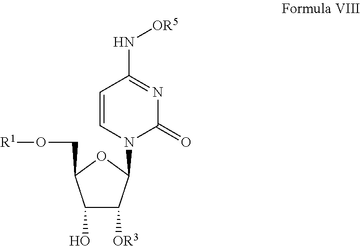

or a tautomer thereof, or a pharmaceutical salt or physiological salt thereof, wherein
R¹, R³, and R⁵, together with the oxygen to which they are attached, are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹, R³, and R⁵ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula IX,

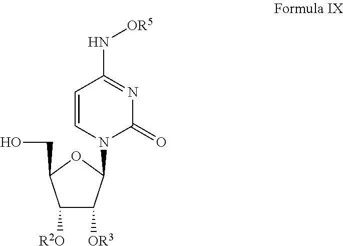

or a tautomer thereof, or a pharmaceutical salt or physiological salt thereof, wherein
R², R³, and R⁵, together with the oxygen to which they are attached, are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R², R³, and R⁵ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula X,

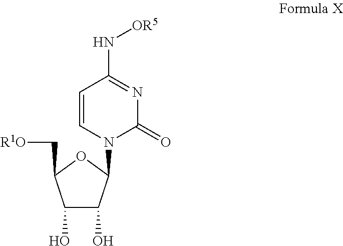

or a tautomer thereof, or a pharmaceutical salt or physiological salt thereof, wherein
—O—R¹ and —O—R⁵ are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹ and R⁵ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula XI,

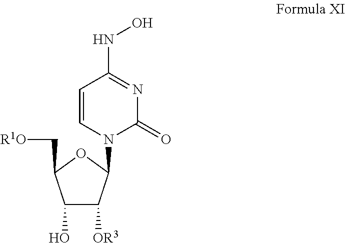

or a tautomer thereof, or a pharmaceutical salt or physiological salt thereof, wherein
—O—R¹ and —O—R³ are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹ and R³ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula XII,

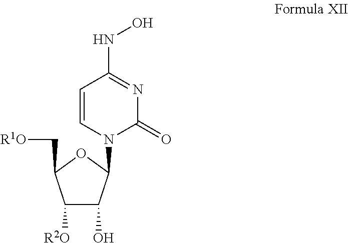

or a tautomer thereof, or a pharmaceutical salt or physiological salt thereof, wherein
—O—R¹ and —O—R² are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R¹ and R² are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula XIII,

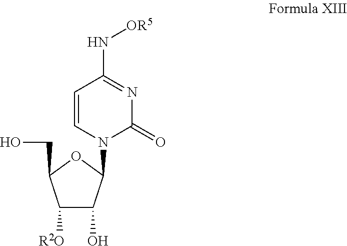

or a tautomer thereof, or a pharmaceutical salt or physiological salt thereof, wherein
—O—R² and —O—R⁵ are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, or BAB-esters, wherein R² and R⁵ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula XIV,

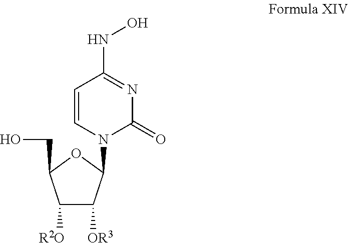

or a tautomer thereof, or a pharmaceutical salt or physiological salt thereof, wherein
—O—R² and —O—R³ are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R² and R³ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula XV,

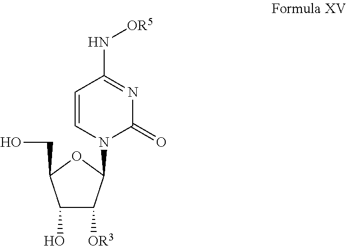

or a tautomer thereof, or a pharmaceutical salt or physiological salt thereof, wherein
—O—R³ and —O—R⁵ are each independently selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R³ and R⁵ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula XVI,

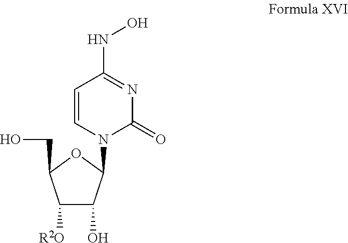

or a tautomer thereof, or a pharmaceutical salt or physiological salt thereof, wherein
—O—R² is selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R² are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula XVII,

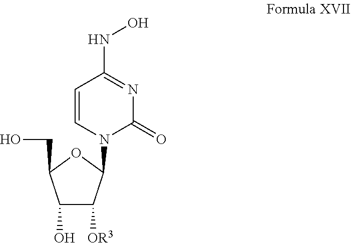

or a tautomer thereof, or a pharmaceutical salt or physiological salt thereof, wherein
—O—R³ is selected from the following: optionally substituted esters, optionally substituted branched esters, optionally substituted carbonates, optionally substituted carbamates, optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R³ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein is a pharmaceutical composition for the treatment of 2019nCoV/SARS-CoV-2 infection, comprising a pharmaceutically acceptable excipient and a compound of Formula XIX,

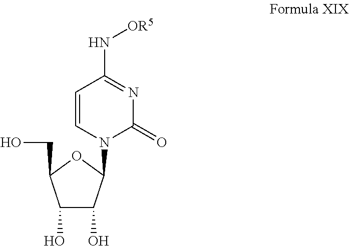

or a tautomer thereof, or a pharmaceutical salt or physiological salt thereof, wherein
—O—R⁵ is selected from the following: optionally substituted thioesters, optionally substituted branched thioesters, optionally substituted thiocarbonates, sulfenyl thiocarbonates, optionally substituted sulfenyl thiocarbonates, 2-hydroxypropanoate ester, optionally substitute 2-hydroxypropanoate ester, optionally substituted S-thiocarbonate, optionally substituted dithiocarbonates, optionally substituted thiocarbamates, optionally substituted oxymethoxycarbonyl, oxymethoxycarbonate, optionally substituted oxymethoxycarbonate, optionally substituted oxymethoxythiocarbonyl, optionally substituted oxymethylcarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, optionally substituted sulfenyl, sulfinyl, sulfonyl, sulfite, sulfate, sulfonamide, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, (acyloxybenzyl)ether, (acyloxybenzyl)ester, PEG ester, PEG carbonate, optionally substituted bis-(acyloxybenzyl)esters, optionally substituted (acyloxybenzyl)esters, or BAB-esters, wherein R⁵ are optionally substituted with one or more, the same or different, R¹⁰;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein. In some examples, R⁵ is selected from the following:

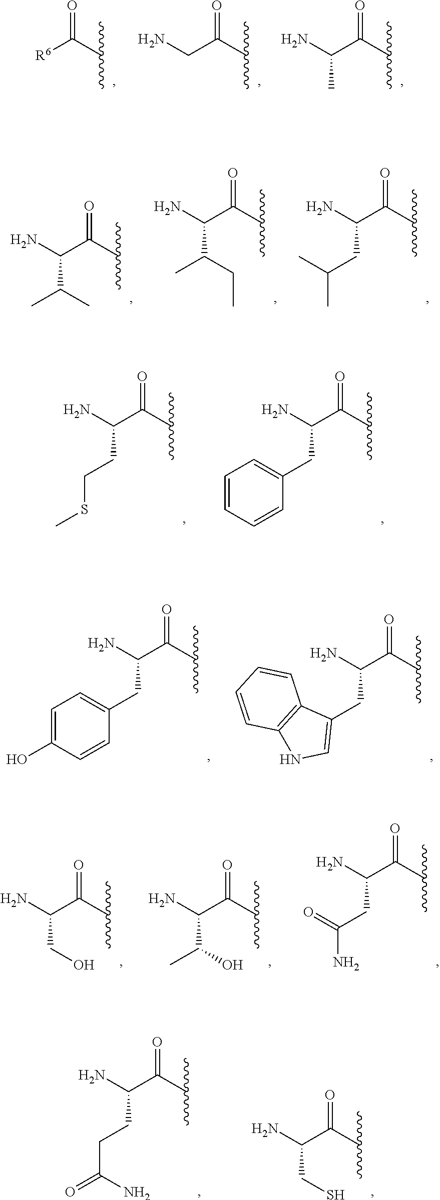

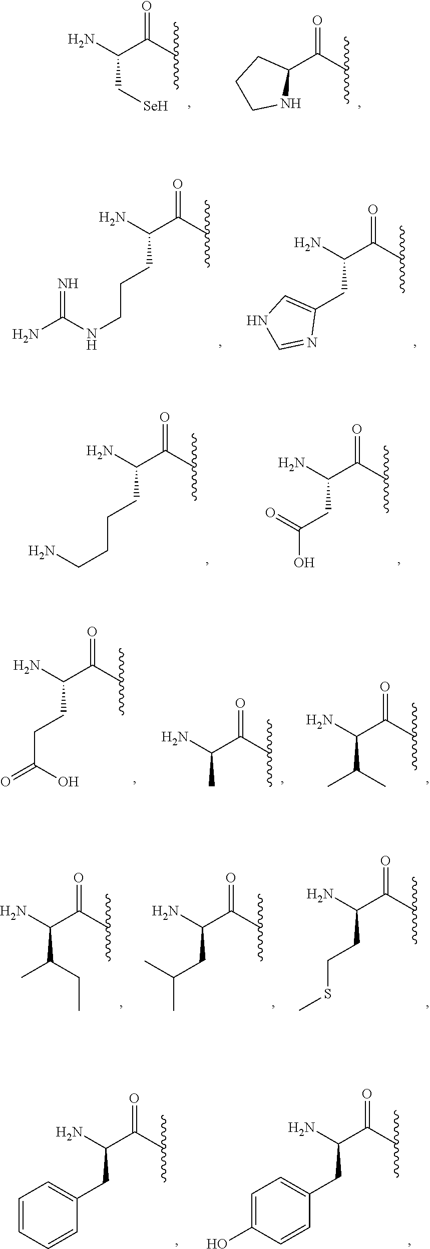

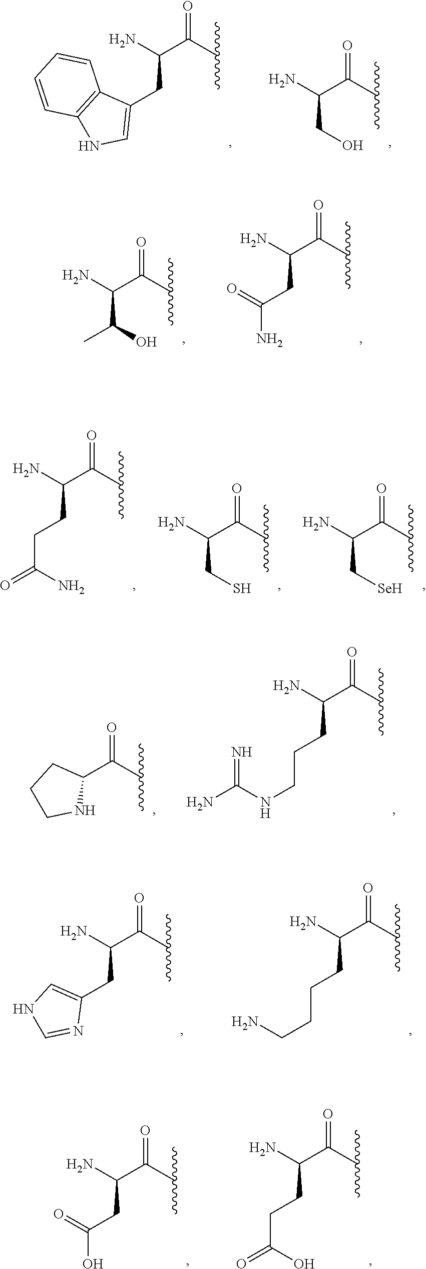

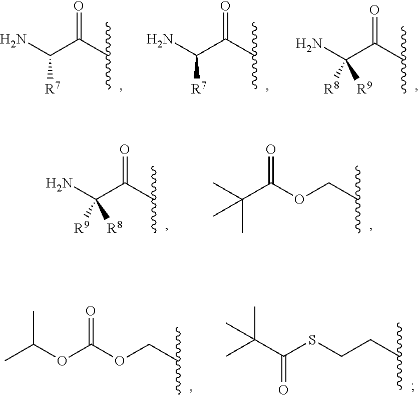

R⁶ is hydrogen, C₂-C₇ n-alkyl, optionally substituted C₈ n-alkyl, C₉-C₂₂ n-alkyl, alkenyl, alkynyl, carbocyclyl, heterocarbocyclyl, aryl, heteroaryl, heterocyclyl, C₃-C₉ cycloalkyl, C₁₁-C₂₂ cycloalkyl, optionally substituted C₁₀ cycloalkyl, cycloalkenyl, —O(C₁-C₆ n-alkyl), —O(optionally substituted C₇ n-alkyl), —O(C₈-C₂₁ n-alkyl), —O(branched alkyl), carbocycloxy, heterocarbocycloxy, aryloxy, heteroaryloxy, heterocycloxy, cycloalkoxy, cycloalkenoxy, —N(C₂-C₂₁ n-alkyl)₂, —N(optionally substituted C₁ alkyl)₂, —NH (optionally substituted C₁ alkyl), —NH(C₂-C₆n-alkyl), —NH (optionally substituted C₇ n-alkyl), —NH(C₈-C₁₅ n-alkyl), —NH (optionally substituted C₁₆ n-alkyl), —NH(C₁₇ n-alkyl), —NH (optionally substituted C₁₈ n-alkyl), —NH(C₁₉-C₂₁ n-alkyl), —NH(branched alkyl), —N(branched alkyl)₂, carbocyclamino, heterocarbocyclamino, optionally substituted arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, cyano, or lipid, wherein R⁶ is optionally substituted with one or more, the same or different, R¹⁰;
R⁷ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R⁸, and R⁹ can form a ring with the α-carbon they are attached to and the amino group attached to the α-carbon; and
R⁸ and R⁹ can form a ring with the α-carbon to which they are attached.

In some examples of the compounds disclosed herein, R¹, R², R³, and R⁵ are each independently selected from H,

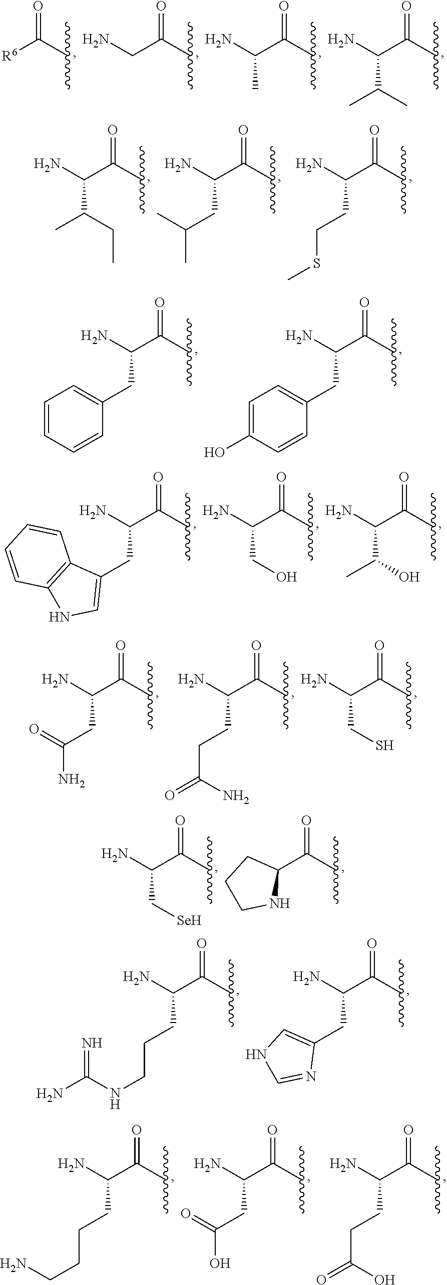

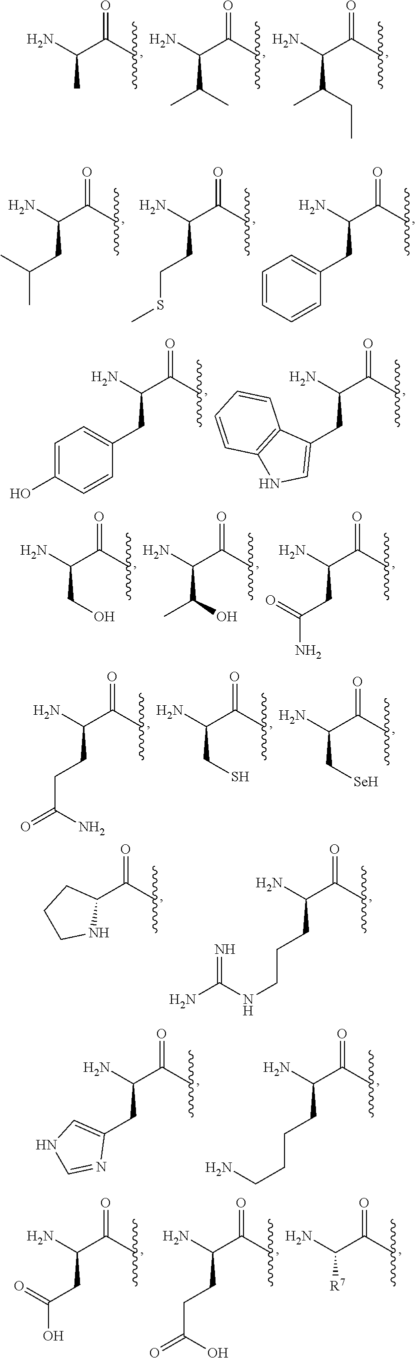

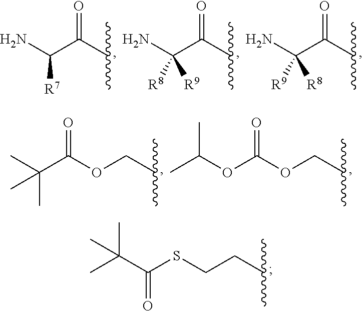

with the proviso that R¹, R², R³, and R⁵ are not all H;
R⁶ is hydrogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, cyano, or lipid, wherein R⁶ is optionally substituted with one or more, the same or different, R¹⁰;
R⁷ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R⁸, and R⁹ can form a ring with the α-carbon they are attached to and the amino group attached to the α-carbon;
R⁸ and R⁹ can form a ring with the α-carbon to which they are attached;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In other examples of compounds disclosed herein, R¹, R², and R³ are each independently selected from the following:

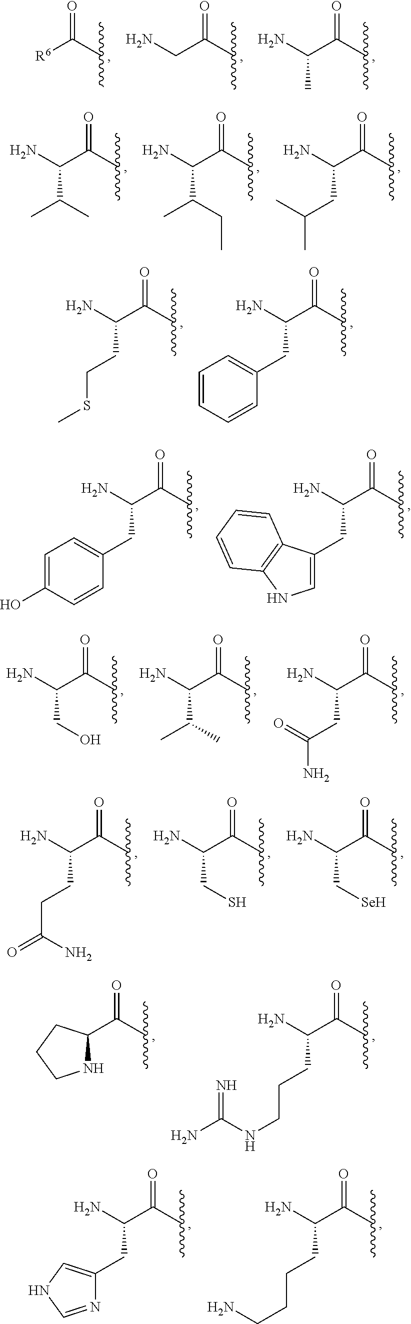

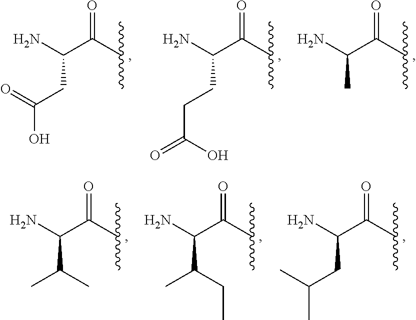

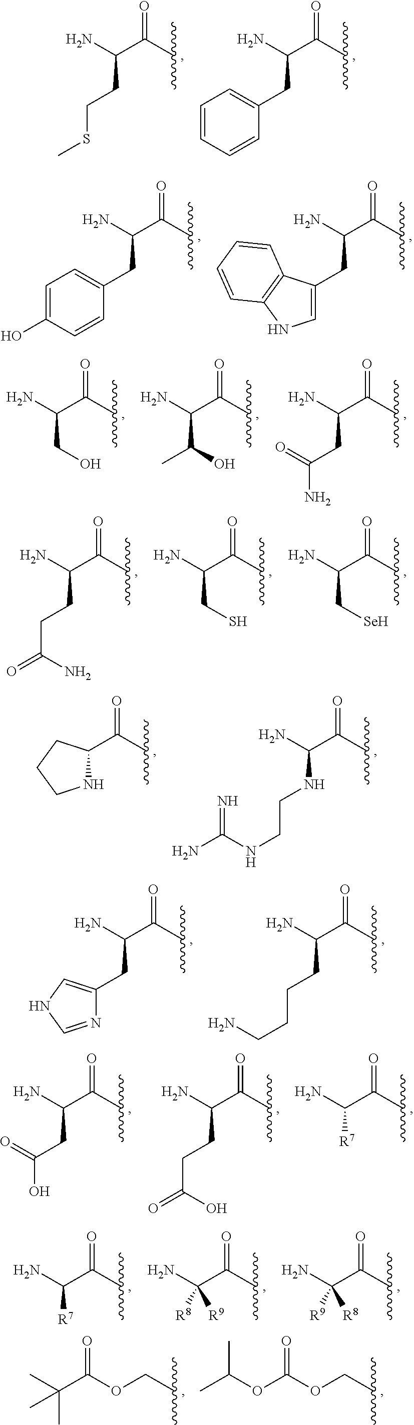

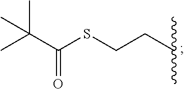 [SMILES: *CCSC(=O)C(C)(C)C]

R⁶ is hydrogen, alkyl, alkenyl, alkynyl, carbocyclyl, heterocarbocyclyl, optionally substituted phenyl, optionally substituted aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, cyano, or lipid, wherein R⁶ is optionally substituted with one or more, the same or different, R¹⁰;
R⁷ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R⁸, and R⁹ can form a ring with the α-carbon they are attached to and the amino group attached to the α-carbon;
R⁸ and R⁹ can form a ring with the α-carbon to which they are attached;
R¹⁰ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹⁰;
R¹¹ is hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

Also disclosed herein pharmaceutical composition for the treatment of COVID-19, comprising a pharmaceutically acceptable excipient and a compound with the following structure:

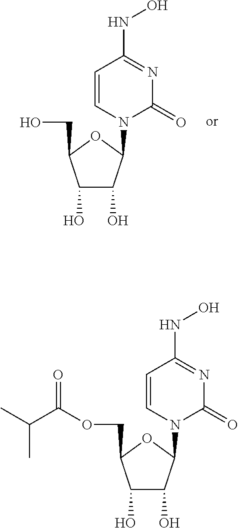 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O]

or a tautomer thereof, or a pharmaceutical salt or physiological salt thereof.

Further, disclosed are pharmaceutical compositions of compounds disclosed herein, further comprising a propellant. The propellant can be compressed air, ethanol, nitrogen, carbon dioxide, nitrous oxide, hydrofluoroalkanes (TFA), 1,1,1,2,-tetrafluoroethane, 1,1,1,2,3,3,3-heptafluoropropane or combinations thereof. A pressurized container comprising a pharmaceutical composition as disclosed herein is also disclosed. The container can be a manual pump spray, inhaler, meter-dosed inhaler, dry powder inhaler, nebulizer, vibrating mesh nebulizer, jet nebulizer, or ultrasonic wave nebulizer.

Also disclosed herein is a method of treating or preventing 2019nCoV/SARS-CoV-2 infection, comprising administering an effective amount of a composition as disclosed herein to a patient in need thereof. The method of treating or preventing 2019nCoV/SARS-CoV-2 infection may comprise administering an effective amount of a compound having the structure:

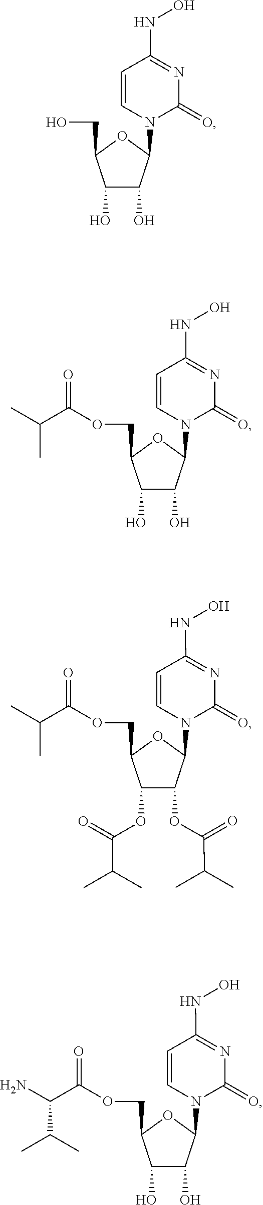 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O ; CC(C)C(=O)OC[C@H]1O[C@@H](n2ccc(NO)nc2=O)[C@H](OC(=O)C(C)C)[C@@H]1OC(=O)C(C)C ; CC(C)[C@H](N)C(=O)OC[C@H]1O[C@@H](n2ccc(NO)nc2=O)[C@H](O)[C@@H]1O]

tautomer thereof, or a pharmaceutical salt or physiological salt thereof, to the patient.

Also disclosed herein is a method of treating or preventing COVID in a patient in need thereof comprising administering an effective amount of a pharmaceutical composition comprising a compound with the structure:

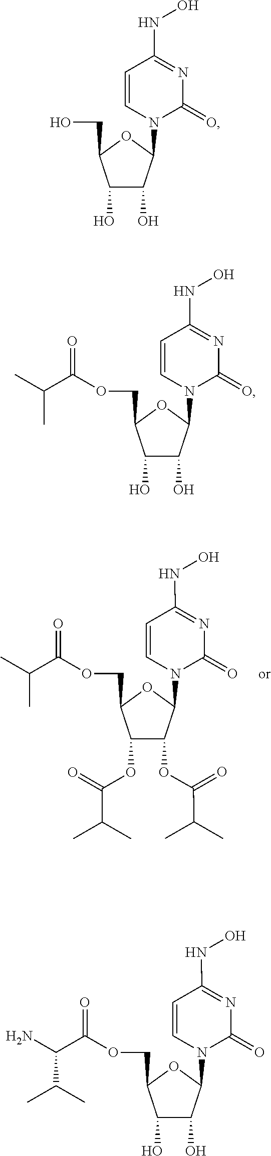 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O ; CC(C)C(=O)OC[C@H]1O[C@@H](n2ccc(NO)nc2=O)[C@H](OC(=O)C(C)C)[C@@H]1OC(=O)C(C)C ; CC(C)[C@H](N)C(=O)OC[C@H]1O[C@@H](n2ccc(NO)nc2=O)[C@H](O)[C@@H]1O]

tautomer thereof, or a pharmaceutical salt or physiological salt thereof, to the patient.

Also disclosed herein is pharmaceutical composition as disclosed herein comprising a compound with the structure:

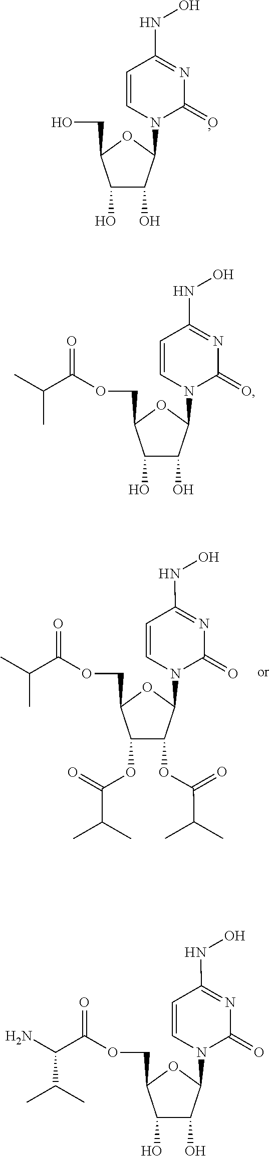 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O ; CC(C)C(=O)OC[C@H]1O[C@@H](n2ccc(NO)nc2=O)[C@H](OC(=O)C(C)C)[C@@H]1OC(=O)C(C)C ; CC(C)[C@H](N)C(=O)OC[C@H]1O[C@@H](n2ccc(NO)nc2=O)[C@H](O)[C@@H]1O]

or a tautomer thereof, or a pharmaceutical salt or physiological salt thereof; a pharmaceutically acceptable excipient or a tautomer thereof, or pharmaceutical salt or physiological salt thereof; further comprising one or more antiviral agents, such as abacavir, acyclovir, acyclovir, adefovir, amantadine, amprenavir, ampligen, arbidol, atazanavir, atripla, balapiravir, BCX4430/Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, GS-5734/Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, or zidovudine and combinations thereof.

Also disclosed herein is a pharmaceutical composition for the treatment of 2019nCoV/SARS-CoV-2 infection, comprising a pharmaceutically acceptable excipient and a compound with the following structure:

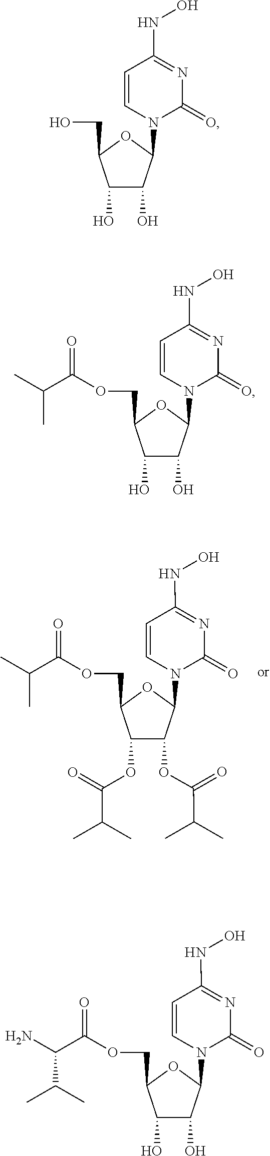 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O ; CC(C)C(=O)OC[C@H]1O[C@@H](n2ccc(NO)nc2=O)[C@H](OC(=O)C(C)C)[C@@H]1OC(=O)C(C)C ; CC(C)[C@H](N)C(=O)OC[C@H]1O[C@@H](n2ccc(NO)nc2=O)[C@H](O)[C@@H]1O]

or a tautomer thereof, or pharmaceutical salt or physiological salt thereof, and one or more antiviral agent selected from the group consisting of abacavir, acyclovir, acyclovir, adefovir, amantadine, amprenavir, ampligen, arbidol, atazanavir, atripla, balapiravir, Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine, zalcitabine, zanamivir, and zidovudine, and combinations thereof.

Also disclosed herein is a compound for the treatment of 2019nCoV/SARS-CoV-2 infection, wherein the compound is:

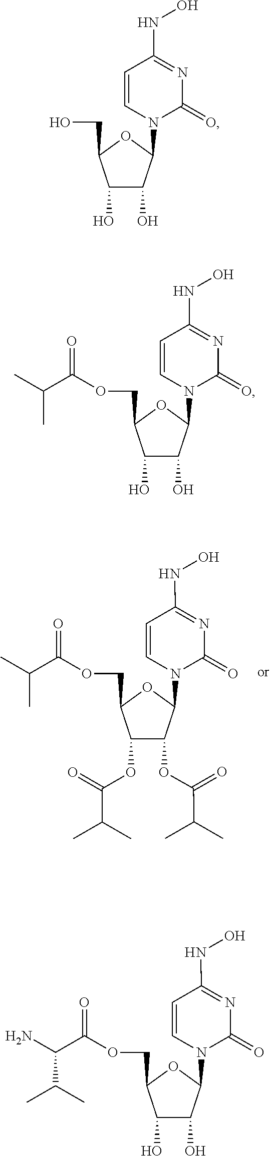 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O ; CC(C)C(=O)OC[C@H]1O[C@@H](n2ccc(NO)nc2=O)[C@H](OC(=O)C(C)C)[C@@H]1OC(=O)C(C)C ; CC(C)[C@H](N)C(=O)OC[C@H]1O[C@@H](n2ccc(NO)nc2=O)[C@H](O)[C@@H]1O]

or a tautomer thereof, or pharmaceutical salt or physiological salt thereof, and one or more antiviral agent selected from the group consisting of abacavir, acyclovir, acyclovir, adefovir, amantadine, amprenavir, ampligen, arbidol, atazanavir, atripla, balapiravir, Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine, zalcitabine, zanamivir, and zidovudine, and combinations thereof.

Also disclosed is a pharmaceutical composition for the treatment of COVID-19, comprising a pharmaceutically acceptable excipient and a compound with the following structure:

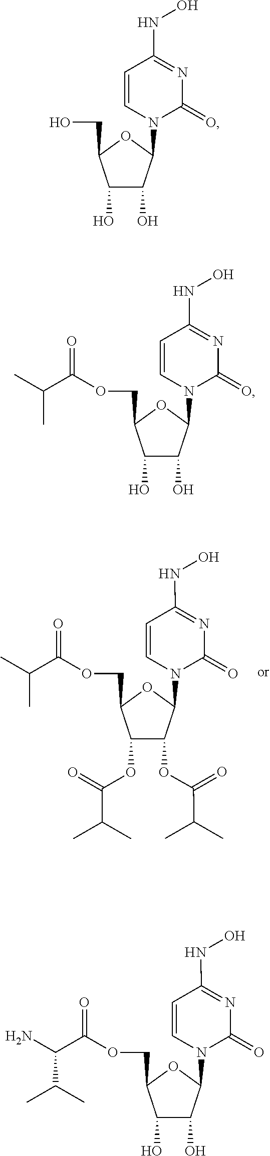 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O ; CC(C)C(=O)OC[C@H]1O[C@@H](n2ccc(NO)nc2=O)[C@H](OC(=O)C(C)C)[C@@H]1OC(=O)C(C)C ; CC(C)[C@H](N)C(=O)OC[C@H]1O[C@@H](n2ccc(NO)nc2=O)[C@H](O)[C@@H]1O]

tautomer thereof, or pharmaceutical salt or physiological salt thereof, and one or more antiviral agent selected from the group consisting of abacavir, acyclovir, acyclovir, adefovir, amantadine, amprenavir, ampligen, arbidol, atazanavir, atripla, balapiravir, Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine, zalcitabine, zanamivir, and zidovudine, and combinations thereof.

Also disclosed is a compound for the treatment of COVID-19, wherein the compound is:

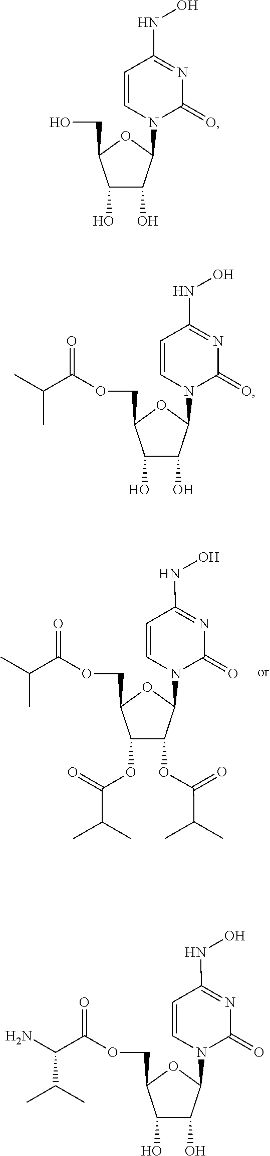 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O ; CC(C)C(=O)OC[C@H]1O[C@@H](n2ccc(NO)nc2=O)[C@H](OC(=O)C(C)C)[C@@H]1OC(=O)C(C)C ; CC(C)[C@H](N)C(=O)OC[C@H]1O[C@@H](n2ccc(NO)nc2=O)[C@H](O)[C@@H]1O]

tautomer thereof, or pharmaceutical salt or physiological salt thereof, and one or more antiviral agent selected from the group consisting of abacavir, acyclovir, acyclovir, adefovir, amantadine, amprenavir, ampligen, arbidol, atazanavir, atripla, balapiravir, Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine, zalcitabine, zanamivir, and zidovudine, and combinations thereof.

Also disclosed is a method of treating a 2019nCoV/SARS-CoV-2 infection, comprising a administering an effective amount of a compound with the following structure:

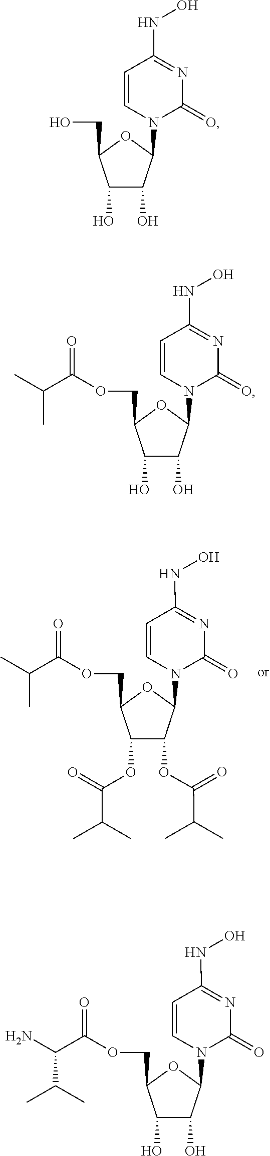 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O ; CC(C)C(=O)OC[C@H]1O[C@@H](n2ccc(NO)nc2=O)[C@H](OC(=O)C(C)C)[C@@H]1OC(=O)C(C)C ; CC(C)[C@H](N)C(=O)OC[C@H]1O[C@@H](n2ccc(NO)nc2=O)[C@H](O)[C@@H]1O]

or a tautomer thereof, or pharmaceutical salt or physiological salt thereof, and one or more antiviral agent selected from the group consisting of abacavir, acyclovir, acyclovir, adefovir, amantadine, amprenavir, ampligen, arbidol, atazanavir, atripla, balapiravir, Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine, zalcitabine, zanamivir, and zidovudine, and combinations thereof.

Also disclosed herein is a method of treating COVID-19, comprising administering an effective amount of a compound with the following structure:

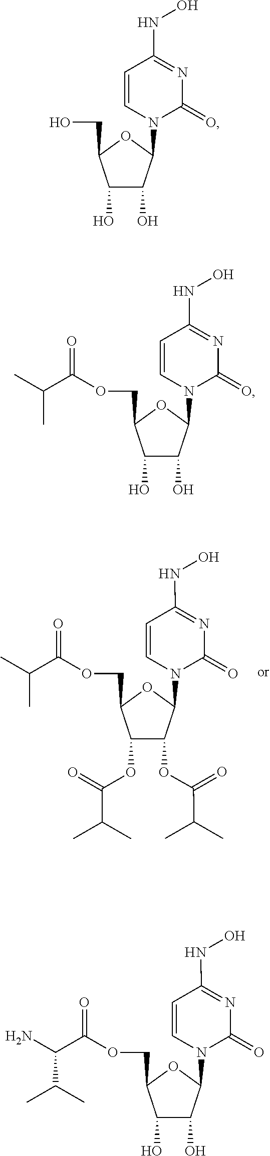 [SMILES: O=c1nc(NO)ccn1[C@@H]1O[C@H](CO)[C@@H](O)[C@H]1O ; CC(C)C(=O)OC[C@H]1O[C@@H](n2ccc(NO)nc2=O)[C@H](O)[C@@H]1O ; CC(C)C(=O)OC[C@H]1O[C@@H](n2ccc(NO)nc2=O)[C@H](OC(=O)C(C)C)[C@@H]1OC(=O)C(C)C ; CC(C)[C@H](N)C(=O)OC[C@H]1O[C@@H](n2ccc(NO)nc2=O)[C@H](O)[C@@H]1O]

tautomer thereof, or pharmaceutical salt or physiological salt thereof, and one or more antiviral agent selected from the group consisting of abacavir, acyclovir, acyclovir, adefovir, amantadine, amprenavir, ampligen, arbidol, atazanavir, atripla, balapiravir, Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine, zalcitabine, zanamivir, and zidovudine, and combinations thereof.

Also disclosed are method of treating or preventing infections caused by 2019-nCoV/SARS-CoV-2 comprising administering to a host in need an effective amount of a compound or composition as disclosed herein. In some examples, the compound can be

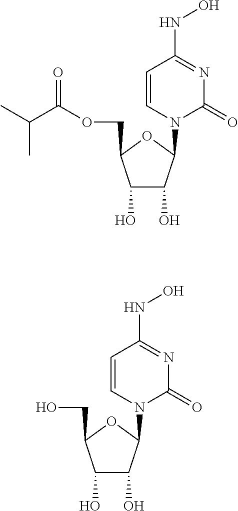 [SMILES: CC(C)C(=O)OC[C@H]1O[C@@H](n2ccc(NO)nc2=O)[C@H](O)[C@@H]1O ; O=c1nc(NO)ccn1[C@@H]1O[C@H](CO)[C@@H](O)[C@H]1O]

or a tautomer thereof, or pharmaceutical salt or physiological salt thereof.

Also disclosed is a method of treating a viral CNS infection in a patient, comprising administering to the patient having the viral CNS infection an effective amount of a composition or compound as disclosed herein. The viral CNS infection can be 2019-nCoV/SARS-CoV-2.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

All chemical reactions were performed in oven-dried glassware under a nitrogen atmosphere, except where noted. Chemicals and solvents were reagent-grade and purchased from commercial suppliers (typically Aldrich, Fisher, Acros, Carbosynth Limited, and Oakwood Chemical) and used as received, excepting where noted. In particular, EIDD-1910, EIDD-1993, and EIDD-2003 were purchased from Carbosynth Limited. Solvents used for reactions (tetrahydrofuran, methanol, acetonitrile, dichloromethane, toluene, pyridine, dimethylformamide) were ≥99.9% anhydrous in all cases. All reactions were followed by thin layer chromatography to completion, unless stated otherwise. Thin layer chromatography analysis was performed on silica gel, using illumination with a UV lamp (254 nm) or staining with KMnO₄ and heating. Manual flash column chromatography was performed with 40-60 micron (60 Å particle size) RediSep R_{f} silica gel, purchased from Teledyne Isco, as the stationary phase. Automated gradient flash column chromatography was performed on a Teledyne Isco CombiFlash Companion; normal phase separations were performed with pre-packed RediSep R_{f} silica gel as the stationary phase, and reverse phase separations were performed with pre-packed RediSep R_{f} C₁₈ High Performance Gold stationary phase. Triphosphate purifications were performed using ion-exchange chromatography, with DEAE (diethylaminoethyl) Sephadex A-25 as the stationary phase, and aqueous TEAB (triethylammonium bicarbonate) as the mobile phase.

¹H NMR spectra were measured on a Varian 400 MHz instrument, and processed using MestReNova software, version 9.0.1. Chemical shifts were measured relative to the appropriate solvent peak: CDCl₃ (δ 7.27), dimethylsulfoxide-d₆ (δ 2.50), CD₃OD (δ 3.31), D₂O (δ 4.79). The following abbreviations were used to describe coupling: s=singlet, d=doublet, t=triplet, q=quartet, p=pentet, m=multiplet, br=broad. ¹³C NMR spectra were measured on a Varian instrument at 100 MHz with chemical shifts relative to the appropriate solvent peak: CDCl₃ (δ 77.0), dimethylsulfoxide-d₆ (δ 39.5), CD₃OD (δ 49.0). ¹⁹F spectra were measured on a Varian instrument at 376 MHz, and ³¹P spectra were measured on a Varian instrument at 162 MHz. Chemical shifts for ¹⁹F spectra, ³¹P spectra, and ¹³C spectra (in D₂O only) were calibrated by MestReNova software using an absolute reference function to the corresponding ¹H NMR spectrum in the same solvent.

Nominal (low resolution) liquid chromatography/mass spectrometry was performed using an Agilent 1200 series LC (UV absorption detector at 254 nm), using a Zorbax Eclipse XDB C₁₈ 4.6×50 mm, 3.5 micron column, eluting with a methanol/water mixture (typically 95/5 isocratic) and an Agilent 6120 liquid chromatography mass spectrometer quadrupole instrument. High resolution mass spectrometry was performed by the Emory University Mass Spectrometry Center with a Thermo LTQ-FTMS using either APCI or ESI.

### Example 1: Synthesis of N4-hydroxycytidine or 1-(3,4-dihydroxy-5-(hydroxymethyl) tetrahydrofuran-2-yl)-4-(hydroxyamino)pyrimidin-2-one (EIDD-1931)

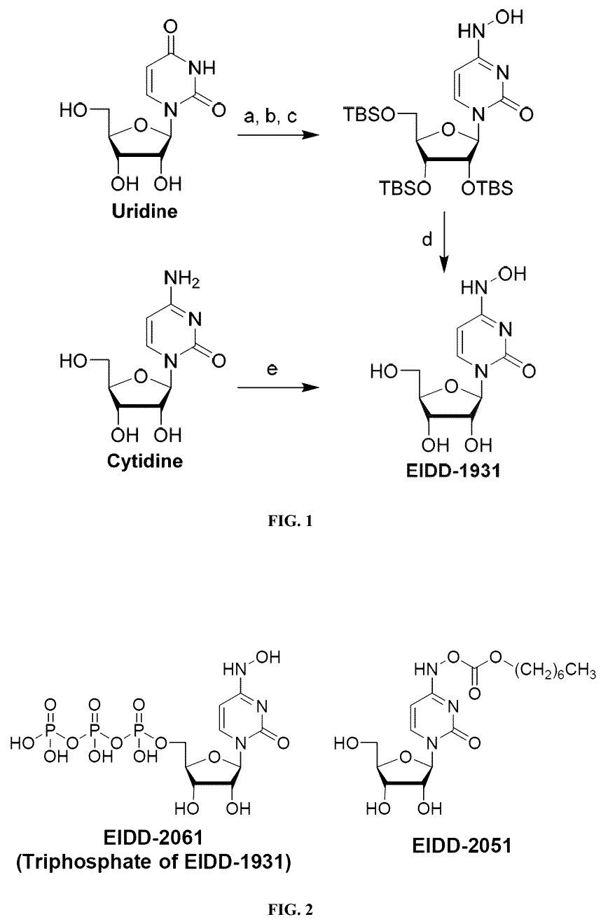

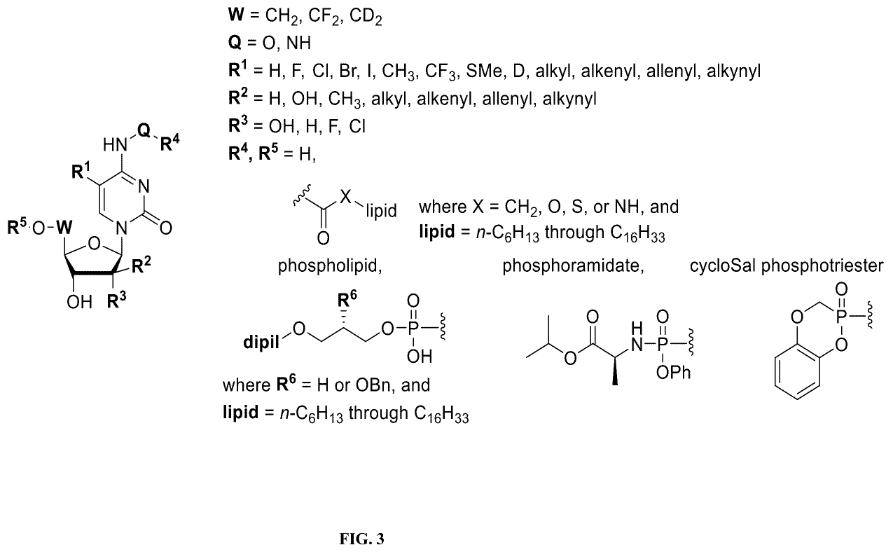

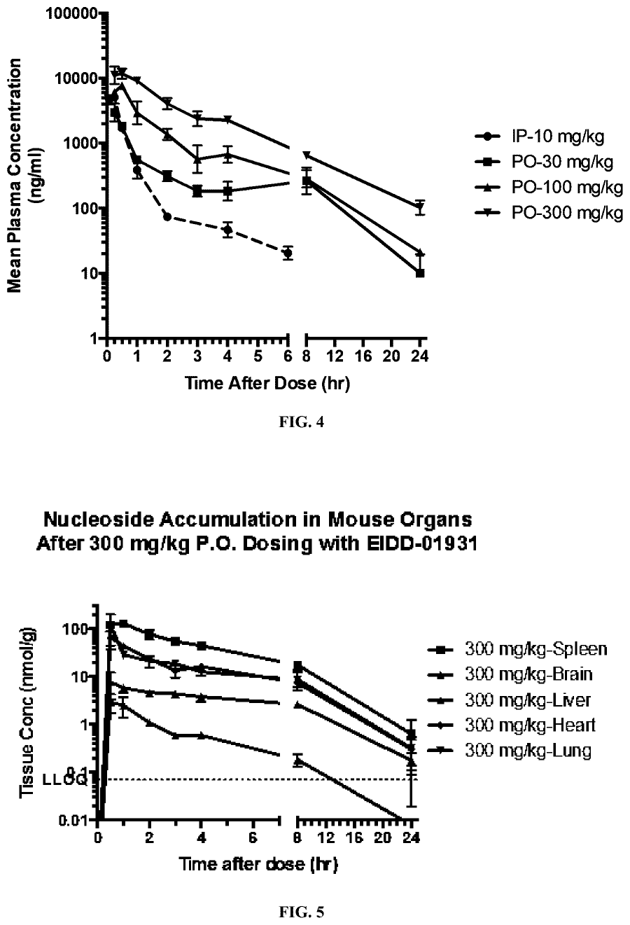

Protection of uridine by persilylation was followed by activation of the 4-position of the nucleobase by a hindered arylsulfonyl group (see FIG. 1). Displacement of this group with hydroxylamine installed the N-4-hydroxy moiety. Global deprotection using one of any number of fluoride sources available gave the desired product.

The compound could be made in one step from cytidine by heating in a pH-adjusted solution of hydroxylamine. Despite being shorter, this route tended to give lower yields and required purification by reverse phase flash column chromatography, limiting its use to producing smaller quantities.

Another synthetic route is as shown below.

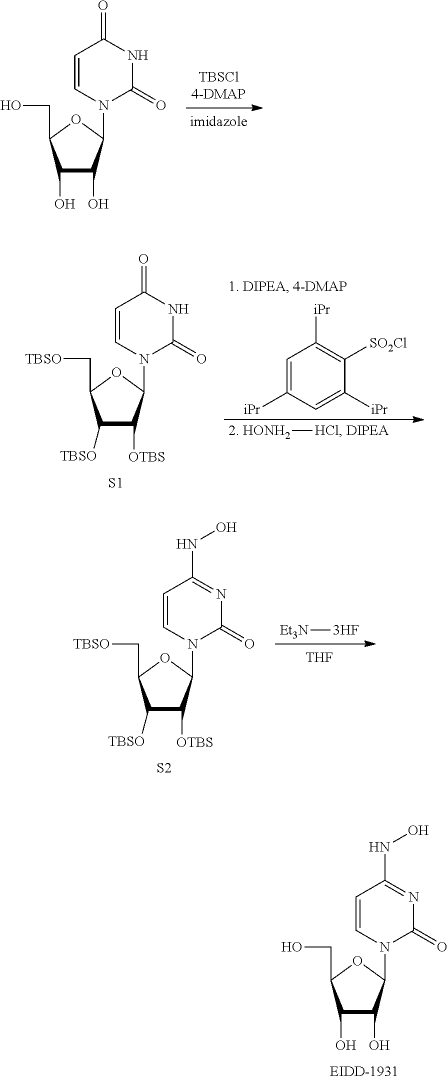

A 2 μL 3-neck flask equipped with an overhead stirrer and nitrogen inlet was charged with uridine (25 g, 102 mmol) and 1 μL of dichloromethane. The resulting solution was cooled to 0° C. and 4-dimethylaminopyridine (1.251 g, 10.24 mmol) and imidazole (27.9 g, 409 mmol) were added sequentially. Tert-butyldimethylsilyl chloride (61.7 g, 409 mmol) was added over 10 minutes, and the resulting mixture was warmed to ambient temperature and stirred for 18 hrs. Water (300 mL) was added to the reaction mixture and stirred at room temperature for 2 h, the layers were separated, and the aqueous layer was extracted with additional dichloromethane. The combined organic layers were washed with brine (1×300 mL), dried over sodium sulfate, filtered and concentrated under reduced pressure to yield 75 g of a clear colorless oil. Purification by flash chromatography (5 to 20% gradient of ethylacetate in hexanes) to yield S1 (45 g, 75%) as a clear, colorless oil, which solidified when dried in vacuo: ¹H NMR (400 MHz, CDCl₃) δ 8.09 (s, 1H), 8.02 (d, J=8.2 Hz, 1H), 5.87 (d, J=3.6 Hz, 1H), 5.67 (dd, J=8.1, 2.2 Hz, 1H), 4.07 (q, J=3.8, 3.3 Hz, 1H), 3.98 (dd, J=11.7, 1.7 Hz, 1H), 3.75 (dd, J=11.7, 1.1 Hz, 1H), 0.94 (s, 9H), 0.90 (s, 9H), 0.88 (s, 9H), 0.13 (s, 3H), 0.12 (s, 3H), 0.08 (s, 3H), 0.07 (s, 3H), 0.07 (s, 3H), 0.06 (s, 3H).

A 1 μL round bottom flask was charged with S1 (28 g, 47.7 mmol) and dichloromethane (700 mL). The solution was cooled to 0° C. using an ice bath; 4-dimethylaminopyridine (0.583 g, 4.77 mmol) and N,N-diisopropylethylamine (41.7 mL, 239 mmol) were added sequentially. 2,4,6-Triisopropylbenzene-1-sulfonyl chloride (28.9 g, 95 mmol) was slowly added to the flask, and after addition was complete, the flask was warmed to ambient temperature and stirred for 18 hrs. The dark orange solution was cooled to 0° C. with an ice bath, and N,N-diisopropylethylamine (24.66 g, 191 mmol) was added via syringe, followed by solid hydroxylamine hydrochloride (13.26 g, 191 mmol) all at once. The mixture was warmed to room temperature and stirred for 3 hrs. The reaction was quenched with water (200 mL), and the resulting layers were separated. The aqueous layer was extracted with dichloromethane (200 mL), and the combined organics were washed with brine, dried over sodium sulfate, and concentrated under reduced pressure to yield a dark orange oil. Purification by flash chromatography (15 to 50% gradient of ethylacetate in hexanes) to yield S2 (19.8 g, 69% over 2 steps) as an oil, which solidified to a semi solid upon drying in vacuo: ¹H NMR (400 MHz, CDCl₃) δ 8.15 (s, 1H), 6.31 (s, 1H), 5.91 (d, J=4.6 Hz, 1H), 5.56 (dd, J=8.2, 2.0 Hz, 1H), 4.07 (m, 2H), 4.02 (m, 1H), 3.91 (dd, J=11.6, 2.4 Hz, 1H), 3.73 (dd, J=11.6, 2.4 Hz, 1H), 0.95 (s, 9H), 0.92 (s, 9H), 0.89 (s, 9H), 0.12 (s, 6H), 0.098 (s, 3H), 0.083 (s, 3H), 0.063 (s, 3H), 0.057 (s, 3H); LRMS m/z 602.3 [M+H]+.

A 50 mL round bottom flask was charged with S2 (23.3 g, 38.7 mmol) and THF (50 mL). Triethylamine trihydrofluoride (6.30 mL, 38.7 mmol) was added all at once, and the mixture was stirred at ambient temperature for 18 hours. The mixture was concentrated under reduced pressure, and the residue was dissolved in a minimal amount of methanol, and this solution was slowly added to an Erlenmeyer flask containing rapidly stirred dichloromethane (500 mL) to precipitate the product; the mixture was stirred at room temperature for 15 minutes. The triturated solid was collected by vacuum filtration and washed with dichloromethane, then ether. The solid was dried in vacuo to yield the title compound (7.10 g, 71%) as a white solid: ¹H NMR (400 MHz, CD₃OD) δ 7.16 (d, J=8.2 Hz, 1H), 5.86 (d, J=5.6 Hz, 1H), 5.59 (d, J=8.2 Hz, 1H), 4.19-4.04 (m, 2H), 3.93 (q, J=3.3 Hz, 1H), 3.77 (dd, J=12.2, 2.9 Hz, 1H), 3.68 (dd, J=12.1, 2.9 Hz, 1H); ¹H NMR (400 MHz, dimethylsulfoxide-d₆) δ 9.95 (s, 1H), 9.46 (s, 1H), 7.02 (d, J=8.2 Hz, 1H), 5.71 (d, J=6.3 Hz, 1H), 5.54 (d, J=7.7 Hz, 1H), 5.23 (d, J=6.0 Hz, 1H), 5.02 (d, J=4.6 Hz, 1H), 4.98 (t, J=5.1 Hz, 1H), 3.95 (q, J=5.9 Hz, 1H), 3.89 (td, J=4.9 Hz, 3.0 Hz, 1H), 3.75 (q, J=3.4 Hz, 1H), 3.50 (qdd, J=11.9 Hz, 5.2 Hz, 3.5 Hz, 2H); ¹³C NMR (101 MHz, dimethylsulfoxide-d₆) δ 150.0, 143.9, 130.5, 98.89, 87.1, 85.0, 72.8, 70.8, 61.8. LRMS m/z 260.1 [M+H]⁺.

### Example 2: Synthesis of EIDD-2061

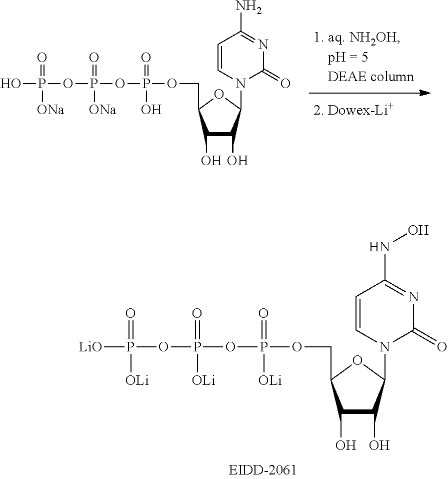

A sealable pressure tube was charged with a stir bar, cytidine triphosphate disodium salt (0.137 g, 0.260 mmol), and a 2 N aqueous hydroxylamine solution adjusted to pH=5 (2.0 mL, 4.0 mmol). After mixing the reagents, the pH of the solution was measured (pH=3) and additional drops of 10% w/w aq. NaOH solution were added to readjust the solution to pH=5. The tube was sealed and heated with stirring at 55° C. for 5 h. The mixture was cooled to room temperature, the sealed tube was opened, and a solution of 100 mM triethylammonium bicarbonate (TEAB) (2 mL) was added. The contents of the tube were transferred to a round bottom flask and concentrated by rotary evaporation. The crude material was taken up in 100 mM TEAB, and chromatography on DEAE followed by lyophilization of the product gave a triethylammonium salt of the desired product.

An ion-exchange column (17 mL CV) of freshly prepared Dowex™ (Li⁺ form) was rinsed with 5 CV water. The prepared triethylammonium salt was taken up in water and eluted through the ion-exchange column. Fractions containing product were combined and lyophilized to give the title compound (0.030 g, 22%) as a fluffy tan solid: ¹H NMR (400 MHz, D₂O) δ 7.19 (d, J=8.3 Hz, 1H), 5.95 (d, J=6.3 Hz, 1H), 5.82 (d, J=8.3 Hz, 1H), 4.42-4.34 (m, 2H), 4.24-4.10 (m, 3H); ³¹P NMR (162 MHz, D₂O) 6-8.5 (br s), −11.2 (d, J=19.6 Hz), −22.0 (t, J=19.3 Hz); LRMS m/z 498.0 [M−H]⁻.

### Example 3: Synthesis of EIDD-2101

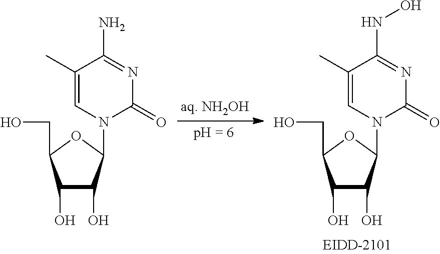

A solution of 5-methylcytidine (0.257 g, 1.00 mmol) in a 2N aq. hydroxylamine solution with pH 6 (8 mL, 16.0 mmol) was heated to 55° C. in a sealed tube with stirring for 5 hrs. The solution was cooled to room temperature, transferred to a round bottom flask, concentrated by rotary evaporation, and coevaporated with methanol (methanol) (2×20 mL). The crude residue was taken up in methanol and immobilized on silica gel. Flash chromatography (2 to 10% gradient of methanol in dichloromethane) provided the title compound (140 mg, 51%) as a light purple solid: ¹H NMR (400 MHz, CD₃OD) δ 6.99 (s, 1H), 5.86 (d, J=5.7 Hz, 1H), 4.23-4.06 (m, 2H), 3.93 (q, J=3.2 Hz, 1H), 3.78 (dd, J=12.1 Hz, 2.8 Hz, 1H), 3.70 (dd, J=12.1 Hz, 3.4 Hz, 1H), 1.79 (s, 3H); ¹³C NMR (100 MHz, CD₃OD) δ 152.0, 146.6, 128.4, 108.4, 89.4, 86.1, 74.4, 71.8, 62.8, 12.9; HRMS calcd. for C₁₀H₁₆O₆N₃ [M+H]⁺: 274.10336, found: 274.10350.

### Example 4: Synthesis of EIDD-2103

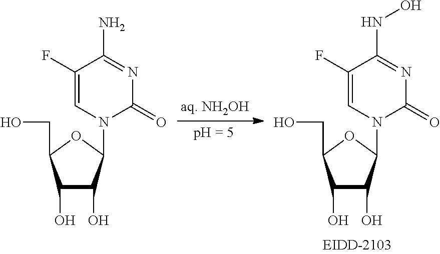

A 2 N solution of hydroxylamine hydrochloride (1.11 g, 16.0 mmol) in water (8 mL) was prepared and adjusted to pH=5 with a small amount of aq. NaOH (10% w/w). A sealable pressure tube was charged with this solution and 5-fluorocytidine (0.261 g, 1.00 mmol), the flask was sealed, and heated with stirring at 55° C. for 16 h. The mixture was cooled to room temperature, transferred to a round bottom flask, and concentrated by rotary evaporation. The crude material was suspended in methanol and immobilized on CELITE® diatomaceous earth. Automated flash chromatography (40 g column, 0 to 20% gradient of methanol in dichloromethane) gave 600 mg of a semipure pink solid. This solid was dissolved in 2 mL water, and automated reverse phase chromatography (43 g column, 5 to 100% gradient of methanol in water) gave the desired product free from organic and inorganic impurities. The solid was dissolved in water, frozen in a dry ice/acetone bath, and lyophilized to provide the title compound (0.066 g, 0.238 mmol, 24% yield) as a white flocculent solid. ¹H NMR (400 MHz, D₂O) δ 7.31 (d, J=7.6 Hz, 1H), 5.87 (dd, J=5.5 Hz, 1.8 Hz, 1H), 4.26 (t, J=5.5 Hz, 1H), 4.19 (t, J=4.8 Hz, 1H), 4.07 (q, J=3.8 Hz, 1H), 3.85 (dd, J=12.8 Hz, 3.1 Hz, 1H), 3.77 (dd, J=12.7 Hz, 4.2 Hz, 1H); ¹³C NMR (100 MHz, D₂O) δ 150.0, 139.7, 137.4, 115.6 (d, J=36.1 Hz), 88.0, 84.2, 72.8, 69.8, 61.0; ¹⁹F NMR (376 MHz, D₂O) 6-164.70 (d, J=7.6 Hz); HRMS calcd. for C₉H₁₃FN₃O₆[M+H]⁺: 278.07829, found: 278.07848.

### Example 5: Synthesis of EIDD-2216

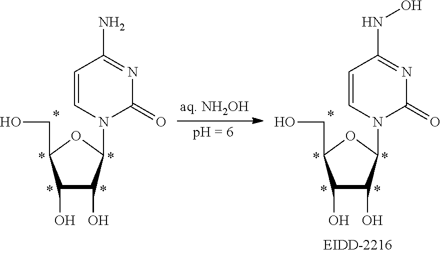

A 5 N solution of hydroxylamine hydrochloride (4.71 g, 67.8 mmol) in water (13.5 mL) was prepared and adjusted to pH=6 with a small amount of aq. NaOH (10% w/w). A sealable pressure tube was charged with this solution and [1′,2′,3′,4′,5′-¹³C₅]cytidine (0.661 g, 2.26 mmol), the flask was sealed, and heated with stirring at 37° C. for 16 h. The mixture was cooled to room temperature (rt), transferred to a round bottom flask, and concentrated by rotary evaporation. The crude material was taken up in water, and automated reverse phase flash chromatography (240 g C₁₈ column, 0 to 100% gradient of acetonitrile in water) removed bulk impurities to give 1.4 g of a wet solid. This solid was dissolved in water, and a second automated reverse phase chromatography (240 g C₁₈ column, 0 to 100% gradient of acetonitrile in water) removed more impurities to give 400 mg semipure material. The material was dissolved in methanol and immobilized on CELITE™ diatomaceous earth. Automated flash chromatography (24 g column, 5 to 25% gradient of methanol in dichloromethane) gave 200 mg of nearly pure product. The solid was dissolved in water, and a final automated reverse phase chromatography (48 g C₁₈ column, 0 to 100% gradient of acetonitrile in water) gave the desired product free from organic and inorganic impurities. The solid was dissolved in water, frozen in a dry ice/acetone bath, and lyophilized to provide the title compound (0.119 g, 20%) as a pale purple flocculent solid, about 95% pure by NMR/LCMS analysis: ¹H NMR (400 MHz, D₂O) δ 7.03 (dd, J=8.2 Hz, 2.2 Hz, 1H), 5.82 (ddd, J=167.5 Hz, 5.3 Hz, 2.9 Hz, 1H), 5.70 (d, J=8.2 Hz, 1H), 4.47-4.30 (br m, 1H), 4.23-4.03 (br m, 1H), 4.00-3.80 (br m, 2H), 3.65-3.50 (br m, 1H); ¹³C NMR (100 MHz, D₂O) δ 151.3, 146.6, 131.3, 98.7, 87.9 (dd, J=43.1 Hz, 4.0 Hz), 84.0 (dd, J=41.5 Hz, 38.0 Hz), 72.5 (dd, J=43.3 Hz, 37.8 Hz), 69.8 (td, J=37.9 Hz, 3.9 Hz), 61.1 (d, J=41.5 Hz); LRMS m/z 265.1 [M+H]⁺.

### Example 6: Synthesis of EIDD-2261

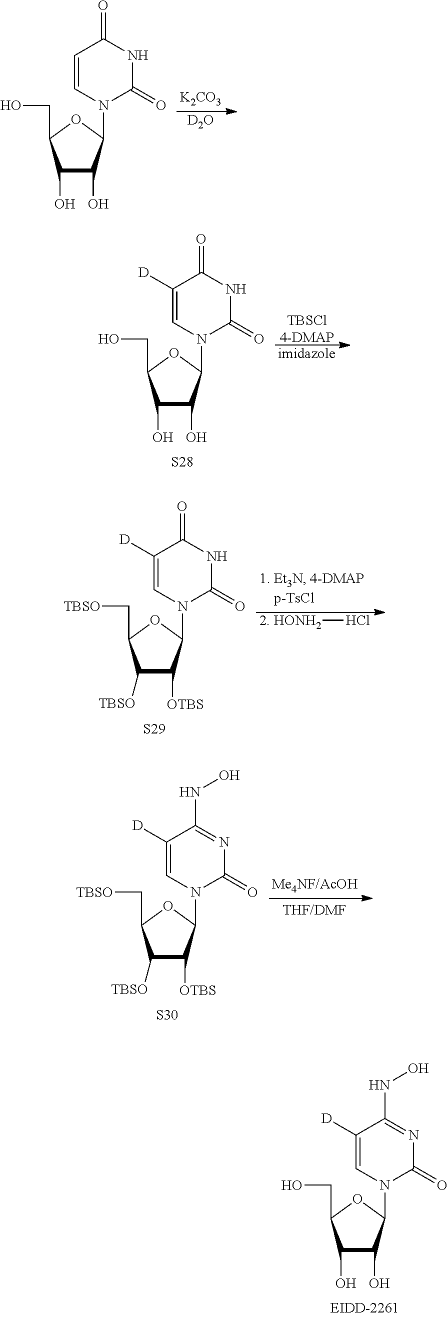

A sealable pressure tube was charged with uridine (1.00 g, 4.09 mmol), K₂CO₃ (0.679 g, 4.91 mmol), and deuterium oxide (8.2 mL). The mixture was purged with nitrogen for 15 minutes, the tubed was sealed, and the contents were heated with stirring at 95° C. for 16 h. The mixture was cooled to room temperature, the tube was unsealed, and the mixture was transferred to a round-bottom flask and concentrated by rotary evaporation. The resulting crude was coevaporated with methanol (×3) to remove water. NMR analysis showed >95% deuterium incorporation at the 5-position on the nucleobase. The light brown solid S28 (1.00 g, 100%) was used in the next step without further purification: ¹H NMR (400 MHz, CD₃OD) δ 7.76 (s, 1H), 5.88 (d, J=4.2 Hz, 1H), 4.17-4.12 (m, 2H), 4.00-3.96 (m, 1H), 3.84 (dd, J=12.3 Hz, 2.8 Hz, 1H), 3.72 (dd, J=12.3 Hz, 3.5 Hz, 1H); ¹³C NMR (100 MHz, CD₃OD) δ 185.6, 177.4, 160.4, 141.1, 91.8, 85.8, 75.9, 71.2, 62.4.

A round bottom flask was charged with S28 (1.00 g, 4.09 mmol) and dichloromethane (8 mL) under nitrogen. The resulting mixture was cooled to 0° C. and 4-dimethylaminopyridine (0.050 g, 0.408 mmol) and imidazole (1.11 g, 16.3 mmol) were added all at once. Tert-butyldimethylsilyl chloride (2.15 g, 14.3 mmol) was added all at once as a solid, the mixture was warmed to ambient temperature, and stirred for 16 hours. Water (25 mL) was added to the reaction mixture, the layers were separated, and the aqueous layer was extracted with dichloromethane (2×25 mL). The combined organic layers were washed with brine (1×25 mL), dried over Na₂SO₄, filtered, and concentrated by rotary evaporation. Automated flash chromatography (40 g column, 0 to 35% gradient of ethyl acetate in hexanes) gave S29 (2.52 g, 84%) as an off-white foam: ¹H NMR (400 MHz, CDCl₃) δ 8.08 (br s, 1H), 8.03 (s, 1H), 5.89 (d, J=3.6 Hz, 1H), 4.12-4.06 (m, 3H), 3.99 (dd, J=11.5 Hz, 1.8 Hz, 1H), 3.76 (d, J=12.0 Hz, 1H), 0.96 (s, 9H), 0.92 (s, 9H), 0.90 (s, 9H), 0.14 (s, 3H), 0.13 (s, 3H), 0.10 (s, 3H), 0.09 (s, 3H), 0.08 (s, 3H), 0.07 (s, 3H); ¹³C NMR (100 MHz, CDCl₃) δ 163.7, 150.3, 140.3, 89.0, 84.3, 76.1, 70.5, 61.6, 26.0 (3C), 25.8 (3C), 25.7 (3C), 18.4, 18.3, 17.9, −4.2, −4.6, −4.8, −4.9, −5.4, −5.6; HRMS calcd. for C₂₇H₅₄DN₂NaO₆Si [M+Na]⁺: 610.32446, found: 610.32482.

To a stirred solution of S29 (0.840 g, 1.43 mmol) in acetonitrile (14.3 mL) at 0° C. under nitrogen, were added sequentially p-toluenesulfonyl chloride (0.545 g, 2.86 mmol), 4-dimethylaminopyridine (0.175 g, 1.43 mmol), and triethylamine (0.80 mL, 5.71 mmol). The mixture was stirred at 0° C. for 2.5 h, at which time hydroxylamine hydrochloride (0.993 g, 14.3 mmol) was added all at once as a solid. The mixture was heated at 50° C. for 3 days, then cooled to room temperature. The reaction mixture was diluted with ethyl acetate (100 mL), then washed with water (2×100 mL) and brine (1×100 mL), dried over Na₂SO₄, filtered, and concentrated by rotary evaporation. Automated flash chromatography (40 g column, 5 to 35% gradient of ethyl acetate in hexanes) produced a mixture of starting material and desired product. A second automated flash chromatography (24 g column, 10 to 40% gradient of ethyl acetate in hexanes), gave S30 (0.332 g, 39%) as an off-white foam: ¹H NMR (400 MHz, CDCl₃) δ 8.37 (br s, 1H), 5.92 (d, J=4.6 Hz, 1H), 4.10-4.05 (m, 2H), 4.04-4.00 (m, 1H), 3.91 (dd, J=11.6 Hz, 2.4 Hz, 1H), 3.73 (dd, J=11.6 Hz, 1.8 Hz, 1H), 0.95 (s, 9H), 0.92 (s, 9H), 0.89 (s, 9H), 0.12 (s, 6H), 0.10 (s, 3H), 0.08 (s, 3H), 0.06 (s, 3H), 0.05 (s, 3H).

A round bottom flask was charged with S30 (0.332 g, 0.551 mmol), tetramethylammonium fluoride (0.196 g, 2.64 mmol), tetrahydrofuran (THF) (8.25 mL), and dimethylformamide (DMF) (2.75 mL) under nitrogen at 0° C. Acetic acid (0.157 mL, 2.75 mmol) was added all at once via syringe. The mixture was warmed to 45° C. and heated with stirring for 4 days, then concentrated by rotary evaporation. Automated flash chromatography (40 g column, 0 to 20% gradient of methanol in dichloromethane) gave the title compound (0.106 g, 74%) as a white solid. Final NMR analysis showed >95% deuterium incorporation at the 5-position of the nucleobase: ¹H NMR (400 MHz, D₂O) δ 7.16 (s, 1H), 5.85 (d, J=5.6 Hz, 1H), 4.14 (t, J=5.5 Hz, 1H), 4.10 (dd, J=5.6 Hz, 3.8 Hz, 1H), 3.93 (q, J=3.4 Hz, 1H), 3.77 (dd, J=12.2 Hz, 2.9 Hz, 1H), 3.68 (dd, J=12.2 Hz, 3.4 Hz, 1H); ¹³C NMR (100 MHz, CD₃OD) δ 151.8, 146.3, 132.1, 89.7, 86.1, 74.6, 71.8, 62.8; HRMS calcd. for C₉H₁₃DN₃O₆[M+H]⁺: 261.09399, found: 261.09371.

### Example 7: Synthesis of EIDD-2345

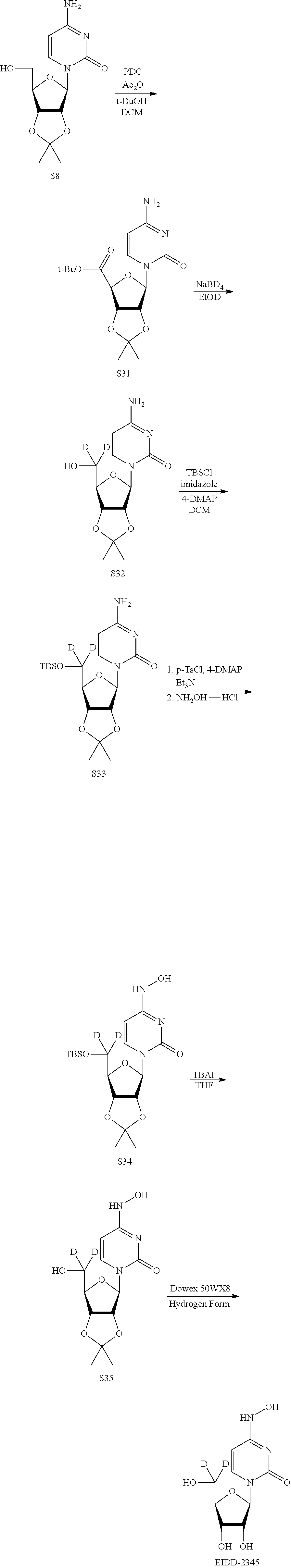

A round bottom flask was charged with S8 (3.13 g, 11.0 mmol) and dichloromethane (75 mL) under nitrogen at room temperature. To this stirred mixture was added sequentially pyridinium dichromate (8.28 g, 22.0 mmol), acetic anhydride (10.4 mL, 110 mmol) and t-butanol (21.1 mL, 220 mmol) at room temperature. The mixture was stirred for 22 hours at room temperature, then washed with water (1×75 mL). The aqueous layer was extracted with dichloromethane (2×75 mL), and the combined organic layers were washed with brine (1×100 mL), dried over Na₂SO₄, filtered, and concentrated by rotary evaporation. The obtained residue was taken up in ethyl acetate and filtered through a plug of CELITE™ diatomaceous earth, followed by washing with ethyl acetate. The filtrate was concentrated by rotary evaporation, and automated flash chromatography (120 g column, 40 to 80% gradient of ethyl acetate in hexanes) gave S31 (3.10 g, 72%) as an off-white foam: ¹H NMR (400 MHz, CDCl₃) δ 8.36 (br s, 1H), 7.42 (d, J=8.0 Hz, 1H), 5.76 (dd, J=8.0 Hz, 2.3 Hz, 1H), 5.59 (s, 1H), 5.27 (dd, J=6.0 Hz, 1.8 Hz, 1H), 5.19 (d, J=6.0 Hz, 1H), 4.62 (d, J=1.8 Hz, 1H), 1.56 (s, 3H), 1.48 (s, 9H), 1.39 (s, 3H).

To a stirred solution of S31 (2.61 g, 7.37 mmol) in deuterated ethanol (75 mL) at room temperature under nitrogen, was added NaBD₄ (1.234 g, 29.5 mmol) in one portion. The mixture was stirred at room temperature for 1 hour, heated to 55° C. for 6 hours, then overnight at room temperature. The mixture was cooled to 0° C., and excess reagent was quenched with deuterated acetic acid. The mixture was concentrated by rotary evaporation to give crude S32 (2.57 g), which was taken directly on to the next step without further purification.

To a stirred suspension of crude S32 (2.00 g impure material, 5.74 mmol) in dichloromethane (70 mL) at 0° C., was added solid imidazole (1.90 g, 27.9 mmol) and 4-dimethylaminopyridine (0.171 g, 1.40 mmol). Solid t-butyldimethylsilyl chloride (2.11 g, 14.0 mmol) was added, and the mixture was warmed to room temperature and stirred for 4 days. The mixture was washed sequentially with water and brine (1×70 mL each), dried over Na₂SO₄, filtered, and concentrated by rotary evaporation. Automated flash chromatography (120 g column, 0 to 35% gradient of ethyl acetate in hexanes) gave S33 (1.42 g, 66% over 2 steps) as a white solid: ¹H NMR (400 MHz, CDCl₃) δ 8.30 (br s, 1H), 7.72 (m, 1H), 5.99 (d, J=2.8 Hz, 1H), 5.69 (dd, J=8.2 Hz, 2.3 Hz, 1H), 4.77 (dd, J=6.1 Hz, 2.9 Hz, 1H), 4.69 (dd, J=6.2 Hz, 2.8 Hz, 1H), 4.33 (d, J=3.0 Hz, 1H), 1.60 (s, 3H), 1.37 (s, 3H), 0.91 (s, 9H), 0.11 (s, 3), 0.10 (s, 3H); ¹³C NMR (100 MHz, CDCl₃) δ 162.7, 149.9, 140.5, 114.1, 102.1, 91.9, 86.5, 85.4, 80.3, 27.4, 25.9 (3C), 25.4, 18.4, −5.4, −5.5; HRMS calcd. for C₁₈H₂₉D₂N₂O₆Si [M+H]⁺: 401.20714, found: 401.20663.

To a stirred solution of S33 (1.42 g, 3.55 mmol) in acetonitrile (35 mL) at 0° C. under nitrogen, was added sequentially p-toluenesulfonyl chloride (1.35 g, 7.09 mmol), 4-dimethylaminopyridine (0.433 g, 3.55 mmol), and triethylamine (9.88 mL, 70.9 mmol). The resulting mixture was stirred at 0° C. for 2.5 hours. Hydroxylamine hydrochloride (2.46 g, 35.5 mmol) was added, and the mixture was heated with stirring at 50° C. for 2 days. The mixture was recooled to room temperature and diluted with ethyl acetate (100 mL), then washed with water (2×50 mL) and brine (1×50 mL), dried over Na₂SO₄, filtered, and concentrated by rotary evaporation. Automated flash chromatography (120 g column, 1 to 3.5% gradient of methanol in dichloromethane) gave S34 (0.416 g, 28%) as an off-white solid: ¹H NMR (400 MHz, CDCl₃) δ 8.36 (br s, 1H), 7.00 (m, 1H), 5.97 (d, J=3.1 Hz, 1H), 5.58 (d, J=8.2 Hz, 1H), 4.77 (dd, J=6.2 Hz, 3.2 Hz, 1H), 4.68 (dd, J=6.3 Hz, 3.2 Hz, 1H), 4.22 (d, J=3.2 Hz, 1H), 1.59 (s, 3H), 1.36 (s, 3H), 0.92 (s, 9H), 0.11 (s, 3H), 0.10 (s, 3H); ¹³C NMR (100 MHz, CDCl₃) δ 149.0, 145.4, 131.4, 114.1, 98.3, 90.8, 85.5, 84.5, 80.2, 27.4, 25.9 (3C), 25.5, 18.4, −5.4, −5.5; HRMS calcd. for C₁₈H₂₉D₂N₃O₆Si [M+H]⁺: 416.21804, found: 416.21827.

To a stirred solution of S34 (0.416 g, 1.00 mmol) in THF (5 mL) at 0° C. under nitrogen, was added a 1.0 μM THF solution of TBAF (1.50 mL, 1.5 mmol), and the resulting mixture was kept at 0° C. for 24 hours. The reaction mixture was concentrated by rotary evaporation, and automated flash chromatography (40 g column, 0 to 8% gradient of methanol in dichloromethane) gave S35 (0.257 g, 85%) as a white solid: ¹H NMR (400 MHz, CD₃OD) δ 7.02 (m, 1H), 5.81 (d, J=3.2 Hz, 1H), 5.58 (d, J=8.2 Hz, 1H), 4.86 (dd, J=6.4 Hz, 3.2 Hz, 1H), 4.79 (dd, J=6.5 Hz, 3.6 Hz, 1H), 4.09 (d, J=3.7 Hz, 1H), 1.54 (s, 3H), 1.34 (s, 3H); ¹³C NMR (100 MHz, CD₃OD) δ 151.3, 146.2, 133.4, 115.2, 99.4, 92.9, 87.2, 84.9, 82.1, 27.6, 25.6; HRMS calcd. for C₁₂H₁₆D₂N₃O₆[M+H]⁺: 302.13157, found: 302.13130.

To a stirred solution of S35 (0.140 g, 0.465 mmol) in methanol (8.4 mL) and water (0.93 mL) at room temperature, was added Dowex 50WX8 hydrogen form (0.30 g), and the mixture was stirred at room temperature for 24 hours. The reaction mixture was filtered, and the filtrate was concentrated by rotary evaporation. Automated flash chromatography (40 g column, 5 to 20% gradient of methanol in dichloromethane) gave the title compound (0.050 g, 41%) as an off-white solid: ¹H NMR (400 MHz, CD₃OD) δ 7.17 (m, 1H), 5.86 (d, J=5.6 Hz, 1H), 5.60 (d, J=8.2 Hz, 1H), 4.15 (t, J=5.5 Hz, 1H), 4.11 (dd, J=5.6 Hz, 3.5 Hz, 1H), 3.94 (d, J=3.8 Hz, 1H); 13C NMR (100 MHz, CD₃OD) δ 151.8, 146.3, 132.2, 99.3, 89.7, 86.0, 74.6, 71.7, HRMS calcd. for C₉H₁₀D₂N₃O₆ [M+H]⁺: 260.08571, found: 260.08578.

### Example 8: Synthesis of EIDD-2898

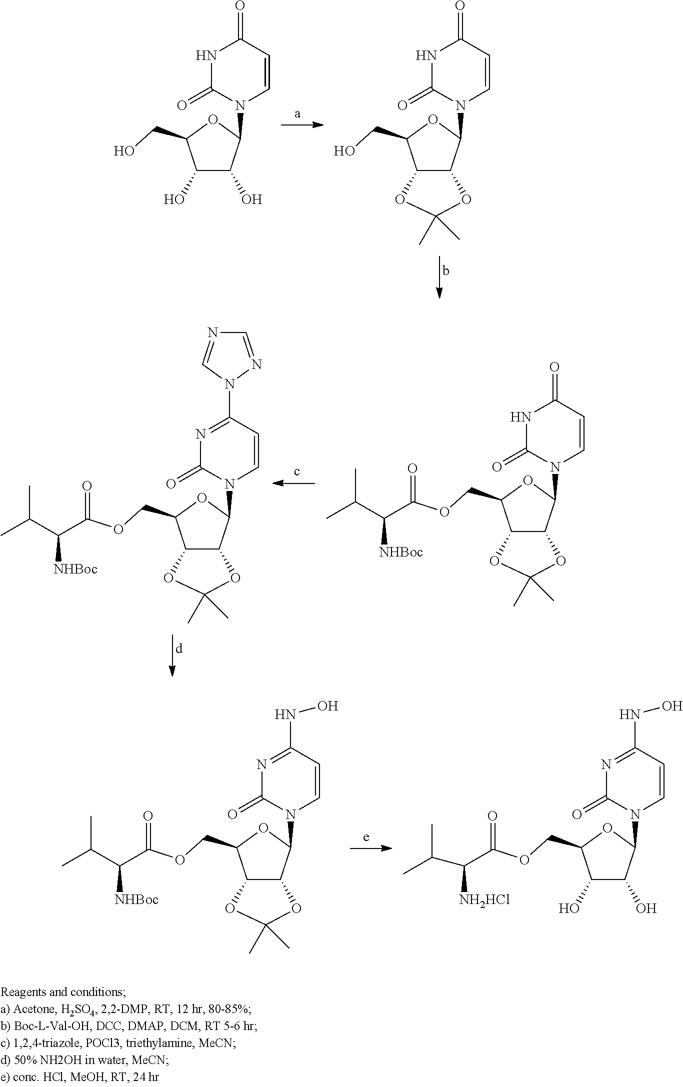

A 2 L 3-neck round bottom flask was charged with 1-[(3R,4S,5R)-3,4-dihydroxy-5-(hydroxymethyl)tetrahydrofuran-2-yl]pyrimidine-2,4-dione (61.4 g, 251.43 mmol) and acetone (1400 mL). The resulting slurry was stirred at room temperature, and sulfuric acid (2 mL was added. Stirring was continued overnight. The clear colorless solution was quenched/adjusted to basic pH with 100 mL of trimethylamine. The crude solution was concentrated under reduced pressure to yield a pale-yellow oil. The residue was dissolved in 600 mL of ethyl acetate and washed with water×2, bicarb×2, water, brine×2 and dried over sodium sulfate. The colorless solution was concentrated under reduced pressure to yield 1-[(3aR,6R,6aR)-6-(hydroxymethyl)-2,2-dimethyl-3a,4,6,6a-tetrahydrofuro[3,4-d][1,3]dioxol-4-yl]pyrimidine-2,4-dione (45 g) as a white solid.

A 200 mL round bottom flask was charged with 1-[(3aR,6R,6aR)-6-(hydroxymethyl)-2,2-dimethyl-3a,4,6,6a-tetrahydrofuro[3,4-d][1,3]dioxol-4-yl]pyrimidine-2,4-dione (2.36 g, 8.3 mmol) and dichloromethane (50 mL). The reaction was stirred until a solution was formed. Next, (2S)-2-(tert-butoxycarbonylamino)-3-methyl-butanoic acid (2.16 g, 9.96 mmol) and N,N-dimethylpyridin-4-amine (0.1 g, 0.8300 mmol) were added. The reaction was cooled to 0° C. with an ice bath. A dichloromethane solution of N,N′-dicyclohexylcarbodiimide (2.06 g, 9.96 mmol) was added slowly. The reaction mixture was allowed to warm to room temperature. Monitored by thin layer chromatography (ethyl acetate).

A precipitate formed after about 1 hr, and no starting material was detected after 3 hrs. The solids were filtered off and rinsed with ethyl acetate. The filtrate was washed with water, brine, dried over sodium sulfate and concentrated under reduced pressure to yield white, gooey solid. The gummy solid was triturated with ether and filtered to remove the solid. The filtrate was concentrated under reduced pressure to yield about 8 g of thick viscous oil. The product was purified by SGC, pooled fractions 6-25 and concentrated under reduced pressure to yield [(3aR,6R,6aR)-4-(2,4-dioxopyrimidin-1-yl)-2,2-dimethyl-3a,4,6,6a-tetrahydrofuro[3,4-d][1,3]dioxol-6-yl]methyl (2S)-2-(tert-butoxycarbonylamino)-3-methyl-butanoate (3.8 g, 7.8592 mmol, 94.667% yield) as a foamy white solid after drying in vacuo.

1,2,4-triazole was taken in anhydrous acetonitrile and stirred at room temperature after 30 min, the reaction mixture was cooled to 0° C. and POC₃ was added dropwise and continued stirring for 2 hr. After 2 hr triethylamine was added dropwise and stirring continued for 1 hr, the reaction mixture was slowly brought to room temperature, and the uridine derived substrate from the above reaction was added as solution in acetonitrile. The reaction mixture stirred at room temperature overnight. After completion of the reaction, the solvent was removed under reduced pressure and taken in dichloromethane and extracted with water. The organic layer was dried over anhydrous sodium sulphate and concentrated under reduced pressure. The crude product was purified by flash column chromatography.

To a solution of the substrate in acetonitrile (10 mL/gm), 50% hydroxylamine in water was added dropwise and stirred at room temperature for 2-3 hrs. After completion of the reaction, solvent was removed under reduced pressure, and the crude product was purified by flash column chromatography using hexane and ethyl acetate as eluent.

1 g of substrate was taken in 20 mL of methanol and treated with 2 mL of conc. HCl (36%), and after 3-4 hr 30% completion was observed. Another 5 mL of conc. HCl was added and stirred overnight. After completion of the reaction, solvent was removed, and the crude product was taken in minimum methanol and added dropwise to excess diethylether; with stirring, product was crashed out of solution and allowed to settle, ether was decanted, and fresh ether was added, stirred, settled and decanted; the same process was repeated two times. After ether was decanted, solid was dried over a rotavap and high vacuum to get free flowing white solid. Ether was trapped in the solid and was difficult to remove. The solid was dissolved in methanol, evaporated and dried to get colorless foam, which still holds methanol. The foam was taken in water, and a purple solution was observed. The purple solution was purified by reverse phase ISCO column chromatography using water and acetonitrile. The fractions containing product were evaporated under reduced pressure and lyophilized to get colorless solid.

### Example 9: Synthesis of EIDD-2800

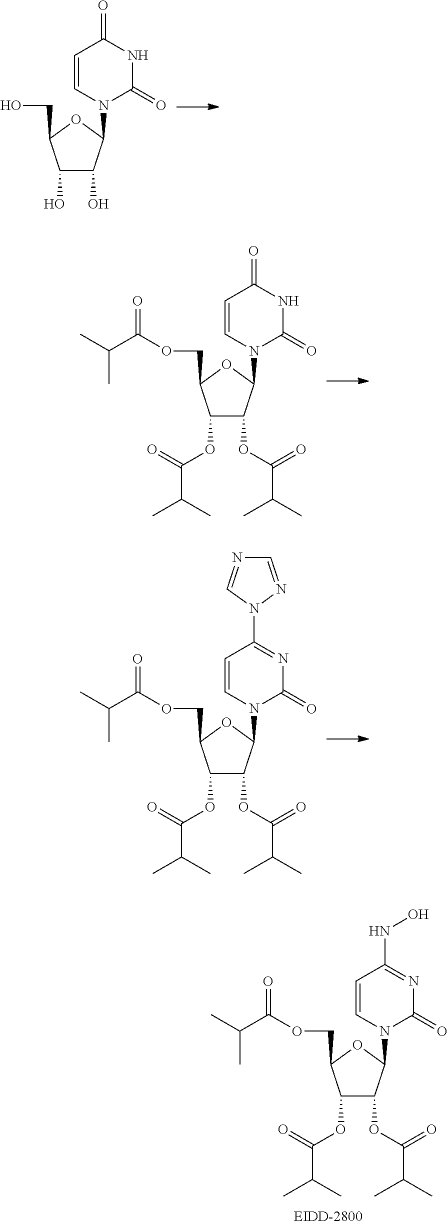

A 3-neck 1 L round bottom flask equipped with an overhead stirrer, temperature probe and addition funnel was charged with uridine (25 g, 102.38 mmol) and ethyl acetate (500 mL). The white slurry was stirred at ambient temperature while triethylamine (71.39 mL, 511.88 mmol) and 4-dimethylaminopyridine (0.63 g, 5.12 mmol) were added to the mixture. The slurry was cooled in an ice bath, and isobutyric anhydride (56.02 mL, 337.84 mmol) was slowly added to the reaction mixture over a 5-minute period. The temperature rose 25° C. during the addition. The resulting slurry was stirred at ambient temperature and monitored by thin layer chromatography. After 1 hour, a clear colorless solution had formed, and thin layer chromatography showed no starting material. The reaction was quenched with 200 mL of water, stirred at room temperature for 20 minutes. The layers were separated, and the organics were washed with water (2×100 mL), saturated aqueous bicarbonate solution (100 mL×2), 100 mL of water, brine (100 mL×2), and then dried over sodium sulfate. The organics were filtered, and the filtrate was concentrated under reduced pressure at 45° C. to yield a yellow oil. The oil was used in the next step without any further purification.

A 2 L 3-neck flask equipped with an argon inlet, overhead stirrer and temperature probe was charged with 1H-1,2,4-triazole (50.88 g, 736.68 mmol), triethylamine (114.17 mL, 818.54 mmol) and acetonitrile (350 mL). The reaction mixture was stirred at room temperature for 20 minutes. An ethyl acetate (350 mL) solution of [(2R,3R,4R)-5-(2,4-dioxopyrimidin-1-yl)-3,4-bis(2-methylpropanoyloxy)tetrahydrofuran-2-yl]methyl 2-methylpropanoate (46.5 g, 102.32 mmol) was added, and the mixture was cooled to <5° C. using an ice bath. Stirring continued for 20 minutes. Next, phosphorous(V)oxychloride (14.35 mL, 153.48 mmol) was added slowly under argon at less than 20° C. over 15 minutes. The reaction was monitored by thin layer chromatography (100% ethyl acetate), starting material (R_{f}=0.89) consumed in less than 2 hours and a new spot due to product (R_{f}=0.78) present. The reaction was quenched with 500 mL of water and 400 mL of ethyl acetate. The quenched reaction was allowed to stir at room temperature for 15 minutes. The layers were separated, and the organic layer was washed with water (2×100 mL), 200 mL of 0.5N HCl, and brine (2×100 mL). The organics were dried over sodium sulfate, filtered and concentrated under reduced pressure to yield [(2R,3R,4R)-3,4-bis(2-methylpropanoyloxy)-5-[2-oxo-4-(1,2,4-triazol-1-yl)pyrimidin-1-yl]tetrahydrofuran-2-yl]methyl 2-methylpropanoate (49 g, 96.93 mmol, 94.735% yield) as a yellow oil. The crude material was used in the next step without further purification.

A 500 mL round bottom flask was charged with [(2R,3R,4R)-3,4-bis(2-methylpropanoyloxy)-5-[2-oxo-4-(1,2,4-triazol-1-yl)pyrimidin-1-yl]tetrahydrofuran-2-yl]methyl 2-methylpropanoate (48.9 g, 96.73 mmol), ethyl acetate (400 mL), and isopropyl alcohol (100 mL). The reaction mixture was stirred at room temperature until all of the starting material was dissolved. The orange solution was treated with hydroxylamine (6.52 mL, 106.41 mmol), and the resulting pale-yellow solution was stirred at room temperature and monitored by thin layer chromatography (ethyl acetate). No starting material was observed after 1 hour. The reaction was quenched with 500 mL of water, and the layers were separated. The organics were washed with 100 mL of water, 100 mL×2 of brine, and then dried over sodium sulfate. The organics were filtered and concentrated under reduced pressure to yield the crude product. The crude product was dissolved in 180 mL of hot methyl tert-butyl ether and allowed to cool to room temperature. Seed crystals were added, and the flask was placed in the freezer. The white solid that formed was collected by filtration, washed with a minimal amount of methyl tert-butyl ether and dried in vacuo to yield the desired product.

### Example 10: Synthesis of EIDD-2801

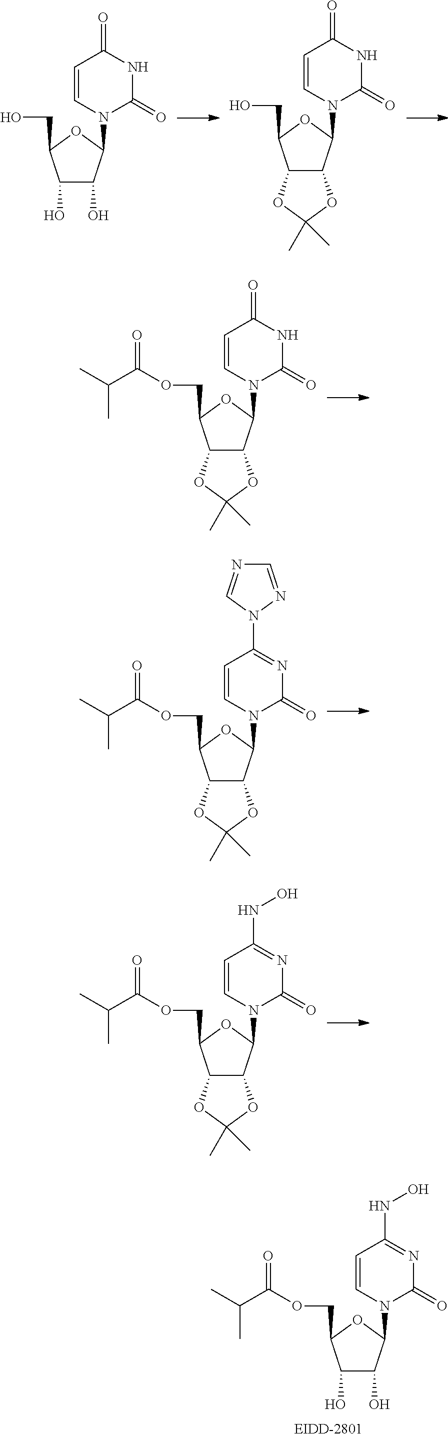

A 1 L round bottom flask was charged with uridine (25 g, 102.38 mmol) and acetone (700 mL). The reaction mixture was allowed to stir at room temperature. The slurry was then treated with sulfuric acid (0.27 mL, 5.12 mmol). Stirring was allowed to continue at room temperature for 18 hours. The reaction was quenched with 100 mL of trimethylamine and was used in the next step without further purification.

A 1 L round bottom flask was charged with the reaction mixture from the previous reaction. Triethylamine (71.09 mL, 510.08 mmol) and 4-dimethylaminopyridine (0.62 g, 5.1 mmol) were then added. The flask was cooled using an ice bath and then 2-methylpropanoyl 2-methylpropanoate (17.75 g, 112.22 mmol) was slowly added. The reaction mixture was allowed to stir at room temperature until the reaction was complete. The reaction mixture was concentrated under reduced pressure, and the residue was dissolved in 600 mL ethyl acetate and washed with saturated aqueous bicarbonate solution×2, water×2 and brine×2. The organics were dried over sodium sulfate and concentrated under reduced pressure to yield a clear colorless oil. The crude product was used in the next step without further purification.

A 1 L round bottom flask was charged with the crude product from above (36 g, 101.59 mmol) and acetonitrile (406.37 mL). The reaction mixture was allowed to stir until all the starting material was dissolved. Next, 1,2,4-triazole (50.52 g, 731.46 mmol) was added followed by the addition of N,N-diethylethanamine (113.28 mL, 812.73 mmol). The reaction mixture was allowed to stir at room temperature until all solids dissolved. The reaction was then cooled to 0° C. using an ice bath. Phosphorous oxychloride (24.44 mL, 152.39 mmol) was added slowly. The slurry that formed was allowed to stir under argon while slowly warming to room temperature. The reaction was then allowed to stir until complete by thin layer chromatography (ethyl acetate). The reaction was then quenched by the addition of 100 mL of water. The slurry then became a dark colored solution, which was then concentrated under reduced pressure. The residue was dissolved in dichloromethane and washed with water and brine. The organics were then dried over sodium sulfate, filtered, and concentrated under reduced pressure. The product was purified by silica gel chromatography (2×330 g columns). All fractions containing product were collected and concentrated under reduced pressure.

A 500 mL round bottom flask was charged with the product from the previous step (11.8 g, 29.11 mmol) and isopropyl alcohol (150 mL). The reaction mixture was allowed to stir at room temperature until all solids dissolved. Next, hydroxylamine (1.34 mL, 43.66 mmol) was added and stirring continued at ambient temperature. When the reaction was complete high performance liquid chromatography (HPLC), some solvent was removed under high vacuum at ambient temperature. The remaining solvent was removed under reduced pressure at 45° C. The resulting residue was dissolved in ethyl acetate and was washed with water and brine. The organics were dried over sodium sulfate, filtered, and concentrated under reduced pressure to yield oil. Crystals formed upon standing at room temperature. The crystals were collected by filtration, washed with ether×3, and dried in vacuo to provide the product as a white solid.

A 200 mL round bottom flask was charged with the product from the previous step (6.5 g, 17.6 mmol) and formic acid (100 mL, 2085.6 mmol). The reaction mixture was allowed to stir at room temperature overnight. The progress of the reaction was monitored by HPLC. The reaction mixture was concentrated under reduced pressure at 42° C. to yield a clear, pale pink oil. Next, 30 mL of ethanol was added. Solvent was then removed under reduced pressure. Methyl tert-butyl ether (50 mL) was added to the solid and heated. Next, isopropyl alcohol was added, and heating was continued until all solid material dissolved (5 mL). The solution was then allowed to cool and stand at room temperature. A solid started to form after about 1 hr. The solids were collected by filtration, washed with methyl tert-butyl ether, and dried in vacuo to yield the EIDD-2801 as a white solid. The filtrate was concentrated under reduced pressure to yield a sticky solid, which was dissolved in a small amount of isopropyl alcohol with heating. The solution was allowed to stand at room temperature overnight. A solid formed in the flask, which was collected by filtration, rinsed with isopropyl alcohol and methyl tert-butyl ether, and dried in vacuo to an additional crop of desired product.

EIDD-2801 (25 g) was dissolved in 250 mL of isopropyl alcohol by heating to 70° C. to give a clear solution. The warm solution was polish filtered and filtrate transferred to 2 L three neck flask with overhead stirrer. It was warmed back to 70° C., and methyl tert-butyl ether (250 mL) was slowly added into the flask. The clear solution was seeded and allowed to cool slowly to room temperature with stirring for 18 hrs. The EIDD-2801 solid that formed was filtered and washed with methyl tert-butyl ether and dried at 50° C. under vacuum for 18 hours. The filtrate was concentrated, redissolved in 50 mL isopropyl alcohol and 40 mL methyl tert-butyl ether by warming to give clear solution and allowed to stand at room temperature to give a second crop of EIDD-2801.

### Example 11

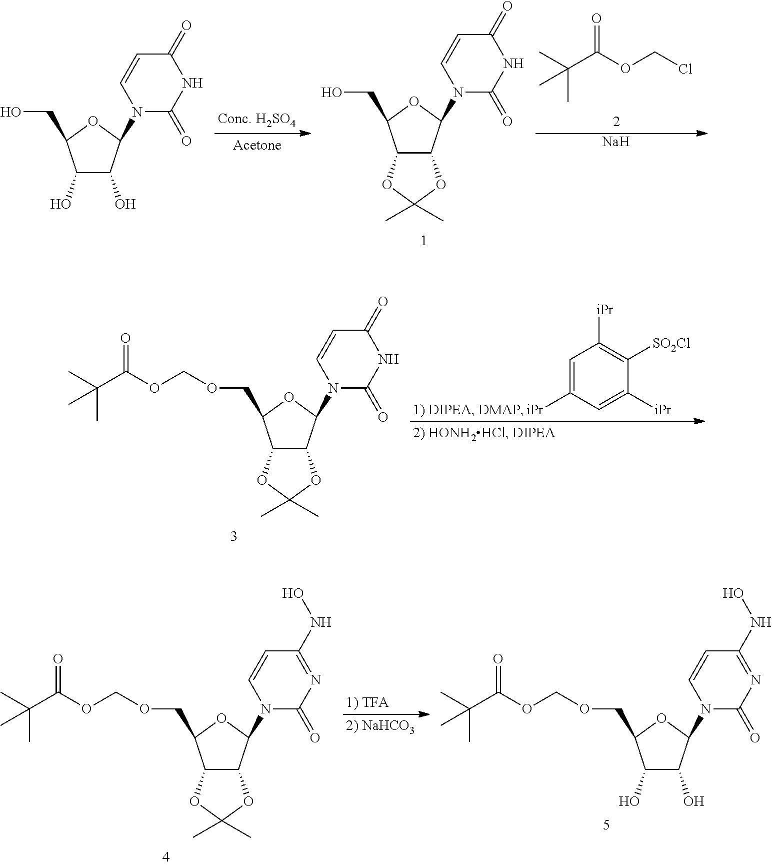

To a stirred solution of uridine (1 eq) in acetone (0.08 M) cooled to 0° C. was added concentrated H₂SO₄ (2.3 eq). The reaction mixture was stirred at room temperature for 4 h and then cooled back to 0° C., triethylamine (10 eq) was added dropwise to neutralize the reaction. Then solvent was removed in vacuo, and the crude material was purified by SiO₂ column chromatography to afford 1.

A solution of 1 (1 eq), chloromethyl pivalate 2 (1.5 eq), NaH (1.5 eq) in dimethylformaide (0.1 M) was stirred at room temperature for 5 h. Then it was cooled to 0° C., and methanol was added. Solvent was removed in vacuo, and the crude material was purified by SiO₂ column chromatography to afford 3. To a solution of 3 (1 eq) in anhydrous dichloromethane (0.1 M) cooled to 0° C. was added diisopropylethylamine (DIPEA) (5 eq) and 4-dimethylaminopyridine (DMAP) (0.1 eq) under argon. Then 2,4,6-triisopropylbenzenesulfonyl chloride (1.5 eq) was added. After the disappearance of 3, hydroxylamine hydrochloride (2.5 eq) was added, and the mixture was stirred at room temperature for 12 h. Then it was diluted with dichloromethane and washed with sat NH₄C₁, sat NaHCO₃ and brine. Organic layer was dried (Na₂SO₄), filtered and concentrated in vacuo. The crude material was purified by SiO₂ column chromatography to afford 4.

To a solution of 4 (1 eq) in anhydrous dichloromethane (0.2 M) cooled to 0° C. was added trifluoroacetic acid (2 eq) under argon. The reaction was monitored by thin layer chromatography. After the disappearance of starting material, it was poured into cold sat NaHCO₃ solution to neutralize trifluoroacetic acid. Then it was extracted with dichloromethane (3 X). Organic layer was dried and concentrated in vacuo. The crude material was purified by SiO₂ column chromatography to afford 5.

### Example 12

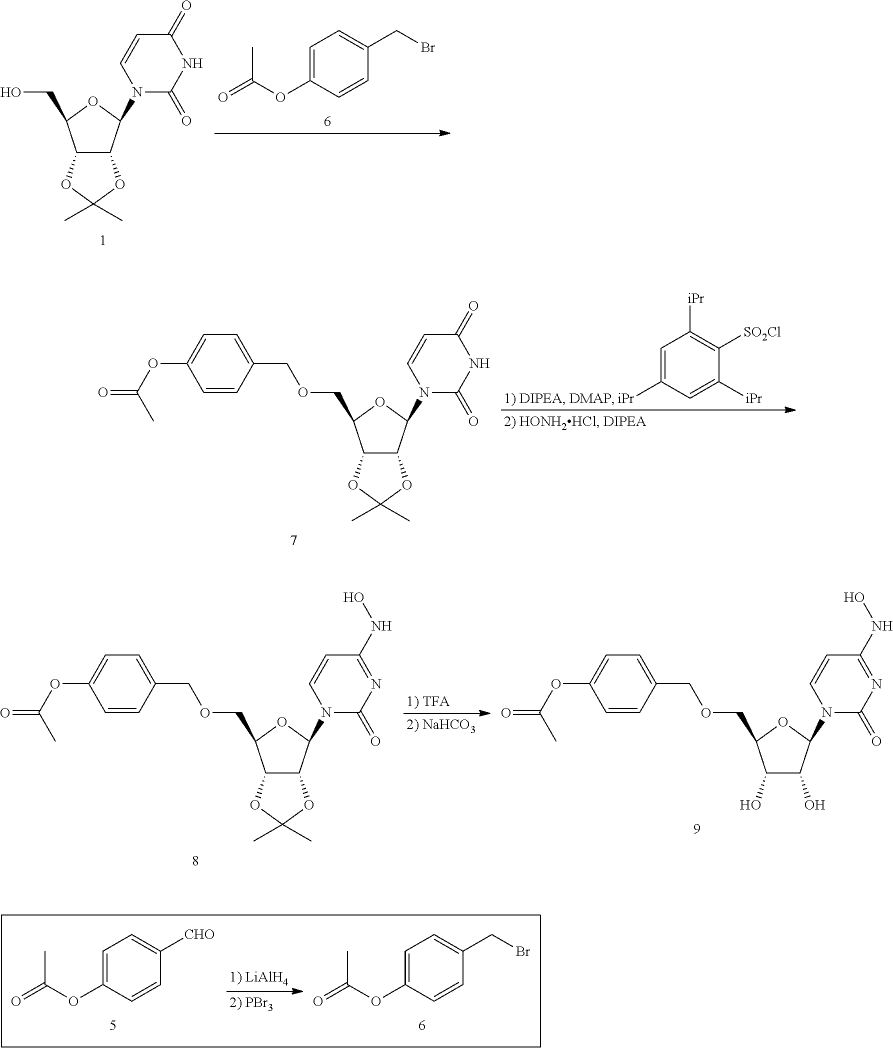

A solution of 1 (1 eq), 4-(bromomethyl)phenyl acetate 6 (1.5 eq), NaH (1.5 eq) in dimethylformamide (0.1 M) was stirred at room temperature for 5 h. Then it was cooled to 0° C., and methanol was added. Solvent was removed in vacuo, and the crude material was purified by SiO₂ column chromatography to afford 7. (Refer to reaction from compound 3 to 4 of Example 11). Compound 6 was synthesized by LiAlH₄ reduction of 5, followed by bromination by PBr₃. Conversion from 7 to 9 follows the same conditions described in Example 11 for the conversion of 3 to 5.

### Example 13

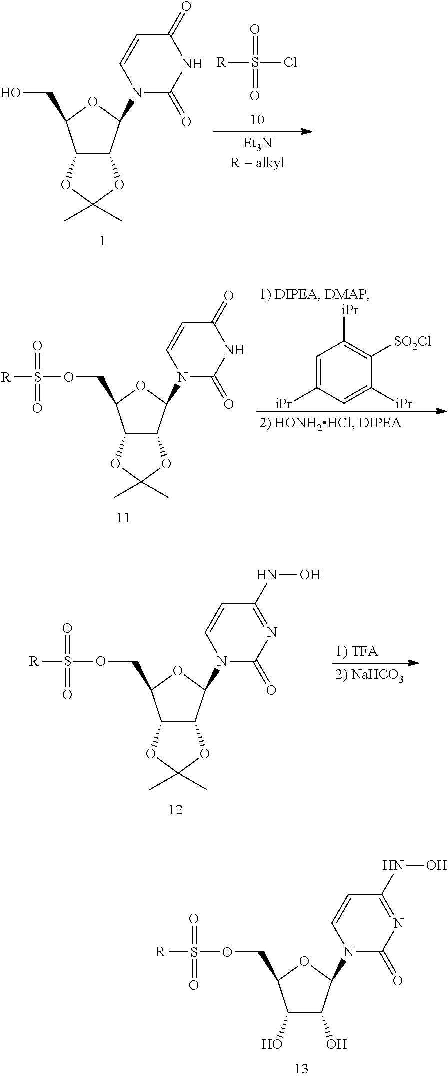

To a stirred solution of 1 (1 eq) in dry dichloromethane (0.5 M) was added triethylamine (2 eq) under argon. This was cooled to 0° C., and then sulfonyl chloride (1.2 eq) was added dropwise. After the disappearance of starting material, it was quenched with sat NH₄C₁. Organic layer was separated, washed with brine once, dried (Na₂SO₄), filtered and concentrated in vacuo. The crude material was purified by SiO₂ column chromatography to afford 11. Conversion from 11 to 13 follows the same conditions described in Example 11 for the conversion of 3 to 5.

### Example 14

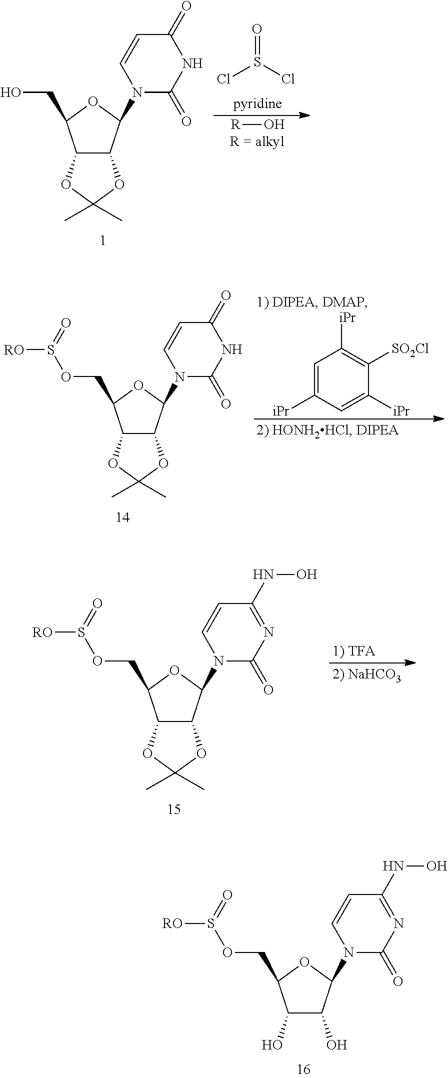

Thionyl chloride (1.05 eq) and pyridine (1.1 eq) were dissolved in ethylacetate. The solution was cooled to 0° C., and then 1 was added dropwise. After 30 min, a solution of R—OH (1.1 eq) and pyridine (1.1 eq) was added dropwise. After another 30 min, the reaction mixture was quenched with water. Organic layer was dried (Na₂SO₄), filtered, and concentrated. The crude material was purified by SiO₂ column chromatography to afford 14. Conversion from 14 to 16 follows the same conditions described in Example 11 for the conversion of 3 to 5.

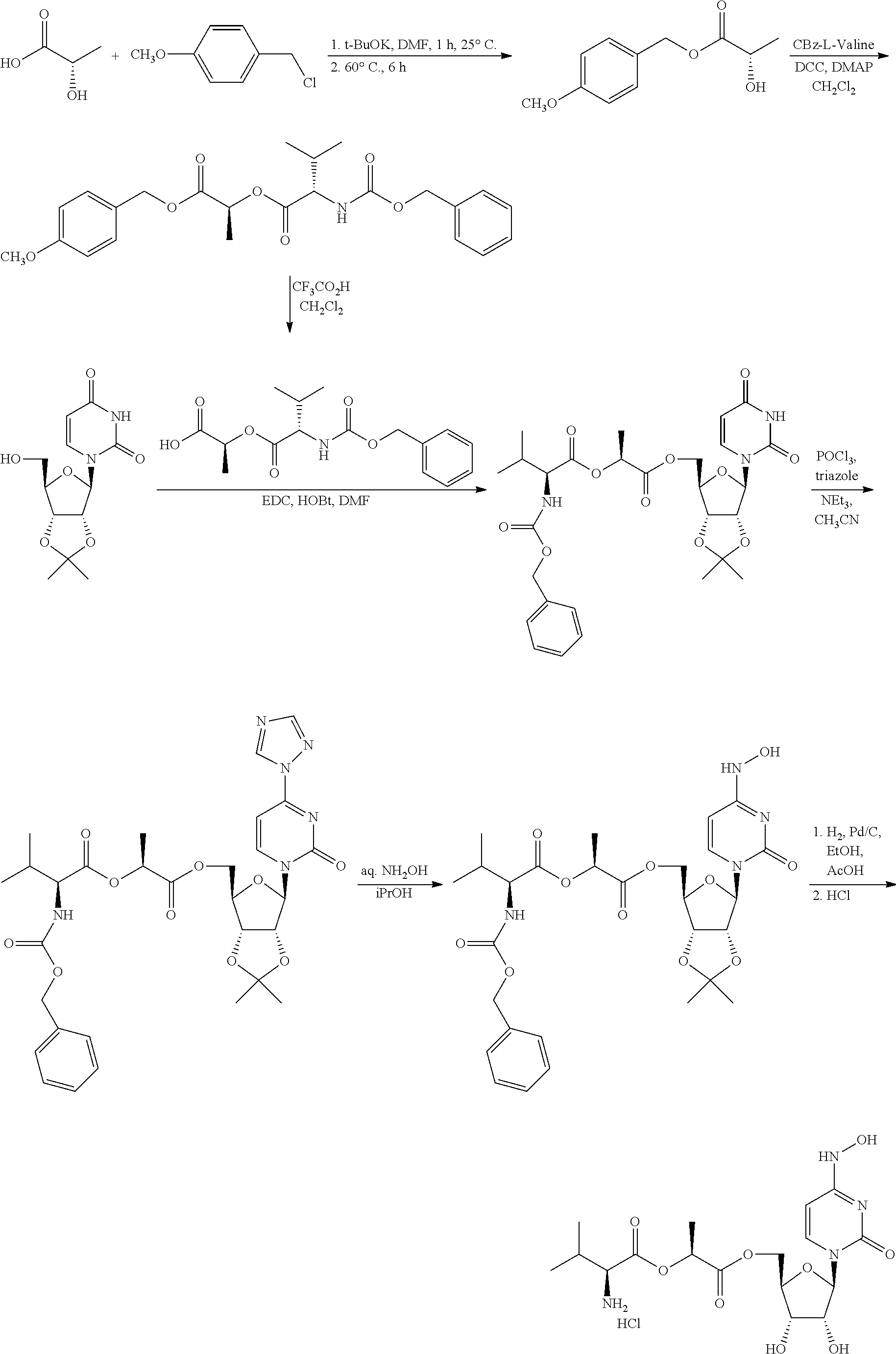

The valacetate prodrug moiety is prepared by a three-step process starting with the protection lactic acid as a para-methoxybenzyl ester, which is coupled to commercially available CBz-protected valine using carbodiimide reaction conditions. The resulting fully protected valacetate intermediate is treated with TFA in deprotection of the pMB ester to give carboxylic acid intermediate (S)-(+)-2-(N-CBz-L-valyloxy)propionic acid. Under standard carbodiimide conditions with EDC (1-Ethyl-3-(3-dimethylaminopropyl)carbodiimide), (S)-(+)-2-(N-CBz-L-valyloxy)propionic acid is coupled to 2′,‘3’—O-isopropylideneuridine. The resulting protected 5′-O-valacetate-uridine intermediate is then converted to 5′-O-valacetate-N⁴-hydroxycytidine by a four-step process, like that used in the synthesis of EIDD-2801, beginning with formation of a 4′-triazole-nucleoside intermediate followed by displacement with hydroxyamine. Hydrogenolysis of the CBz-group followed by HCl deprotection of the isopropylidene gives 5′-O-valacetate-N⁴-hydroxycytidine as an HCl salt.

### Example 16

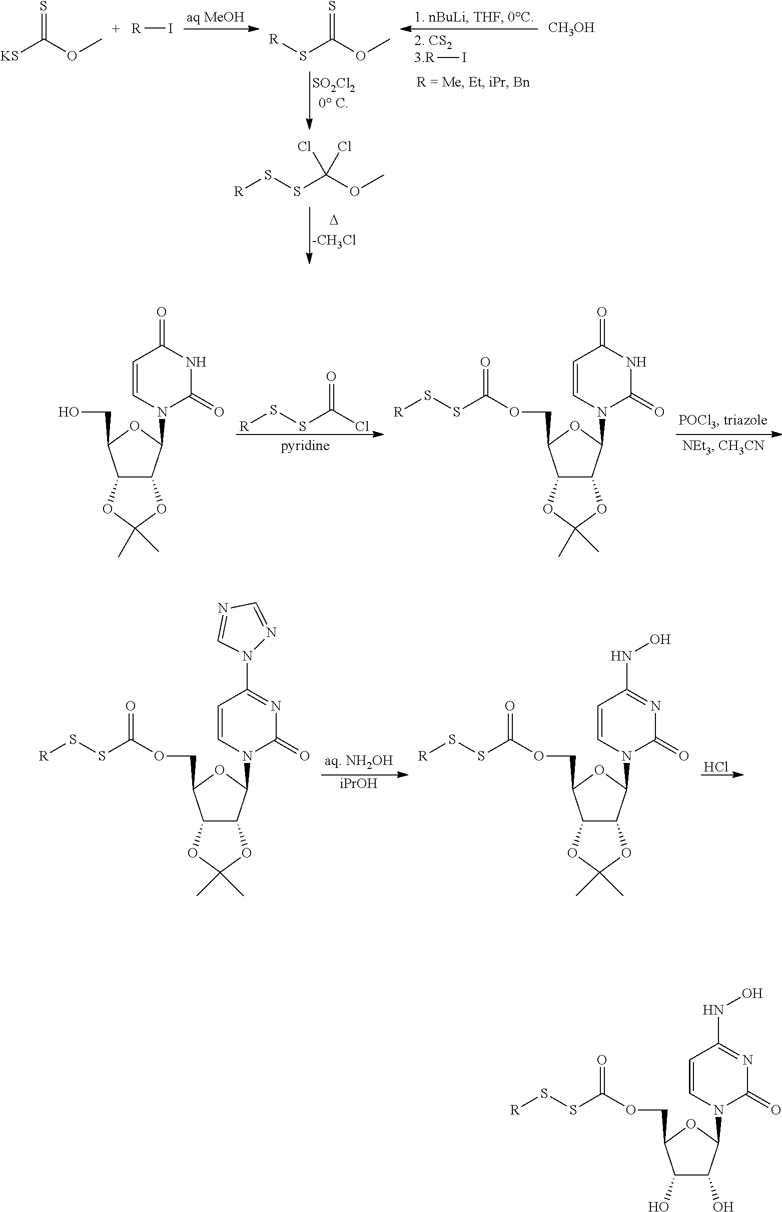

The sulfenyl thiocarbonate prodrug moiety is prepared as an activated alkylsulfenyl thiocarbonyl chloride in a three-step process from commercially available potassium methylxanthate. Upon treatment with an alkyl iodide in aqueous methanol, potassium methylxanthate is converted to its respective S-alkyl-O-methyl-dithiocarbonate. Alternatively, the S-alkyl-O-methyl-dithiocarbonate intermediate is also prepared by generating lithium methoxide in tetrahydrofuran followed by addition of carbon disulfide and alkyl iodide sequentially. In reaction with sulfuryl chloride at 0° C., the S-alkyl-O-methyl-dithiocarbonate intermediate undergoes a double chlorination followed by rearrangement to give a methoxydichloroalkyldisulfanyl intermediate that under thermal conditions (25-100° C.) eliminates methylchloride to afford the corresponding alkylsulfenyl thiocarbonyl chloride. Commercially available 2′,‘3’—O-isopropylideneuridine is acylated under standard conditions in pyridine with alkylsulfenyl thiocarbonyl chloride. The resulting 5′-O-alkylsulfenyl thiocarbonyl-2′,‘3’—O-isopropylideneuridine intermediate is further elaborated to 5′-O-alkylsulfenyl thiocarbonyl-N⁴-hyroxycytidine in three step process starting with activation as the 4-triazole intermediate followed by treatment with hydroxylamine and deprotection as in the synthesis of EIDD-2801.

### Example 17: General Synthesis for Deuteration

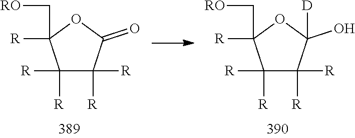

The lactone 389 (0.0325 mol) was added to a dry flask under an argon atmosphere and was then dissolved in dry THF (250 mL). The solution as then cooled to −78° C., and a DIBAL-D solution in toluene (0.065 mol) was dropwise. The reaction was allowed to stir at −78° C. for 3-4 hours. The reaction was then quenched with the slow addition of water (3 mL). The reaction was then allowed to stir while warming to room temperature. The mixture was then diluted with two volumes of diethyl ether and was then poured into an equal volume of saturated sodium potassium tartrate solution. The organic layer was separated, dried over MgSO4, filtered, and concentrated under reduced pressure. The residue was purified on silica eluting with hexanes/ethyl acetate. The resulting lactol 390 was then converted to an acetate or benzolyate and subjected to cytosine coupling conditions and then further elaborated to N-hydroxycytidine.

### Example 18: N4-Hydroxycytidine Coronaviridae Activity

CPE Assay—Confluent or near-confluent cell culture monolayers in 96-well disposable microplates were prepared. Cells were maintained in Minimum Essential Medium or Dulbecco's Modified Eagle Medium supplemented with Fetal Bovine Serum (FBS) as required for each cell line. For antiviral assays, the same medium was used but with FBS reduced to 2% or less and supplemented with 50-μg/mL gentamicin. The test compound was prepared at four serial log₁₀ concentrations, usually 0.1, 1.0, 10, and 100 μM. Five microwells were used per dilution: three for infected cultures and two for uninfected toxicity cultures. Controls for the experiment consist of six microwells that were infected (coronavirus controls) and six that were untreated (cell controls). The coronavirus control and cell control wells were on every microplate. In parallel, a known active drug was tested as a positive control drug using the same method as was applied for test compounds. The positive control was tested with each test run.

The assay was set up by first removing growth media from the 96-well plates of cells. Then the test compound was applied in 0.1 mL volume to wells at 2× concentration. Coronavirus, normally at <100 50% cell culture infectious doses (CCID₅₀) in 0.1 mL volume, was placed in those wells designated for virus infection. Medium devoid of virus was placed in toxicity control wells and cell control wells. Plates were incubated at 37° C. with 5% CO₂ until marked CPE (>80% CPE for most virus strains) was observed in virus control wells. The plates were then stained with 0.011% neutral red for approximately two hours at 37° C. in a 5% CO₂ incubator. The neutral red medium was removed by complete aspiration, and the cells were rinsed 1× with phosphate buffered solution (PBS) to remove residual dye. The PBS was completely removed, and the incorporated neutral red was eluted with 50% Sorensen's citrate buffer/50% ethanol for at least 30 minutes. Neutral red dye penetrates into living cells, thus, the more intense the red color, the larger the number of viable cells present in the wells. The dye content in each well was quantified using a 96-well spectrophotometer at 540 nm wavelength. The dye content in each set of wells was converted to a percentage of dye present in untreated control wells using a MICROSOFT EXCEL™ computer-based spreadsheet and normalized based on the untreated virus control. The 50% effective (EC₅₀, virus-inhibitory) concentrations and 50% cytotoxic (CC₅₀, cell-inhibitory) concentrations were then calculated by linear regression analysis. The quotient of CC₅₀ divided by EC₅₀ gave the selectivity index (SI) value.

VYR Assay—This assay involved similar methodology to that described above with the following differences. Eight half-log₁₀ concentrations of inhibitor were tested for antiviral activity and cytotoxicity per 96-well microplate. After sufficient virus replication occureds, a sample of supernatant was taken from each infected well (three replicate wells were pooled) for virus titer determination. The VYR test was a direct determination of how much the test compound inhibited virus replication. Virus that was replicated in the presence of test compound was titrated and compared to virus from untreated, infected controls. Titration of the pooled viral samples (collected as described above) was performed by endpoint dilution. This was accomplished by making serial log₁₀ dilutions of virus and plating each dilution into 3 or 4 replicate wells with fresh monolayers of cells. Plates were then incubated, and cells were scored for presence or absence of virus after distinct CPE was observed. Plotting the log₁₀ of the inhibitor concentration versus log₁₀ of virus produced at each concentration allowed calculation of the 90% (one log₁₀) effective concentration (EC₉₀) by linear regression. Results are shown in Table 1.

TABLE 1

| | | EC₅₀ | EC₉₀ | CC₅₀ |
|---|---|---|---|---|
| Virus | Cell Line | (μM) | (μM) | (μM) |
| MERS | Vero E6 | <0.80 | <0.80 | 20 |
| SARS | Vero76 | <0.4 | | 252 |
| SARS | Vero76 | <0.4 | | 144 |
| SARS | Vero76 | | 0.56 | 76 |
| SARS | Vero76 | 2.2 | | 76 |
| SARS | Vero E6 | <0.80 | <0.80 | 20 |
| HCoV | HEL | 1.28 | | 100 |
| HCoV | HEL | 5.6 | | 36 |
| HCoV | HEL | | <0.128 | 192 |
| HCoV | HEL | 0.228 | | 192 |
| HCoV | Vero76 | <0.4 | | 400 |
| HCoV | Vero E6 | <0.4 | | 400 |
| HCoV | HEL | 1.28 | | 100 |
| HCoV | HEL | 4 | | 60 |
| HCoV | HEL | | 0.4 | 232 |
| HCoV | HEL | 0.212 | | 232 |
| HCov | Vero76 | 12.8 | | 400 |
| HCoV | Vero76 | | 0.32 | 44 |
| HCov | Vero76 | 0.44 | | 44 |

### Example 19: Methods for Pharmacokinetic Studies in Cynomolgus Macaques

Eight cynomolgus macaques (4 males/4 females) were dosed by oral gavage with a single dose of EIDD-1931 or a prodrug conjugate as shown in Table 2. One week washout periods were allowed between doses. Blood samples were collected after each dosing event at predose, and 0.25, 0.5, 1, 2, 3, 4, 6, 8, 12, 18 and 24 hrs post dose.

TABLE 2
Study design for pharmacokinetic evaluation
of EIDD-1931 and 4 prodrug conjugates

| | | # Animals | Dose level | Dose level | Feeding |
|---|---|---|---|---|---|
| Grp # | Compound | (M/F) | mmol/kg | (mg/Kg) | State |
| 1 | EIDD-1931 | 4/4 | 0.4 | 100 | Fasted |
| 2 | EIDD-1931 | 4/4 | 0.4 | 100 | Fed |
| 3 | EIDD-2800 | 4/4 | 0.4 | 180 | Fed |
| 4 | EIDD-2801 | 4/4 | 0.4 | 130 | Fed |
| 5 | EIDD-2776 | 4/4 | 0.4 | 175 | Fed |
| 6 | EIDD-2898 | 4/4 | 0.4 | 160 | Fed |

Aliquots of Plasma were extracted with acetonitrile that included ¹³C₅ EIDD-1931 as an Internal Standard. Samples were then vortexed and centrifuged in a Sorvall RT1 centrifuge (Thermno Fisher, Waltham, MA) at 3,500 RPM for 10 minutes. The supernatant was transferred to a microcentrifuge tube and centrifuged again in a Biofuge pico centrifuge (Heraeus, Hanau, Germany) for 10 minutes at 13,000 rpm. The remaining supernatant was then transferred to an HPLC vial for analysis.

LC-MS/MS conditions for EIDD-2898. HPLC separation was performed on an Agilent 1200 system (Agilent Technologies, Santa Clara, CA, USA). An Atlantis HILIC Silica column, 50×4.6 mm, 5 μm particle size (Waters Corporation, Milford, MA, USA) was used for the separation of EIDD-1931, EIDD-2898 and ¹³C₅ EIDD-1931 (used as internal standard) with isocratic mode (70:30) with acetonitrile in 100 mM ammonium acetate buffer, pH 5.0 at a flow rate of 1.0 mL/min over 2 minutes. Mass Spectrometry analysis was performed on a QTrap 5500 Mass Spectrometer (AB Sciex, Framingham, MA) using Positive Mode Electrospray Ionization (ESI) in Multiple Reaction Monitoring (MRM) Mode. An eight-point standard curve prepared in blank plasma covered concentrations range of 10 to 10,000 ng/mL. Separately prepared quality-control samples of 30, 500 and 5000 ng/mL in blank plasma were analyzed at the beginning of each sample set to ensure accuracy and precision within 20%. Calibration in each matrix showed linearity with an R² value of >0.99. Data analysis was performed using Analyst Software (AB Sciex, Framingham).

LC-MS/MS conditions for EIDD-2800 and EIDD-2801. HPLC separation was performed on an Agilent 1200 system (Agilent Technologies, Santa Clara, CA, USA). An Acclaim HILIC-1 Mixed Mode column, 150×4.6 mm, 5 μm particle size (Thermo Fisher, Waltham, MA) was used for the separation of EIDD-1931, EIDD-2800, EIDD-2801, and ¹³C₅ EIDD-1931 (used as internal standard) with isocratic mode (90:10) with acetonitrile in 100 mM ammonium acetate buffer, pH 5.0 at a flow rate of 1.0 mL/min over 5 minutes. Mass Spectrometry analysis was performed on a QTrap 5500 Mass Spectrometer (AB Sciex, Framingham, MA) using Negative Mode Electrospray Ionization (ESI) in Multiple Reaction Monitoring (MRM) Mode. An eight-point standard curve prepared in blank plasma covered concentrations range of 10 to 10,000 ng/mL. Separately prepared quality-control samples of 30, 500 and 5000 ng/mL in blank plasma were analyzed at the beginning of each sample set to ensure accuracy and precision within 20%. Data analysis was performed using Analyst Software (AB Sciex, Framingham).

### Example 20: Pharmacokinetic Parameters from Cynomolgus Macaques

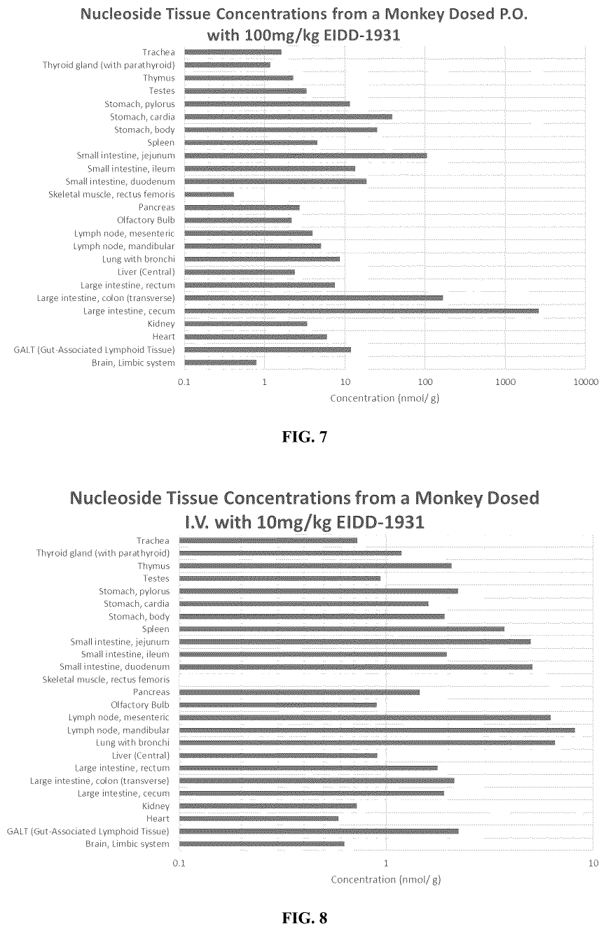

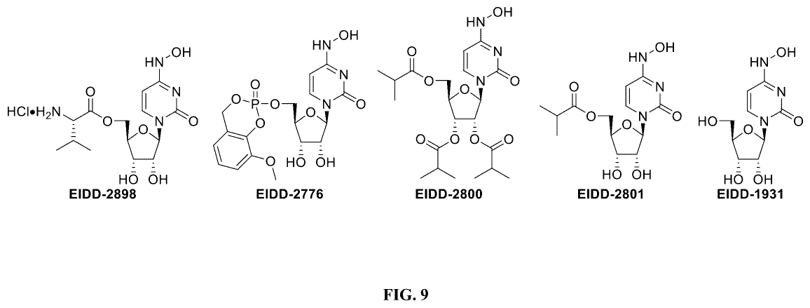

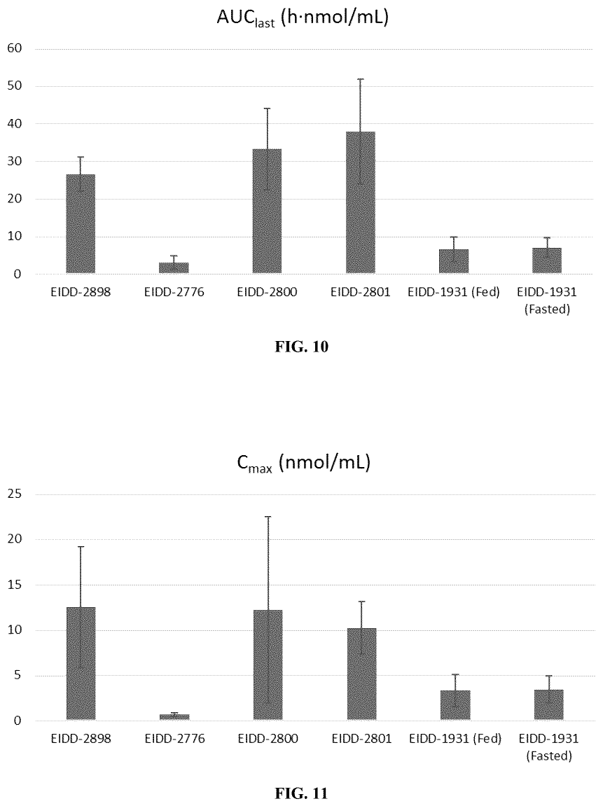

As can be seen from FIGS. 7 through 11, these data show that after administration by oral gavage to cynomolgus macaques, the parent ribonucleoside is unexpectedly sequestered, largely unchanged, in the enterocytes of the gut. This results in the low apparent bioavailability of the compound in cynomolgus macaques. However, when administered via i.v. injection, the compound is widely distributed. As a result of these studies, it appears that EIDD-1931 has low bioavailability in cynomolgus monkeys as a result of inefficient transit/release from intestinal and stomach linings to circulating blood.

The low bioavailability of EIDD-1931 in cynomolgus macaques can be successfully addressed by utilizing chemically and/or enzymatically cleavable prodrug moieties that facilitate the movement of EIDD-1931 across the gut wall into the circulating blood. Three prodrugs, EIDD-2800, EIDD-2801, and EIDD-2898, significantly improved the bioavailability if EIDD-1931 by 4-8 fold in cynomolgus macaques as can be seen from FIGS. 7 through 11.

Additional results are shown in Tables 3 and 4.

TABLE 3
Pharmacokinetic Parameters from Male Cynomolgus Macaques

| Compound | tₘₐₓ | Cₘₐₓ | AUC_{0->24h} | CL | t_{1/2} | F* |
|---|---|---|---|---|---|---|
| Dosed | (h) | (nmol/mL) | (h · nmol/mL) | (L/h*kg) | (h) | (%) |
| EIDD-1931 | 0.75 ± 0.28 | 3.31 ± 1.82 | 5.75 ± 1.99 | 70.1 ± 18.7 | 1.2 ± 1.2 | ~3 |
| EIDD-2800 | 0.37 ± 0.14 | 16.3 ± 13.2 | 38.9 ± 7.58 | 9.1 ± 1.3 | 5.5 ± 4.2 | ~27 |
| EIDD-2801 | 2 ± 0.81 | 8.08 ± 1.32 | 31.7 ± 7.82 | 13 ± 3.7 | 1.8 ± 0.91 | ~22 |
| EIDD-2898 | 2.3 ± 0.96 | 9.1 ± 2.7 | 26.1 ± 5.2 | 16.4 ± 3.1 | 0.53 ± 0.16 | ~18 |
| EIDD-2776 | 5 ± 1.2 | 0.58 ± 0.21 | 2.6 ± 0.65 | 142 ± 37.3 | 0.97 ± 0.21 | ~2 |

TABLE 4
Pharmacokinetic Parameters from Female Cynomolgus Macaques

| Compound | tₘₐₓ | Cₘₐₓ | AUC_{0->24h} | CL | t_{1/2} | F* |
|---|---|---|---|---|---|---|
| Dosed | (h) | (nmol/mL) | (h · nmol/mL) | (L/h*kg) | (h) | (%) |
| EIDD-1931 | 0.87 ± 0.75 | 3.31 ± 1.99 | 7.21 ± 4.21 | 65.7 ± 31.6 | 0.78 ± 0.2 | ~3 |
| EIDD-2800 | 0.31 ± 0.12 | 8.10 ± 5.06 | 27.4 ± 11.5 | 15.9 ± 7.7 | 4.4 ± 1.2 | ~16 |
| EIDD-2801 | 1.25 ± 0.5 | 12.3 ± 2.33 | 43.8 ± 17.0 | 10.3 ± 5.6 | 1.9 ± 1.3 | ~26 |
| EIDD-2898 | 1.3 ± 0.5 | 15.9 ± 8.1 | 26.9 ± 4.8 | 15.9 ± 3.2 | 0.55 ± 0.25 | ~15 |
| EIDD-2776 | 3 ± 2.4 | 0.69 ± 0.26 | 3.3 ± 2.7 | 158 ± 85.5 | 1.2 ± 0.41 | ~2 |

### Example 21: Methods for Pharmacokinetic Studies in Ferrets

EIDD-2801 and vehicle control were delivered via single oral gavage (P.O.). EIDD-2801 and vehicle control were delivered via oral gavage (P.O.) twice a day (BID). The first dose was at (−3 hrs) relative to virus challenge; the second dose at 0 hrs, and then every 12 hrs thereafter for 3.5 days; total 8 doses. The vehicle used consisted of 1% methylcellulose in water (w/v). Female 6-8 month old outbred ferrets (Mustela putoriusfuro), acquired from Triple F Farms, weighing 0.8-1.0 kg, were used for PK and efficacy studies:
Pharmacokinetics: 8 ferrets total (2 groups, 4 ferrets/group)
Efficacy testing: Prophylactic dosing against A/Netherlands/602/2009 (H1N1) NL/09; 5×10⁴ TCID₅₀/animal intranasally—12 ferrets total (3 groups, 4 ferrets/group) Pharmacokinetic study: EIDD-2801 was administered as a suspension by oral gavage in 3.5 mL total volume, followed by catheter flushing with MIRACLEVET solution. Blood samples were collected from the anterior vena cava. At 72 hrs pre-dose, 0.5 mL of blood was collected from each animal. After dosing, blood samples (0.3 mL) were collected at 0.25, 0.5, 1, 2, 4, 6, 8, and 24 hours in ice-cold Li Heparin tubes for plasma. Plasma was prepared within 1 hr after blood collection and was stored for up to 12 hours on ice before being transferred to −80° C. freezer. Samples were analyzed by LC/MS/MS.

### Example 22: Pharmacokinetic Parameters from Ferrets

Pharmacokinetic parameters for EIDD-1931 in ferrets after single doses of EIDD-2801 are shown in Table 5.

TABLE 5
Pharmacokinetic Parameters from Ferrets

| Dose | Cₘₐₓ | AUC_{inf} | t_{1/2} |
|---|---|---|---|
| mg/kg | (nmol/mL) | (h · nmol/mL) | (h) |
| 4 | 3.5 ± 1.5 | 13.2 ± 4.8 | 8.2 ± 1.7 |
| 20 | 15.4 ± 1.9 | 73 ± 32 | 4.7 ± 1.3 |
| 128 | 100 ± 22 | 322 ± 43 | 5.1 ± 0.8 |
| 512 | 209 ± 106 | 791 ± 391 | 4.2 ± 0.6 |

### Example 23: Methods for Pharmacokinetic Studies in Mice

ICR (CD-1), 7-8 weeks old mice were acclimated for −1 week after receipt. The mice were weighed to ±1 gram the day or morning before dosing to calculate dosing volumes. EIDD-2801 was completely dissolved in 5 mL of Solution A (PEG 400/Tween 80 (90%/10%)) with warming and vortexing and then was diluted with 5 mL of Solution B (30% Solutol/10% DMA). Mice were dosed p.o. There were 3 mice/group, to be sampled at 8 different time points: 0.25, 0.50, 1, 2, 3, 4, 8, and 24 hrs. Blood was collected at all 7 time points. Blood was obtained by retro-orbital bleeding under isoflurane anesthesia. Each mouse was sampled once (300 μL) and blood transferred immediately to Li heparin microtainers on ice water. The Li-Heparin tubes with blood were gently inverted 2 or 3 times to mix well; then placed in a rack in ice water until able to centrifuge (≤1 hour). Tubes were spun at˜2000×g for 10 min in a refrigerated centrifuge to separate plasma from RBCs. Plasma was immediately transferred to Eppendorf tubes, which were then placed in ice water. All samples were frozen on dry ice within ˜1 hr. Samples were stored at −80° C. prior to analysis by LC/MS/MS.

Plasma pharmacokinetic parameters for EIDD-1931 and EIDD-2898 in mice after single doses of EIDD-2898 are shown in Table 6.

TABLE 6
Pharmacokinetic Parameters from Mice

| EIDD-2989 Dose | | tₘₐₓ | Cₘₐₓ | AUC_{inf} | t_{1/2} |
|---|---|---|---|---|---|
| mg/kg | Analyte | (h) | (nmol/mL) | (h · nmol/mL) | (h) |
| 15 | 1931 | 0.25 | 11 | 10.2 | 2.9 |
| | 2898 | 0.08 | 23.1 | 8.23 | 0.34 |
| 225 | 1931 | 0.5 | 69.3 | 83.4 | 4.2 |
| | 2989 | 0.5 | 7.61 | 9.57 | 3.1 |
| 750 | 1931 | 0.5 | 71.3 | 228.9 | 5.2 |
| | 2989 | 0.25 | 7.3 | 21.9 | 6.7 |

Plasma pharmacokinetic parameters for EIDD-1931 in mice after a single dose of EIDD-2800 (180 mg/kg) is shown in Table 7. No EIDD-2800 (parent) was observed at any time point.

TABLE 7
Pharmacokinetic Parameters from Mice

| | tₘₐₓ | Cₘₐₓ | AUC_{inf} | t_{1/2} |
|---|---|---|---|---|
| Analyte | (h) | (nmol/mL) | (h · nmol/mL) | (h) |
| EIDD-1931 | 0.5 | 11.4 | 42.5 | 1.86 |

### Example 24: Methods for Pharmacokinetic Studies in Rats

Male Sprague Dawley (SD) rats, between 225-249 g in weight, were acclimated for at least two days before the experiment. The day before the experiment, the rats were weighed to determine average dosing volume of EIDD-2801. For dosing by oral gavage, EIDD-2801 was dissolved in 10% PEG 400, 2.5% Cremophor RH40 in water at 64 mg/mL and dosed at 5 mL/kg. Three rats were euthanized at each time by asphyxiation with carbon dioxide. Tissues and plasmas were collected 1, 2, 4, 6, 8, and 24 hours post-dose. One rat was dosed with the vehicle and euthanized by asphyxiation 6 hours post-dose. Plasma was collected from each animal by snipping the aorta to collect approximately 0.3 mL of whole blood into a lithium heparin tube. Blood was centrifuged at 2000×g for 10 min at 5° C. Plasma was then transferred to a 1.5 mL micro-centrifuge tube and stored at −80° C. until analysis. The brain, spleen, lung, kidney, liver, and heart were collected from each rat. Tissues were snap frozen in liquid nitrogen and stored at −80° C. 30-70 mg pieces of frozen animal tissue were weighed in 2 mL reinforced tubes and the weights were recorded. Samples were homogenized in 70% acetonitrile in water that included ¹³C₅-labelled-EIDD-1931 and ¹³C₅-labelled-EIDD-1931-TP as internal standards at 4° C. using an Omni bead-ruptor (Omni International, Inc., Kennesaw, GA). Homogenates were transferred to 2 mL micro-centrifuge tubes and centrifuged for 5 minutes at 15,000 rpm in an Eppendorf 5415D centrifuge (Eppendorf, Hamburg, Germany) to remove large solids. The supernatant was then transferred to a new 2 mL micro-centrifuge tube and centrifuged again in an Eppendorf 5415D centrifuge for 10 minutes at 15,000 rpm to remove any remaining solids. The remaining supernatant was transferred to a liquid chromatography mass spectrometer (LCMS) vial and analyzed via LCMS-MS.

Aliquots of rat plasma were extracted with acetonitrile that included ¹³C₅-labeled-EIDD-1931 as an Internal Standard. Samples were clarified by centrifugation in an Eppendorf 5415D centrifuge for 10 minutes at 15,000 rpm. The clarified supernatants were transferred to HPLC vials for analysis using qualified method BAM-106.

Samples were maintained at 4° C. in a Leap Pal Autosampler (CTC Analytics AG, Zwingen, Switzerland). HPLC separation was performed on an Agilent 1200 system (Agilent Technologies, Santa Clara, CA, USA) equipped with a column oven, UV lamp, and binary pump. For tissue samples, a SeQuant ZIC-pHILIC (100×4.6 mm, 5 μm) column (Merck Millipore, Burlington, MA, USA) was used for the separation of EIDD-1931, EIDD-2781, EIDD-2061, ATP, ¹³C₅-labelled-EIDD-1931, and ¹³C₅-labelled-EIDD-1931-TP. Mobile Phase A consisted of 25 mM ammonium bicarbonate buffer in HPLC grade water pH 9.8 and Mobile phase B consisted of pure acetonitrile. An 8.5-minute isocratic HPLC method at 35% mobile phase A was performed to separate the analytes. Mass Spectrometry analysis was performed on a QTRAP 5500 Mass Spectrometer (AB Sciex, Framingham, MA, USA) using negative mode Electrospray Ionization (ESI) in Multiple Reaction Monitoring (MRM) Mode. An Acclaim Polar Advantage II (3.0×50 mm, 3 μm particle size) column (Thermo Fisher Scientific, Waltham, MA) was used for the analysis of EIDD-2801. Mobile phase A consisted of 100 mM ammonium formate buffer in HPLC grade water and mobile phase B consisted of pure acetonitrile. A gradient method was employed from 5-100% mobile phase B over 3 minutes. Mass Spectrometry analysis was performed on an QTRAP 5500 Mass Spectrometer (AB Sciex, Framingham, MA, USA) using positive mode Electrospray Ionization (ESI) in Multiple Reaction Monitoring (MRM) Mode. For plasma samples, a SeQuant ZIC-pHILIC (100×4.6 mm, 5 μm) column (Merck Millipore, Burlington, MA, USA) was used for the separation of EIDD-1931, EIDD-2801, and ¹³C₅-labelled-1931. Mobile Phase A consisted of 25 mM ammonium bicarbonate buffer in HPLC grade water pH 9.8 and Mobile phase B consisted of pure acetonitrile. A 4.5-minute isocratic HPLC method at 35% mobile phase A was performed to separate the analytes. Mass Spectrometry analysis was performed on a QTRAP 5500 Mass Spectrometer (AB Sciex, Framingham, MA, USA) using negative mode Electrospray Ionization (ESI) in Multiple Reaction Monitoring (MRM) Mode. Data analysis was performed using Analyst Software (AB Sciex, Framingham, MA, USA).

### Example 25: Methods for Pharmacokinetic Studies in Dogs

Experimentally non-naïve dogs (from Marshall Biosciences) between the ages of 6.5 to 6.8 months, weighing between 7.1 to 7.95 kg were acclimated to their environment for at least three days prior to the first dosing event. Subsequent dosing events were executed after a 7-day washout period. Dogs were weighed at least once before each dose event to determine the dosing volume. EIDD-1931 was dissolved in sterile saline at 8 mg/mL for I.V. dosing. For oral dosing, EIDD-2801 was resuspended in 1% (v/v) methylcellulose in water at 6, 20, and 60 mg/mL. For I.V. dosing, dogs were dosed with a 1 mL/kg dose volume, and dogs dosed P.O. were dosed with a 5 mL/kg dose volume. Blood samples collected from dogs dosed by oral gavage were collected pre-dose, 0.25, 0.50, 1, 2, 3, 4, 8, 12, 18, and 24 hours post-dose. Blood samples collected from dogs dosed intravenously were collected pre-dose, 0.083, 0.25, 0.50, 1, 2, 4, 6, 8, 12, and 24 hours post-dose. Blood samples were collected from the jugular and/or cephalic vein into lithium-heparin microtainer tubes, centrifuged at 2000×g for 10 min at 5° C., and the plasmas were transferred into fresh tubes and stored at −80° C. before processing for quantitation by LC-MS/MS. 50 μL aliquots of dog plasma were extracted with 950 μL of acetonitrile that included ¹³C₅-labeled-EIDD-1931 as an Internal Standard. Samples were clarified by centrifugation at 20,000×g at 4° C. for 5 min. The clarified supernatants were transferred to HPLC vials for analysis. Samples were maintained at 4° C. in a Leap Pal Autosampler (CTC Analytics AG, Zwingen, Switzerland). HPLC separation was performed on an Agilent 1200 system (Agilent Technologies, Santa Clara, CA, USA) equipped with a column oven, UV lamp, and binary pump. A SeQuant ZIC-pHILIC (100×4.6 mm, 5 μm) column (Merck Millipore, Burlington, MA, USA) was used for the separation of EIDD-1931, EIDD-2801, and ¹³C₅-labeled-EIDD-1931. Mobile Phase A consisted of 25 mM ammonium bicarbonate buffer in HPLC grade water pH 9.8 and Mobile phase B consisted of pure acetonitrile. A 4-minute isocratic HPLC method at 35% mobile phase A was performed to separate the analytes. Mass Spectrometry analysis was performed on an QTRAP 5500 Mass Spectrometer (AB Sciex, Framingham, MA, USA) using Negative Mode Electrospray Ionization (ESI) in Multiple Reaction Monitoring (MRM) Mode. Data analysis was performed using Analyst Software (AB Sciex, Framingham, MA, USA). PK parameters are calculated using the Phoenix WinNonLin 6.4 (Build 6.4.0.768) Non-compartmental analysis tool (Certara, Princeton, NJ, USA). Bioavailability of EIDD-2801 is calculated by comparing the exposure (AUC-inf) of EIDD-1931 after EIDD-2801 oral dosing with the exposure of EIDD-1931 after intravenous dosing with EIDD-1931 using the formula below.

$$Oral Bioavailability = \frac{{Dose}_{I . V .}}{{Dose}_{P . O .}} \times \frac{{AUC}_{P . O .}}{{AUC}_{I . V .}}$$

Plasma pharmacokinetic parameters for EIDD-1931 in dogs after a single dose of EIDD-2800 (140 mg/kg) is shown in Table 8. No EIDD-2800 (parent) was observed at any time point.

TABLE 8
Pharmacokinetic Parameters from Dogs

| | tₘₐₓ | Cₘₐₓ | AUC_{inf} | t_{1/2} |
|---|---|---|---|---|
| Analyte | (h) | (nmol/mL) | (h · nmol/mL) | (h) |
| EIDD-1931 | 1.4 ± 0.5 | 112.8 ± 21.1 | 497.7 ± 40.4 | 4.8 ± 1 |

### Example 26: Pharmacokinetic Parameters from Mice

Test article was incubated in triplicate at 1.00 μM in pooled mixed gender human plasma (BioIVT, K₂EDTA), in pooled male CD-1 mouse plasma (BioIVT, K₂EDTA), in pooled male Sprague-Dawley rat plasma (BioIVT, lithium heparin). Incubations were performed in 13×100 mm glass culture tubes. Samples were placed in a water bath shaker set at 37° C. and shaken at 150 rpm. Procaine, Benfluorex or Enalapril (1 μM, each) were run in parallel as a positive controls for human, mouse or rat plasma activity, respectively.

Aliquots of 100 μL were taken at the following time-points: 0, 5, 15, 30, 60, and 120 minutes. These aliquots were mixed with 400 μL of 100% acetonitrile in 1.7-mL conical polypropylene microcentrifuge tubes. Samples were vortexed for about 10 seconds and then clarified by centrifugation (2 minutes at 15,000 g). Supernatants were analyzed by LC-MS/MS.

HPLC separation was performed on an Agilent 1200 system (Agilent Technologies, Santa Clara, CA, USA) equipped with a column oven, UV lamp, and binary pump. A Thermo Hypercarb PGC (150×4.6 mm, 5 μm) column (ThermoFisher, Waltham, MA USA) was used for the separation. Mobile Phase A consisted of 100 mM Ammonium Bicarbonate buffer in HPLC grade Water (pH 10) and Mobile phase B consisted of neat acetonitrile. A gradient 0-85% of B was run for 3 minutes followed by 0% B for 4 minutes was used for the separation. Mass Spectrometry analysis was performed on a Triple Quad 5500 Mass Spectrometer (AB Sciex, Farmingham, MA, USA) using Negative Mode Electrospray Ionization (ESI) in Multiple Reaction Monitoring (MRM) Mode. Data analysis was performed using Analyst Software (AB Sciex, Farmingham, MA, USA).

Analyte concentrations were calculated based on standard curve. Half-lives (t_{1/2}) were calculated by plotting the natural logarithm of the analyte concentration vs. time and obtaining the slope of the line. Assuming first-order kinetics, the elimination rate constant, k, is the negative (−) of the slope of the plot (ln [μM] vs. time). Half-life (t_{1/2}) (min)=−0.693/(slope).

### Example 27: Plasma and Liver Microsome Stability for EIDD-2800, 2801, and 2898

Test article was incubated in triplicate at 1.00 μM in 100 mM phosphate buffer (pH 7.4), Phase I cofactors (NADPH Regenerating System) and 0.5 mg (total protein) from pooled gender human liver microsomes (BioIVT), pooled male CD-1 mouse liver microsomes (XenoTech) or pooled male Sprague-Dawley rat liver microsomes (BioIVT). Incubations were performed in 13×100 mm glass culture tubes. Samples were placed in a water bath shaker set at 37° C. and shaken at 150 rpm. Verapamil (1 μM) was run in parallel as a positive control.

HPLC separation was performed on an Agilent 1200 system (Agilent Technologies, Santa Clara, CA, USA) equipped with a column oven, UV lamp, and binary pump. A Thermo Hypercarb PGC (150×4.6 mm, 5 μm) column (ThermoFisher, Waltham, MA USA) was used for the separation. Mobile Phase A consisted of 100 mM Ammonium Bicarbonate buffer in HPLC grade Water (pH 10) and Mobile phase B consisted of neat acetonitrile. A gradient 0-85% of B was run for 3 minutes followed by 0% B for 4 minutes were used for the separation. Mass Spectrometry analysis was performed on a Triple Quad 5500 Mass Spectrometer (AB Sciex, Framingham, MA, USA) using Negative Mode Electrospray Ionization (ESI) in Multiple Reaction Monitoring (MRM) Mode. Data analysis was performed using Analyst Software (AB Sciex, Framingham, MA, USA).

Analyte concentrations were calculated based on Standard curve. Half-lives (t_{1/2}) were calculated by plotting the natural logarithm of the analyte concentration vs. time and obtaining the slope of the line. Assuming first-order kinetics, the elimination rate constant, k, is the negative (−) of the slope of the plot (ln [μM] vs. time). Half-life (t_{1/2}) (min)=−0.693/(slope).

TABLE 9

| Substrate | Species | Plasmat½ (min) | Liver Microsomes t½ (min) |
|---|---|---|---|
| EIDD-2800 | Mouse | 1 | <1 |
| | Monkey | 2 | 2 |
| | Human | 1 | 1 |
| EIDD-2801 | Mouse | 1 | 2 |
| | Rat | 1 | 5 |
| | Dog | 192 | 1 |
| | Monkey | 24 | 1 |
| | Human | 63 | 73 |
| EIDD-2898 | Mouse | 144 | 6 |
| | Monkey | 138 | 13 |
| | Human | 198 | 14 |

### Example 28: Pharmacokinetic Parameters from Mice

Female ICR (CD-1™) mice (from Envigo, NJ), 6-8 weeks of age, were used in the studies. Drug was administered by oral gavage (PO) in 240 mM citrate buffer pH 3±0.3 or intraperitoneally (IP) in saline. Blood samples were collected at 0.08, 0.25, 0.5, 1, 2, 4, 8, and 24 h post IP administration, and at 0.25, 0.5, 1, 2, 3, 4, 8, and 24 h post oral administration. Plasmas were prepared within 30 min after collection by centrifugation at 2000 g for 10 min at 4° C. Mouse organs (lung, spleen liver, kidney, heart and brain) were collected from all mice immediately following blood collection starting from 0.5 h post dose. The tissues were immediately snap-frozen in liquid nitrogen and stored at −80° C. until they were analyzed by LC-MS/MS.

Frozen mouse tissues (˜50 mg) were extracted with 0.45 ml of cold 70% acetonitrile in water by homogenization in an Omni Bead-Ruptor (Omni International, Kennesaw, GA). The extracts were centrifuged for 10 min at 2000 g. The supernatants were transferred to micro-centrifuge tubes and centrifuged again for 10 min at 14,000 g. The clarified supernatants were transferred to HPLC vials and Internal Standards were added.

HPLC separation was performed on an Agilent 1200 system (Agilent Technologies, Santa Clara, CA, USA). A SeQuant ZIC-pHILIC 5-μm, 100 by 4.6 mm column (The Nest group, USA) was used for separations. HPLC separation of tissue extracts was performed using a linear gradient mode of acetonitrile (85-40%) in 25 mM ammonium bicarbonate buffer, pH 9.4 at a flow rate of 0.75 ml/min over 9 min. Mass spectrometry analysis was performed on a QTrap 5500 Mass Spectrometer (AB Sciex, Framingham, MA) using Negative Mode Electrospray Ionization (ESI) in Multiple Reaction Monitoring (MRM) Mode. For organ tissue analysis seven-point standard curves were prepared in blank tissue lysates spanning concentrations from 1.49 to 1490 ng/mL. Calibration in each matrix showed linearity with an R² value of >0.99. Data analysis was performed using Analyst Software (AB Sciex, Framingham). Pharmacokinetic parameters were calculated using Phoenix WinNonlin Non-compartmental analysis software (Certara, Princeton, NJ).

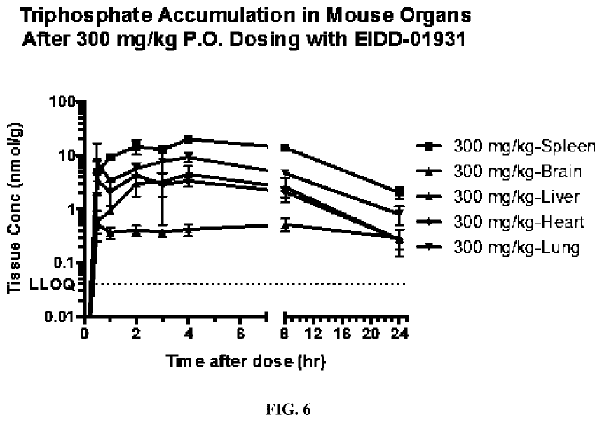

Tissue pharmacokinetic parameters for EIDD-1931 and EIDD-2061 (EIDD-1931-5′-triphosphate) in mice after single doses of EIDD-2898 is shown in Table 10 and FIGS. 5 and 6.

TABLE 10
Tissue Pharmacokinetic Parameters from Mice

| | | Spleen | | Brain | | Lung | |
|---|---|---|---|---|---|---|---|
| EIDD-2898 Dose | | AUC_{0->t} | Cₘₐₓ | AUC_{0->t} | Cₘₐₓ | AUC_{0->t} | Cₘₐₓ |
| mg/kg | Analyte | (h · nmol/g) | (nmol/g) | (h · nmol/g) | (nmol/g) | (h · nmol/g) | (nmol/g) |
| 225 | 1931 | 536.4 | 285.1 | 202.4 | 12.6 | 113.4 | 76.7 |
| | 2061 | 110.8 | 9.9 | 63.1 | 3.5 | 35.0 | 2.5 |
| 750 | 1931 | 1420.8 | 373.1 | 107.9 | 18.8 | 386.9 | 82.4 |
| | 2061 | 257.0 | 24.7 | 64.1 | 5.5 | 120.2 | 10.9 |

### Example 29: Protocol for Evaluating EIDD-2801 Prophylactic Treatment in a Mouse Model of SARS Infection

Female and male 20-week-old C₅₇BL/6J mice were used after a five day or greater acclimation period in BSL3. For each sex, animals were randomly assigned to treatment groups and individually marked with ear punches. The virus stock utilized for these studies was derived from the infectious clone of the mouse adapted SARS-CoV MA15 (MA15) strain. After electroporation of Vero E6 cells with viral genomic RNA from SARS MA15, supernatant was harvested when the monolayer exhibited >80% CPE. The resultant stock was passaged twice on Vero E6 cells to generate a working stock with a titer of 6.3×10⁷ pfu/ml.

The large left lung lobe of each mouse was harvested into a 2 mL screw cap tube containing glass beads and 1 mL PBS. This sample was frozen at −80° C. until the plaque assay was performed. 24 hr prior to performing the plaque assay, 6-well plates of Vero E6 cells were seeded at 500,000 cells/well/2 mL. Cells were incubated at 37° C. in 5% CO₂ for 24 hr. On the day of the assay, lungs were homogenized using a Roche Magnalyzer, lung homogenates were clarified via centrifugation at >10,000×g, serially diluted in PBS, added to monolayers of Vero E6 cells, and incubated at 37° C. with 5% CO₂ for 1 hr after which cells were overlayed with medium containing 0.8% agarose. Two days later, monolayers were stained with neutral red viability stain to aid in plaque visualization. The numbers of plaques per virus diluted were enumerated to generate the plaque forming units per lung lobe (pfu/lobe).

Equivalent numbers of male and female 20-25-week-old SPF C₅₇BL/6 J (Stock 000664 Jackson Labs) were used for these studies. Mice were randomly assigned to each treatment group. Groups to be infected with SARS-CoV were comprised of 10 mice (5 male/5 female). To control for potential effects associated with oral dosing on animal weight or pulmonary function, as well as the effect of the tested compound, two smaller “sham” infected groups will also be included (n=6, 3 males and 3 females each). EIDD-2801 or vehicle control was delivered via oral gavage (P.O.) twice a day (BID). The first dose was initiated at −2 hr relative to virus challenge; the second dose was at 12 hpi, and then every 12 hrs thereafter for 5 days; total 10 doses. Mice were anaesthetized with a mixture of ketamine/xylazine prior to intranasal infection with a dose of 1×10⁴ plaque forming units (PFU) of SARS-CoV MA15 strain in 0.05 mL diluted in PBS at time 0 hpi. All mice were weighed daily, and a subset of mice were assayed by whole body plethysmography (4 mice 2 males and 2 females per treatment group) to determine pulmonary function daily for 5 days post infection. Following sacrifice at Day 5 post infection, lungs were assessed for lung hemorrhage score. Tissue was then removed for virus lung titer and pathology. The large left lobe was harvested for virus lung titer, and the lower right lobe was harvested for pathology.

Whole body plethysmography: Pulmonary function was monitored once daily via whole-body plethysmography (Buxco Respiratory Solutions, DSI Inc.). Mice destined for this analysis were chosen prior to infection. Briefly, after a 30-minute acclimation time in the plethysmograph, data for 11 parameters was recorded every 2 seconds for 5 minutes.

Statistical analysis: All statistical data analysis was performed in Graphpad Prism 7. Statistical significance for each endpoint was determined with specific statistical tests. For each test, a p-value <0.05 was considered significant. For percent starting weight and whole body plethysmography, we performed a two-way ANOVA and Dunnet's multiple comparison test. For lung hemorrhage and virus lung titer, we performed a one-way ANOVA with a Kruskall-Wallace multiple comparison test.

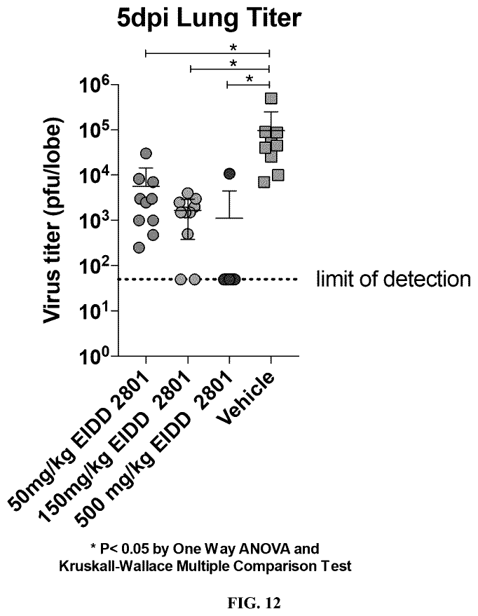

Mice infected with SARS were treated prophylactically with EIDD-2801. Effect of treatment on lung viral titers can be found in FIG. 12.

### Example 30: Protocol for Evaluating EIDD-2801 Time of Treatment in a Mouse Model of SARS Infection

Female and male 25-29-week-old C₅₇BL/6J mice were used after a five day or greater acclimation period in BSL3. For each sex, animals were randomly assigned to treatment groups and individually marked with ear punches. The virus stock utilized for these studies was derived from the infectious clone of the mouse adapted SARS-CoV MA15 (MA15) strain that was generated in the Baric laboratory. After electroporation of Vero E6 cells with viral genomic RNA from SARS MA15, supernatant was harvest when the monolayer exhibited >80% CPE. The resultant stock was passaged twice on Vero E6 cells to generate a working stock with a titer of 6.3×10⁷ pfu/ml. The lower right lung lobe of each mouse was harvested into a 2 mL screw cap tube containing glass beads and 1 mL PBS. This sample was frozen at −80° C. until the plaque assay was performed. 24 hr prior to performing the plaque assay, 6-well plates of Vero E6 cells were seeded at 500,000 cells/well/2 ml. Cells were incubated at 37° C. in 5% CO₂ for 24 hr. On the day of the assay, lungs were homogenized using a Roche Magnalyzer, lung homogenates were clarified via centrifugation at >10,000×g, serially diluted in PBS, added to monolayers of Vero E6 cells, and incubated at 37° C. with 5% CO₂ for 1 hr after which cells were overlayed with medium containing 0.8% agarose. Two days later, monolayers were stained with neutral red viability stain to aid in plaque visualization. The numbers of plaques per virus diluted were enumerated to generate the plaque forming units per lung lobe (pfu/lobe). Equivalent numbers of male and female 25-29-week-old SPF C₅₇BL/6 J were used for these studies. Mice were randomly assigned to each treatment group. Groups to be infected with SARS-CoV were comprised of 10 mice (5 male/5 female). EIDD-2801 or vehicle control was delivered via oral gavage (P.O.) twice a day (BID). We initiated dosing at −2 hr, +12 hr, +24 hr or +48 hr relative to virus challenge. Mice were anaesthetized with a mixture of ketamine/xylazine prior to intranasal infection with a dose of 1×10⁴ plaque forming units (PFU) of SARS-CoV MA15 strain in 0.05 mL diluted in PBS at time 0 hpi. All mice were weighed daily, and a subset of mice were assayed by whole body plethysmography (4 females per treatment group) daily to determine pulmonary function. Following sacrifice at 5 dpi, lungs were assessed for lung hemorrhage score. Tissue was then removed for virus lung titer and pathology. The large left lobe was harvested for pathology and the lower left lobe was harvested for virus titer. Pulmonary function was monitored once daily via whole-body plethysmography (Buxco Respiratory Solutions, DSI Inc.). Mice destined for this analysis were chosen prior to infection. Briefly, after a 30-minute acclimation time in the plethysmograph, data for 11 parameters was recorded every 2 seconds for 5 minutes. All statistical data analysis was performed in Graphpad Prism 7. Statistical significance for each endpoint was determined with specific statistical tests. For each test, a p-value <0.05 was considered significant. For percent starting weight and whole body plethysmography, we performed a two-way ANOVA and Dunnet's multiple comparison test. For lung hemorrhage and virus lung titer, we performed a one-way ANOVA with a Kruskall-Wallace multiple comparison test.

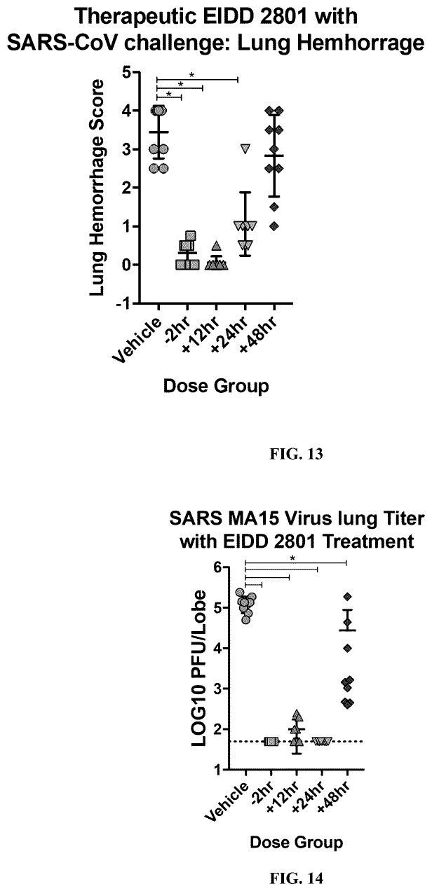

Mice infected with SARS were treated with EIDD-2801. Effect of treatment on lung hemorrhage scores and lung viral titers can be found in FIGS. 13 and 14, respectively.

### Example 31: Protocol for Evaluating EIDD-2801 Therapeutic Treatment in a Mouse Model of MERS Infection

Female and male 10-11-week-old C₅₇BL/6 J 288/330 DPP4 mice created and bred by the Baric Laboratory were used after a five day or greater acclimation period in BSL3. For each sex, animals were randomly assigned to treatment groups and individually marked with ear punches. The virus stock utilized for these studies was derived from a plaque purified isolate of the mouse adapted MERS-CoV p35C₄ (MERS) strain that was generated in the Baric laboratory. After plaque purification, virus was passaged twice on Vero CC81 cells. The resultant stock titer was of 1.1×10⁸ pfu/ml. The lower right lung lobe of each mouse was harvested into a 2 mL screw cap tube containing glass beads and 1 mL PBS. This sample was frozen at −80° C. until the plaque assay was performed. 24 hr prior to performing the plaque assay, 6-well plates of Vero CC81 cells were seeded at 500,000 cells/well/2 ml. Cells were incubated at 37° C. in 5% CO₂ for 24 hr. On the day of the assay, lungs were homogenized using a Roche Magnalyzer, lung homogenates were clarified via centrifugation at >10,000×g, serially diluted in PBS, added to monolayers of Vero CC81 cells, and incubated at 37° C. with 5% CO₂ for 1 hr after which cells were overlayed with medium containing 0.8% agarose. Three days later, monolayers were stained with neutral red viability stain to aid in plaque visualization. The number of plaques per virus diluted were enumerated to generate the plaque forming units per lung lobe (pfu/lobe). Equivalent numbers of 10-11-week-old C₅₇BL/6 J 288/330 DPP4 mice were randomly assigned to each treatment group for these studies. Each group was comprised of 10 mice (5 male/5 female). EIDD-2801 or vehicle control was delivered via oral gavage (P.O.) twice a day (BID) beginning at −2 hr and then every 12 hr thereafter. Mice were anaesthetized with a mixture of ketamine/xylazine prior to intranasal infection with a dose of 5×10⁴ plaque forming units (PFU) of MERS strain in 0.05 mL diluted in PBS at time 0 hpi. All mice were weighed daily, and a subset of mice were assayed by whole body plethysmography (4 females per treatment group) daily to determine pulmonary function. Following sacrifice at 5 dpi, lungs were assessed for lung hemorrhage score. Tissue was then removed for virus lung titer and pathology. The large left lobe was harvested for pathology and the lower left lobe was harvested for virus titer. Pulmonary function was monitored once daily via whole-body plethysmography (Buxco Respiratory Solutions, DSI Inc.). Mice destined for this analysis were chosen prior to infection. Briefly, after a 30-minute acclimation time in the plethysmograph, data for 11 parameters was recorded every 2 seconds for 5 minutes.

All statistical data analysis was performed in Graphpad Prism 7. Statistical significance for each endpoint was determined with specific statistical tests. For each test, a p-value <0.05 was considered significant. For percent starting weight and whole-body plethysmography, we performed a two-way ANOVA and Dunnet's multiple comparison test. For lung hemorrhage, we performed a one-way ANOVA with a Kruskall-Wallace multiple comparison test.

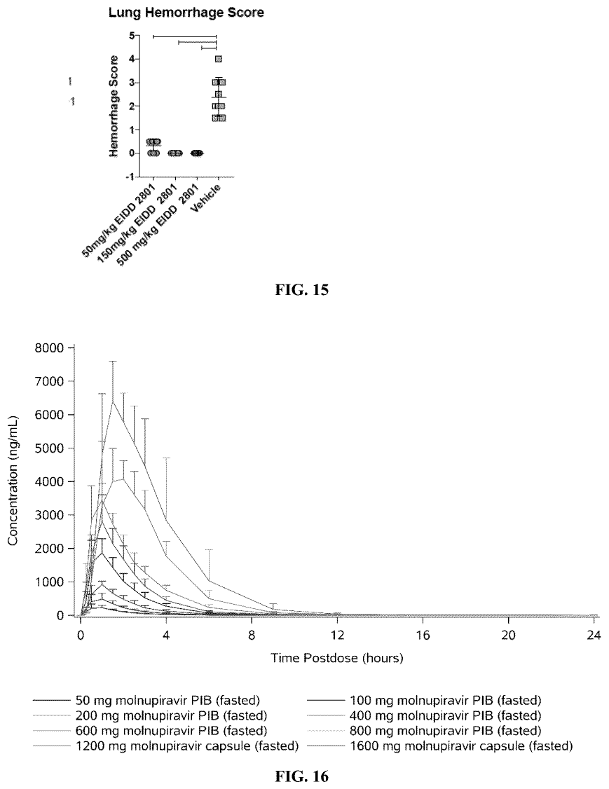

Mice infected with MERS were treated with EIDD-2801. Effect of treatment on lung hemorrhage scores can be found in FIG. 15.

### Example 32: Method for Evaluating Cell Uptake and Metabolism of EIDD-2801 in Vero Cells

Three 24-well plates were plated with primary vero cells at a seeding density of 0.350×10⁶/mL viable cells per well. The plates were incubated at 37° C./5% CO₂ overnight to allow the cells to attach. A 40 mM solution of EIDD-2801 in 100% dimethylsulfoxide was prepared. From the 40 mM stock solution, a 20 μM solution of EIDD-2801 was prepared in 25 mL of complete Dulbeccos' Modified Eagle Medium. For compound treatment plates, the media was aspirated, and 1.0 mL of 20 μM EIDD-2801 in complete Dulbeccos' Modified Eagle Medium was added to the appropriate wells. A separate plate of cells was prepared with no compound added. The plates were then incubated at 37° C./5% CO₂ for the following time points: 1, 2, 3, 4, 6, 16 and 24 hours. The non-treated plate was sampled at 0 hrs. After incubation at the desired time points, cells were washed 2× with 1.0 mL of DPBS. Cells were extracted by adding 500 μL of 70% acetonitrile/30% water spiked with the internal standard to each well treated with EIDD-2801. The non-treated blank plate was extracted with 500 μL of 70% acetonitrile/30% water per well. The samples were pipetted up and down several times. The samples were transferred to labeled microcentrifuge tubes. The samples were centrifuged at 16,000×g for 10 minutes at 4° C. 300 μL of supernatant was transferred to labeled HPLC vials, and the samples were analyzed by LC-MS/MS. Results are shown in Table 11.

TABLE 11

| Analyte | Cₘₐₓ | tₘₐₓ | AUC_{0->t} |
|---|---|---|---|
| Analyte | (pmol/M Cells) | (h) | (pmol · h/M cells) |
| EIDD-1931 (Nuc) | 13.5 | 24 | 228.6 |
| EIDD-2061 (TP) | 872.0 | 16 | 13850 |
| EIDD-2801 (Parent) | 121.2 | 3 | 1724 |

### Example 33: Method for Evaluating Cell Uptake and Metabolism of EIDD-2801 in Huh-7 Cells

Four 24-well plates were plated with Huh-7 cells at a seeding density of 0.35×10⁶/mL viable cells per well. The plates were incubated at 37° C./5% CO₂ overnight to allow the cells to attach. A 40 mM stock solution of EIDD-2801 was prepared in 100% dimethylsulfoxide. From the 40 mM solution, a 20 μM solution of EIDD-2801 in 25 mL of complete Dulbeccos' Modified Eagle Medium was prepared by pipetting 12.5 μL of EIDD-2801 into the media. For compound treatment plates, the media was aspirated, and 1.0 mL of 20 μM EIDD-2801 solution in complete Dulbeccos' Modified Eagle Medium was added to the appropriate wells. A separate plate of cells had no compound added and was aspirated and replaced with media without compound. The plates were incubated at 37° C./5% CO₂ for the following time points: 1, 2, 3, 4, 6, 16 and 24 hours. A non-treated plate was 0 hrs sample. After incubation at the desired time points, cells were washed 2× with 1.0 mL of DPBS. Cells were extracted by adding 500 μL of 70% acetonitrile/30% water spiked with the internal standard to each well treated with EIDD-2801. The non-treated blank plate was extracted with 500 μL of 70% acetonitrile/30% water per well without an internal standard. The samples were pipetted up and down several times. The samples were transferred to labeled microcentrifuge tubes. The samples were centrifuged at 16,000×g for 10 minutes at 4° C. 350 μL of supernatant was transferred to labeled 5 mL tubes or if samples were not being dried down put in labeled HPLC vials. Samples were analyzed by LC-MS/MS. Results are shown in Table 12.

TABLE 12

| Analyte | Cₘₐₓ | tₘₐₓ | AUC_{0->t} |
|---|---|---|---|
| Analyte | (pmol/M Cells) | (h) | (pmol · h/M cells) |
| EIDD-1931 (Nuc) | 29.0 | 24 | 449.2 |
| EIDD-2061 (TP) | 1113.3 | 24 | 14640 |
| EIDD-2801 (Parent) | 77.5 | 2 | 1025 |

### Example 34: Method for Evaluating Cell Uptake and Metabolism of EIDD-2801 in HepG2 Cells

Three 24-well plates were plated with primary vero cells at a seeding density of 0.350×10⁶/mL viable cells per well. The plates were incubated at 37° C./5% CO₂ overnight to allow the cells to attach. A 40 mM stock solution of EIDD-2801 in 100% dimethylsulfoxide was prepared. From the 40 mM solution, a 20 μM solution of EIDD-2801 was prepared in 25 mL of complete RPMI media. For compound treatment plates, the media was aspirated, and 1.0 mL of 20 μM EIDD-2801 in complete RPMI media was added to the appropriate wells. A separate plate of cells was prepared with no compound added. The plates were then incubated at 37° C./5% CO₂ for the following time points: 1, 2, 3, 4, 6, 16 and 24 hours. The non-treated plate was sampled at 0 hrs. After incubation at the desired time points, cells were washed 2× with 1.0 mL of DPBS. Cells were extracted by adding 500 μL of 70% acetonitrile/30% water spiked with the internal standard to each well treated with EIDD-2801. The non-treated blank plate was extracted with 500 μL of 70% acetonitrile/30% water per well. The samples were pipetted up and down several times. The samples were transferred to labeled microcentrifuge tubes. The samples were centrifuged at 16,000×g for 10 minutes at 4° C. 300 μL of supernatant was transferred to labeled HPLC vials, and the samples were analyzed by LC-MS/MS. Results are shown in Table 13.

TABLE 13

| | Cₘₐₓ | tₘₐₓ | AUC_{0->t} |
|---|---|---|---|
| Analyte | (pmol/M Cells) | (h) | (pmol · h/M cells) |
| EIDD-1931 (Nuc) | 13.4 | 16 | 249.8 |
| EIDD-2061 (TP) | 470.3 | 16 | 299.8 |
| EIDD-2801 (Parent) | 18.9 | 3 | 360.3 |

### Example 35: Method for Evaluating Cell Uptake and Metabolism of EIDD-2801 in CEM Cells

Three 24-well plates were plated with primary vero cells at a seeding density of 2×10⁶/mL viable cells per well. The plates were incubated at 37° C./5% CO₂ overnight to allow the cells to attach. A 40 mM stock solution of EIDD-2801 in 100% dimethylsulfoxide was prepared. From the 40 mM solution, a 40 μM solution of EIDD-2801 was prepared in 25 mL of complete RPMI media. For compound treatment plates, the media was aspirated, and 1.0 mL of M EIDD-2801 in complete RPMI media was added to the appropriate wells. A separate plate of cells was prepared with no compound added. The plates were then incubated at 37° C./5% CO₂ for the following time points: 1, 2, 3, 4, 6, 16 and 24 hours. The non-treated plate was sampled at 0 hrs. After incubation at the desired time points, cells were washed 2× with 1.0 mL of DPBS. Cells were extracted by adding 500 μL of 70% acetonitrile/30% water spiked with the internal standard to each well treated with EIDD-2801. The non-treated blank plate was extracted with 500 μL of 70% acetonitrile/30% water per well. The samples were pipetted up and down several times. The samples were transferred to labeled microcentrifuge tubes. The samples were centrifuged at 16,000×g for 10 minutes at 4° C. 300 μL of supernatant was transferred to labeled HPLC vials, and the samples were analyzed by LC-MS/MS. Results are shown in Table 14.

TABLE 14

| | Cₘₐₓ | tₘₐₓ | AUC_{0->t} |
|---|---|---|---|
| Analyte | (pmol/M Cells) | (h) | (pmol · h/M cells) |
| EIDD-1931 (Nuc) | 0.3 | 3 | 5.8 |
| EIDD-2061 (TP) | 171.3 | 24 | 2355 |
| EIDD-2801 (Parent) | 5.4 | 4 | 85.3 |

### Example 36: EIDD-1931 COVID-19 (SARS-2) Activity

The same protocol used in Example 18 was followed test the activity of EIDD-1931 against SARS-CoV2. Results are shown in Table 15.

TABLE 15

| | Cell | EC₅₀ | EC₉₀ | CC₅₀ |
|---|---|---|---|---|
| Virus | Line | (μM) | (μM) | (μM) |
| COVID-19 | Vero | 0.3 | — | >10 |
| SARS/COVID-19 | Huh-7 | 0.07 | — | — |
| nsp12 chimera-luc | | | | |

### Example 37: Phase I Human Data

Single and multiple doses of EIDD-2801 were evaluated in a first-in-human, phase 1, randomized, double-blind, placebo-controlled study in healthy volunteers, which included evaluation of the effect of food on pharmacokinetics. See Painter, W., et al. Hurnan Safety, Tolerability, and Pharmacokinetics of a Novel Broad-Spectrum Oral Antiviral Compound, Molnupiravir, with Activity Against SARS-CoV-2, MedRxiv, Dec. 14, 2020 https://doi.org/10.1101/2020.12.10.20235747, which is incorporated by reference herein in its entirety.

Eligible subjects were randomized in a 3:1 ratio to either EIDD-2801 or placebo in the single- and multiple-ascending-dose parts of the study. Each cohort comprised 8 subjects, with single oral doses of 50 to 1600 mg administered in the single-ascending-dose part and twice-daily (BID) doses of 50 to 800 mg administered for 5.5 days in the multiple-ascending-dose part. Subjects were followed for 14 days following completion of dosing for assessments of safety, tolerability, and pharmacokinetics. Subjects in the food-effect evaluation were randomized in a 1:1 ratio to either 200 mg EIDD-2801 in the fed state followed by 200 mg EIDD-2801 in the fasted state, or vice versa, with a 14-day washout period between doses. A capsule formulation was used in all parts of the study, with the exception of single ascending doses ≤800 mg, where an oral solution formulation was used.

Sixty-four subjects received a single dose of between 50 and 1600 mg EIDD-2801 or placebo; 55 subjects received between 50 and 800 mg EIDD-2801 or placebo BID for 5.5 days; and 10 subjects received a single dose of 200 mg EIDD-2801 in the fed state followed by a single dose of 200 mg EIDD-2801 in the fasted state after a washout period of 14 days, or vice versa. Additionally, 1 subject in the multiple-ascending-dose part received 800 mg EIDD-2801 BID for 3 days, but the subject was discontinued from dosing by the investigator on Day 4. All subjects completed the protocol-specified study procedures and assessments.

Subjects were aged between 19 and 60 years, with a mean body mass index between 24.4 and 25.4 kg/m². The majority of subjects were white and male. There were no other notable differences in subject demography between cohorts, except for age, where the mean age was higher in the food-effect evaluation cohort, the 50-mg EIDD-2801 single-dose cohort, and in the 100-mg EIDD-2801 multiple-dose cohort.

Adverse events were graded using the Division of Microbiology and Infectious Diseases (DMID) toxicity grading, dated March 2014, Single ascending doses. Overall, 37.5% of subjects reported an adverse event (Table 16). There were no apparent dose-related trends, with a greater proportion of subjects reporting adverse events following administration of placebo (43.8%) than following administration of EIDD-2801 (35.4%). Only 1 moderate adverse event (headache; Grade 2) was reported following administration of EIDD-2801, which occurred at the 400-mg dose level. One subject also reported moderate adverse events (nausea and headache; Grade 2) following administration of placebo. No severe (Grade 3) adverse events were reported. The most frequently reported adverse event was headache, which was reported by 18.8% of subjects who were administered placebo and 12.5% of subjects who were administered EIDD-2801.

Multiple ascending doses. Overall, 44.6% of subjects reported an adverse event (Table 17). There were no apparent dose-related trends, with a greater proportion of subjects reporting adverse events following administration of placebo (50.0%) than following administration of EIDD-2801 (42.9%). With the exception of 1 subject who reported moderate (Grade 2) events of oropharyngeal pain, pain in extremity, and influenza-like illness, all adverse events were mild (Grade 1) in severity. The most frequently reported adverse event was diarrhea, which was reported by 7.1% of subjects who were administered EIDD-2801 and 7.1% of subjects who were administered placebo. One subject discontinued study drug administration on Day 4 because of an adverse event of mild, truncal, maculopapular, pnritic rash following multiple BID doses of 800 mg EIDD-2801, which was considered by the investigator to be related to the study drug. Following discontinuation, the subject was administered potent topical steroid treatment and anti-histamines, and pruritis and rash had both resolved within 18 days.

Food-effect evaluation, Three subjects in the food-effect evaluation each reported 1 adverse event, all of which were mild (Grade 1) in severity.

TABLE 16

| | | Placebo | 50 mg | 100 mg | 200 mg | 400 mg | 600 mg | 800 mg | 1200 mg | 1600 mg |
|---|---|---|---|---|---|---|---|---|---|---|
| | | (N = 16) | (N = 6) | (N = 6) | (N = 6) | (N = 6) | (N = 6) | (N = 6) | (N = 6) | (N = 6) |
| Overall (n, [nE]) | | 7 [12] | 2 [4] | — | 3 [4] | 3 [5] | 4 [7] | 2 [5] | 1 [2] | 2 [8] |
| Severity | Mild | 7 [10] | 2 [4] | | 3 [4] | 3 [4] | 4 [7] | 2 [5] | 1 [2] | 2 [8] |
| | (Grade 1) | | | | | | | | | |
| | Moderate | 1 [2] | — | — | — | 1 [1] | — | — | — | — |
| | (Grade 2) | | | | | | | | | |
| | Severe | — | — | — | — | — | — | — | — | — |
| | (Grade 3) | | | | | | | | | |

| | | | | | | | | | |
|---|---|---|---|---|---|---|---|---|---|
| Related | 2 [4] | 1 [1] | — | 2 [3] | — | 1 [1] | — | — | — |

Preferred Terms Reported By More Than 1 Subject (n)

| | | | | | | | | | |
|---|---|---|---|---|---|---|---|---|---|
| Headache | 3 | 1 | — | — | 1 | 3 | 1 | — | — |
| [symbol] site pain* | 1 | — | — | — | 1 | 1 | — | — | — |
| Nausea | 1 | — | — | 1 | — | 1 | — | — | — |
| Rhinorrhoea | 1 | — | — | — | — | — | 1 | 1 | — |
| Back pain | — | — | — | — | — | 1 | 1 | — | — |
| Feeling hot | — | — | — | — | 2 | — | — | — | — |
| Pain in extremity | 1 | 1 | — | — | — | — | — | — | — |

Abbreviations: n = number of subjects with an adverse event; nE = number of advserse events; N = number of subjects.
*venous cannula site pain. [symbol]
indicates data missing or illegible when filed

TABLE 17

| | | Placebo | 50 mg | 100 mg | 200 mg | 300 mg | 400 mg | 600 mg | 800 mg |
|---|---|---|---|---|---|---|---|---|---|
| | | BID | BID | BID | BID | BID | BID | BID | BID |
| | | (N = 14) | (N = 6) | (N = 6) | (N = 6) | (N = 6) | (N = 6) | (N = 6) | (N = 6) |
| Overall (n, [nE]) | | 7 [11] | 2 [2] | 3 [3] | 3 [9] | 2 [3] | 3 [5] | 2 [2] | 3 [5] |
| Severity | Mild | 7 [11] | 2 [2] | 3 [3] | 3 [6] | 2 [3] | 3 [5] | 2 [2] | 3 [5] |
| | (Grade 1) | | | | | | | | |
| | Moderate | — | — | — | 3 [3] | — | — | — | — |
| | (Grade 2) | | | | | | | | |
| | Severe | — | — | — | — | — | — | — | — |
| | (Grade 3) | | | | | | | | |
| Related | | 3 [4] | — | — | 2 [3] | 1 [1] | — | 1 [1] | 3 [4] |

Preferred Terms Reported By More Than 1 Subject (n)

| | | | | | | | | |
|---|---|---|---|---|---|---|---|---|
| Diarrhoea | 1 | — | — | 1 | — | — | 1 | 1 |
| Back pain | — | — | 2 | — | 1 | — | — | — |
| Headache | — | — | 1 | — | 2 | — | — | — |
| Somnolence | 2 | — | — | — | — | 1 | — | — |

Abbreviations: BID = twice daily: n = number of subjects with an adverse event; nE = number of adverse events; N = number of subjects.

There were no serious adverse events reported in this study and there were no trends of increased frequency or severity of adverse events with higher doses of EIDD-2801.

There were no clinically significant findings or dose-related trends in clinical laboratory, vital signs, and electrocardiogram data. The dose-limiting toxicity in one Investigational New Drug-enabling study (in the dog, the most sensitive species) was bone marrow toxicity and included reversible reductions in platelet counts; however, no clinically significant changes in hematological parameters were seen in this study.

Dose escalations were discontinued before a maximum tolerated dose was reached because plasma exposures that were expected to be efficacious based on scaling from animal models of seasonal and pandemic influenza were exceeded.

### Pharmacokinetics

Single ascending doses. Concentrations of EIDD-2801 were generally not quantifiable at doses up to 800 mg, with the exception of the 0.25-hour timepoint after doses of 600 and 800 mg, where concentrations were quantifiable in 5 and 4 subjects, respectively, and the 0.5-hour timepoint after a dose of 800 mg, where concentrations were quantifiable in all subjects. At doses of 1200 and 1600 mg, concentrations of EIDD-2801 were quantifiable at 1 or more timepoints between 0.25 and 1.5 hours postdose in all subjects. EIDD-2801 pharmacokinetic parameters were not calculable for doses ≤400 mg; however, at doses ≥600 mg, maximum observed concentration (Cₘₐₓ), time of Cₘₐₓ (tₘₐₓ), and time of last quantifiable concentration were calculable. Following administration of between 600 and 1600 mg EIDD-2801, values of mean Cₘₐₓ were up to 13.2 ng/mL and values of median tₘₐₓ were between 0.25 and 0.75 hours, It should be noted that EIDD-2801 concentrations represented only approximately 0.2% of EIDD-1931 concentrations and tₘₐₓ of EIDD-2801 occurred at the first sampling timepoint for the 600-mg dose level, and therefore Cₘₐₓ may have been underestimated. At doses of ≥800 mg, trace amounts of EIDD-2801 were detected in the urine, which represented approximately 0.002% of the dose.

Following oral administration of EIDD-2801 at doses up to 800 mg, EIDD-1931 appeared rapidly in plasma, with a median tₘₐₓ of 1.00 hour postdose in all dose cohorts, after which plasma concentrations declined in an essentially monophasic manner with geometric mean terminal elimination half-lives (t_{1/2}) of between 0.910 and 1.29 hours postdose (Table 18 and FIGS. 16-18). However, at doses of 1200 and 1600 mg, median tₘₐₓ was delayed, with median tₘₐₓ occurring at 1.75 and 1.50 hours, respectively. Plasma concentrations at doses of 1200 and 1600 mg were quantifiable, along with a second slower elimination phase, where mean t_{1/2} was longer with values of 1.81 and 4.59 hours, respectively.

TABLE 18

| | 50 mg | 100 mg | 200 mg | 400 mg | 600 mg | 800 mg | 1200 mg | 1600 mg |
|---|---|---|---|---|---|---|---|---|
| Parameter | (N = 6) | (N = 6) | (N = 6) | (N = 6) | (N = 6) | (N = 6) | (N = 6) | (N = 6) |
| AUCₗₐₛₜ (ng · h/mL) | 415 (27.4) | 917 | 1810 | 4000 | 6120 | 8720 | 13800 | 20700 |
| | | (27.5) | (20.0) | (20.2) | (21.6) | (10.4) | (11.7) | (31.4) |
| AUC_{inf} (ng · h/mL) | 432 (26.5) | 932 | 1830 | 4010 | 6130 | 8740 | 13800 | 20700 |
| | | (27.0) | (19.5) | (20.2) | (21.4) | (10.4) | (11.8) | (31.4) |
| Cₘₐₓ (ng/mL) | 223 (46.2) | 454 | 926 | 1850 | 2720 | 3640 | 4500 | 6350 |
| | | (42.2) | (12.6) | (22.7) | (27.0) | (13.4) | (17.9) | (20.6) |
| tₘₐₓ (h) | 1.00 | 1.00 | 1.00 | 1.00 | 1.00 | 1.00 | 1.75 | 1.50 |
| | (0.517- | (0.500- | (0.500- | (0.500- | (1.00- | (0.500- | (1.00- | (1.00- |
| | 1.00) | 1.50) | 1.00) | 1.00) | 1.00) | 1.00) | 2.50) | 2.00) |
| t_{1/2} (h) | 0.945 | 0.907 | 1.02 | 1.03 | 1.06 | 1.29 | 1.81 | 4.59 |
| | (12.1) | (10.1) | (16.4) | (8.86) | (10.3) | (7.10) | (73.5) | (71.6) |
| Ae₀₋₂₄ (mg) | 0.323 | 1.03 | 1.51 | 5.47 | 14.4 | 18.0 | 53.7 | 84.4 |
| | (53.6) | (68.1) | (86.2) | (108) | (47.7) | (14.7) | (41.4) | (29.9) |
| Fe₀₋₂₄ (%) | 0.820 | 1.31 | 0.958 | 1.74 | 3.05 | 2.86 | 5.69 | 6.70 |
| | (53.6) | (68.1) | (86.2) | (108) | (47.7) | (14.7) | (41.4) | (29.9) |

Geometric means (percentage coefficient of variation) are presented, with the exception of tₘₐₓ where medians (minimum-maximum) are presented.
Abbreviations: Ae₀₋₂₄ = amount of the dose administered recovered in urine from time 0 to 24 hours postdose; AUC_{inf} = area under the plasma concentration-time curve from time 0 extrapolated to infinity; AUCₗₐₛₜ = area under the plasma concentration-time curve from time 0 to the last measurable non-zero concentration; Cₘₐₓ = maximum observed concentration; Fe₀₋₂₄ = percentage of the dose administered recovered in urine from time 0 to 24 hours postdose; N = number of subjects; t_{1/2} = apparent terminal elimination half-life; tₘₐₓ = time of the maximum observed concentration.

The plasma concentration-time profiles were generally well defined, with the percentage of area under the plasma concentration-time curve from time 0 extrapolated to infinity (AUC_{inf}) that was extrapolated being <10% for all subjects. When assessed using a power model (ln[parameter]=intercept+slope×ln[dose]+random error), mean Cₘₐₓ increased in a dose-proportional manner, with the 90% confidence interval containing unity. Similarly, mean AUC_{inf} increased in an approximately dose-proportional manner; however, the lower bound of the 90% confidence interval was slightly above unity (Table 19).

TABLE 19

| | | | Between-subject | |
|---|---|---|---|---|
| | | Slope (90% | Geometric | |
| | | Confidence | Coefficient of | Lack of Fit |
| Parameter | n | Interval) | Variation | P-value |
| AUC_{inf} (ng · h/mL) | 36 | 1.07 (1.02, 1.13) | 21.5 | 0.9724 |
| AUCₗₐₛₜ (ng · h/mL) | 36 | 1.09 (1.03, 1.15) | 21.9 | 0.9750 |
| Cₘₐₓ (ng/mL) | 36 | 1.01 (0.927, 1.08) | 29.8 | 0.9996 |

The amount of EIDD-1931 excreted in urine from time 0 to 24 hours postdose (Ae₀₋₂₄) increased supraproportionally with dose, and there was a similar trend for apparent clearance (CL_{R}) to increase. Between 0.820% (at the 50-mg dose level) and 6.70% (at the 1600-mg dose level) of the dose was excreted in urine as EIDD-1931, and the majority of the total amount was generally excreted within the first 4 hours postdose.

Multiple ascending doses. Concentrations of EIDD-2801 were generally not quantifiable at doses ≤400 mg BID and pharmacokinetic parameters were not calculable. Concentrations of EIDD-2801 were quantifiable in 4 subjects at either 0.5 or 1 hour postdose on Day 1 and in 3 subjects at 0.5 hours postdose on Day 6 at the 600-mg BI) dose level. At the 800-mg dose level, concentrations of EIDD-2801 were quantifiable from all except 1 subject at 0.5 hours postdose on Days 1 and 6, but at no other timepoints, consistent with single ascending doses.

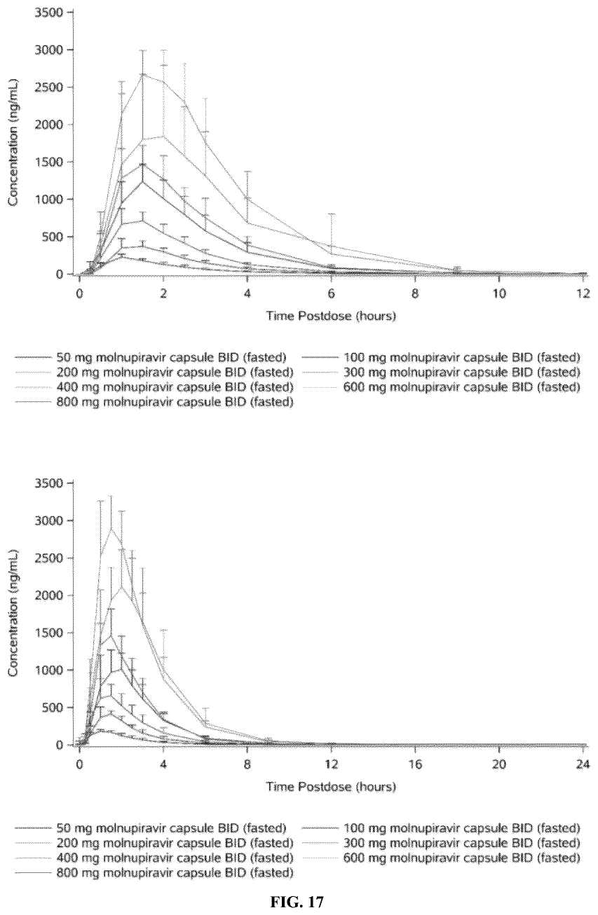

Following oral administration of EIDD-2801, EIDD-1931 appeared rapidly in plasma, with a median tₘₐₓ in all dose cohorts of between 1.00 and 1.75 hours postdose across both Days 1 and 6 (Table 20 and FIG. 17). For all dose levels, plasma concentrations declined in an essentially monophasic manner on Day 1, with mean t_{1/2} ranging from 0.918 to 1.18 hours. Similarly, plasma concentrations declined in an essentially monophasic manner on Day 6 for subjects at dose levels ≤200 mg 131) and for the majority of subjects at the 300- and 400-mg BID dose levels. Contrastingly, for 1 subject at each of the 300- and 400-mg dose levels and for all subjects at the 600- and 800-mg BID dose levels, there was the emergence of a second, slower elimination phase on Day 6, which was reflected in an increase in the mean t_{1/2} with increasing dose at doses ≥200 mg. Of note, at the 600-mg BID dose level, the lack of a clearly defined terminal elimination phase confounded the evaluation of t_{1/2} for the majority of subjects. At the 800-mg BID dose level, the mean t_{1/2} was 7.08 hours.

TABLE 20

| | Day 1 | | | | | | | Day 6 | | | | | | |
|---|---|---|---|---|---|---|---|---|---|---|---|---|---|---|
| | 50 mg | 100 mg | 200 mg | 300 mg | 400 mg | 600 mg | 800 mg | 50 mg | 100 mg | 200 mg | 300 mg | 400 mg | 600 mg | 800 mg |
| Parameter | (N = 6) | (N = 6) | (N = 6) | (N = 6) | (N = 6) | (N = 6) | (N = 6) | (N = 6) | (N = 6) | (N = 6) | (N = 6) | (N = 6) | (N = 6) | (N = 5) |
| AUCₜ | 461 | 854 | 1660 | 3080 | 3800 | 6110 | 8190 | 432 | 968 | 1730 | 2960 | 3710 | 7110 | 8330 |
| (ng · h/mL) | (15.7) | (19.8) | (15.3) | (17.3) | (19.5) | (26.9) | (21.5) | (14.9) | (15.3) | (25.2) | (16.2) | (21.6) | (28.2) | (17.9) |
| AUCₗₐₛₜ | 444 | 835 | 1640 | 3080 | 3790 | 6110 | 8180 | 414 | 947 | 1720 | 2980 | 3730 | 7250 | 8450 |
| (ng · h/mL) | (17.3) | (19.9) | (15.5) | (17.4) | (19.5) | (26.9) | (21.5) | (16.2) | (15.7) | (26.0) | (16.3) | (21.6) | (28.1) | (18.5) |
| AUC_{inf} | 461 | 855 | 1660 | 3090 | 3800 | 6680* | 8200 | — | — | — | — | — | — | — |
| (ng · h/mL) | (15.7) | (19.8) | (15.3) | (17.4) | (19.5) | (17.6) | (21.6) | | | | | | | |
| Cₘₐₓ | 223 | 395 | 766 | 1280 | 1530 | 2160 | 2770 | 188 | 434 | 742 | 1100 | 1470 | 2240 | 2970 |
| (ng/mL) | (19.4) | (18.5) | (16.3) | (15.2) | (23.2) | (31.4) | (13.3 | (8.67) | (14.0) | (32.1) | (20.6) | (20.9) | (20.9) | (16.8) |
| tₘₐₓ (h) | 1.00 | 1.25 | 1.50 | 1.50 | 1.50 | 1.75 | 1.75 | 1.00 | 1.25 | 1.50 | 1.50 | 1.50 | 1.75 | 1.50 |
| | (1.00- | (1.00- | (1.00- | (1.00- | (1.00- | (1.00- | (1.50- | (1.00- | (1.00- | (0.500- | (1.00- | (1.00- | (1.50- | (1.00- |
| | 1.00) | 2.03) | 1.50) | 1.50) | 6.00) | 6.00) | 2.50) | 1.50) | 1.50) | 1.50) | 2.00) | 1.50) | 2.50) | 2.02) |
| MRcₘₐₓ | NC | NC | NC | NC | NC | 329* | 512 | NC | NC | NC | NC | NC | NC | 413† |
| | | | | | | (87.7) | (30.6) | | | | | | | (28.1) |
| t_{1/2} (h) | 0.937 | 0.918 | 0.960 | 1.09 | 1.05 | 1.16* | 1.18 | 0.968 | 0.970 | 1.24 | 1.71 | 1.20* | NC | 7.08¶ |
| | (14.0) | (9.08) | (10.4) | (17.7) | (13.1) | (3.50) | (7.28) | (15.5) | (15.8) | (36.4) | (47.1) | (9.58) | | (154) |
| RA_{AUC} | — | — | — | — | — | — | — | 0.938 | 1.13 | 1.04 | 0.961 | 0.977 | 1.16 | 1.09 |
| | | | | | | | | (7.80) | (9.25) | (18.0) | (14.7) | (11.7) | (12.2) | ( 11.8) |
| RAcₘₐₓ | — | — | — | — | — | — | — | 0.843 | 1.10 | 0.969 | 0.861 | 0.962 | 1.04 | 1.09 |
| | | | | | | | | (16.0) | (11.4) | (23.8) | (14.3) | (18.5) | (20.0) | (7.15) |
| Ae₀₋₁₂ (mg) | 0.391 | 0.993 | 1.38 | 3.37 | 4.57 | 11.9 | 22.7 | 0.336 | 0.915 | 1.76 | 3.14 | 5.32 | 16.4 | 18.9 |
| | (55.7) | (96.9) | (81.8) | (57.4) | (57.9) | (22.9) | (34.4) | (64.5) | (48.0) | (85.5) | (63.4) | (47.5) | (39.9) | (81.6) |
| Fe₀₋₁₂ (%) | 0.993 | 1.26 | 0.879 | 1.43 | 1.45 | 2.52 | 3.61 | 0.854 | 1.16 | 1.12 | 1.33 | 1.69 | 3.48 | 3.00 |
| | (55.7) | (96.9 | (81.8) | (57.4) | (57.0) | (22.9) | (34.4) | (64.5) | (48.0) | (85.5) | (63.4) | (47.5) | (39.9) | (81.6) |

*N = 5;
† N = 3
¶ N = 4.
Abbreviations: Ae₀₋₁₂ = amount of dose administered recovered in urine from time 0 to 12 hours postdose; AUCₜ = area under the plasma concentration-time curve during a dosing interval; AUC_{inf} = area under the plasma concentration-time curve from time 0 extrapolated to infinity: AUCₗₐₛₜ = area under the plasma concentration-time curve from time 0 to the last measurable non-zero concentration; Cₘₐₓ = maximum observed concentration; Fe₀₋₁₂ = percentage of the dose administered recovered in urine from time 0 to 12 hours postdose; MR = metabolite ratio; N = number of subjects; NC = not calculated; RA = accumulation ratio; t_{1/2} = apparent terminal elimination half-life; tₘₐₓ = time of the maximum observed concentration.

There was no evidence of accumulation, with the geometric mean accumulation ratios based on area under the plasma concentration-time curve during a dosing interval (AUCₜ) and Cₘₐₓ between 0.938 and 1.16, and between 0.843 and 1.10, respectively, across all dose levels.

On Day 1, when assessed using the power model, mean Cₘₐₓ and AUC_{inf} increased in an approximately dose-proportional manner. However, the upper bound of the 90% confidence interval for Cₘₐₓ was slightly below unity and the lower bound of the 90% confidence interval for AUC_{inf} was slightly above unity (Table 21). On Day 6, mean Cₘₐₓ increased in a dose-proportional manner, with the 90% confidence interval containing unity. Similarly, mean AUC_{τ} increased in an approximately dose-proportional manner; however, the lower bound of the 90% confidence interval was slightly above unity (Table 21).

TABLE 21

| | Day 1 | | | | Day 6 | | | |
|---|---|---|---|---|---|---|---|---|
| | | Slope | Between-subject | | | Slope | Between-subject | |
| | | (90% | Geometric | Lack of | | (90% | Geometric | Lack of |
| | | Confidence | Coefficient of | Fit | | Confidence | Coefficient of | Fit |
| Parameter | n | Interval) | Variation | P-value | n | Interval) | Variation | P-value |
| AUC_{inf} | 53 | 1.10 | 19.6 | 0.3149 | — | — | — | — |
| (ng · h/mL) | | (1.06, 1.14) | | | | | | |
| AUCₗₐₛₜ | 54 | 1.11 | 20.8 | 0.4483 | — | — | — | — |
| (ng · h/mL) | | (1.07, 1.15) | | | | | | |
| AUCₜ | — | — | — | — | 41 | 1.08 | 20.5 | 0.3670 |
| (ng · h/mL) | | | | | | (1.02, 1.14) | | |
| Cₘₐₓ | 54 | 0.957 | 20.1 | 0.7011 | 41 | 0.971 (0.915, | 20.3 | 0.7798 |
| (ng/mL) | | (0.916, 0.998) | | | | 1.03) | | |

AUC_{inf} on Day 1 for the multiple-dose cohorts, where a capsule formulation was administered, was similar to those for the corresponding single-dose cohorts where a solution formulation was administered, with geometric mean ratios of between 0.91 and 1.09. Geometric mean Cₘₐₓ was slightly lower following dosing with the capsule formulation, with geometric mean ratios of between 0.76 and 1.00, and a trend to smaller ratios at higher doses. Median tₘₐₓ occurred up to 0.75 hours later following administration of the capsule formulation, with the difference being greatest at doses ≥600 mg BID. Thus, it appears that the extent of absorption is similar for the solution and capsule formulations, but the rate of absorption appears to be slightly slower for the capsules. However, these data should be interpreted with caution because this was not a crossover study.

Between 0.854% and 3.61% of the dose was excreted in urine as EIDD-1931 on both Days 1 and 6, and, similar to single doses, the majority was excreted in the first 4 hours postdose (Table 19). There was no consistent dose-related trend in the percentage of dose administered recovered in urine during a dosing interval (Fe_{0-τ}) or CL_{R} at doses ≤200 mg BID. However, there was a trend for Fe_{0-τ}, and CL_{R} to increase with dose at doses >200 mg BID, with a 4-fold increase in dose from 200 to 800 mg BID resulting in a 16-fold increase in the amount of the dose recovered in urine during a dosing interval (Ae_{0-τ}) on Day 1 and an 11-fold increase in Ae_{0-τ} on Day 6.

Food effect. Concentrations of EIDD-2801 were generally not quantifiable and pharmacokinetic parameters were not calculable. Concentrations of EIDD-1931 were quantifiable at 0.25 hours postdose for 2 subjects in the fasted state, but no subjects in the fed state. The first quantifiable concentrations in the fed state were between 0.5 and 1.5 hours postdose.

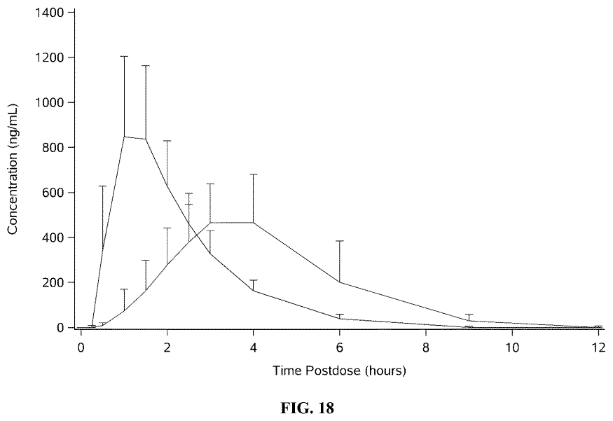

Following administration of 200 mg EIDD-2801 in the fed state, tₘₐₓ of EIDD-1931 occurred later, with a median of 3.00 hours postdose versus 1.00 hour postdose (Table 22 and FIG. 18). Generally, the slower absorption and later tₘₐₓ in the fed state was reflected in lower Cₘₐₓ; however, 1 subject had similar profiles for both treatments. Mean Cₘₐₓ was approximately 36% lower in the fed state compared to the fasted state, but exposure (assessed by AUC_{inf}) was similar for both fed and fasted states and demonstrated that the extent of absorption was similar. Following Cₘₐₓ, concentrations of EIDD-1931 declined in an essentially monophasic manner in both the fed and fasted state and remained quantifiable until between 9 and 15 hours postdose in the fed state and between 6 and 9 hours in the fasted state. The mean t_{1/2} was similar between fed and fasted treatments, with values of 1.09 and 0,977 hours, respectively. Urine pharmacokinetic parameters were similar to those reported for single ascending doses,

TABLE 22

| Parameter | 200 mg Fasted (N = 10) | | 200 mg Fed (N = 10) | |
|---|---|---|---|---|
| AUCₗₐₛₜ (ng · h/mL) | 1950 | (29.9) | 1870 | (29.0) |
| AUC_{inf} (ng · h/mL) | 1980 | (29.3) | 1890 | (28.8) |
| Cₘₐₓ (ng/L) | 893 | (36.8) | 575 | (27.7) |
| tₘₐₓ (h) | 1.00 | (1.00-2.50) | 3.00 | (2.00-4.00) |
| t_{1/2} (h) | 0.977 | (13.0) | 1.09 | (16.3) |
| FE_{AUC/inf} | — | | 0.955 | (9.7) |
| FE_{Cmax} | — | | 0.644 | (22.5) |
| Ae₀₋₂₄ (mg) | 1.76 | (45.6) | 1.63 | (54.2) |
| Fe₀₋₂₄ (%) | 1.12 | (45.6) | 1.04 | (54.2) |

Geometric means (percentage coefficient of variation) are presented, with the exception of tₘₐₓ where medians (minimum-maximum) are presented.
Abbreviations: Ae₀₋₂₄ = amount of the dose administered recovered in urine from time 0 to 24 hours postdose; AUC_{inf} = area under the plasma concentration-time curve from time 0 extrapolated to infinity; AUCₗₐₛₜ = area under the plasma concentration-time curve from time 0 to the last measurable non-zero concentration; Cₘₐₓ = maximum observed concentration; Fe₀₋₂₄ = percentage of the dose administered recovered in urine from time 0 to 24 hours postdose; FE_{AUCinf} = ratio of area under the plasma concentration-time curve from time 0 extrapolated to infinity (fed:fasted); FE_{Cmax} = ratio of maximum observed concentration (fed:fasted); t_{1/2} = apparent terminal elimination half-life; tₘₐₓ = time of the maximum observed concentration.

There is a significant need for an antiviral drug against coronaviruses with pandemic potential that is generally safe and well tolerated and can be easily administered in the outpatient setting. The oral route of administration of EIDD-2801 makes it appropriate and convenient for administration to outpatients.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-77. (canceled)

78. A method of treating SARS-CoV-2 infection in a patient in need thereof, comprising orally administering to the patient a compound with the following structure: 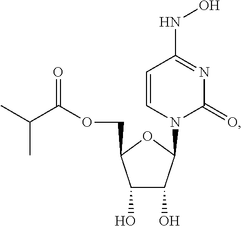 [SMILES: CC(C)C(=O)OC[C@H]1O[C@@H](n2ccc(NO)nc2=O)[C@H](O)[C@@H]1O]
or a tautomer thereof,
wherein the compound is administered to the patient in an amount of 800 mg twice a day.

79. The method of claim 78, wherein the pharmaceutical composition is administered to the patient for five days.

80. The method of claim 78, comprising administering pharmaceutical compositions comprising the compound to the patient, wherein each pharmaceutical composition comprises the compound in an amount of 200 mg.

81. The method of claim 80, wherein each pharmaceutical composition comprises a lubricant comprising magnesium stearate, calcium stearate, stearic acid, glycerol behenate, polyethylene glycol, talc, mineral oil, or a combination thereof;
a disintegrant, a binder, and/or a coating comprising starch, gelatin, a sugar, polyethylene glycol, a wax, acacia, tragacanth, sodium alginate, sodium carboxymethylcellulose, hydroxypropyl cellulose, pregelatinized starch, clays, cellulose, alginine, gums, kaolin, cross-linked PVP, or combination thereof; and
a diluent comprising dicalcium phosphate dihydrate, calcium sulfate, lactose, sucrose, mannitol, sorbitol, microcrystalline cellulose, kaolin, sodium chloride, dry starch hydrolyzed starches, pregelatinized starch, silicone dioxide, titanium oxide, powdered cellulose, crystalline cellulose, magnesium aluminum silicate, or a combination thereof.

82. The method of claim 81, wherein the lubricant comprises magnesium stearate.

83. The method of claim 81, wherein the diluent comprises microcrystalline cellulose.

84. The method of claim 81, wherein the binder comprises hydroxypropyl cellulose.

85. The method of claim 81, wherein the lubricant comprises magnesium stearate, the diluent comprises microcrystalline cellulose, and the binder comprises hydroxypropyl cellulose.

86. The method of claim 81, wherein the pharmaceutical compositions are capsules.

87. The method of claim 78, wherein the SARS-CoV2 infection is in the CNS of the patient.

88. A pharmaceutical composition comprising a compound with the following structure: 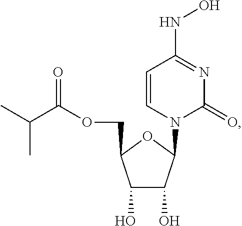 [SMILES: CC(C)C(=O)OC[C@H]1O[C@@H](n2ccc(NO)nc2=O)[C@H](O)[C@@H]1O]
or a tautomer thereof,
wherein the pharmaceutical composition comprises a lubricant comprising magnesium stearate, calcium stearate, stearic acid, glycerol behenate, polyethylene glycol, talc, mineral oil, or a combination thereof;
a disintegrant, a binder, and/or a coating comprising starch, gelatin, a sugar, polyethylene glycol, a wax, acacia, tragacanth, sodium alginate, sodium carboxymethylcellulose, hydroxypropyl cellulose, pregelatinized starch, clays, cellulose, alginine, gums, kaolin, cross-linked PVP, or combination thereof; and
a diluent comprising dicalcium phosphate dihydrate, calcium sulfate, lactose, sucrose, mannitol, sorbitol, microcrystalline cellulose, kaolin, sodium chloride, dry starch hydrolyzed starches, pregelatinized starch, silicone dioxide, titanium oxide, powdered cellulose, crystalline cellulose, magnesium aluminum silicate, or a combination thereof.

89. The composition of claim 88, wherein the lubricant comprises magnesium stearate.

90. The composition of claim 88, wherein the diluent comprises microcrystalline cellulose.

91. The composition of claim 88, wherein the binder comprises hydroxypropyl cellulose.

92. The composition of claim 88, wherein the lubricant comprises magnesium stearate, the diluent comprises microcrystalline cellulose, and the binder comprises hydroxypropyl cellulose.

93. The composition of claim 88, wherein the pharmaceutical compositions are capsules.

94. The composition of claim 88, wherein the composition comprising the compound in an amount of 200 mg.
